(19) United States
(12) Patent Application Publication
(10) Pub. No.: US 2025/0111889 A1
(43) Pub. Date: Apr. 3, 2025

(54) DE NOVO DESIGNED MACROCYCLIC OLIGOAMIDES

(71) Applicant: UNIVERSITY OF WASHINGTON, Seattle, WA (US)

(72) Inventors: David BAKER, Seattle, WA (US); Patrick SALVESON, Seattle, WA (US); Adam MOYER, Seattle, WA (US); Asim BERA, Seattle, WA (US)

(21) Appl. No.: 18/728,223

(22) PCT Filed: Jan. 12, 2023
(86) PCT No.: PCT/US2023/060534

§ 371 Date: Jul. 11, 2024

Related U.S. Application Data
(60) Provisional application No. 63/299,633, filed on Jan. 14, 2022.
(60) Provisional application No. 63/369,441, filed on Jul. 26, 2022.

(51) Int. Cl. G16B 15/00; C07K 4/00; G16B 40/00

(52) U.S. Cl. CPC G16B 15/00; C07K 4/00; G16B 40/00

(57) ABSTRACT

Three to four residue non-naturally occurring macrocycle oligoamide comprising a chemotype of 3 or 4 monomer residues selected from the group consisting of a, b, c, d, e, f, g, h, I, j, k, l, m, n, o, p, q, r, s, t, u, and v monomers as defined in Table 1 are provided, and methods for design of such macrocycle oligoamides

### CROSS REFERENCE

This application claims priority to U.S. Provisional Application Ser. No. 63/299,633 filed Jan. 14, 2022 and 63/369,441 filed Jul. 26, 2022, each incorporated by reference herein in its entirety.

### BACKGROUND

Computational methods that enable the rapid and comprehensive exploration of the space of possible macrocycles could greatly facilitate the discovery of compounds with novel bioactivities, but such methods do not currently exist. Random searching through combinations of backbone torsion angles and subsequent sequence design is intractable when many different backbones are included.

### DESCRIPTION OF THE FIGURES

FIG. 1. Overview of macrocycle discovery approach. (A) Chemical structures of monomeric building blocks used to construct small drug-like macrocycles: amino acids (a, b, g, d, e), aminobenzoic acids (c, f, j), aminomethylbenzoic acids (h, k, n), aminophenylacetic acids (i, l, o), aminomethylphenylacetic acids (m, p, q), oxazolidines/thiazolidines (r), oxazole/thiazoles (t), thioethers (s), aminomethylpicolinic acids (u), and triazoles (v). The backbone atoms are colored to highlight the differences between building blocks. (B) Assembly and identification of low-energy conformers of cyclo-(Bpro-Ben2-Bpro-Ben2) beginning from calculated potential energy surfaces of Ac-Bpro-NHMe and Ac-Ben2-NMe2. Combining these low-energy conformers yields thousands of Ac-Bpro-Ben2-NMe2 dimers. The 6D transform between the planes defined by the terminal amides is calculated for each dimer, and then binned. All pairs of dimers that close one another are identified in a quick lookup and the resulting ensemble of macrocycle conformers is minimized

FIG. 2. Chemical and structural diversity of small de novo macrocycles. (A) Distribution of unique sequences sampled across all 4-residue macrocyclic chemotypes. The 15 most populated chemotypes are labeled. (B) Torsional diversity of sampled 4-residue macrocycle chemotypes. Heat map pixels represent 4-residue macrocycle chemotypes generated from the two residue fragments on the x and y axes. The inset at top right shows the full map over all 224 monomer chemotype combinations; a 10×10 subset of this is blown up in the main panel for clarity. Examples of two representative torsion bins are shown in the boxes at left for two unique chemotypes. (C) Principal moment of inertia distributions (second row) and representative conformers (bottom) sampled by macrocycles constructed from different chemotypes (top row).

FIG. 3. X-ray and NMR structures of locally encoded macrocycles are very close to the design models. Rows A-E crystal structures. Column I, chemical structure; Column II design model; Column III: Surface representation of design models; Column IV: Superposition of the design model and experimentally determined coordinates; Column V: chemical properties and apparent permeabilities; the number of hydrogen bond donors (HBDonor) and hydrogen bond acceptors (HBAcceptor) was determined using rdkit. ahah was modeled with phenylalanines and chemically synthesized with pyridylalanines (row E). Rows F-H, NMR structures. In column II, in place of the design model we show ROEs used for distance-constrained ensemble generation. Experimental structure to design model RMSDs are reported over all backbone atoms for X-ray structures, and over ensemble average backbone coordinates for NMR structures.

FIG. 4. X-ray crystallographic and NMR ensemble structures of hydrogen bond-containing macrocycles are very close to their design models. Columns I-V are as in FIG. 3. Column II rows E-G: ROEs used for distance-constrained ensemble generation. Arrows are as in FIG. 3.

FIG. 5. 130 chemicals for which potential energy surfaces were calculated. The chemotype bin for each chemical is listed next to its abbreviated name.

FIG. 6. Chemotype distribution of unique sequences sampled across all 4-residue macrocycles discovered in our expansive search (left), all 3- and 4-residue macrocycles in the Cambridge Structural Database (middle), and all 3- and 4-residue macrocycles in the PubChem database (right).

FIG. 7. Many conformers of many sequences within a chemotype disproportionately sample distinct backbone torsion bins. (A) Cartoon of the torsion binning scheme. Each conformer of each sequence belonging to a given chemotype (aaa in this example) is built and its backbone torsion angles are measured and binned on 60-degree intervals. The number of conformers that the 16 most densely sampled bins are displayed in the resulting bar chart. (B) The 16 most sampled bins for several 3-residue and 4-residue macrocycles. The non-uniform sampling is apparent.

FIG. 8. Approximately one half of the unique macrocycles enumerated here obey the rule of five. (A) Distribution of unique sequences obeying (Ro5) and disobeying (bRo5) Lipinski's rule of 5. (B) Distribution of macrocycles molecular weight and calculated atomic log(P). (C) Histogram showing the distribution of hydrogen bond acceptor atoms in each macrocycle. (D) Histogram showing the distribution of hydrogen bond donor atoms in each macrocycle.

FIG. 9. Impact of sidechain substitution on molecular shape. (A) Principal moment of inertia analysis of every atom in the lowest energy conformer for a handful of sequences across three chemotypes. Distinct preferences for PMI ratios are apparent within each chemotype. (B) Superposition of the lowest energy conformers of the macrocycles aaap-PRO-PHE_pp-mTIC-AMACBEN3 and aaap-PRO-mPHE_pp-mTIC-AMACBEN3. These diastereomers orient their phenylalanine and Tic monomers in a similar arrangement despite the differing alpha stereocenters within their respective phenylalanine monomers. This results in macrocycles that have very similar shapes despite the epimerization and distinct torsional bins they populate.

FIG. 10. The low-energy conformer identified from the hash-generated ensembles are very similar to the X-ray crystallographic structure of the macrocycle. The backbone atoms are highlighted per FIG. 1. In each of the energy landscapes, RMSD is calculated to the lowest energy conformer sampled in the ensemble. Each dot represents the endpoint of an AIMNet minimization of a single conformer generated by the hash-based assembly. The predicted low-energy conformers for each macrocycle are depicted as sticks. Crystallographic coordinates for each macrocycle are depicted as sticks. For cyclotrisarcosine, the X-ray crystallographic structure contains two distinct conformers of the macrocycle in the asymmetric unit. These conformers are related to one-another through a mirror inversion. These two conformers are sampled in the hash-generated ensemble and are nearly identical in energy. These two predicted conformers are superimposed on the asymmetric unit of cyclotrisarcosine.

FIG. 11. Energy landscapes of macrocycles whose unminimized ensembles sample few torsion bins frequently contain deep energy minima that sample a single state with Pnear>0.8. Ensembles of 3 residue macrocycles were minimized (1735 sequences from macrocycles that sampled<5 bins, 889 from macrocycles that sampled>100 bins) and the quality of the resulting landscapes were assessed using Pnear. Energy landscapes of macrocycles whose unminimized ensembles sample many torsion bins often contain shallow energy minima that sample multiple states.

FIG. 12. The NMR ensemble or X-ray crystallographic structures of three designed macrocycles are not close to their predicted low-energy conformation. Design models are depicted in sticks and spheres. The 20 lowest-energy conformers from unconstrained MD simulations are depicted as lines. X-ray crystallographic structures are depicted as sticks. The distance-constrained ensemble of aaag contains three distinct conformers that all differ from the predicted low energy conformer of the molecule. The distance-constrained ensemble of aabs contains a single conformer, at the level of backbone torsion angles, which is stabilized by a hydrogen bond between the carbonyl of the aminobutyic acid monomer and the NH of the cysteine-containing monomer.

FIG. 13. Cis-amides are a recurrent motif amongst 4-residue macrocycles constructed of primarily alpha amino acids. (A) Fraction of conformers across each sequence within the specified chemotype containing only trans amides or at least 1 cis-amide. cis-Amides are present in many of the conformers of macrocycles sampled amongst chemotypes comprised of three alpha-amino acids and one unnatural amino acid, especially for chemotypes constructed from backbones that contain many fixed torsion angles (e.g. 3-aminobenzoic acid/f containing macrocycles or 4-aminomethylphenylacetic acid/q containing macrocycles). Long unconstrained backbones, such as epsilon-amino acids (e), and backbones with obvious turn-inducing motifs, such as 2-aminobenzoic acids (c) and 1,2- or 1,3-aminomethylphenylacetic acids (m, p respectively), alleviate the strain associated with cis amides, which primarily involve tertiary amides. (B) Ramachandran plot of every alpha amino acids with a cis-amide from chemotypes in panel A. points represent N-alkylated amino acids (e.g. N-methylalanine), points represent proline-like amino acids, and points represent N-proto amino acids (e.g. alanine). These tertiary amides populate 4 distinct regions of ramachandran space, regardless if they are from proline-like residues, N-methylated residues, or peptoids; the same regions of ramachandran space are also sampled by N-proto amino acids that contain cis amides. Representative conformers of D-proline from within two of the four discrete bins are depicted as sticks. These conformers promote the rapid turning of the backbone amongst these macrocycles. These conformers are not privileged to proline-like amino acids, but are instead sampled by most alpha amino acids within these macrocycles. These specific combinations of phi and psi torsion angles, in conjunction with the cis amide, yield conformers of alpha amino acids that promote rapid turning of the backbone within a single residue; the same regions of ramachandran space, but with trans amides, produce much more extended conformers of alpha amino acids.

FIG. 14. aaam mimics the spacing and orientation of residues at i,i+4 of alpha-helices. X-ray crystallographic structure of aaam (left) superimposed on an idealized alpha-helix (right).

FIG. 15. PAMPA results for all prepared macrocycles. The dashed line is placed at Pₐₚₚ=1×10⁻⁶ cm/s.

FIG. 16. Impact of hashtable resolution on quality of macrocyclic closures in two residue macrocycles. Hashtables for each monomer were constructed at high (0.5 Å, 10 degree) medium (0.75 Å, 12.5 degree), low (1.0 Å, 15 degree) and very low (2.0 Å, 30 degree) Cartesian and origin resolutions. The error in NCO alignment in each of the resulting conformers were calculated and plotted as KDE normalized histograms. The higher the resolution of the hashtables, the lower the RMSD error in fragment alignment.

FIG. 17. Identification of candidate low energy sequences for each torsion bin sampled by base residues in a chemotype. (A) Diagram of algorithm to identify potential low energy sequences. (B) Example of process used to identify lowest energy rotamer at each monomer within a single torsion bin representative of the aaas-SAR_pp-SAR-AGLY-SUGA macrocycle. First the backbone torsion angles of the bin representative are calculated. Next, at each monomer, the lowest energy rotamer is identified for each substitution possible at the monomer, given the backbone torsion angles in the torsion bin representative. Finally, the candidate sequence is constructed by identifying the substitution with the lowest energy at each monomer.

FIG. 18. Residual of the linear regression model fit to predict AIMNet energies from sequence composition (top). Zscore analysis for all 8,280 macrocycles used to construct the linear regression model (bottom). Macrocycles whose Pnear>0.8, and Zscore<−2.0 were manually inspected and a subset were chemically synthesized.

FIG. 19. Analytical ultra performance liquid chromatography (UPLC) and Liquid chromatography-mass spectrometry (LCMS) mass spectrum of an exemplary aas macrocyclic oligoamide.

FIG. 20. Analytical UPLC and LCMS mass spectrum of an exemplary akak macrocyclic oligoamide.

FIG. 21. Analytical UPLC and LCMS mass spectrum of an exemplary ahah macrocyclic oligoamide.

FIG. 22. Analytical UPLC and LCMS mass spectrum of an exemplary aabc (1) macrocyclic oligoamide.

FIG. 23. Analytical UPLC and LCMS mass spectrum of an exemplary aaam (1) macrocyclic oligoamide.

FIG. 24. Analytical UPLC and LCMS mass spectrum of an exemplary aaam macrocyclic oligoamide.

FIG. 25. Analytical UPLC and LCMS mass spectrum of an exemplary aaap macrocyclic oligoamide.

FIG. 26. Analytical UPLC and LCMS mass spectrum of an exemplary aaaq macrocyclic oligoamide.

FIG. 27. Analytical UPLC and LCMS mass spectrum of an exemplary aabc (2) macrocyclic oligoamide.

FIG. 28. Analytical UPLC and LCMS mass spectrum of an exemplary aalm macrocyclic oligoamide.

FIG. 29. Analytical HPLC and LCMS mass spectrum of an exemplary aaam (4) macrocyclic oligoamide.

FIG. 30. Analytical HPLC and LCMS mass spectrum of an exemplary aaam (5) macrocyclic oligoamide.

FIG. 31. Analytical HPLC and LCMS mass spectrum of an exemplary aaam (6) macrocyclic oligoamide.

FIG. 32. Analytical HPLC and LCMS mass spectrum of an exemplary aaam (8) macrocyclic oligoamide.

FIG. 33. Analytical HPLC and LCMS mass spectrum of an exemplary aabs (1) macrocyclic oligoamide.

FIG. 34. Analytical HPLC and LCMS mass spectrum of an exemplary aaap (1) macrocyclic oligoamide.

FIG. 35. Analytical HPLC and LCMS mass spectrum of an exemplary aaap (2) macrocyclic oligoamide.

FIG. 36. Analytical HPLC and LCMS mass spectrum of an exemplary aaap (3) macrocyclic oligoamide.

FIG. 37. Analytical HPLC and LCMS mass spectrum of an exemplary asbc (3) macrocyclic oligoamide.

FIG. 38. Analytical UPLC and LCMS mass spectrum of an exemplary aabb (1) macrocyclic oligoamide.

FIG. 39. Analytical UPLC and LCMS mass spectrum of an exemplary aaag (1) macrocyclic oligoamide.

FIG. 40. Analytical UPLC and LCMS mass spectrum of an exemplary aaag (4) macrocyclic oligoamide.

FIG. 41. Analytical HPLC and LCMS mass spectrum of an exemplary aabg (1) macrocyclic oligoamide.

FIG. 42. Analytical HPLC and LCMS mass spectrum of an exemplary aabi (2) macrocyclic oligoamide.

FIG. 43. Analytical HPLC and LCMS mass spectrum of an exemplary aabi (3) macrocyclic oligoamide.

FIG. 44. Analytical HPLC and LCMS mass spectrum of an exemplary aabi (4) macrocyclic oligoamide.

FIG. 45. Analytical HPLC and LCMS mass spectrum of an exemplary aabi (5) macrocyclic oligoamide.

FIG. 46. Analytical UPLC and LCMS mass spectrum of an exemplary bbbb (3) macrocyclic oligoamide.

FIG. 47. Analytical UPLC and LCMS mass spectrum of an exemplary aagb (1) macrocyclic oligoamide.

FIG. 48 shows an example macrocycle computation system.

FIG. 49 is a flow diagram of an example process for generating a set of conformations of a monomer.

FIG. 50 is a flow diagram of an example process for adaptively sampling a space of structure parameters for a monomer.

FIG. 51 illustrates an example of adaptively sampling a space of structure parameters for a monomer to determine a set of conformations of the monomer.

FIG. 52 is a flow diagram of an example process for generating data defining a set of conformations of a molecular fragment based on possible conformations of the monomers in the monomer sequence of the molecular fragment.

FIG. 53 shows an example macrocycle identification system.

FIG. 54 is a flow diagram of an example process for identifying macrocycles using discretized initial-to-terminal transformations and discretized terminal-to-initial transformations.

FIG. 55 is a flow diagram of an example process for predicting whether a molecule that can adopt a set of macrocycle conformations will adopt a rigid macrocycle conformation.

FIG. 56 illustrates a first molecular fragment and a second molecular fragment having conformations that jointly form a closed loop.

FIG. 57 illustrates a superposition of the backbones of a set of conformations of a molecular fragment.

FIG. 58 shows a translation vector defining a translation from an initial end of a molecular fragment to a terminal end of the molecular fragment.

FIG. 59 shows an initial-to-terminal transformation dictionary and a terminal-to-initial transformation dictionary.

Like reference numbers and designations in the various drawings indicate like elements.

### SUMMARY

In a first aspect, the disclosure provides 3 to 4 residue non-naturally occurring macrocycle oligoamide comprising a chemotype of 3 or 4 monomer residues selected from the group consisting of a, b, c, d, e, f, g, h, I, j, k, l, m, n, o, p, q, r, s, t, u, and v monomers as defined in Table 1, or a salt thereof. In one embodiment,
(a) monomer designations a, b, g, d, and e are amino acids, wherein
   (i) monomer designation a comprises or consists of all L- and D-proteinogenic amino acids as well as all L- and D-non-canonical alpha amino acids; peptoids (N-alkylated glycines), and enantiomers thereof;
   (ii) monomer designation b comprises or consists of all B3-amino acids that contain L- or D-proteinogenic side chains, and enantiomers thereof;
   (iii) monomer designation d comprises or consists of delta-amino acids and enantiomers thereof;
   (iv) monomer designation g comprises or consists of gamma amino acids and enantiomers thereof; and
   (v) monomer designation e comprises or consists of epsilon amino acids and enantiomers thereof;
(b) monomer designations c, f, j comprise or consist of aminobenzoic acids and enantiomers thereof;
(c) monomer designations h, k, n comprise or consist of aminomethylbenzoic acids and enantiomers thereof;
(d) monomer designations i, l, o comprise or consist of aminophenylacetic acids and enantiomers thereof;
(e) monomer designations m, p, q comprise or consist of aminomethylphenylacetic acids and enantiomers thereof;
(f) monomer designation r comprises or consists of oxazolidines and thiazolidines that include all L- and D-proteinogenic side chains substituted on the second atom in the backbone, and enantiomers thereof, both for the oxazolidinone and thiazolidinone;
(g) monomer designation t comprises or consists of oxazole and thiazoles that include all L- and D-proteinogenic side chains substituted on the second atom in the backbone, and enantiomers thereof, both for the oxazolidine and thiazolidinine;
(h) monomer designation s comprises or consists of thioethers and enantiomers thereof;
(i) monomer designation comprises or consists of aminomethylpicolinic acids and enantiomers thereof; and
(j) monomer designation v comprises or consists of triazoles and enantiomers thereof.

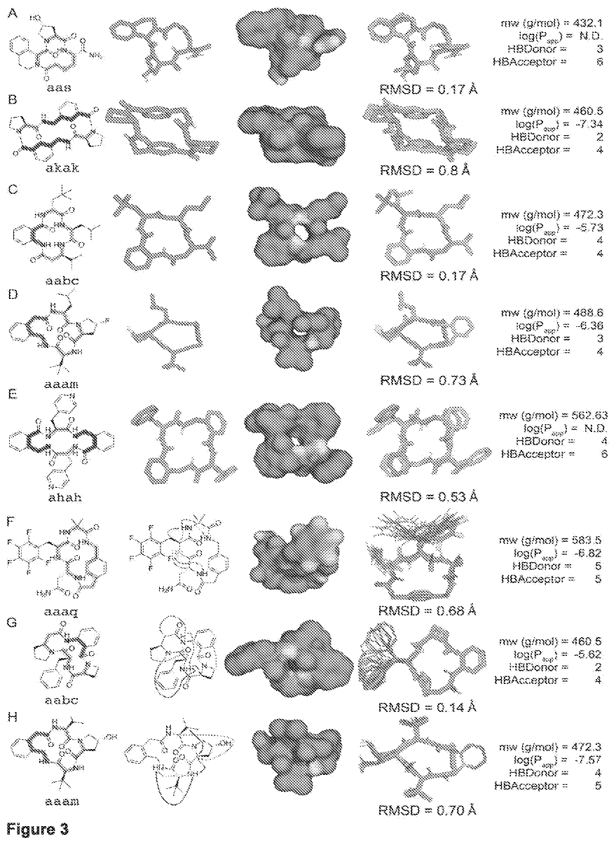

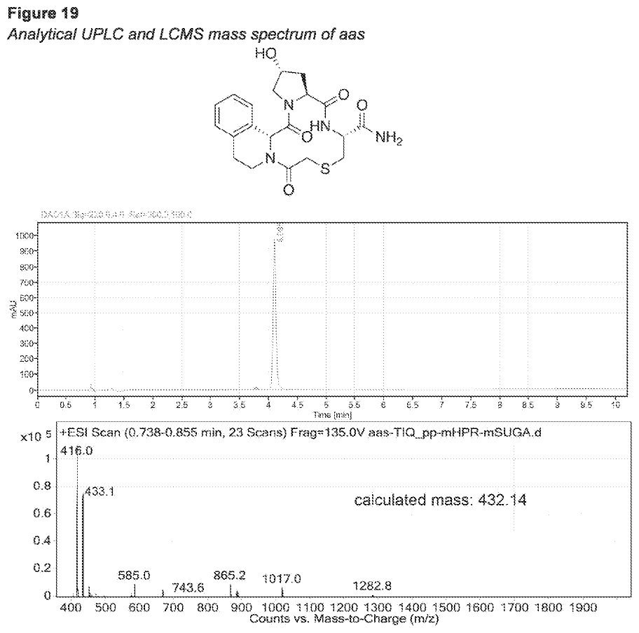

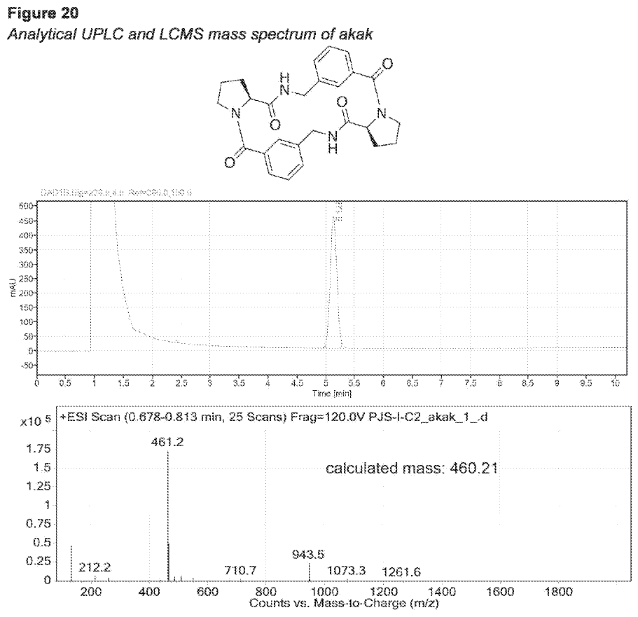

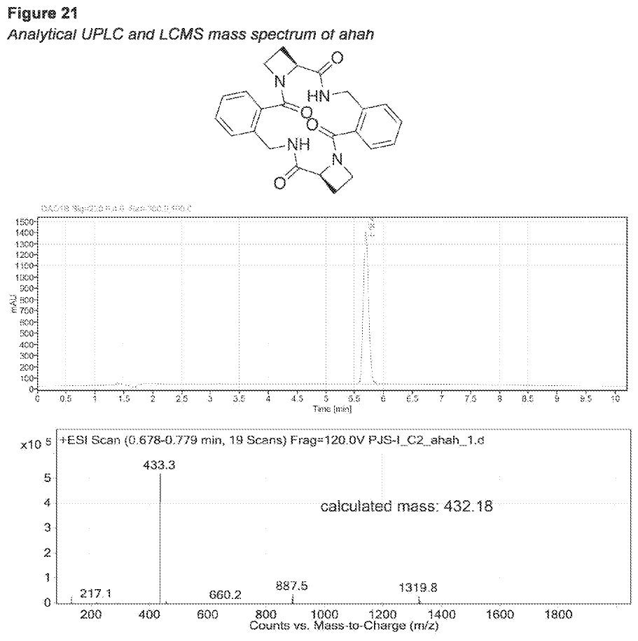

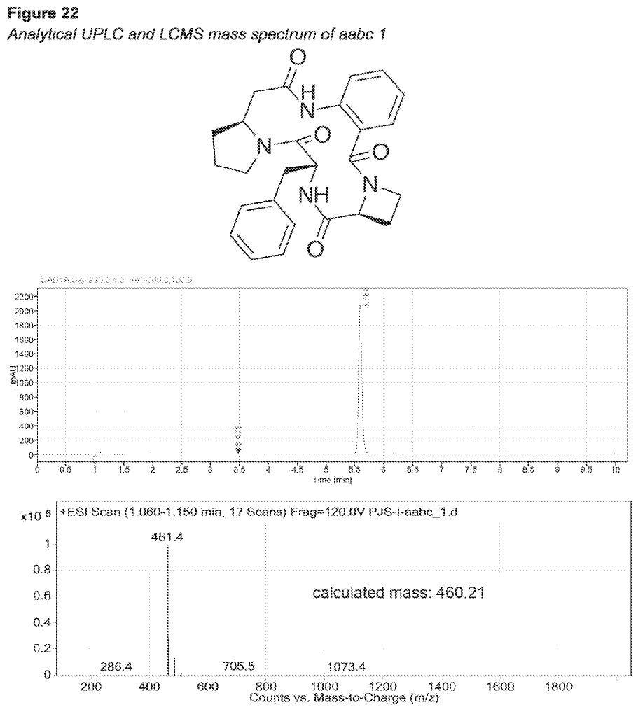

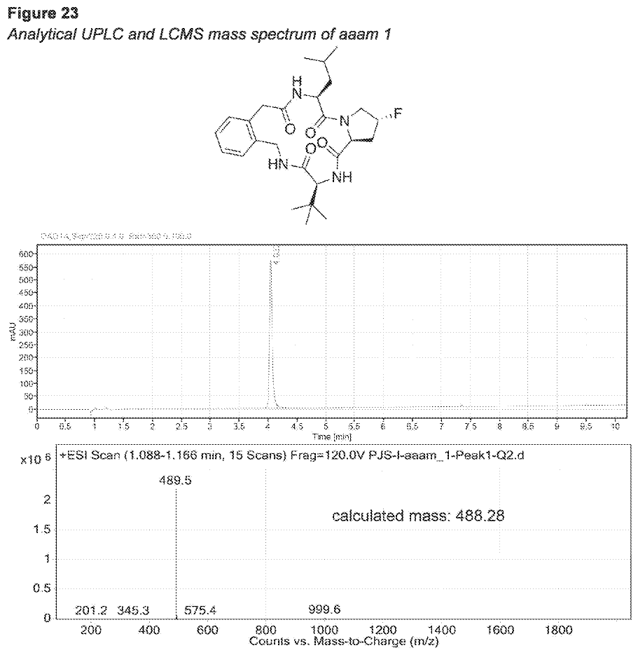

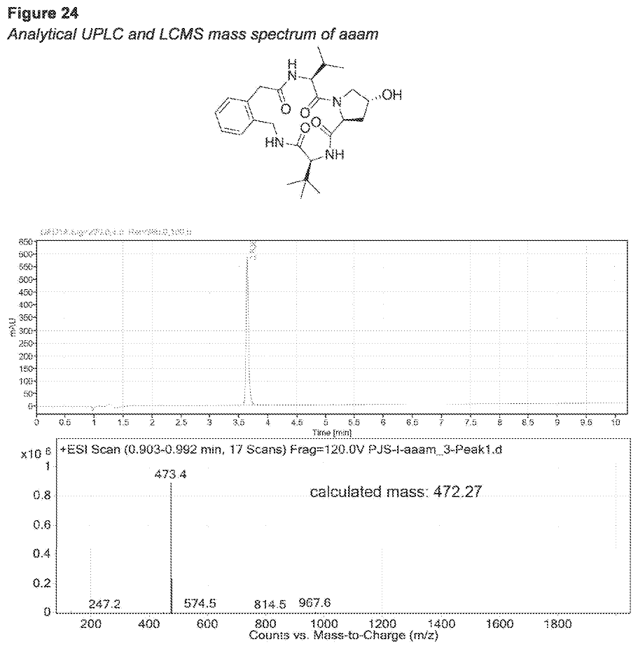

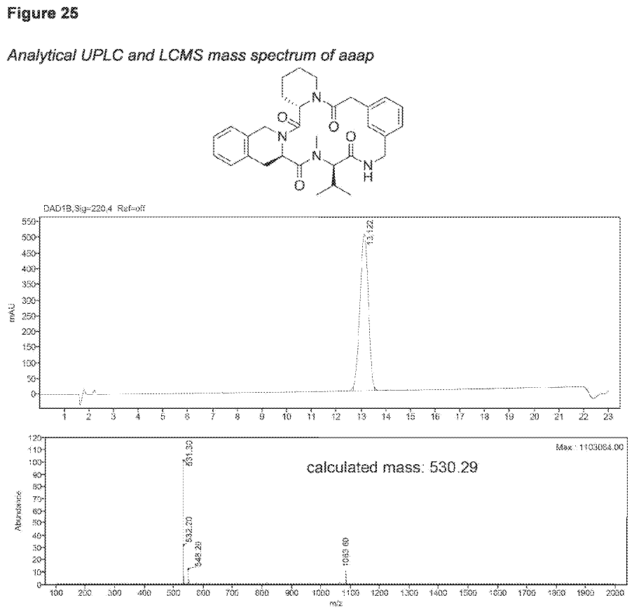

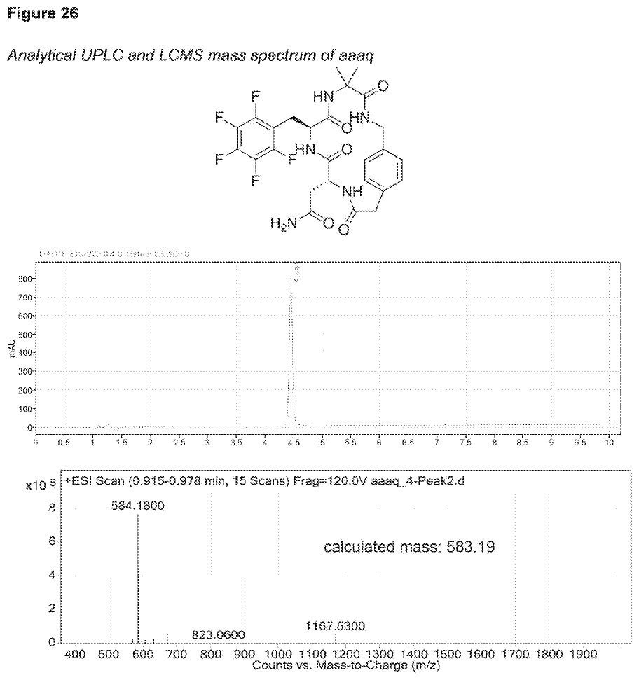

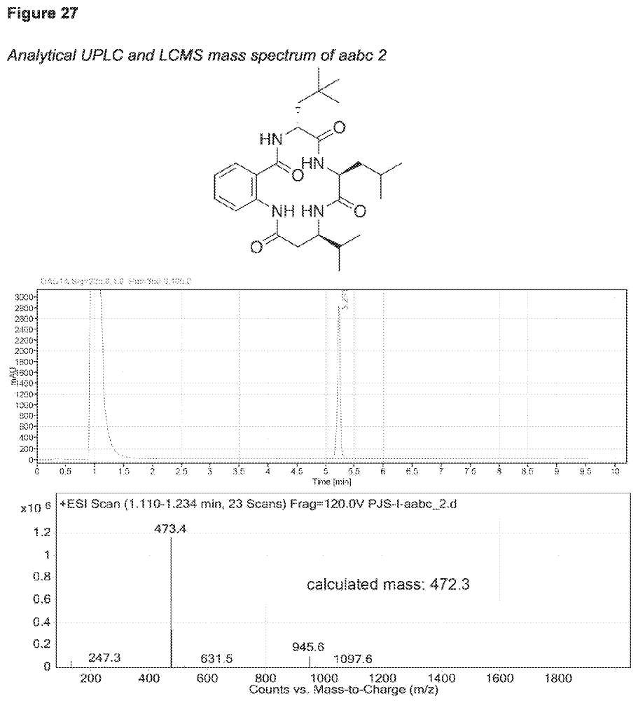

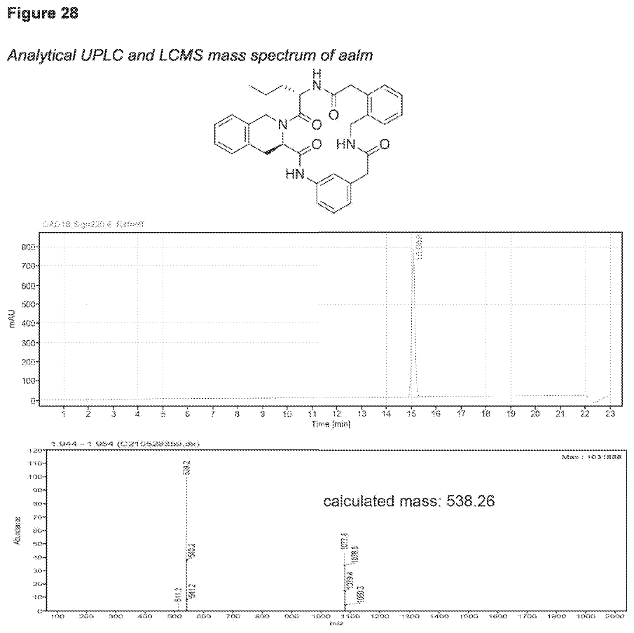

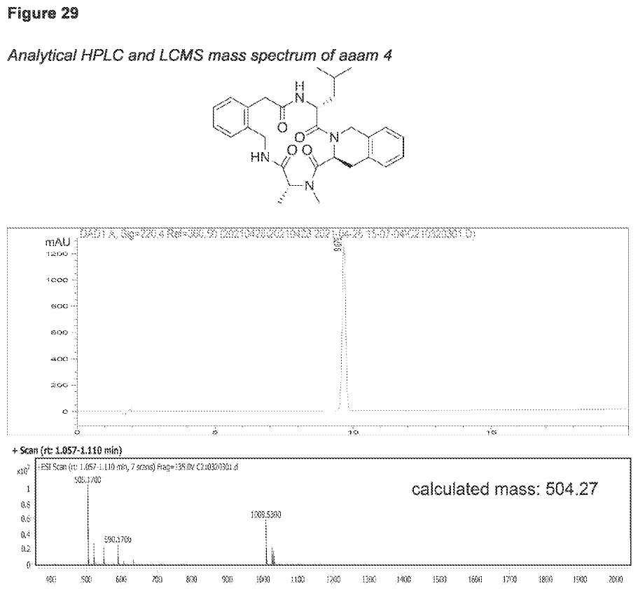

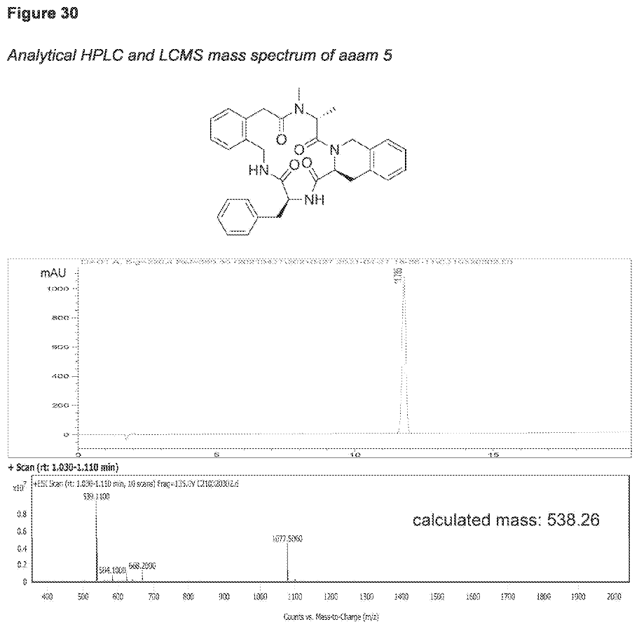

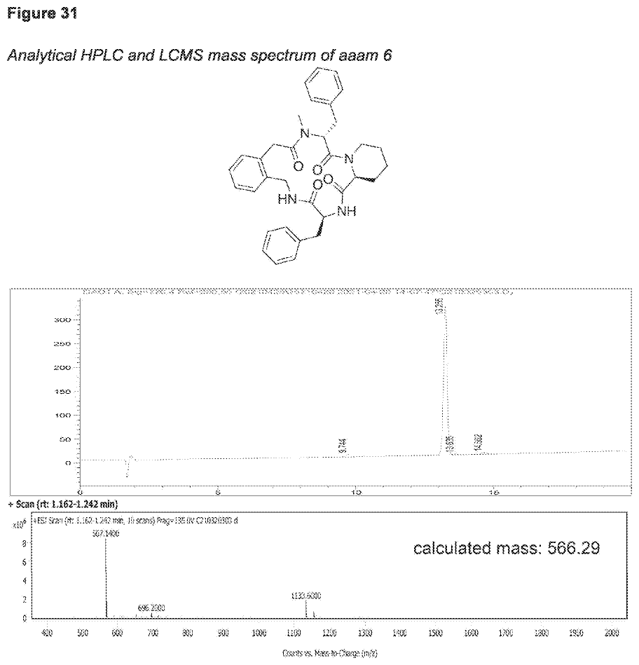

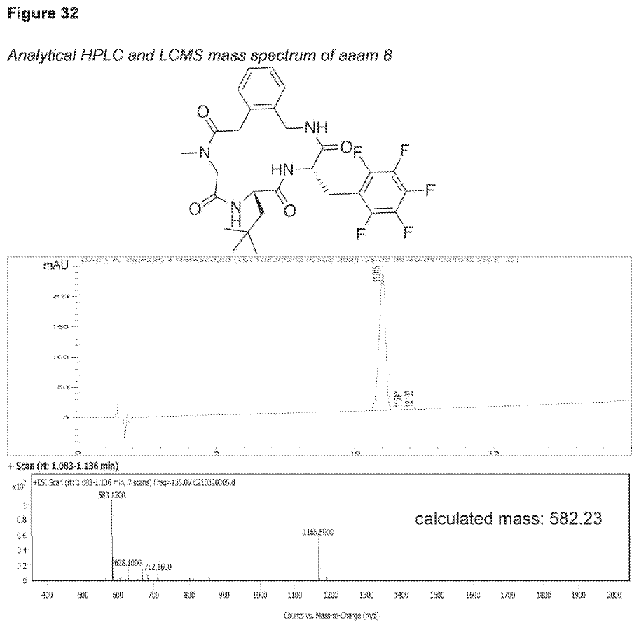

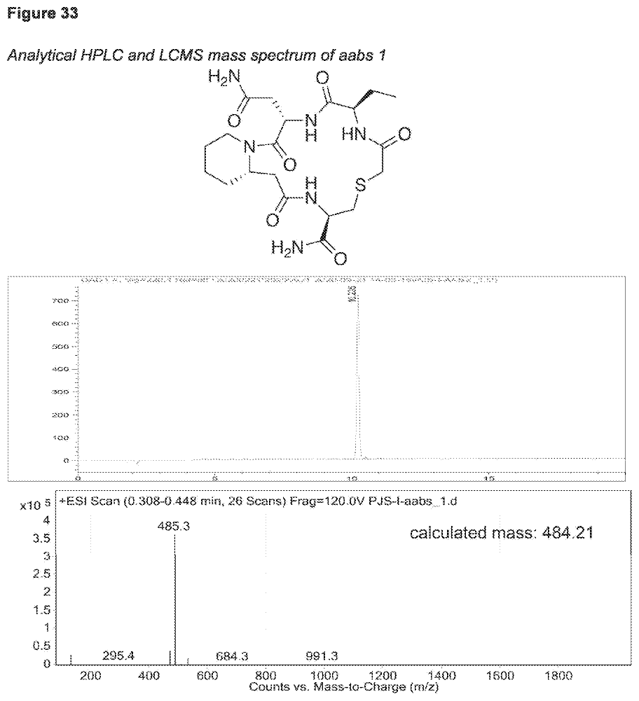

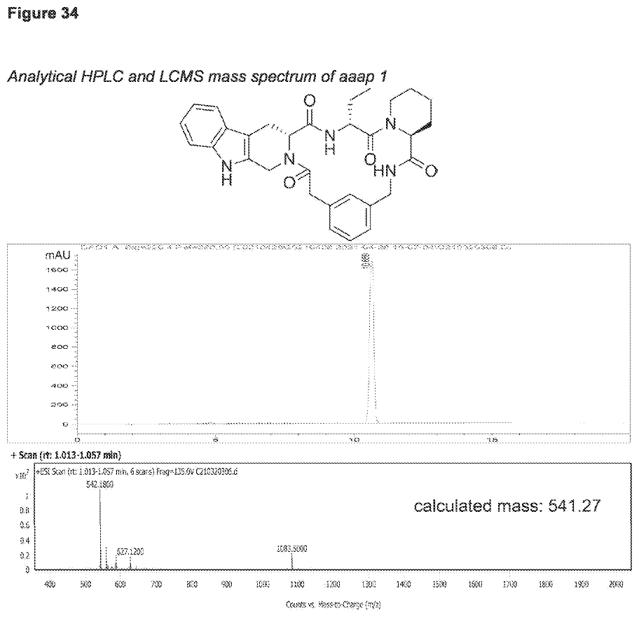

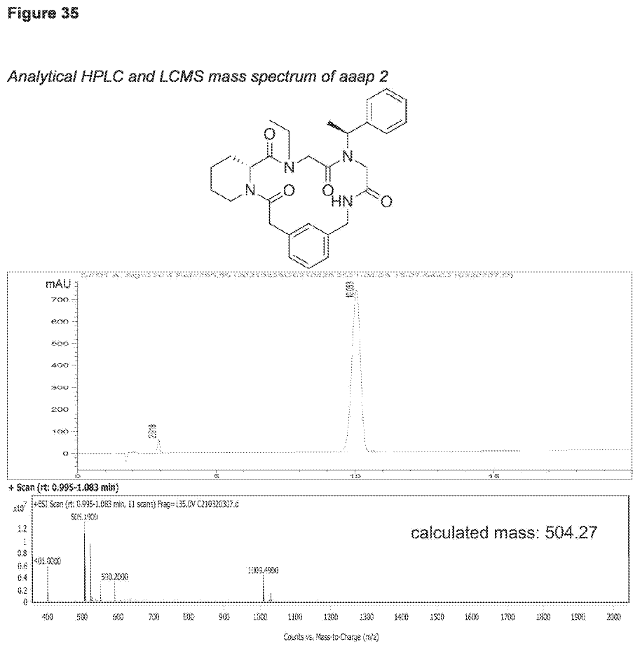

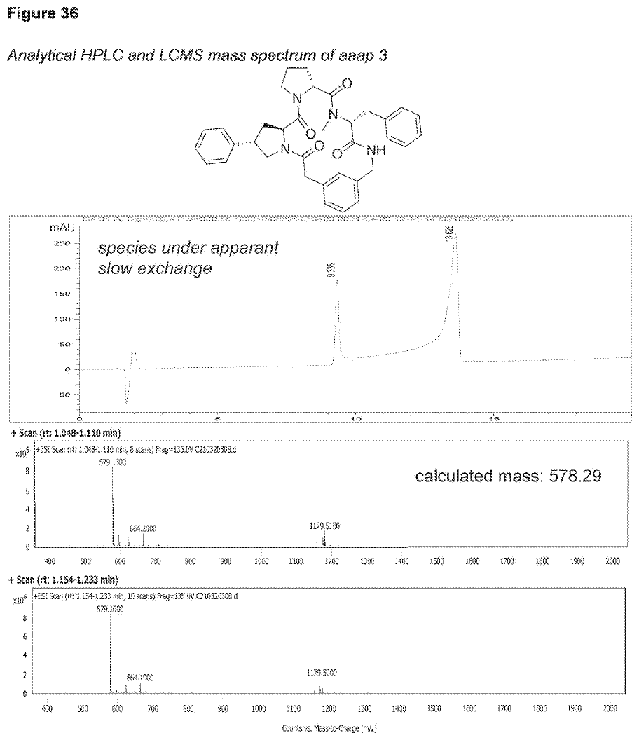

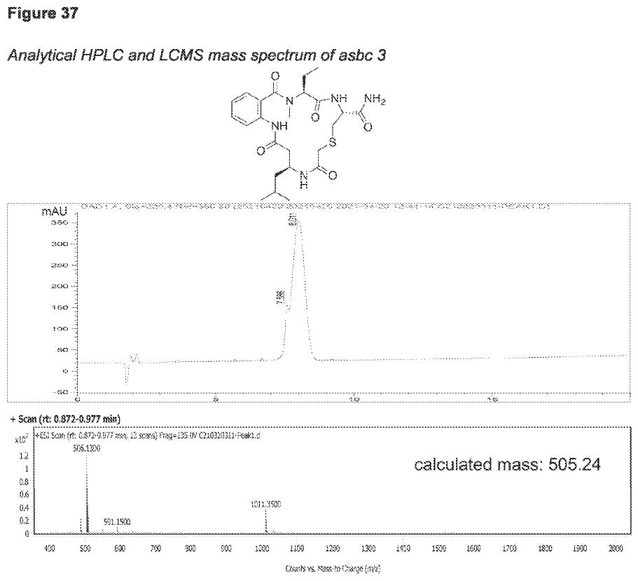

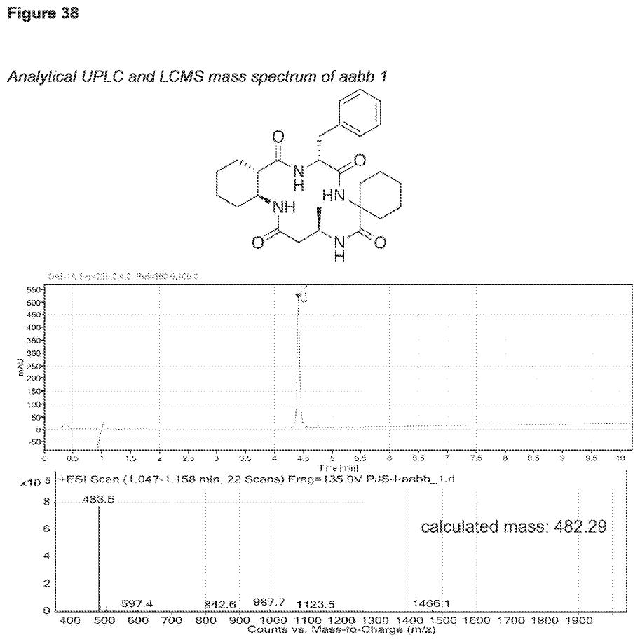

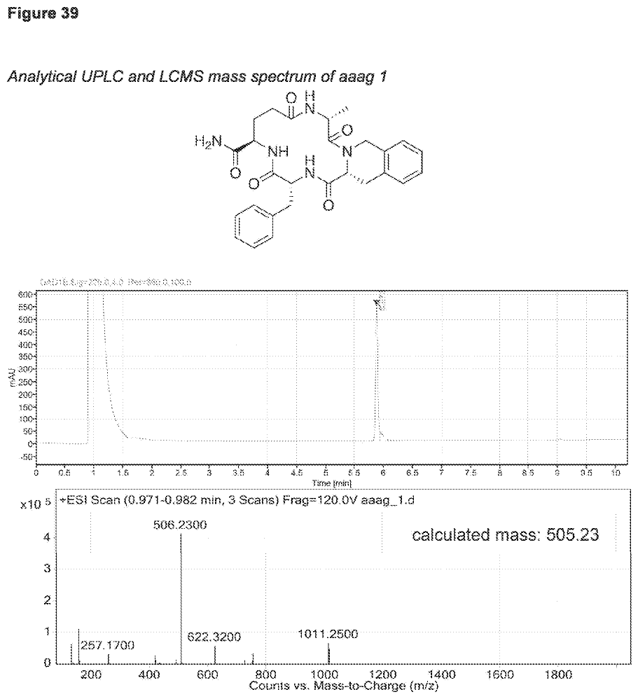

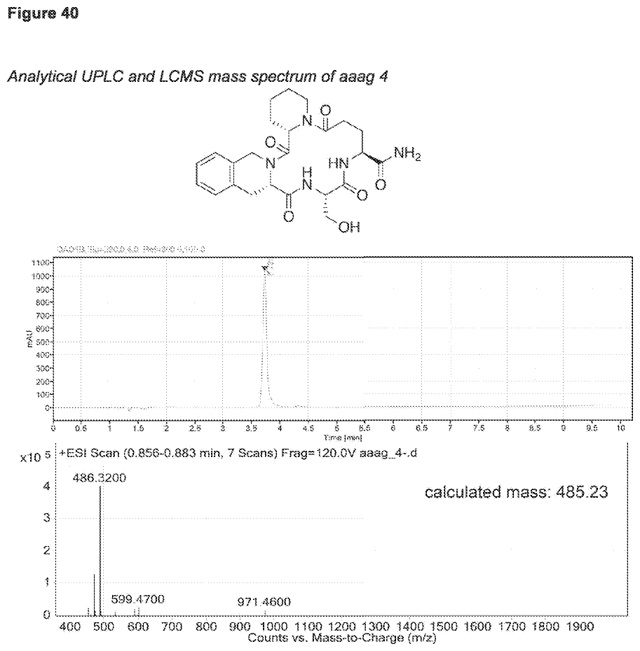

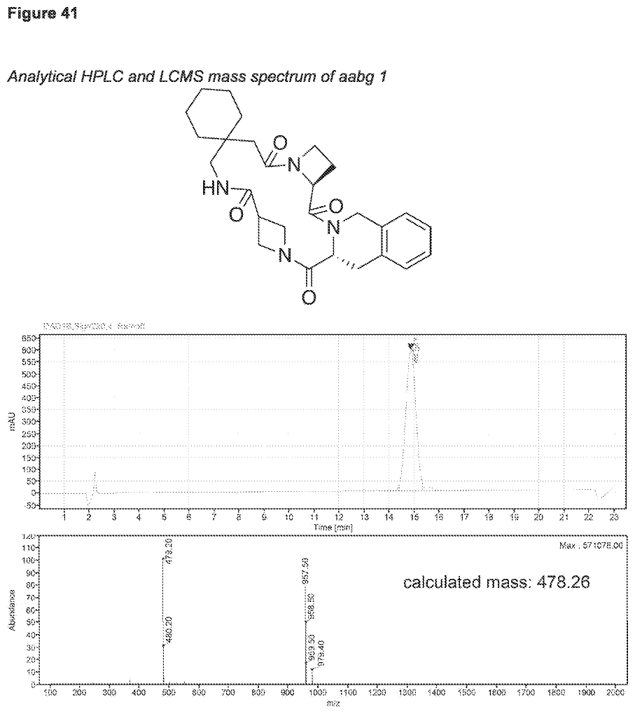

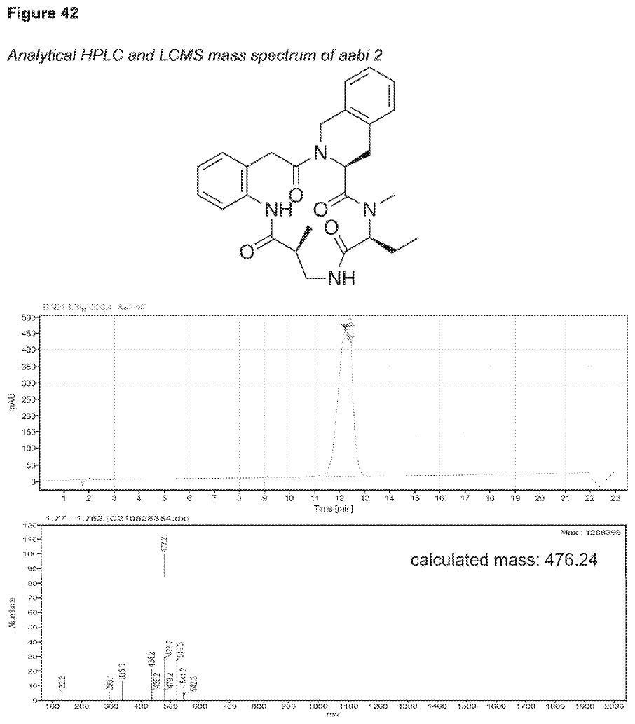

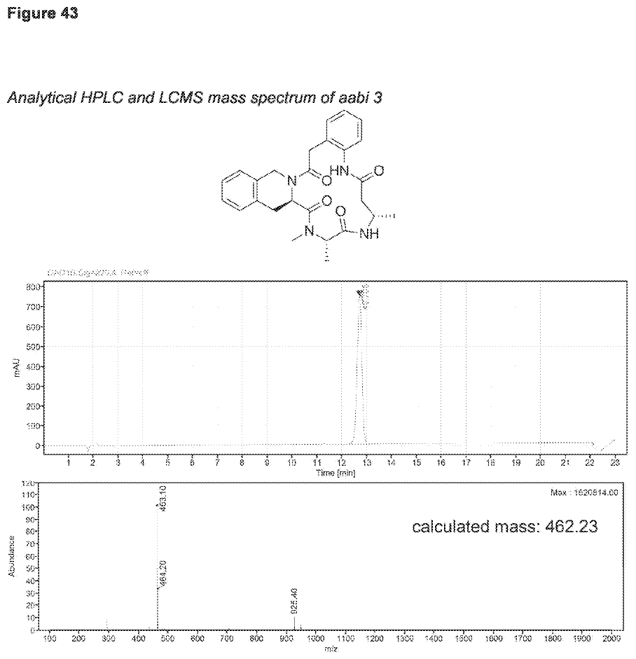

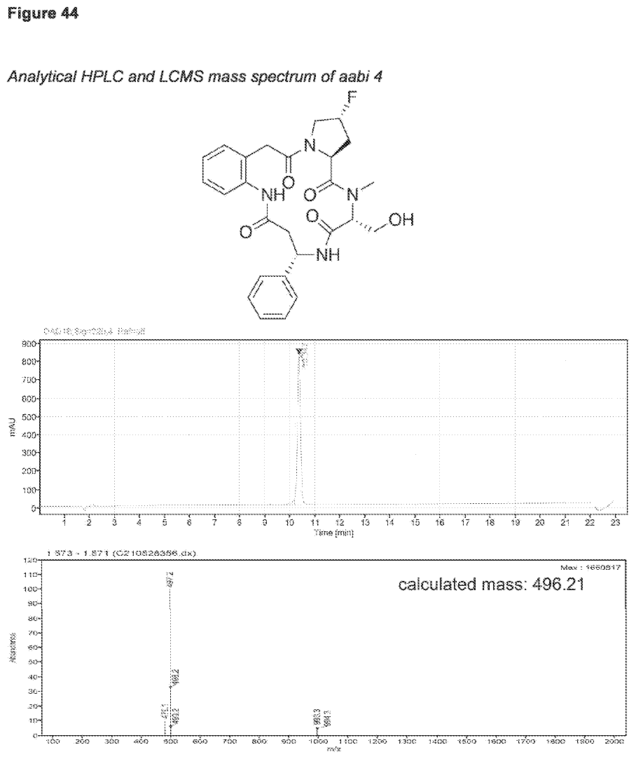

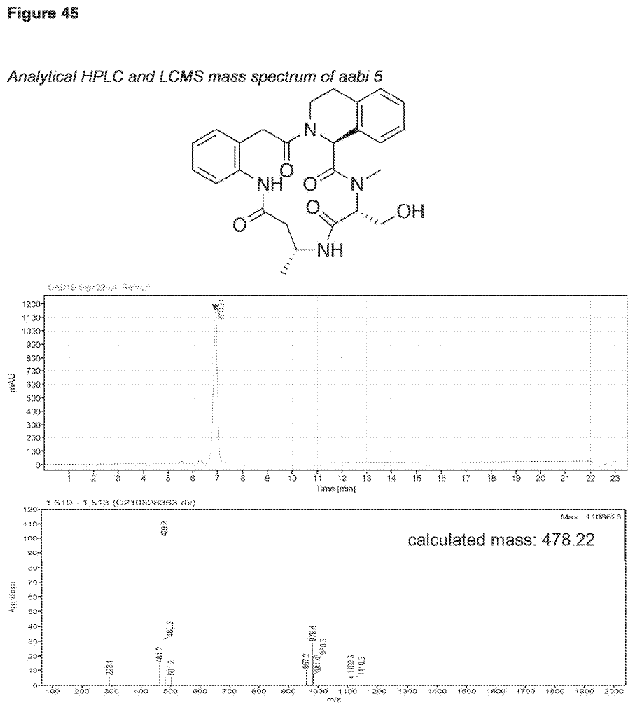

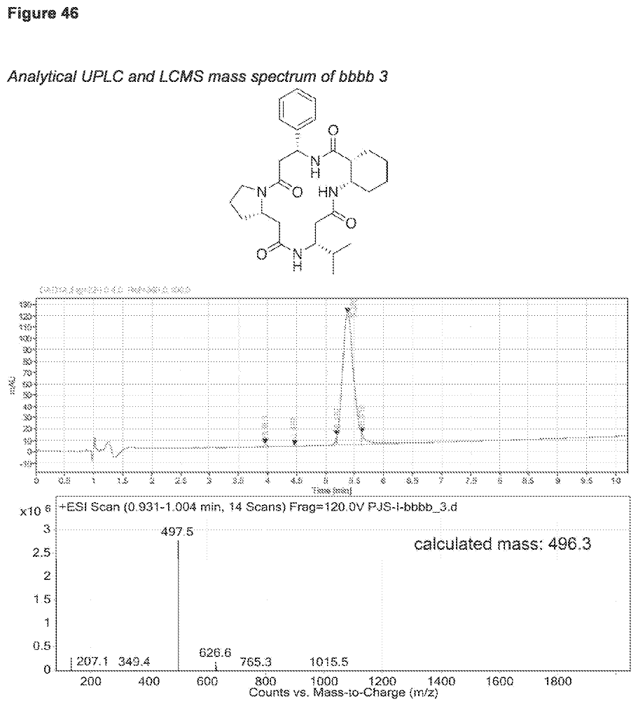

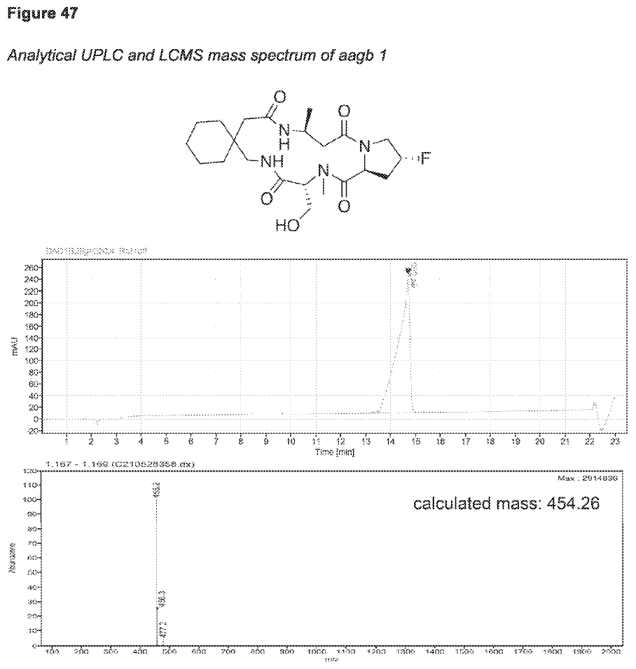

In another embodiment, the macrocycle oligoamide comprises a chemotype selected from the group of chemotypes listed in Table 2, or circularly permuted versions thereof. In a further embodiment, the macrocyclic oligoamide includes at least one monomer not falling within monomer designation a; or at least one residue not falling within monomer designation a, b, g, d, e. In a further embodiment, the macrocyclic oligoamide is membrane permeable. In various embodiments, the macrocyclic oligoamide comprises a structure of any macrocyclic oligoamide disclosed in any figure herein, circularly permuted versions thereof, or salt thereof; or wherein the macrocyclic oligoamide comprises or consists of a structure of any macrocyclic oligoamide shown in any one of FIGS. 3-4, 12, and 19-47, or salt thereof; or comprising or consisting of the structure of any one of compounds 1-218 in Table 3, or salts thereof. In other embodiments, the macrocyclic oligoamide comprises a substitution of 1, 2, 3, or all 4 monomer subunits.

In one embodiment, the disclosure provides a library, comprising 10, 50, 100, 500, 1000, 5000, 10,000, 25, 000, 35,000, or more macrocyclic oligoamides of any embodiment or combination of embodiments herein. In another embodiment, the disclosure provides methods for using the macrocyclic oligoamides and/or library of any embodiment for any suitable purpose, including but not limited to panning the library to identify one or more macrocyclic oligoamide that binds to a compound of interest, therapeutic treatments, diagnostic methods, and/or adding reactive moieties for any use.

In another aspect, the disclosure provides a method performed by one or more computers for identifying macrocycle conformations of a molecule comprising a first molecular fragment and a second molecular fragment, the method comprising:
obtaining a set of conformations of the first molecular fragment;
determining, for each conformation of the first molecular fragment, values of a set of parameters of an initial-to-terminal transformation defining a translation and a rotation of an initial end of the first molecular fragment relative to a terminal end of the first molecular fragment in the conformation;
obtaining a set of conformations of the second molecular fragment;
determining, for each conformation of the second molecular fragment, values of a set of parameters of a terminal-to-initial transformation defining a translation and a rotation of a terminal end of the second molecular fragment relative to an initial end of the second molecular fragment in the conformation; and
processing the respective parameter values of the initial-to-terminal transformations and the terminal-to-initial transformations to identify a set of one or more macrocycle conformations of the molecule,
   wherein each macrocycle conformation of the molecule comprises respective conformations of the first molecular fragment and the second molecular fragment that jointly form a closed loop.

In one embodiment, processing the respective parameter values of the initial-to-terminal transformations and the terminal-to-initial transformations to identify the set of one or more macrocycle conformations of the molecule comprises, for each macrocycle conformation:
determining that the parameter values of the initial-to-terminal transformation for the conformation of the first molecular fragment and the parameter values of the terminal-to-initial transformation for the conformation of the second molecular fragment satisfy a loop closure criterion. In another embodiment, determining that the parameter values of the initial-to-terminal transformation for the conformation of the first molecular fragment and the parameter values of the terminal-to-initial transformation for the conformation of the second molecular fragment satisfy a loop closure criterion comprises:
determining that a discretization of the parameter values of the initial-to-terminal transformation for the conformation of the first molecular fragment are equal to a discretization of the parameter values of the terminal-to-initial transformation for the conformation of the second molecular fragment. In another embodiment, the method further comprises:
determining, for each macrocycle conformation, a respective energy value associated with the macrocycle conformation;
identifying a macrocycle conformation having a minimum energy value among the macrocycle conformations;
determining, for each macrocycle conformation, a respective similarity measure between: (i) the macrocycle conformation, and (ii) the macrocycle conformation having the minimum energy value; and
generating a prediction for whether the molecule comprising the first molecular fragment and the second molecular fragment adopts a rigid macrocycle conformation based on the energy values and the similarity measures for the macrocycle conformations.

In a further embodiment, the method further comprises physically synthesizing the molecule. In one embodiment, the method further comprises
determining one or more conformations of the physically synthesized molecule; and
comparing the conformations of the physically synthesized molecule to the identified macrocycle conformations of the molecule.

The disclosure also provides systems comprising:
one or more computers; and
one or more storage devices communicatively coupled to the one or more computers, wherein the one or more storage devices store instructions that, when executed by the one or more computers, cause the one or more computers to perform operations of the respective method of any embodiment disclosed herein.

The disclosure further provides one or more non-transitory computer storage media storing instructions that when executed by one or more computers cause the one or more computers to perform operations of the respective of any embodiment disclosed herein.

### DETAILED DESCRIPTION

All references cited are herein incorporated by reference in their entirety.

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), valine (Val; V), and alpha-aminoisobutyric acid (AIB, B). Amino acid residues in D-form are noted with a “D” preceding the amino acid residue abbreviation. Amino acid residues in L-form are noted with just the amino acid residue abbreviation, noting that Glycine and alpha-aminoisobutyric acid are non-chiral.

As used herein “salts” refers to both acid and base addition salts.

All embodiments of any aspect of the invention can be used in combination, unless the context clearly dictates otherwise.

In a first aspect, the disclosure provides non-naturally occurring 3 to 4 residue macrocycle oligoamides comprising a chemotype of 3 or 4 monomer residues selected from the group consisting of a, b, c, d, e, f, g, h, I, j, k, l, m, n, o, p, q, r, s, t, u, and v monomers as defined herein. The macrocyclic oligoamides of the disclosure may be used, by way of non-limiting example, as membrane permeable “shuttles” to move attached “cargo” across cell membranes. and as catalysts in chemical reactions.

As used herein a macrocycle is a cyclic macromolecule. The macrocycles disclosed herein are oligoamides, in that each of the 3 or 4 monomer residues comprise an amide group as well as a carboxylic acid. As disclosed in the examples, the inventors have shown that the chemotype of the macrocycles, regardless of its side-chain substitution, defines the macrocycle's globular shape. The macrocycles are organized into different chemotypes according to similar substructures within their composite monomers, as described herein.

Each monomer is assigned a single character that defines the number, hybridization, and elemental identity of the bonded atoms along the backbone, starting from the N-terminal amide nitrogen, and ending at the C-terminal amide carbon. This path of atoms is defined as the shortest path along bonded atoms between the amide nitrogen and carbonyl carbon, in cases where there are multiple paths between these start and end atoms.

Table 1 describe the required atoms and their hybridization along the backbone that a monomer must contain to belong to a specific chemotype. Any substitution of these required atoms is allowed, so long as the atomic number and hybridization of the atoms in the backbone are not changed by the substitution. By way of non-limiting example: L-alanine, D-alanine, L-proline and D-proline are all substitutions on the “a” designation. However, azaglycine (see pubs.acs.org/doi/10.1021/acs.joc.9b02539 for structure) would not belong to “a”, as the atomic numbers change to [7, 7, 6] along the backbone.

TABLE 1

| monomer | | |
|---|---|---|
| designation | atomic numbers | atomic hybridization |
| A | 7, 6, 6 | [SP2, SP3, SP2] or |
| | | [SP2, SP2, SP2] |
| B | 7, 6, 6, 6 | SP2, SP3, SP3, SP2 |
| C | 7, 6, 6, 6 | SP2, SP2, SP2, SP2 |
| D | 7, 6, 6, 6, 6, 6 | SP2, SP3, SP3, SP3, SP3, SP2 |
| E | 7, 6, 6, 6, 6, 6, 6 | SP2, SP3, SP3, SP3, SP3, SP3, SP2 |
| F | 7, 6, 6, 6, 6 | SP2, SP2, SP2, SP2, SP2 |
| G | 7, 6, 6, 6, 6 | SP2, SP3, SP3, SP3, SP2 |
| H | 7, 6, 6, 6, 6 | SP2, SP3, SP2, SP2, SP2 |
| I | 7, 6, 6, 6, 6 | SP2, SP2, SP2, SP3, SP2 |
| J | 7, 6, 6, 6, 6, 6, | SP2, SP2, SP2, SP2, SP2, SP2 |
| K | 7, 6, 6, 6, 6, 6 | SP2, SP3, SP2, SP2, SP2, SP2 |
| L | 7, 6, 6, 6, 6, 6 | SP2, SP2, SP2, SP2, SP3, SP2 |
| M | 7, 6, 6, 6, 6, 6 | SP2, SP3, SP2, SP2, SP3, SP2 |
| N | 7, 6, 6, 6, 6, 6, 6 | SP2, SP3, SP2, SP2, SP2, SP2, SP2 |
| O | 7, 6, 6, 6, 6, 6, 6 | SP2, SP2, SP2, SP2, SP2, SP3, SP2 |
| P | 7, 6, 6, 6, 6, 6, 6 | SP2, SP3, SP2, SP2, SP2, SP3, SP2 |
| Q | 7, 6, 6, 6, 6, 6, 6, 6 | SP2, SP3, SP2, SP2, SP2, SP2, SP3, |
| | | SP2 |
| R | 7, 6, 6, 7, 6, 6 | SP2, SP3, SP2, SP2, SP3, SP2 |
| S | 7, 6, 6, 16, 6, 6 | SP2, SP3, SP3, SP3, SP3, SP2 |
| T | 7, 6, 6, 7, 6, 6 | SP2, SP3, SP2, SP2, SP2, SP2 |
| U | 7, 6, 6, 7, 6, 6 | SP2, SP2, SP2, SP2, SP2, SP2 |
| V | 7, 6, 6, 6, 7, 6, 6 | SP2, SP3, SP2, SP2, SP2, SP3, SP2 |

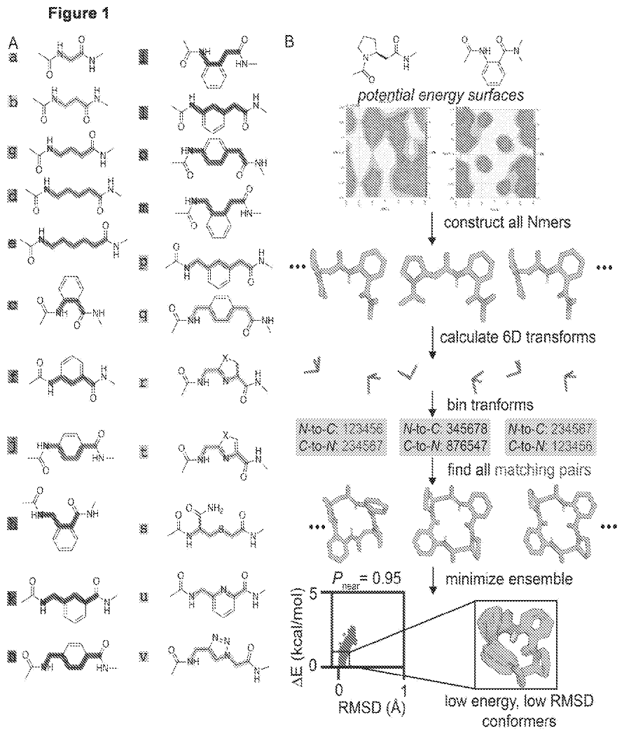

FIG. 1A show the required backbone structure for each monomer designation. For each monomer designation, any compound that includes the highlighted backbone structure shown in FIG. 1 falls within that designation. The non-highlighted portions of monomers shown in FIG. 1A are not a requirement for a monomer of that monomer designation.

In some embodiments, the monomer designations are as follows:
amino acids (monomer designations a, b, g, d, and e):
   a includes all L- and D-proteinogenic amino acids as well as all L- and D-non-canonical alpha amino acids; peptoids (N-alkylated glycines); and enantiomers thereof.
   b includes all B3-amino acids that contain L- or D-proteinogenic side chains, and enantiomers thereof. B3 amino acids are beta amino acids where the side chain is on the beta-carbon in the backbone. The beta carbon is the carbon that is adjacent to the amide nitrogen.
   d for delta-amino acids and enantiomers thereof,
   g for gamma amino acids and enantiomers thereof, and
   e for epsilon amino acids and enantiomers thereof,
aminobenzoic acids (monomer designations: c, f, j),
aminomethylbenzoic acids and enantiomers thereof (monomer designations: h, k, n),
aminophenylacetic acids and enantiomers thereof (monomer designations: i, l, o),
aminomethylphenylacetic acids and enantiomers thereof (monomer designations: m, p, q),
oxazolidines/thiazolidines (monomer designation: r) includes all L- and D-proteinogenic side chains substituted on the second atom in the backbone, and enantiomers thereof, both for the oxazolidinone and thiazolidinone
oxazole/thiazoles (monomer designation: t) includes all L- and D-proteinogenic side chains substituted on the second atom in the backbone, and enantiomers thereof, both for the oxazolidine and thiazolidinine
thioethers (monomer designation: s),
aminomethylpicolinic acids and enantiomers thereof (monomer designation: u), and
triazoles and enantiomers thereof (monomer designation: v).

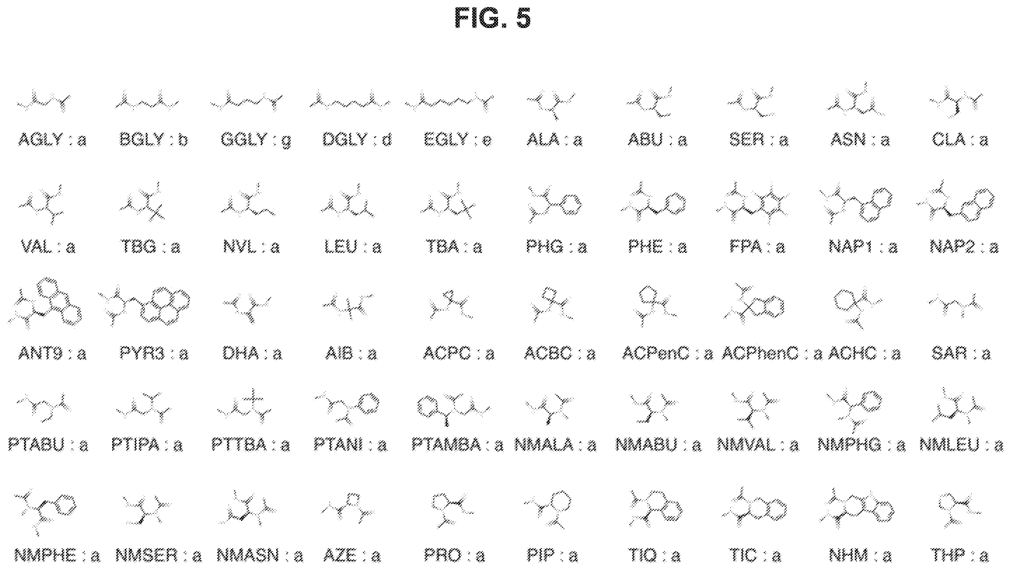

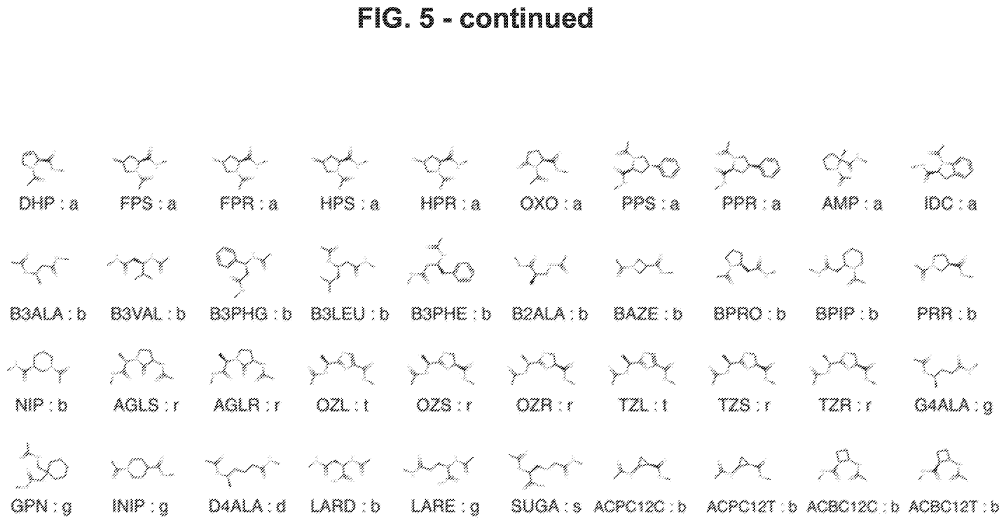

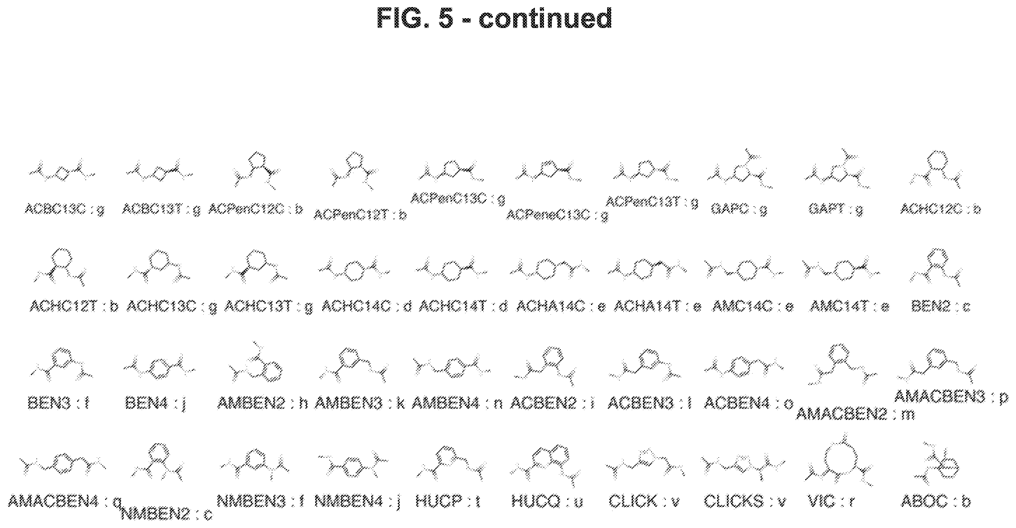

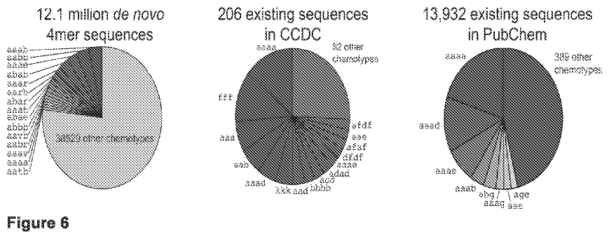

Further exemplary monomer embodiments comprise, but are not limited to, the residues shown in FIG. 5. The monomer designation (also referred to herein as a “bin”) is shown next to each compound. Below each monomer, a unique 3-8 letter code is used to refer to it, as well as its respective chemotype bin, using the rules in Table 1.

In another embodiment, the monomers are selected from the group consisting of the following, as defined by chemical structure in FIG. 5 and Table 5:
Monomer designation “a” selected from the group consisting of AGLY (glycine), ALA (alanine), ABU (aminobutyric acid), SER (serine), ASN (asparagine), CLA (chloroalanine), VAL (valine), TBG (tert-butylglycine), NVL (norvaline), LEU (leucine), TBA (tertbutylalanine), PHG (phenylglycine), PHE (phenylalanine), FPA (perfluorophenylalanine), NAP1, NAP2, ANT9, PYR3, DHA (dehydroalanine), AIB (aminoisobutric acid), ACPC (1-aminocyclopropane carboxylic acid), ACBC (1-aminocyclobutane caboxyclic acid), ACPenC, ACPhenC, ACHC (1-aminocyclohexane carboxylic acid), SAR (sarcosine), PTABU, PTIPA, PTTBA, PTANI, PTAMBA, NMALA (Nmethyl alanine), NMABU (nmethylaminobutyric acid), NMVAL (nmethyl valine), NMPHG (nmethylphenylglcine), NMLEU (nmethyl leucine), NMPHE (nmethyl phenylalanine), NMSER (Nmethyl serine), NMASN (nmethyl asparigine), AZE (azetidine), PRO (proline), PIP (pipercolic acid), TIQ, TIC, NHM, THP, DHP (dehydroproline), FPS, FPR, HPS (gamma-S-hydroxyproline), HPR (gamma-S-hydroxyproline), OXO, PPS (gamma-S-phenylproline), PPR (gamma-R-phenylproline), AMP (alphamethylproline), and IDC, and enantiomers thereof;
Monomer designation “b” selected from the group consisting of BGLY (beta alanine), B3ALA (beta-3-homoalanine), B3VAL (beta-3-homovaline), B3PHG (beta-3-phenylalanine), B3PHE (beta-3-homophenylalanine), B2ALA (beta-2-alanine), BAZE (3-azetidinecarboxlic acid), BPRO (beta-3-homoproline), BPIP (beta-3-homopipercolic acid), PRR, NIP (nipecotic acid), LARD, ACPC12C, ACPC12T, ACBC12C, ACBC12T, ACPenC12C, ACPenC12T, ACHC12C, ACHC12T, and ABOC, and enantiomers thereof;
Monomer designation “c” selected from the group consisting of BEN2 (2-aminobenzoic acid) and NMBEN2 (2-Nmethylamino-benzoic acid);
Monomer designation “d” selected from the group consisting of D4ALA, DGLY (5-aminopentanoic acid), ACHC14C, and ACHC14T;
Monomer designation “e” selected from the group consisting of EGLY, ACHA14C, ACHA14T, AMC14C, and AMC14T;
Monomer designation “f” selected from the group consisting of BEN3 and NMBEN3;
Monomer designation “g” selected from the group consisting of GGLY, G4ALA, GPN, INIP, LARE, ACBC 13C, ACBC13T, ACPenC13C, ACPeneC13C, ACPenC13t, GAPC, GAPT, ACHC13C, and ACHC13T;
Monomer designation “h” is AMBEN2 (2-aminomethylbenzoic acid);
Monomer designation “i” is ACBEN2 (2-aminophenylacetic acid);
Monomer designation “j” selected from the group consisting of BEN4 (4-aminobenzoic acid) and NMBEN4 (4-nmethyl-aminobenzoic acid);
Monomer designation “k” is AMBEN3 (3-aminomethylbenozic acid);
Monomer designation “1” is ACBEN3 (3-aminophenylacetic acid);
Monomer designation “m” is AMACBEN2 (2-aminomethylphenylacetic acid);
Monomer designation “n” is AMBEN4 (4-aminomethylbenozic acid);
Monomer designation “o” is ACBEN4 (3-aminophenylacetic acid);
Monomer designation “p” is AMACBEN3 (3-aminomethylphenylacetic acid);
Monomer designation “q” is AMACBEN4 (4-aminomethylphenylacetic acid);
Monomer designation “r” selected from the group consisting of AGLS, AGLR, OZS, OZR, TZS, TZR, and VIC, and enantiomers thereof;
Monomer designation “s” is SUGA;
Monomer designation “t” selected from the group consisting of OZL, TZL, and HUCP, and enantiomers thereof;
Monomer designation “u” is HUCQ; and/or
Monomer designation “v” selected from the group consisting of CLICK and CLICKS.

To assign the chemotype of a macrocycle oligoamides of the disclosure, first the designations of its composite monomers are determined using Table 1. Then, the resulting string of monomer designations is circularly permuted into the lowest-priority string, determined by alphabetization. For example, the macrocycle cyclo-(glycine-glycine-glycine-beta_glycine) (SEQ ID NO:1) has the macrocycle chemotype of aaab. The aaab chemotype can be circularly permuted into the following 4 identical chemotypes: aaba, abaa, baaa, aaab. Alphabetizing this list results in the following list: aaab, aaba, abaa, baaa. Thus, each recited chemotype below includes circularly permuted versions thereof. The first bin in the alphabetized list is taken as the macrocyclic chemotype for the chemical.

The macrocycles of the disclosure may comprise any non-naturally occurring chemotype of 3 or 4 monomer residues selected from the group consisting of a, b, c, d, e, f, g, h, I, j, k, l, m, n, o, p, q, r, s, t, u, and v. In one embodiment, the macrocycle oligoamides comprising a chemotype selected from the group listed in Table 2, or circularly permuted versions thereof.

TABLE 2
Exemplary chemotypes

aaaf, aaah, aaai, aaaj, aaaq, aaaraaau, aabc, aabf, aabh, aabi, aabj, aabk, aabl, aabm, aabn, aabp, aabq,
aabr, aabs, aabt, aabu, aabv, aacb, aacc, aace, aacf, aacg, aach, aaci, aack, aacl, aacm, aacn, aacp, aacq,
aacr, aacs, aact, aacu, aacv, aadc, aadf, aadg, aadh, aadi, aadj, aadk, aadl, aadm, aadn, aadp, aadq, aadr,
aadt, aadu, aadv, aaec, aaef, aaeh, aaei, aaej, aaek, aael, aaem, aaen, aaep, aaeq, aaer, aaes, aaet, aaeu,
aaev, aafb, aafc, aafd, aafe, aaff, aafg, aafh, aafi, aafj, aafk, aafm, aafp, aafq, aafr, aafs, aaft, aafu, aafv,
aagc, aagf, aagh, aagi, aagj, aagl, aagm, aagn, aago, aagp, aagq, aagr, aags, aagt, aagu, aagv, aah, aahb,
aahc, aahd, aahe, aahf, aahg, aahh, aahi, aahl, aahm, aahn, aaho, aahp, aahq, aahr, aahs, aaht, aahu, aahv,
aai, aaib, aaic, aaid, aaie, aaif, aaig, aaih, aaii, aaik, aail, aaim, aain, aaip, aaiq, aair, aais, aait, aaiv, aajb,
aajc, aajf, aajg, aajh, aajj, aajm, aajt, aak, aakb, aakc, aakd, aake, aakf, aakg, aakh, aaki, aakj, aakl, aakm,
aakp, aakq, aakr, aaks, aakt, aaku, aakv, aal, aalb, aalc, aald, aale, aalf, aalg, aalh, aali, aalj, aalk, aall,
aalm, aaln, aalp, aalq, aalr, aals, aalt, aalu, aalv, aamb, aamc, aamd, aame, aamf, aamg, aamh, aami, aamj,
aaml, aamm, aamn, aamo, aamp, aamq, aamr, aams, aamt, aamv, aan, aanb, aanf, aanj, aant, aao, aace,
aaof, aaoj, aap, aapb, aapc, aapd, aape, aapf, aapg, aaph, aapi, aapj, aapk, aapl, aapm, aapn, aapp, aapq,
aapr, aaps, aapt, aapu, aapv, aaq, aaqb, aaqc, aaqd, aaqe, aaqf, aaqg, aaqh, aaqi, aaqj, aaqk, aaql, aaqm,
aaqn, aaqp, aaqq, aaqr, aaqs, aaqt, aaqu, aaqv, aar, aarb, aarc, aard, aare, aarf, aarg, aarh, aari, aarj, aark,
aarl, aarm, aarn, aarp, aarq, aars, aaru, aarv, aasc, aasd, aase, aasf, aasg, aash, aasi, aask, aasl, aasm, aasn,
aaso, aasp, aasq, aasr, aast, aasu, aasv, aat, aatb, aatc, aatd, aate, aatf, aatg, aath, aati, aatj, aatk, aatl, aatm,
aatn, aatp, aatq, aats, aatu, aatv, aau, aaub, aauc, aaud, aaue, aauf, aaug, aauh, aaui, aauj, aaum, aaup,
aauq, aaur, aaut, aauu, aauv, aavb, aavc, aavd, aave, aavf, aavg, aavh, aavi, aavj, aavk, aavl, aavm, aavn,
aavp, aavq, aavr, aavs, aavt, aavu, aavv, abaf, abah, abai, abaj, abak, abal, abam, aban, abap, abaq, abar,
abas, abat, abau, abav, abbc, abbf, abbg, abbh, abbi, abbj, abbk, abbl, abbm, abbn, abbo, abbp, abbq,
abbr, abbs, abbt, abbu, abbv, abcb, abcc, abcd, abce, abcf, abcg, abch, abci, abcj, abck, abcl, abcm,
abcn, abcp, abcq, abcr, abcs, abct, abcu, abcv, abde, abdf, abdg, abdh, abdi, abdj, abdk, abdl, abdm, abdn,
abdp, abdq, abdr, abds, abdt, abdu, abdv, abeb, abec, abee, abef, abeg, abeh, abei, abej, abek, abel, abem,
aben, abeo, abep, abeq, aber, abes, abet, abeu, abev, abf, abfb, abfc, abfd, abfe, abff, abfg, abfh, abfi,
abfj, abfk, abfm, abfn, abfp, abfq, abfr, abfs, abft, abfu, abfv, abgb, abgc, abgd, abgf, abgg, abgh, abgi,
abgj, abgk, abgl, abgm, abgn, abgo, abgp, abgq, abgr, abgs, abgt, abgu, abgv, abh, abhb, abhc, abhd,
abhe, abhf, abhg, abhh, abhi, abhk, abhl, abhm, abhn, abho, abhp, abhq, abhr, abhs, abht, abhu, abhv,
abi, abib, abic, abid, abie, abif, abig, abih, abii, abik, abil, abim, abin, abio, abip, abiq, abir, abis, abit,
abiv, abj, abjb, abjc, abje, abjf, abjg, abjh, abjj, abjm, abjp, abjr, abjs, abit, abju, abjv, abk, abkb, abkc,
abkd, abke, abkf, abkg, abkh, abki, abkj, abkk, abkl, abkm, abkn, abkp, abkq, abkr, abks, abkt, abku,
abkv, abl, ablb, ablc, abld, able, ablf, ablg, ablh, abli, ablj, ablk, abll, ablm, abln, ablp, ablq, ablr, abls,
ablt, ablu, ablv, abm, abmb, abmc, abmd, abme, abmf, abmg, abmh, abmi, abmj, abml, abmm, abmn,
abmo, abmp, abmq, abmr, abms, abmt, abmv, abn, abnb, abnc, abnf, abng, abnh, abnj, abns, abnt, abo,
abob, abod, aboe, abof, aboh, aboi, aboj, abop, aboq, abor, abos, abot, abou, abov, abp, abpb, abpc, abpd,
abpe, abpf, abpg, abph, abpi, abpj, abpk, abpl, abpm, abpn, abpo, abpp, abpq, abpr, abps, abpt, abpu,
abpv, abq, abqb, abqc, abqd, abqe, abqf, abqg, abqh, abqi, abqj, abqk, abql, abqm, abqn, abqp, abqq,
abqr, abqs, abqt, abqu, abqv, abr, abrb, abrc, abrd, abre, abrf, abrg, abrh, abri, abrj, abrk, abrl, abrm, abrn,
abro, abrp, abrq, abrr, abrs, abrt, abru, abrv, abs, absb, absc, absd, abse, absf, absg, absh, absi, absk, absl,
absm, absn, abso, absp, absq, absr, abss, abst, absu, absv, abt, abtb, abtc, abtd, abte, abtf, abtg, abth, abti,
abtj, abtk, abtl, abtm, abtn, abtp, abtq, abtr, abts, abtt, abtu, abtv, abu, abub, abuc, abud, abue, abuf, abug,
abuh, abui, abuj, abul, abum, abun, abup, abuq, abur, abus, abut, abuu, abuv, abv, abvb, abvc, abvd, abve,
abvf, abvg, abvh, abvi, abvk, abvl, abvm, abvn, abvo, abvp, abvq, abvr, abvs, abvt, abvu, abvv, acae,
acaf, acag, acah, acai, acak, acam, acap, acaq, acar, acas, acat, acau, acav, acbb, acbc, acbd, acbe, acbf,
acbg, acbh, acbi, acbj, acbk, acbl, acbm, acbn, acbp, acbq, acbr, acbs, acbt, acbu, acbv, accb, accc, accd,
acce, accf, accg, acci, acck, accl, accm, accp, accq, accr, acct, accv, acdb, acdc, acdd, acde, acdf, acdg,
acdh, acdi, acdl, acdm, acdp, acdq, acdr, acdt, acdu, aceb, acec, aced, acee, acef, aceg, aceh, acei, acej,
acek, acel, acem, acen, acep, aceq, acer, aces, acet, aceu, acev, acf, acfb, acfc, acfd, acfe, acff, acfg, acfh,
acfi, acfj, acfm, acfp, acfr, acfs, acft, acfu, acfv, acg, acgb, acgc, acgd, acge, acgf, acgg, acgh, acgi, acgj,
acgk, acgl, acgm, acgp, acgq, acgr, acgs, acgt, acgu, acgv, ach, achb, achc, achd, ache, achf, achg, achh,
achi, achl, achm, achp, achq, achr, achs, acht, achu, achv, aci, acib, acic, acid, acie, acig, acih, acii, acik,
acil, acim, acin, acip, aciq, acir, acis, acit, aciv, acj, acjf, acjh, acjj, acjm, acjs, ack, ackb, ackc, ackd,
acke, ackf, ackg, ackh, acki, ackj, ackm, ackr, acks, ackt, acku, acl, aclb, aclc, acld, acle, aclf, aclg, aclh,
acli, aclj, aclk, acll, aclm, acln, aclp, aclq, aclr, acls, aclt, aclu, aclv, acm, acmb, acmd, acme, acmf, acmi,
acml, acmm, acmp, acmq, acmr, acms, acn, acnf, acnj, acns, aco, acoe, acof, acor, acot, acp, acpb, acpc,
acpd, acpe, acpf, acpg, acph, acpi, acpj, acpk, acpl, acpm, acpn, acpp, acpq, acpr, acpt, acpu, acpv, acq,
acqb, acqc, acqd, acqe, acqf, acqg, acqh, acqi, acqj, acql, acqm, acqn, acqp, acqq, acqr, acqs, acqt, acqu,
acqv, acr, acrb, acrc, acrd, acre, acrf, acrg, acrh, acri, acrj, acrl, acrm, acrn, aerp, acrq, acrr, aers, acrt,
acru, acrv, acs, acsb, acsc, acsd, acse, acsf, acsg, acsh, acsi, acsk, acsl, acsm, acsn, acso, acsq, acsr, acst,
acsu, acsv, act, actb, actc, actd, acte, actf, actg, acth, acti, actj, actl, actm, actp, actr, acts, actt, actu, acu,
acub, acuc, acut, acuu, acv, acvb, acvc, acvd, acve, acvf, acvg, acvh, acvi, acvk, acvl, acvm, acvn, acvp,
acvq, acvr, acvs, acvt, acvu, acvv, adaf, adah, adai, adaj, adal, adam, adan, adap, adaq, adar, adat, adau,
adav, adbc, adbd, adbf, adbg, adbh, adbi, adbj, adbk, adbl, adbm, adbn, adbo, adbp, adbq, adbr, adbs,
adbt, adbu, adbv, adc, adcb, adcc, adcd, adce, adcf, adcg, adch, adci, adck, adcl, adcm, adcn, adco, adcp,
adcq, adcr, adcs, adct, adcv, addc, addf, addg, addh, addj, addk, addl, addm, addn, addo, addp, addq,
addr, addt, addv, adec, adee, adef, adeg, adeh, adei, adej, adek, adel, adem, aden, adeo, adep, adeq, ader,
ades, adet, adeu, adev, adf, adfb, adfc, adfd, adfe, adff, adfg, adfh, adfi, adfj, adfk, adfl, adfm, adfn, adfp,
adfq, adfr, adfs, adft, adfu, adfv, adgb, adgc, adgd, adge, adgf, adgg, adgh, adgi, adgj, adgk, adgl, adgm,
adgo, adgp, adgq, adgr, adgs, adgt, adgu, adgv, adh, adhb, adhc, adhd, adhe, adhf, adbg, adhh, adhi, adhk,
adhl, adhm, adhn, adho, adhp, adhq, adhr, adhs, adht, adhv, adi, adib, adic, adid, adie, adif, adig, adih,
adii, adik, adil, adim, adin, adio, adip, adiq, adir, adis, adit, adiv, adj, adjb, adjc, adje, adjf, adjg, adjh,
adjj, adjm, adjp, adjr, adjs, adjt, adju, adjv, adk, adkb, adkc, adkd, adke, adkg, adkh, adki, adkj, adkk,
adkl, adkp, adkq, adkr, adkt, adkv, adl, adlb, adlc, adld, adle, adlf, adlg, adlj, adlk, adll, adln, adlp, adlq,
adlr, adlt, adlu, adlv, admb, admc, admd, adme, admf, admg, admj, adml, admm, admq, admr, adn, adnb,
adnc, adne, adnf, adnj, adnm, adnr, adnt, ado, adob, adoe, adof, adog, adoj, adok, adoq, ador, adot, adou,
adov, adp, adpb, adpc, adpd, adpe, adpf, adpg, adpj, adpk, adpl, adpm, adpn, adpo, adpp, adpq, adpr,
adpt, adpv, adq, adqb, adqc, adqd, adqe, adqf, adqg, adqh, adqi, adqj, adqk, adql, adqm, adqn, adqp,
adqq, adqr, adqs, adqt, adqu, adqv, adr, adrb, adrc, adrd, adre, adrf, adrg, adrh, adri, adrj, adrk, adrl,
adrm, adrn, adro, adrp, adrq, adrr, adrs, adrt, adru, adrv, ads, adsb, adsc, adsd, adse, adsg, adsi, adsl, adso,
adsp, adsq, adsr, adst, adt, adtb, adtc, adtd, adte, adtf, adtg, adth, adti, adtj, adtk, adtl, adtm, adtn, adtp,
adtq, adtr, adts, adtt, adtu, adtv, adu, adub, adud, adue, aduh, aduk, adul, adup, aduq, adur, adut, adv,
advb, advc, advd, adve, advf, advg, advh, advi, advj, advk, advl, advm, advn, advo, advp, advq, advr,
advs, advt, advu, advv, aeah, aeai, aeaj, aeak, aeal, aeam, aean, aeao, aeap, aeaq, aear, aeas, aeat, aeau,
aeav, aebb, aebc, aebd, aebf, aebh, aebi, aebj, aebk, aebl, aebm, aebn, aebo, aebp, aebq, aebr, aebs, aebt,
aebu, aebv, aec, aecb, aecc, aecd, aece, aecf, aecg, aeci, aecj, aeck, aecl, aecm, aecn, aecp, aecq, aecr,
aecs, aect, aecv, aedb, aedc, aedf, aedh, aedi, aedj, aedk, aedl, aedm, aedn, aedp, aedq, aedr, aedt, aedu,
aedv, aeeb, aeec, aeed, aeee, aeef, aeeg, aehh, aeei, aeej, aeek, aeel, aeem, aeen, aeeo, aeep, aeeq, aeer,
aees, aeet, aeeu, aeev, aef, aefb, aefe, aefd, aefe, aeff, aefg, aefh, aefi, aefj, aefk, aefl, aefm, aefn, aefo,
aefp, aefq, aefr, aefs, aeft, aefu, aefv, aegb, aegc, aegd, aege, aegf, aegg, aegh, aegi, aegj, aegk, aegl,
aegm, aegn, aego, aegp, aegq, aegr, aegs, aegt, aegu, aegv, aeh, aehb, aehc, aehd, aehe, aehf, aehg, achh,
aehi, aehj, aehk, aehl, aehm, aehn, aeho, aehp, aehq, aehr, aehs, aeht, aehu, aehv, aei, aeib, aeic, aeid,
aeie, aeif, aeig, aeih, aeii, aeik, aeil, aeim, aein, aeio, aeip, aeiq, aeir, aeis, aeit, aeiv, aej, aejb, aejc, aejd,
aeje, aejf, aejh, aeji, aejj, aejp, aejq, aejr, aejs, aejt, aeju, aejv, aek, aekb, aekc, aekd, aeke, aekf, aekg,
aekh, aeki, aekj, aekk, aekl, aekm, aekn, aeko, aekp, aekq, aekr, aeks, aekt, aeku, aekv, ael, aelb, aelc,
aeld, aele, aelf, aelg, aelh, aeli, aelj, aelk, aell, aelm, aeln, aelp, aelq, aelr, aelt, aelu, aelv, aem, aemb,
aemd, aeme, aemf, aemg, aemi, aemj, aeml, aemm, aemn, aemo, aemp, aemq, aemr, aems, aemt, aemv,
aen, aenb, aenc, awne, aenf, aeng, aenh, aeni, aenj, aenm, aenp, aenq, aenr, aens, aent, aenu, aenv, aeo,
aeob, aeod, aeoe, aeof, aeog, aeoh, aeoi, aeoj, aeok, aeol, aeom, aeon, aeoq, aeor, aeos, aeot, aeov, aep,
aepb, aepc, aepd, aepe, aepf, aepg, aeph, aepi, aepj, aepk, aepl, aepm, aepn, aepo, aepp, aepq, aepr, aeps,
aept, aepu, aepv, aeq, aeqb, aeqc, aeqd, aeqe, aeqf, aeqg, aeqh, aeqi, aeqj, aeqk, aeql, aeqm, aeqn, aeqo,
aeqp, aeqq, aeqr, aeqs, aeqt, aeqv, aer, aerb, aerc, aerd, aere, aerf, aerg, aerh, aeri, aerj, aerk, aerl, aerm,
aern, aero, aerp, aerq, aerr, aers, aert, aerv, aes, aesb, aesc, aesd, aese, aesf, aesg, aesi, aesj, aesk, aesl,
aesm, aesn, aeso, aesp, aesq, aesr, aess, aest, aesv, aet, aetb, aetc, aetd, aete, aetf, aetg, aeth, aeti, aetj,
aetk, aetl, aetm, aetn, aeto, aetp, aetq, aetr, aett, aetv, aeu, aeub, aeud, aeue, acuf, acug, aeuh, aeui, aeuj,
aeuk, aeul, aeum, aeun, aeup, aeuq, aeur, aeut, aeuv, aev, aevb, aevc, aevd, aeve, aevf, aevg, aevh, aevi,
aevj, aevk, aevl, aevm, aevn, aevo, aevp, aevq, aevr, aevs, aevt, aevv, afah, afai, afaj, afak, afal, afam,
afan, afap, afaq, afar, afas, afat, afau, afav, afb, afbb, afbc, afbd, afbe, afbf, afbg, afbh, afbi, afbj, afbk,
afbl, afbm, afbn, afbo, afbp, afbq, afbr, afbs, afbt, afbu, afbv, afc, afcb, afcc, afcd, afce, afcf, afcg, afch,
afci, afcj, afck, afcl, afcm, afcn, afcp, afcq, afcr, afcs, afct, afcu, afcv, afd, afdb, afdc, afdd, afde, afdf,
afdg, afdh, afdi, afdj, afdk, afdl, afdm, afdn, afdp, afdq, afdr, afdt, afdv, afe, afeb, afed, afee, afef, afeg,
afeh, afei, afej, afek, afel, afem, afen, afeo, afep, afeq, afer, afes, afet, afeu, afev, aff, affb, affc, affd, affe,
afff, affg, affh, affi, affj, affk, affl, affm, affn, affp, affq, affr, affs, afft, affu, affv, afg, afgb, afgc, afgd,
afge, afgf, afgg, afgh, afgi, afgj, afgk, afgl, afgm, afgn, afgo, afgp, afgq, afgr, afgs, afgt, afgu, afgv, afh,
afhb, afhd, afhe, afhf, afhg, afhi, afhj, afhl, afhn, afho, afhp, afhq, afhr, afhs, afht, afhu, afhv, afi, afib,
afic, afid, afie, afif, afig, afii, afik, afil, afim, afin, afip, afiq, afir, afis, afit, afiv, afj, afjb, afjc, afjd, afje,
afjf, afjg, afjh, afji, afjj, afjm, afjp, afjr, afjs, afjt, afju, afjv, afk, afkb, afkc, afkd, afke, afkf, afkg, afkh,
afki, afkj, afkl, afkm, afkn, afkp, afkq, afkr, afkt, afkv, afl, aflb, afld, afle, aflf, aflg, aflj, aflk, afln, aflq,
aflr, aflt, aflv, afm, afmb, afmd, afme, afmf, afmg, afmh, afmj, afml, afmm, afmo, afmp, afmq, afmr,
afms, afmt, afn, afnf, afo, afp, afpb, afpc, afpd, afpe, afpf, afpg, afph, afpi, afpj, afpk, afpl, afpm, afpn,
afpo, afpp, afpq, afpr, afpt, afpv, afq, afqb, afqc, afqd, afqe, afqf, afqg, afqh, afqi, afqj, afqk, afql, afqm,
afqn, afqp, afqq, afqr, afqt, afqv, afr, afrb, afrc, afrd, afre, afrf, afrg, afrh, afri, afrj, afrk, afrl, afrm, afrn,
afro, afrp, afrq, afrr, afrs, afrt, afru, afrv, afs, afsb, afsd, afse, afsf, afsg, afsi, afsk, afsl, afsm, afsn, afso,
afsp, afsq, afsr, afst, afsu, afsv, aft, aftb, aftc, aftd, afte, aftf, aftg, afth, afti, aftj, aftk, aftl, aftm, aftn, aftp,
aftq, aftr, afts, aftt, aftu, aftv, afu, afub, afuc, afud, afue, afuf, afug, afuh, afui, afuj, afuk, aful, afum,
afun, afup, afuq, afur, afut, afuv, afv, afvb, afvc, afvd, afve, afvf, afvg, afvh, afvi, afvj, afvk, afvl, afvm,
afvn, afvo, afvp, afvq, afvr, afvt, afvv, agah, agai, agaj, agak, agal, agam, agan, agao, agap, agaq, agar,
agas, agat, agau, agav, agbc, agbf, agbg, agbh, agbi, agbj, agbk, agbl, agbm, agbn, agbo, agbp, agbq,
agbr, agbs, agbt, agbu, agbv, agc, agcb, agcc, agcd, agce, agcf, agcg, agch, agci, agcj, agck, agcl, agcm,
agcn, agcp, agcq, agcr, agcs, agct, agcu, agcv, agdc, agdf, agdh, agdi, agdj, agdk, agdl, agdm, agdn, agdp,
agdq, agdr, agds, agdt, agdu, agdv, agec, aged, agef, ageh, agei, agej, agek, agel, agem, agen, ageo, agep,
ageq, ager, ages, aget, ageu, agev, agf, agfb, agfc, agfd, agff, agfg, agfh, agfi, agfj, agfk, agfl, agfm, agfn,
agfp, agfq, agfr, agfs, agft, agfu, agfv, aggb, aggc, aggf, aggg, aggh, aggi, aggj, aggk, aggl, aggm, aggn,
aggo, aggp, aggq, aggr, aggs, aggt, aggu, aggv, agh, aghb, aghc, aghd, aghe, aghf, aghg, aghi, aghk, aghl,
aghm, aghn, agho, aghp, aghq, aghr, aghs, aght, aghu, aghv, agi, agib, agic, agid, agie, agif, agig, agih,
agii, agik, agil, agim, agin, agio, agip, agiq, agir, agis, agit, agiv, agj, agjb, agjc, agje, agjf, agjg, agjh,
agji, agjj, agjm, agjp, agjr, agjs, agjt, agju, agjv, agk, agkb, agkc, agkd, agke, agkf, agkg, agkh, agki,
agkj, agkk, agkl, agkm, agkn, agkp, agkq, agkr, agkt, agku, agkv, agl, aglb, aglc, agld, agle, aglf, aglg,
aglh, agli, aglj, aglk, agll, aglm, agln, aglp, aglq, aglr, agls, aglt, aglu, aglv, agm, agmb, agmc, agmd,
agme, agmf, agmg, agmh, agmi, agmj, agmk, agml, agmm, agmn, agmo, agmp, agmq, agmr, agms, agmt,
agmv, agn, agnb, agnc, agne, agnf, agng, agnh, agni, agnj, agnm, agnp, agnq, agnr, agns, agnt, agnu,
agnv, ago, agob, agoc, agoe, agof, agog, agoh, agoi, agoj, agom, agop, agor, agos, agot, agou, agov, agpb,
agpc, agpd, agpe, agpf, agpg, agph, agpj, agpk, agpl, agpm, agpn, agpo, agpp, agpq, agpr, agps, agpt,
agpu, agpv, agq, agqb, agqc, agqd, agqe, agqf, agqg, agqh, agqi, agqj, agqk, agql, agqm, agqn, agqo,
agqp, agqq, agqr, agqs, agqt, agqu, agqv, agr, agrb, agrc, agrd, agre, agrf, agrg, agrh, agri, agrj, agrk,
agrl, agrm, agrn, agro, agrp, agrq, agrr, agrs, agrt, agru, agrv, ags, agsb, agsc, agsd, agse, agsf, agsg, agsh,
agsk, agsl, agsm, agsn, agso, agsp, agsq, agsr, agss, agst, agsv, agt, agtb, agtc, agtd, agte, agtf, agtg, agth,
agti, agtj, agtk, agtl, agtm, agtn, agto, agtp, agtq, agtr, agts, agtt, agtu, agtv, agu, agub, aguc, agud, ague,
aguf, agug, aguh, agui, aguj, aguk, agul, agum, agun, agup, aguq, agur, agut, aguu, aguv, agv, agvb, agvc,
agvd, agve, agvf, agvg, agvh, agvi, agvj, agvk, agvl, agvm, agvn, agvo, agvp, agvq, agvr, agvs, agvt,
agvv, ahah, ahai, ahak, aham, ahap, ahaq, ahar, ahat, ahau, ahav, ahb, ahbb, ahbc, ahbd, ahbe, ahbf, ahbg,
ahbh, ahbi, ahbk, abbl, ahbm, ahbp, ahbq, ahbr, ahbs, ahbt, ahbu, ahbv, ahc, ahcb, ahcc, ahcd, ahce, ahcf,
ahcg, ahcj, ahck, ahcm, ahcp, ahcq, ahcr, ahcs, ahct, ahcv, ahd, abdb, ahdc, ahdd, ahde, ahdf, ahdg, abdh,
abdi, ahdj, ahdk, ahdl, ahdm, ahdp, ahdq, ahdr, ahds, ahdt, ahdu, ahdv, ahe, aheb, ahec, ahed, ahee, ahef,
aheg, aheh, ahei, ahej, ahek, ahel, ahem, ahep, aheq, aher, ahes, ahet, aheu, ahev, ahf, ahfb, ahfc, ahfe,
ahff, ahfg, ahfh, ahfi, ahfj, ahfm, ahfp, ahfr, ahfs, ahft, ahfu, ahg, ahgb, ahgc, ahgd, ahge, ahgf, ahgg,
ahgh, ahgi, ahgj, ahgk, ahgl, ahgm, ahgn, ahgp, ahgq, ahgr, ahgs, ahgt, ahgu, ahgv, ahh, ahhb, ahhd,
ahhe, ahhf, ahhg, ahhi, ahhk, ahhl, ahhm, ahhp, ahhq, ahhr, ahht, ahhu, ahhv, ahi, ahib, ahic, ahid, ahie,
ahif, ahig, ahih, ahii, ahik, ahil, ahim, ahin, ahip, ahiq, ahir, ahis, ahit, ahiv, ahj, ahjf, ahjj, ahk, ahkb,
ahkc, ahkd, ahke, ahkf, ahkg, ahkh, ahki, ahkj, ahkl, ahkm, ahkp, ahkq, ahkr, ahkt, ahku, ahkv, ahl, ahlb,
ahlc, ahld, ahle, ahlf, ahlg, ahlh, ahli, ahlj, ahlk, ahll, ahlm, ahln, ahlp, ahlq, ahlr, ahls, ahlt, ahlu, ahlv,
ahm, ahmb, ahmc, abmd, ahme, abmf, ahmg, ahmi, ahml, abmm, abmn, ahmt, abn, ahnc, abnf, ahng,
ahnj, ahnm, ahns, ahnt, aho, ahob, ahod, ahoe, ahof, ahog, ahoh, ahoi, ahoj, ahom, ahop, ahoq, ahor,
ahos, ahot, ahou, ahov, ahp, ahpb, ahpc, ahpd, ahpe, ahpf, ahpg, ahph, ahpi, ahpj, ahpk, ahpl, ahpm, ahpn,
ahpp, ahpq, ahpr, ahps, ahpt, ahpu, ahpv, ahq, ahqb, ahqc, ahqd, ahqe, ahqf, ahqg, ahqh, ahqi, ahqj, ahqk,
ahql, ahqm, ahqn, ahqp, ahqq, ahqr, ahqs, ahqt, ahqu, ahqv, ahr, ahrb, ahrc, ahrd, ahre, ahrf, ahrg, ahrh,
ahri, ahrj, abrk, ahrl, abrm, ahrp, ahrq, ahrr, abrs, ahrt, ahru, abrv, ahs, ahsb, ahsd, ahse, absg, absb, ahsl,
absm, ahsn, ahsp, ahsq, ahsr, ahst, aht, ahtb, ahtc, ahtd, ahte, ahtf, ahtg, ahth, ahti, ahtj, ahtl, ahtm, ahtp,
ahtq, ahtr, ahts, ahtt, ahtu, ahtv, ahu, ahub, ahuc, ahue, ahuf, ahug, ahuj, ahup, ahur, ahus, ahut, ahv,
ahvb, ahvc, ahvd, ahve, ahvf, ahvg, ahvh, ahvi, ahvk, ahvl, ahvm, ahvn, ahvp, ahvq, ahvr, ahvt, ahvu,
ahvv, aiai, aiaj, aiak, aiam, aian, aiap, aiaq, aiar, aias, aiat, aiau, aiav, aib, aibb, aibc, aibd, aibe, aibf,
aibg, aibh, aibi, aibj, aibk, aibl, aibm, aibn, aibp, aibq, aibr, aibs, aibt, aibu, aibv, aic, aicb, aicc, aice,
aicf, aicg, aici, aicl, aicq, aicr, aict, aicv, aid, aidb, aidc, aidd, aide, aidf, aidg, aidh, aidi, aidj, aidk, aidl,
aidm, aidn, aidp, aidq, aidr, aids, aidt, aidu, aidv, aie, aieb, aiec, aied, aiee, aief, aieg, aieh, aiei, aiej, aiek,
aiem, aien, aiep, aieq, aier, aies, aiet, aieu, aiev, aif, aifb, aifd, aife, aiff, aifh, aifi, aifj, aifm, aifr, aifs,
aift, aifu, aig, aigb, aigc, aigd, aige, aigf, aigg, aigh, aigi, aigj, aigk, aigl, aigm, aign, aigp, aigq, aigr, aigs,
aigt, aigu, aigv, aih, aihb, aihc, aihd, aihe, aihf, aihg, aihh, aihi, aihk, aihl, aihm, aihq, aihr, aihs, aiht,
aihu, aii, aiib, aiic, aiid, aiie, aiif, aiig, aiih, aiii, aiik, aiil, aiin, aiip, aiiq, aiir, aiit, aiiv, aij, aik, aikb, aikc,
aikd, aike, aikf, aikg, aikh, aiki, aikj, aikk, aikm, aikp, aikq, aikr, aiks, aikt, aiku, aikv, ail, ailb, ailc, aild,
aile, ailf, ailg, ailh, aili, ailj, ailk, aill, ailm, ailn, ailp, ailq, ailr, ails, ailt, ailu, ailv, aim, aimb, aimc, aimd,
aime, aimf, aimg, aimh, aimi, aimj, aiml, aimm, aimq, aimr, aimt, ain, ainc, aine, ainf, aing, ainh, ainj,
ainm, ains, aint, aio, aiob, aiod, aioe, aiof, aioh, aioi, aioj, aiop, aioq, aior, aiot, aiou, aiov, aip, aipb, aipc,
aipd, aipe, aipf, aipg, aiph, aipi, aipj, aipk, aipl, aipm, aipn, aipp, aipq, aipr, aips, aipt, aipu, aipv, aiq,
aiqb, aiqc, aiqd, aiqe, aiqf, aiqg, aiqh, aiqi, aiqj, aiqk, aiql, aiqm, aiqn, aiqp, aiqq, aiqr, aiqs, aiqt, aiqu,
aiqv, air, airb, airc, aird, aire, airf, airg, airh, airi, airj, airk, airl, airm, airn, airp, airq, airr, airs, airt, airu,
airv, ais, aisb, aisc, aisd, aise, aisf, aisg, aish, aisi, aisk, aisl, aism, aisn, aisq, aisr, aiss, aist, aisu, aisv, ait,
aitb, aitc, aitd, aite, aitf, aitg, aith, aiti, aitj, aitl, aitm, aitp, aitq, aitr, aitt, aitu, aitv, aiu, aiv, aivb, aivc,
aivd, aive, aivf, aivg, aivh, aivi, aivk, aivl, aivm, aivn, aivp, aivq, aivr, aivs, aivt, aivu, aivv, ajaj, ajak,
ajam, ajan, ajap, ajaq, ajar, ajat, ajav, ajb, ajbb, ajbc, ajbd, ajbe, ajbf, ajbg, ajbi, ajbj, ajbk, ajbl, ajbm,
ajbn, ajbq, ajbr, ajbs, ajbt, ajbu, ajbv, ajcb, ajcd, ajcf, ajcg, ajcj, ajck, ajcn, ajcp, ajcq, ajcr, ajct, ajcv, ajd,
ajdb, ajdd, ajde, ajdn, ajdq, ajdv, aje, ajed, ajee, ajef, ajeg, ajek, ajel, ajen, ajeq, ajer, ajet, ajev, ajf, ajfb,
ajfc, ajfd, ajfe, ajff, ajfg, ajfh, ajfi, ajfj, ajfl, ajfm, ajfn, ajfp, ajfq, ajfr, ajft, ajfu, ajfv, ajg, ajgb, ajgd, ajge,
ajgf, ajgg, ajgj, ajgl, ajgn, ajgo, ajgq, ajgr, ajgt, ajgv, ajh, ajhb, ajhd, ajhe, ajhf, ajhg, ajhk, ajhl, ajhn,
ajho, ajhq, ajhr, ajht, ajhv, aji, ajid, ajie, ajig, ajik, ajil, ajin, ajip, ajiq, ajir, ajit, ajiv, ajjb, ajje, ajjf, ajjg,
ajji, ajjj, ajjm, ajjs, ajk, ajke, ajkq, ajkv, ajl, ajlq, ajm, ajmb, ajme, ajmf, ajmg, ajmh, ajmj, ajml, ajmn,
ajmo, ajmq, ajmr, ajmt, ajmv, ajn, ajp, ajpd, ajpe, ajpf, ajpg, ajpk, ajpl, ajpn, ajpo, ajpq, ajpr, ajpt, ajpv,
ajq, ajqd, ajqe, ajqj, ajqk, ajql, ajqn, ajqq, ajqt, ajqv, ajr, ajrb, ajrd, ajre, ajrf, ajrg, ajrh, ajri, ajrj, ajrk, ajrl,
ajrm, ajrn, ajrp, ajrq, ajrr, ajrt, ajrv, ajs, ajsb, ajsd, ajse, ajsf, ajsg, ajsk, ajsl, ajsn, ajsq, ajsr, ajst, ajt, ajtb,
ajtd, ajte, ajtf, ajtg, ajth, ajti, ajtj, ajtk, ajtl, ajtm, ajtn, ajtq, ajtr, ajtt, ajtv, ajub, ajud, ajue, ajuf, ajug, ajuh,
ajui, ajuk, ajul, ajum, ajun, ajuq, ajur, ajut, ajuv, ajv, ajvd, ajve, ajvf, ajvg, ajvk, ajvl, ajvn, ajvq, ajvr,
ajvt, ajvv, akak, akal, akam, akan, akap, akaq, akar, akas, akat, akau, akav, akb, akbb, akbc, akbd, akbe,
akbf, akbg, akbh, akbi, akbj, akbk, akbl, akbm, akbn, akbo, akbp, akbq, akbr, akbs, akbt, akbu, akbv,
akc, akcb, akcd, akce, akcf, akcg, akch, akci, akcj, akck, akcl, akcm, akcp, akcq, akcr, akcs, akct, akcv,
akd, akdb, akdc, akdd, akde, akdf, akdg, akdj, akdk, akdl, akdp, akdq, akdr, akdt, akdv, ake, akeb, akec,
aked, akee, akef, akeg, akeh, akei, akej, akek, akel, akem, aken, akeo, akep, akeq, aker, akes, aket, akeu,
akev, akf, akfb, akfc, akfd, akff, akfg, akfh, akfi, akfj, akfk, akfm, akfp, akfq, akfr, akfs, akft, akfu, akfv,
akg, akgb, akgc, akgd, akge, akgf, akgg, akgh, akgi, akgj, akgk, akgl, akgm, akgn, akgo, akgp, akgq,
akgr, akgs, akgt, akgu, akgv, akh, akhb, akhc, akhd, akhe, akhf, akhg, akhh, akhi, akhj, akhk, akhl, akhm,
akhn, akho, akbp, akhq, akhr, akht, akhv, aki, akib, akic, akid, akie, akif, akig, akih, akii, akik, akil, akim,
akin, akio, akip, akiq, akir, akis, akit, akiv, akj, akjb, akjc, akjd, akje, akjf, akig, akjh, akji, akjk, akjm,
akjp, akjr, akjs, akjt, akju, akjv, akk, akkb, akkd, akke, akkf, akkg, akki, akkk, akkl, akkm, akkn, akkp,
akkq, akkr, akkt, akku, akkv, akl, aklb, akld, akle, aklf, aklg, aklj, akik, akll, akln, aklp, aklq, aklr, aklt,
aklu, aklv, akm, akmb, akmd, akme, akmf, akmg, akmh, akmj, akmk, akml, akmm, akmn, akmo, akmp,
akmq, akmr, akms, akmt, akn, aknb, aknf, aknj, aknu, ako, akp, akpb, akpc, akpd, akpe, akpf, akpg, akph,
akpj, akpk, akpl, akpm, akpn, akpo, akpp, akpq, akpr, akpt, akpv, akq, akqb, akqc, akqd, akge, akqf,
akqg, akqh, akqi, akqj, akqk, akql, akqm, akqn, akqo, akqp, akqq, akqr, akqs, akqt, akqu, akqv, akr, akrb,
akrc, akrd, akre, akrf, akrg, akrh, akri, akrj, akrk, akrl, akrm, akrn, akro, akrp, akrq, akrr, akrt, akru, akrv,
aks, aksb, aksc, aksd, akse, aksg, aksi, akso, aksp, aksq, aksr, akst, akt, aktb, aktc, aktd, akte, aktf, aktg,
akth, akti, aktj, aktk, aktl, aktm, aktn, akto, aktp, aktq, aktr, akts, aktt, aktu, aktv, aku, akub, akuc, akud,
akue, akuf, akug, akuh, akul, akuo, akuq, akur, akut, akuv, akv, akvb, akvc, akvd, akve, akvf, akvg, akvh,
akvi, akvk, akvl, akvm, akvn, akvo, akvp, akvq, akvr, akvt, akvv, alal, alam, alan, alao, alap, alaq, alar,
alas, alat, alav, alb, albb, albc, albd, albe, albf, albg, albh, albi, albj, albk, albl, albm, albn, albo, albp,
albq, albr, albs, albt, albu, albv, alc, alcb, alcd, alce, alef, aleg, alck, alcl, alcm, alcn, alco, alcp, alcq, alcr,
alcs, alct, alcv, ald, aldb, alde, aldd, alde, aldf, aldg, aldj, aldk, aldl, aldn, aldo, aldp, aldq, aldr, alds, aldt,
aldv, ale, aleb, alec, aled, alee, alef, aleg, aleh, alei, alej, alek, alel, alem, alen, aleo, alep, aleq, aler, ales,
alet, aleu, alev, alf, alfb, alfc, alfd, alfe, alff, alfg, alfh, alfi, alfj, alfk, alfl, alfm, alfn, alfp, alfq, alfr, alfs,
alft, alfu, alfv, alg, algb, algc, algd, alge, algf, algg, algh, algi, algj, algk, algl, algm, algn, algo, algp,
algq, algr, algs, algt, algu, algv, alh, albb, alhc, alhd, alhe, albf, alhg, alhi, albk, alhl, alhm, alhn, alho,
alhp, alhq, alhr, alhs, alht, alhv, ali, alib, alic, alid, alie, alif, alig, alih, alii, alik, alil, alim, alin, alio, alip,
aliq, alir, alis, alit, aliv, alj, aljb, aljc, aljd, alje, aljf, aljg, aljh, alji, aljj, aljk, alim, aljp, aljq, aljr, aljs, aljt,
alju, aljv, alk, alkb, alkc, alkd, alke, alkf, alkg, alkh, alki, alkk, alkl, alkm, alkn, alkp, alkq, alkr, alkt,
alkv, all, allb, allc, alld, alle, allf, allg, allh, alli, allj, allk, alll, alln, allp, allq, allr, allt, allu, allv, alm,
almb, almc, almd, alme, almf, almg, almh, almi, almk, alml, almm, almp, almr, alms, almt, almv, aln,
alnb, alnc, alnd, alne, alnf, alng, alnh, alni, alnj, alnm, alnp, alnq, alnr, alns, alnt, alnu, alnv, alo, alod,
aloe, alok, alog, alor, alot, alp, alpb, alpc, alpd, alpe, alpf, alpg, alph, alpi, alpj, alpk, alpl, alpm, alpn,
alpo, alpp, alpq, alpr, alpt, alpv, alq, alqb, alqc, alqd, alqe, alqf, alqg, alqh, alqi, alqj, alqk, alql, alqm,
alqn, alqo, alqp, alqq, alqr, alqt, alqu, alqv, alr, alrb, alrc, alrd, alre, alrf, alrg, alrh, alri, alrj, alrk, alrl,
alrm, alrn, alro, alrp, alrq, alrr, alrt, alru, alrv, als, alsb, alsc, alse, alsg, alsi, alsl, also, alsp, alsq, alsr, alst,
alsv, alt, altb, altc, altd, alte, altf, altg, alth, alti, altj, altk, altl, altm, altn, alto, altp, altq, altr, altt, altu,
altv, alu, alub, alud, alue, aluf, alug, aluk, alul, aluo, alup, aluq, alur, alut, aluv, alv, alvb, alve, alvd, alve,
alvf, alvg, alvh, alvi, alvj, alvk, alvl, alvm, alvn, alvo, alvp, alvq, alvr, alvs, alvt, alvv, amam, amao,
amap, amaq, amar, amas, amat, amau, amav, ambb, ambc, ambd, ambe, ambf, ambg, ambh, ambi, ambj,
ambk, ambl, ambm, ambn, ambo, ambp, ambq, ambr, ambs, ambt, ambu, ambv, amc, amcb, amcc, amce,
amcf, amcg, amch, amci, amcl, amcm, amcp, amcq, amcr, amct, amcv, amd, amdb, amdc, amdf, amdg,
amdh, amdi, amdm, amdr, amds, amdt, amdu, amdv, ameb, amec, amce, amef, ameg, ameh, amei, amem,
amep, ameq, amer, ames, amet, amev, amf, amfb, amfc, amfd, amfe, amff, amfg, amfh, amfi, amfj, amfk,
amfm, amfp, amfq, amfr, amfs, amft, amfu, amfv, amgb, amgc, amgd, amge, amgf, amgg, amgh, amgi,
amgj, amgk, amgm, amgp, amgq, amgr, amgs, amgt, amgu, amgv, amh, amhb, amhc, amhd, amhg, amhi,
amhm, amhp, ambr, amht, amhv, ami, amib, amic, amig, amih, amii, amik, amim, amip, amiq, amir,
amis, amit, amiv, amj, amjb, amjc, amjf, amjg, amjh, amjj, amjr, amjt, amju, amk, amkf, amkh, amkm,
aml, amlb, amlc, amld, amle, amlf, amlg, amlh, amli, amlj, amlk, amlm, amlp, amlq, amlr, amls, amlt,
amlu, amlv, amm, ammb, ammc, amn, amnc, amng, amnm, amns, amo, amob, amoc, amoe, amof, amog,
amoh, amoi, amoj, amom, amop, amoq, amor, amos, amot, amou, amov, amp, ampb, ampc, ampd, ampe,
ampf, amph, ampj, ampk, ampm, ampp, ampq, ampr, ampt, ampv, amq, amqb, amqc, amqd, amqf, amqg,
amqh, amqm, amqs, amqt, amr, amrb, amrc, amrd, amre, amrf, amrg, amrh, amri, amrj, amrm, amrn,
amrp, amrq, amrr, amrs, amrt, amru, amrv, ams, amsb, amsg, amsi, amsp, amsr, amst, amt, amtc, amtf,
amtj, amts, amu, amv, amvb, amve, amvg, amvh, amvk, amvm, amvq, amvs, amvt, anan, anap, anaq,
anar, anat, anav, anb, anbb, anbc, anbd, anbe, anbf, anbg, anbh, anbi, anbj, anbk, anbl, anbm, anbn, anbo,
anbp, anbq, anbr, anbt, anbv, anc, ancb, ancd, ance, ancf, ancj, anck, ancn, ancp, ancq, ancv, and, ande,
andk, andq, andv, ane, aned, anee, anef, aneg, anej, anek, anel, anen, aneo, aneq, aner, anet, anev, anf,
anfb, anfc, anfd, anfe, anff, anfg, anfh, anfi, anfj, anfk, anfl, anfm, anfn, anfp, anfq, anfr, anfs, anft, anfv,
ang, angd, ange, angg, angj, angl, angn, ango, angq, angr, angs, angt, angv, anh, anhb, anhd, anhe, anhf,
anhg, anhj, anhk, anhl, anhn, anho, anhq, anhr, anht, anhv, ani, anie, anig, anik, anil, anin, anip, aniq,
anir, aniv, anjb, anjc, anjd, anje, anjf, anjg, anjh, anji, anjj, anjk, anjm, anjp, anjq, anjr, anjs, anjt, anju,
anjv, ank, ankd, anke, ankk, ankq, ankt, ankv, anl, anle, anlq, anm, anmb, anmd, anme, anmf, anmg,
anmj, anml, anmm, anmo, anmq, anmr, anms, anmt, anmv, ann, ano, anp, anpd, anpe, anpf, anpg, anpj,
anpk, anpl, anpn, anpo, anpp, anpq, anpr, anpt, anpv, anq, anqe, anqf, anqg, anqk, anql, anqn, anqq, anqr,
anqt, anqv, anr, anrb, anrd, anre, anrf, anrg, anrk, anrl, anrn, anro, anrp, anrq, anrr, anrt, anrv, ans, ansb,
ansd, anse, ansf, ansg, ansl, ansm, ansn, anso, ansq, ansr, anst, ansv, ant, antb, antd, ante, antf, antg, antj,
antk, antl, antn, anto, antp, antq, antr, antt, antv, anub, anud, anue, anuf, anug, anuh, anuk, anul, anun,
anup, anuq, anur, anut, anuv, anv, anvd, anve, anvf, anvg, anvk, anvl, anvn, anvo, anvq, anvr, anvt, anvv,
aoao, aoap, aoaq, aoar, aoav, aob, aobe, aobf, aobg, aobl, aobn, aobo, aobq, aobr, aobt, aobv, aoc, aoce,
aocg, aocl, aoco, aocp, aocq, aocr, aocv, aod, aode, aodk, aodn, aodo, aodp, aodq, aodv, aoe, aoeb, avec,
aoed, aoee, aoef, aoeg, aoeh, aoei, aoej, aoek, aoel, aoem, aoen, aoeo, aoep, aoeq, aoer, aoet, aoeu, aoev,
aof, aofb, aofc, aofd, aofe, aoff, aofg, aofh, aofi, aofk, aofl, aofm, aofn, aofo, aofp, aofq, aofr, aofs, aoft,
aofu, aofv, aog, aoge, aogl, aogm, aogn, aogp, aogq, aogr, aogt, aogu, aogv, aoh, aohd, aohe, aohg, aohl,
aohn, aoho, aohp, aohq, aohr, aoht, aohv, aoi, aoib, aoid, aoie, aoif, aoig, aoik, aoil, aoin, aoio, aoip, aoiq,
aoir, aois, aoiv, aoj, aojb, aojc, aojd, aoje, aojf, aojg, aojh, aoji, aojm, aojn, aojp, aojq, aojr, aojt, aoju,
aojv, aok, aoke, aokf, aokk, aokl, aokn, aoko, aokq, aokr, aokt, aokv, aol, aold, aole, aoll, aoln, aolo,
aolq, aolr, aolv, aom, aomp, aomq, aomv, aon, aone, aonq, aonr, aont, aonv, aoo, aop, aopd, aope, aopf,
aopg, aopl, aopo, aopq, aopr, aopt, aopv, aoq, aoqb, aoqd, aoqe, aoqf, aoqg, aoqk, aoql, aoqn, aoqo, aoqp,
aoqq, aoqr, aoqt, aoqv, aor, aorb, aord, aore, aorf, aorg, aorj, aork, aorl, aorn, aoro, aorp, aorq, aorr, aort,
aorv, aos, aosb, aosi, aosq, aosr, aosv, aot, aotb, aotd, aote, aotf, aotg, aotk, aotl, aotn, aoto, aotp, aotq,
aotr, aott, aotv, aou, aoue, aouf, aoug, aouk, aoul, aouo, aouq, aour, aout, aouv, aov, aovd, aove, aovg,
aovk, aovl, aovn, aovo, aovq, aovr, aovs, aovt, aovv, apap, apaq, apar, apas, apat, apau, apav, apb, apbb,
apbc, apbd, apbe, apbf, apbg, apbh, apbi, apbj, apbk, apbl, apbm, apbn, apbo, apbp, apbq, apbr, apbs,
apbt, apbu, apbv, apc, apcb, apcc, apcd, apce, apcf, apcg, apch, apci, apck, apcl, apcm, apcn, apcp, apcq,
apcr, apcs, apct, apcv, apd, apdb, apdc, apdd, apde, apdf, apdg, apdh, apdi, apdj, apdk, apdl, apdm, apdn,
apdp, apdq, apdr, apds, apdt, apdv, ape, apeb, apec, aped, apee, apef, apeg, apeh, apei, apej, apek, apel,
apem, apen, apco, apep, apeq, aper, apes, apet, apeu, apev, apf, apfb, apfc, apfd, apfe, apff, apfg, apfh,
apfi, apfj, apfk, apfl, apfm, apfn, apfp, apfq, apfr, apfs, apft, apfu, apfv, apg, apgb, apgc, apgd, apge,
apgf, apgg, apgh, apgi, apgj, apgk, apgl, apgm, apgn, apgo, apgp, apgq, apgr, apgs, apgt, apgu, apgv,
aph, aphb, aphc, aphd, aphe, aphf, aphg, aphh, aphi, aphk, aphl, aphm, aphn, apho, aphp, aphq, aphr,
aphs, apht, aphv, api, apib, apic, apid, apie, apif, apig, apih, apii, apik, apil, apim, apin, apip, apiq, apir,
apis, apit, apiv, apj, apjb, apjc, apjd, apjf, apig, apjb, apjj, apjk, apjp, apjr, apjs, apjt, apju, apjv, apk,
apkb, apkc, apkd, apke, apkf, apkg, apkh, apki, apkk, apkl, apkm, apkn, apkp, apkq, apkr, apks, apkt,
apku, apkv, apl, aplb, aplc, apld, aple, aplf, aplg, aplh, apli, aplj, aplk, apll, aplm, apln, aplo, aplp, aplq,
aplr, apls, aplt, aplu, aplv, apm, apmb, apmc, apmf, apmg, apmi, apmo, apmp, apmq, apmr, apmt, apn,
apnb, apnc, apnd, apne, apnf, apng, apnh, apni, apnj, apnm, apnp, apnq, apnr, apns, apnt, apnu, apnv,
apo, apob, apod, apoe, apof, apog, apoh, apoi, apoj, apok, apol, apom, apon, apop, apoq, apor, apos, apot,
apou, apov, app, appb, appc, appd, appe, appf, appg, apph, appj, appk, appl, appm, appn, appo, appp,
appq, appr, apps, appt, appu, appv, apq, apqb, apqc, apqd, apqe, apqf, apqg, apqh, apqi, apqj, apqk, apql,
apqm, apqn, apqo, apqp, apqq, apqr, apqs, apqt, apqu, apqv, apr, aprb, aprc, aprd, apre, aprf, aprg, aprh,
apri, aprj, aprk, aprl, aprm, aprn, aprp, aprq, aprr, aprs, aprt, apru, aprv, aps, apsb, apsc, apse, apsg, apsi,
apsl, apsq, apsr, apt, aptb, aptc, aptd, apte, aptf, aptg, apth, apti, aptj, aptk, aptl, aptm, aptn, apto, aptp,
aptq, aptr, apts, aptt, aptu, aptv, apu, apub, apud, apue, apug, apuj, apun, apuo, apup, apuq, apur, aput,
apuv, apv, apvb, apvc, apvd, apve, apvf, apvg, apvh, apvi, apvj, apvk, apvl, apvm, apvn, apvo, apvp,
apvq, apvr, apvs, apvt, apvu, apvv, aqaq, aqar, aqas, aqat, aqau, aqav, aqb, aqbb, aqbc, aqbd, aqbe, aqbf,
aqbg, aqbh, aqbi, aqbj, aqbk, aqbl, aqbm, aqbn, aqbo, aqbp, aqbq, aqbr, aqbs, aqbt, aqbu, aqbv, aqc, aqcb,
aqcc, aqcd, aqce, aqcf, aqcg, aqch, aqci, aqcj, aqck, aqcl, aqcm, aqcn, aqco, aqcp, aqcq, aqcr, aqcs, aqct,
aqcv, aqd, aqdb, aqdc, aqdd, aqde, aqdf, aqdg, aqdh, aqdi, aqdj, aqdk, aqdl, aqdm, aqdn, aqdo, aqdp,
aqdq, aqdr, aqds, aqdt, aqdv, aqe, aqeb, aqec, aqed, aqee, aqef, aqeg, aqeh, aqei, aqej, aqek, aqel, aqem,
aqen, aqeo, aqep, aqeq, aqer, aqes, aqet, aqeu, aqev, aqf, aqfb, aqfc, aqfd, aqfe, aqff, aqfg, aqfh, aqfi,
aqfj, aqfk, aqfl, aqfm, aqfn, aqfp, aqfq, aqfr, aqfs, aqft, aqfu, aqfv, aqg, aqgb, aqgc, aqgd, aqge, aqgf,
aqgg, aqgh, aqgi, aqgj, aqgk, aqgl, aqgm, aqgn, aqgo, aqgp, aqgq, aqgr, aqgs, aqgt, aqgu, aqgv, aqh,
aqhb, aqhc, aqhd, aqhe, aqhf, aqhg, aqhh, aqhi, aqhj, aqhk, aqhl, aqhm, aqhn, aqho, aqhp, aqhq, aqhr,
aqhs, aqht, aqhv, aqi, aqib, aqic, aqid, aqie, aqif, aqig, aqih, aqii, aqij, aqik, aqil, aqim, aqin, aqio, aqip,
aqiq, aqir, aqis, aqit, aqiv, aqj, aqjb, aqjc, aqjd, aqje, aqjf, aqjg, aqjh, aqji, aqjj, aqjk, aqjl, aqjm, aqjp,
aqjq, aqjr, aqjs, aqjt, aqju, aqjv, aqk, aqkb, aqkc, aqkd, aqke, aqkf, aqkg, aqkh, aqki, aqkk, aqkl, aqkm,
aqkn, aqkp, aqkq, aqkr, aqks, aqkt, aqkv, aql, aqlb, aqlc, aqld, aqle, aqlf, aqlg, aqlh, aqli, aqlj, aqlk, aqll,
aqlm, aqln, aqlo, aqlp, aqlq, aqlr, aqls, aqlt, aqlu, aqlv, aqm, aqmb, aqmc, aqmd, aqme, aqmf, aqmg,
aqmh, aqmi, aqmk, aqml, aqmm, aqmn, aqmo, aqmp, aqmq, aqmr, aqmt, aqmv, aqn, aqnb, aqnd, aqne,
aqnf, aqng, aqnh, aqni, aqnj, aqnk, aqnm, aqnp, aqnq, aqnr, aqns, aqnt, aqnu, aqnv, aqo, aqod, aqoe, aqof,
aqog, aqoj, aqok, aqol, aqon, aqoq, aqor, aqot, aqov, aqp, aqpb, aqpc, aqpd, aqpe, aqpf, aqpg, aqph, aqpi,
aqpj, aqpk, aqpl, aqpm, aqpn, aqpo, aqpp, aqpq, aqpr, aqpt, aqpv, aqq, aqqb, aqqc, aqqd, aqqe, aqqf,
aqqg, aqqh, aqqi, aqqj, aqqk, aqql, aqqm, aqqn, aqqo, aqqp, aqqq, aqqr, aqqs, aqqt, aqqu, aqqv, aqr, aqrb,
aqrc, aqrd, aqre, aqrf, aqrg, aqrh, aqri, aqrj, aqrk, aqrl, aqrm, aqrn, aqro, aqrp, aqrq, aqrr, aqrt, aqru, aqrv,
aqs, aqsb, aqsc, aqsd, aqse, aqsf, aqsg, aqsh, aqsi, aqsj, aqsl, aqsm, aqso, aqsp, aqsr, aqst, aqsv, aqt, aqtb,
aqtc, aqtd, aqte, aqtf, aqtg, aqth, aqti, aqtj, aqtk, aqtl, aqtm, aqtn, aqto, aqtp, aqtq, aqtr, aqtt, aqtu, aqtv,
aqu, aqub, aquc, aqud, aque, aquf, aqug, aquh, aqui, aquj, aqun, aquo, aqup, aquq, aqur, aqut, aquv, aqv,
aqvb, aqvc, aqvd, aqve, aqvf, aqvg, aqvh, aqvi, aqvj, aqvk, aqvl, aqvm, aqvn, aqvo, aqvp, aqvq, aqvr,
aqvt, aqvu, aqvv, arar, aras, arat, arau, arav, arb, arbb, arbc, arbd, arbe, arbf, arbg, arbh, arbi, arbj, arbk,
arbl, arbm, arbn, arbo, arbp, arbq, arbr, arbs, arbt, arbu, arbv, arc, arcb, arcc, arcd, arce, arcf, arcg, arch,
arci, arcj, arck, arcl, arcm, arcn, arcp, arcq, arcr, arcs, arct, arcv, ard, ardb, ardc, ardd, arde, ardf, ardg,
ardj, ardk, ardl, ardm, ardn, ardp, ardq, ardr, ards, ardt, ardv, are, areb, arec, ared, aree, aref, areg, areh,
arei, arej, arek, arel, arem, aren, areo, arep, areq, arer, ares, aret, areu, arev, arf, arfb, arfc, arfd, arfe, arff,
arfg, arfh, arfi, arfj, arfk, arfl, arfm, arfn, arfp, arfq, arfr, arfs, arft, arfu, arfv, arg, argb, argc, argd, arge,
argf, argg, argh, argi, argj, argk, argl, argm, argo, argp, argq, argr, args, argt, argu, argv, arh, arhb, arhc,
arhd, arhe, arhf, arhg, arhh, arhi, arhj, arhk, arhl, arhm, arhn, arho, arhp, arhq, arhr, arhs, arht, arhv, ari,
arib, aric, arid, arie, arif, arig, arih, arii, arik, aril, arim, arin, arip, ariq, arir, aris, arit, ariv, arj, arjb, arjc,
arjd, arje, arjf, arjg, arjh, arji, arjj, arjk, arjm, arjp, arjq, arjr, arjs, arjt, arju, arjv, ark, arkb, arkc, arkd,
arke, arkf, arkg, arkh, arki, arkk, arkl, arkm, arkn, arkp, arkq, arkr, arks, arkt, arku, arkv, arl, arlb, arlc,
arld, arle, arlf, arlg, arlh, arli, arlj, arlk, arll, arlm, arln, arlp, arlq, arlr, arls, arlt, arlu, arlv, arm, armb,
armc, armd, arme, armf, armg, armh, armi, armk, arml, armm, armo, armp, armq, armr, arms, armt, arn,
arnb, arne, arnf, arni, arnj, arnm, arnr, arns, arnt, arnu, arnv, aro, arob, aroe, arof, arog, aroj, arok, arop,
aroq, aror, arot, arou, arov, arp, arpb, arpc, arpd, arpe, arpf, arpg, arph, arpi, arpj, arpk, arpl, arpm, arpn,
arpo, arpp, arpq, arpr, arpt, arpu, arpv, arq, arqb, arqc, arqd, arqe, arqf, arqg, arqh, arqi, arqj, arqk, arql,
arqm, arqn, arqo, arqp, arqq, arqr, arqs, arqt, arqu, arqv, arr, arrb, arrc, arrd, arre, arrf, arrg, arrh, arri,
arrj, arrk, arrl, arrm, arrn, arro, arrp, arrq, arrs, arru, arrv, ars, arsb, arsc, arsd, arse, arsg, arsh, arsi, arsl,
arsm, arsn, arsp, arsq, arsr, arst, arsv, art, artb, artc, artd, arte, artf, artg, arth, arti, artj, artk, artl, artm,
artn, arto, artp, artq, arts, artu, artv, aru, arub, aruc, arud, arue, aruf, arug, aruh, arui, arul, arum, arun,
arup, aruq, arur, arut, aruv, arv, arvb, arvc, arvd, arve, arvf, arvg, arvh, arvi, arvj, arvk, arvl, arvm, arvn,
arvo, arvp, arvq, arvr, arvs, arvt, arvu, arvv, asas, asat, asau, asav, asb, asbb, asbc, asbd, asbe, asbf, asbg,
asbh, asbi, asbk, asbl, asbm, asbp, asbq, asbr, asbs, asbt, asbu, asbv, asc, ascb, ascc, ascd, asce, ascf, ascg,
asci, asck, ascl, ascm, ascp, ascq, ascr, asct, ascv, asd, asdb, asdc, asdd, asde, asdf, asdg, asdk, asdm,
asdp, asdr, asds, asdt, asdv, aseb, asec, ased, asce, asef, aseg, aseh, asei, asek, asem, asen, asep, aseq,
aser, aset, aseu, asev, asf, asfb, asfc, asfd, asfe, asff, asth, asfi, asfj, asfk, asfl, asfm, asfp, asfq, asfr, asfs,
asft, asfu, asfv, asg, asgb, asgc, asgd, asge, asgf, asgg, asgh, asgi, asgk, asgl, asgm, asgp, asgq, asgr, asgs,
asgt, asgu, asgv, ash, ashb, ashd, ashf, ashg, ashh, ashi, ashk, ashl, ashm, ashp, ashq, ashr, ashs, asht,
ashu, ashv, asi, asib, asic, asid, asie, asig, asih, asii, asik, asil, asim, asip, asiq, asir, asit, asiv, asj, asjf,
ask, askb, askc, askd, aske, askf, askg, askh, aski, askm, askr, askt, askv, asl, aslb, aslc, asld, asle, aslf,
aslg, aslh, asli, aslj, aslk, asll, aslm, asln, aslp, aslq, aslr, asls, aslt, aslu, aslv, asm, asmb, asmc, asmf,
asmg, asmi, asn, asnb, asnc, asnd, asnf, asng, asnh, asnj, asnm, asnr, asns, asnt, asnu, aso, asob, asod,
asoe, asof, asog, asoi, asoj, asol, asom, ason, asop, asoq, asor, asos, asot, asou, asov, asp, aspb, aspc,
aspd, aspe, aspf, asph, aspi, aspj, aspm, aspq, aspr, asps, aspt, aspu, aspv, asq, asqb, asqc, asqd, asqe,
asqf, asqg, asqh, asqi, asqj, asqk, asql, asqm, asqu, asqp, asqq, asqr, asqs, asqt, asqu, asqv, asr, asrb, asrc,
asrd, asre, asrf, asrg, asrh, asri, asrj, asrk, asrm, asrn, asrp, asrq, asrr, asrs, asrt, asru, asrv, ass, asse, assf,
assg, assm, assq, assr, asss, asst, assv, ast, astb, astc, aste, astf, astg, asth, asti, astj, astk, astm, astp, astr,
asts, astt, astu, astv, asu, asub, asuc, asuh, asuj, asup, asur, asut, asv, asvb, asvc, asvd, asve, asvf, asvg,
asvh, asvi, asvk, asvm, asvp, asvq, asvr, asvs, asvt, asvu, asvv, atau, atav, atb, atbb, atbc, atbd, atbe, atbf,
atbg, atbh, atbi, atbj, atbk, atbl, atbm, atbn, atbo, atbp, atbq, atbr, atbs, atbt, atbu, atbv, atc, atcb, atcc,
atcd, atce, atcf, atcg, atch, atci, atcj, atck, atcl, atcm, atcn, atcp, atcq, atcr, atcs, atct, atcu, atcv, atd, atdb,
atdc, atdd, atde, atdf, atdg, atdj, atdk, atdl, atdm, atdp, atdq, atdr, atds, atdt, atdv, ate, ateb, atec, ated,
atee, atef, ateg, ateh, atei, atej, atek, atel, atem, aten, ateo, atep, ateq, ater, ates, atet, ateu, atev, atf, atfb,
atfc, atfd, atfe, atff, atfg, atfh, atfi, atfj, atfk, atfl, atfm, atfp, atfq, atfr, atfs, atft, atfu, atfv, atg, atgb, atgc,
atgd, atge, atgf, atgg, atgh, atgi, atgj, atgk, atgl, atgm, atgo, atgp, atgq, atgr, atgs, atgt, atgu, atgv, ath,
athb, athc, athd, athe, athf, athg, athh, athi, athj, athk, athl, athm, athn, atho, athp, athq, athr, aths, atht,
athv, ati, atib, atic, atid, atie, atif, atig, atih, atii, atik, atil, atim, atin, atip, atiq, atir, atis, atit, ativ, atj, atjb,
atjc, atjd, atje, atjf, atig, atjh, atji, atjj, atjk, atjm, atjp, atjq, atjr, atjs, atjt, atju, atjv, atk, atkb, atkc, atkd,
atke, atkf, atkg, atkh, atki, atkk, atkl, atkm, atkp, atkq, atkr, atks, atkt, atku, atkv, atl, atlb, atlc, atld, atle,
atlf, atlg, atlh, atli, atlj, atlk, atll, atlm, atln, atlp, atlq, atlr, atls, atlt, atlu, atlv, atm, atmb, atmd, atme,
atmf, atmg, atmi, atmk, atml, atmm, atmo, atmp, atmq, atmr, atms, atmt, atn, atnb, atnc, atnf, atnj, atns,
atnt, ato, atoe, atof, atoj, atoq, atp, atpb, atpc, atpd, atpe, atpf, atpg, atph, atpi, atpk, atpl, atpm, atpn, atpo,
atpp, atpq, atpr, atps, atpt, atpv, atq, atqb, atqc, atqd, atqe, atqf, atqg, atqh, atqi, atqj, atqk, atql, atqm,
atqn, atqp, atqq, atqr, atqs, atqt, atqu, atqv, atr, atrb, atrc, atrd, atre, atrf, atrg, atrh, atri, atrj, atrk, atrl,
atrm, atrn, atrp, atrq, atrr, atrs, atrt, atru, atrv, ats, atsb, atsc, atsd, atse, atsg, atsh, atsi, atsl, atsm, atso,
atsq, atsr, atsv, att, attb, attc, attd, atte, attf, attg, atth, atti, attj, attk, attl, attm, attn, atto, attp, attq, attr,
atts, attt, attu, attv, atu, atub, atuc, atud, atue, atuf, atug, atuh, atui, atul, atum, atup, atuq, atur, atut, atuv,
atv, atvb, atvc, atvd, atve, atvf, atvg, atvh, atvi, atvj, atvk, atvl, atvm, atvn, atvo, atvp, atvq, atvr, atvs,
atvt, atvu, atvv, auav, aub, aubb, aubc, aubd, aube, aubf, aubg, aubh, aubi, aubk, aubl, aubm, aubp, aubq,
aubr, aubs, aubt, aubu, aubv, auc, aucb, aucc, aucg, aucl, aucp, aucq, aud, audb, audf, audg, audq, audr,
ane, aueb, aued, auee, auef, aueg, auei, auem, auep, aueq, auer, aues, aueu, auev, auf, aufb, aufc, aufe,
auff, aufg, aufh, aufi, aufj, aufk, aufm, aufp, aufr, aufs, aufv, aug, augb, augd, auge, augf, augg, augh,
augi, augm, augr, augs, augt, augu, augv, auh, auhb, auhf, auhg, auhh, auhk, auhl, auhm, aui, auib, auic,
auil, auim, auir, auit, auj, aujb, aujf, aujg, aujj, aujm, aujr, aujs, aujt, auju, auk, aukc, aukd, auke, auki,
aukm, aukq, aukr, auks, aukt, aukv, aul, aulb, aulc, auld, aule, aulf, aulg, auli, aulj, aulk, aulm, aulp, aulq,
aulr, ault, aulv, aum, aumi, aumm, aumr, aun, aunf, auo, aup, aupb, aupd, aupe, aupg, aupi, aupm, aupq,
aupr, aupt, aupv, auq, auqb, auqc, auqd, auqe, auqf, auqg, auqh, auqi, auqj, auqk, auql, auqm, auqq, auqr,
auqs, auqt, auqv, aur, aurb, aurc, aurd, aure, aurf, aurg, aurh, auri, aurj, aurm, aurq, aurr, aurs, aurt, auru,
aus, ausb, ausi, aut, autb, autc, autd, aute, autf, autg, auth, auti, autj, autm, autq, autr, auts, autt, autu, auu,
auut, auv, auvb, auvc, auvd, auve, auvf, auvg, auvh, auvi, auvk, auvm, auvn, auvq, auvr, auvs, auvt, auvv,
avb, avbb, avbc, avbd, avbe, avbf, avbg, avbh, avbi, avbj, avbk, avbl, avbm, avbn, avbo, avbp, avbq,
avbr, avbs, avbt, avbu, avbv, avc, avcb, avcc, avcd, avce, avcf, avcg, avch, avci, avcj, avck, avcl, avcm,
avcn, avco, avcp, avcq, avcr, avct, avcv, avd, avdb, avdc, avdd, avde, avdf, avdg, avdh, avdi, avdj, avdk,
avdl, avdm, avdn, avdo, avdp, avdq, avdr, avds, avdt, avdv, ave, aveb, avec, aved, avee, avef, aveg, aveh,
avei, avej, avek, avel, avem, aven, aveo, avep, aveq, aver, aves, avet, aveu, avev, avf, avfb, avfc, avfd,
avfe, avff, avfg, avfh, avfi, avfj, avfk, avfl, avfm, avfn, avfp, avfq, avfr, avfs, avft, avfu, avfv, avg, avgb,
avgc, avgd, avge, avgf, avgg, avgh, avgi, avgj, avgk, avgl, avgm, avgn, avgo, avgp, avgq, avgr, avgs,
avgt, avgu, avgv, avh, avhb, avhc, avhd, avhe, avhf, avhg, avhh, avhi, avhj, avhk, avhl, avhm, avhn,
avho, avhp, avhq, avhr, avhs, avht, avhu, avhv, avi, avib, avic, avid, avie, avif, avig, avih, avii, avij, avik,
avil, avim, avin, avio, avip, aviq, avir, avis, avit, aviv, avj, avjb, avjf, avjj, avjs, avjt, avk, avkb, avkc,
avkd, avke, avkf, avkg, avkh, avki, avkj, avkk, avkl, avkm, avkn, avkp, avkq, avkr, avks, avkt, avku,
avkv, avl, avlb, avlc, avld, avle, avlf, avlg, avlh, avli, avlj, avlk, avll, avlm, avln, avlo, avlp, avlq, avlr,
avls, avlt, avlu, avlv, avm, avmb, avmc, avmd, avme, avmf, avmg, avmi, avmj, avmk, avml, avmo, avmp,
avmr, avmt, avn, avnb, avnc, avnd, avne, avnf, avng, avnh, avni, avnj, avnk, avnm, avnp, avnq, avnr,
avns, avnt, avnu, avnv, avo, avob, avod, avoe, avof, avog, avoh, avoi, avoj, avok, avol, avom, avon, avop,
avoq, avor, avos, avot, avou, avov, avp, avpb, avpc, avpd, avpe, avpf, avpg, avph, avpi, avpj, avpk, avpl,
avpm, avpn, avpo, avpp, avpq, avpr, avps, avpt, avpu, avpv, avq, avqb, avqc, avqd, avqe, avqf, avqg,
avqh, avqi, avqj, avqk, avql, avqm, avqn, avqo, avqp, avqq, avqr, avqs, avqt, avqu, avqv, avr, avrb, avrc,
avrd, avre, avrf, avrg, avrh, avri, avrj, avrk, avrl, avrm, avrn, avro, avrp, avrq, avrr, avrs, avrt, avru, avrv,
avs, avsb, avsc, avsd, avse, avsf, avsg, avsh, avsi, avsj, avsk, avsl, avsm, avsn, avso, avsp, avsq, avsr,
avss, avst, avsv, avt, avtb, avtc, avtd, avte, avtf, avtg, avth, avti, avtj, avtk, avtl, avtm, avtn, avto, avtp,
avtq, avtr, avts, avtt, avtu, avtv, avu, avub, avud, avue, avuf, avug, avuh, avuj, avul, avup, avuq, avur,
avut, avuv, avv, avvb, avvc, avvd, avve, avvf, avvg, avvh, avvi, avvj, avvk, avvl, avvm, avvn, avvo,
avvp, avvq, avvr, avvs, avvt, avvu, avvv, bbbd, bbbh, bbbi, bbbj, bbbk, bbbl, bbbm, bbbn, bbbo, bbbp,
bbbq, bbbr, bbbs, bbbt, bbbu, bbbv, bbc, bbcd, bbch, bbci, bbcj, bbck, bbcl, bbcm, bbcn, bbco, bbcp,
bbcq, bbcr, bbcs, bbct, bbcu, bbcv, bbdc, bbde, bbdf, bbdg, bbdh, bbdi, bbdj, bbdk, bbdl, bbdm, bbdn,
bbdo, bbdp, bbdq, bbdr, bbds, bbdt, bbdu, bbdv, bbee, bbef, bbeg, bbeh, bbei, bbej, bbek, bbel, bbem,
bben, bbeo, bbep, bbeq, bber, bbes, bbet, bbeu, bbev, bbf, bbfd, bbfe, bbff, bbfg, bbfh, bbfi, bbfj, bbfk,
bbfl, bbfm, bbfn, bbfp, bbfq, bbfr, bbfs, bbft, bbfu, bbfv, bbgd, bbgf, bbgh, bbgj, bbgk, bbgl, bbgm, bbgn,
bbgo, bbgp, bbgq, bbgr, bbgs, bbgt, bbgu, bbgv, bbh, bbhc, bbhd, bbhe, bbhf, bbhg, bbhh, bbhi, bbhj,
bbhk, bbhl, bbhm, bbhn, bbho, bbhp, bbhq, bbhr, bbhs, bbht, bbhu, bbhv, bbi, bbic, bbid, bbie, bbif, bbih,
bbik, bbil, bbim, bbin, bbio, bbip, bbiq, bbir, bbis, bbit, bbiv, bbj, bbjc, bbjf, bbjg, bbjh, bbjj, bbjm, bbjp,
bbjr, bbjs, bbjt, bbju, bbjv, bbk, bbkc, bbkd, bbke, bbkf, bbkg, bbkh, bbki, bbkj, bbkk, bbkl, bbkm, bbkn,
bbkp, bbkq, bbkr, bbks, bbkt, bbku, bbkv, bbl, bblc, bbld, bble, bblf, bblg, bblh, bbli, bblj, bblk, bbll,
bblm, bbln, bblp, bblq, bblr, bbls, bblt, bblu, bblv, bbm, bbmc, bbmd, bbme, bbmf, bbmg, bbmh, bbmi,
bbmj, bbml, bbmm, bbmo, bbmp, bbmq, bbmr, bbms, bbmt, bbmv, bbn, bbnc, bbnd, bbne, bbnf, bbng,
bbnh, bbnj, bbnm, bbnp, bbnr, bons, bbnt, bbnu, bbo, bboc, bbod, bboe, bbof, bbog, bboh, bboi, bboj,
bbok, bbom, bbon, bbop, bbog, bbor, bbos, bbot, bbou, bbov, bbp, bbpc, bbpd, bbpe, bbpf, bbpg, bbph,
bbpi, bbpj, bbpk, bbpl, bbpm, bbpn, bbpo, bbpp, bbpq, bbpr, bbps, bbpt, bbpu, bbpv, bbq, bbqc, bbqd,
bbqe, bbqf, bbqg, bbqh, bbqi, bbqj, bbqk, bbql, bbqm, bbqn, bbqo, bbqp, bbqq, bbqr, bbqs, bbqt, bbqu,
bbqv, bbr, bbrc, bbrd, bbre, bbrf, bbrg, bbrh, bbri, bbrj, bbrk, bbrl, bbrm, bbrn, bbro, bbrp, bbrq, bbrr,
bbrs, bbrt, bbru, bbrv, bbs, bbsc, bbsd, bbse, bbsf, bbsg, bbsh, bbsi, bbsk, bbsl, bbsm, bbsn, bbso, bbsp,
bbsq, bbsr, bbss, bbst, bbsu, bbsv, bbt, bbte, bbtd, bbte, bbtf, bbtg, bbth, bbti, bbtj, bbtk, bbtl, bbtm, bbtn,
bbto, bbtp, bbtq, bbtr, bbts, bbtt, bbtu, bbtv, bbu, bbuc, bbud, bbue, bbuf, bbug, bbuh, bbui, bbuj, bbuk,
bbul, bbum, bbun, bbup, bbuq, bbur, bbut, bbuu, bbuv, bbv, bbvc, bbvd, bbve, bbvf, bbvg, bbvh, bbvi,
bbvk, bbvl, bbvm, bbvn, bbvo, bbvp, bbvq, bbvr, bbvs, bbvt, bbvu, bbvv, bcbd, bcbg, bcbh, bcbi, bcbj,
bcbk, bcbl, bcbm, bcbn, bcbo, bcbp, bcbq, bcbr, bcbs, bcbt, bcbu, bcbv, bcc, bccd, bcch, bcci, bcck, bccl,
bcco, bccp, bccq, bccr, bccs, bcct, bccv, bcd, bcdc, bcdd, bcde, bcdf, bcdg, bcdh, bcdi, bcdj, bcdk, bcdl,
bcdm, bcdo, bcdp, bcdq, bcdr, bcds, bcdt, bcdu, bcdv, bce, bced, bcee, bcef, bceg, bceh, bcei, bcej, bcek,
bcel, bcem, bcen, bcep, bceq, bcer, bces, bcet, bceu, bcev, bcf, bcfd, bcfe, bcff, bcfg, bcfh, bcfi, bcfj,
bcfm, bcfp, bcfq, bcfr, bcfs, bcft, bcfu, bcfv, bcg, bcgd, bcge, bcgf, bcgg, bcgh, bcgi, bcgj, bcgk, bcgl,
bcgm, bcgn, bcgp, bcgq, bcgr, bcgs, bcgt, bcgu, bcgv, bch, bchc, bchd, bche, bchf, bchg, bchh, bchi,
bchk, bchl, bchm, bcho, bchp, bchq, bchr, bchs, bcht, bchu, bchv, bci, bcic, bcid, bcie, bcig, bcih, bcii,
bcik, bcil, bcin, bcio, bcip, bciq, bcir, bcis, bcit, bciv, bcj, bcjc, bcjf, bcjg, bcjh, bcjm, bcjr, bcjs, bcjt,
bcju, bck, bckc, bckd, bcke, bckf, bckg, bckh, bcki, bckj, bckl, bckm, bckp, bckq, bckr, bcks, bckt, bcku,
bckv, becl, bclc, bcld, bcle, bclf, bclg, bclh, bcli, bclj, bclk, bcll, bclm, bcln, bclp, bclq, bclr, bcls, bclt,
bclu, bclv, bcm, bcmc, bcmd, bcme, bcmf, bcmg, bcmh, bcmi, bcmj, bcml, bcmm, bcmp, bcmr, bcms,
bcmt, bcmv, bcn, bcnc, bcnf, bcng, bcnh, bcnj, bcnm, bcnp, bcnr, bcns, bcnt, bcnu, bcnv, bco, bcoe, bcof,
bcog, bcoi, bcoj, bcog, bcor, boot, bcou, bcp, bcpc, bcpd, bcpe, bcpf, bcpg, bcph, bcpi, bcpj, bcpk, bcpl,
bcpm, bcpn, bcpo, bcpp, bcpq, bcpr, bcps, bcpt, bcpu, bcpv, bcq, bcqc, bcqd, bcqe, bcqf, bcqg, bcqh,
bcqi, bcqj, bcqk, bcql, bcqm, bcqn, bcqp, bcqq, bcqr, bcqs, bcqt, bcqu, bcqv, bcr, bcrc, bcrd, bcre, bcrf,
bcrg, bcrh, bcri, bcrj, bcrk, bcrl, bcrm, bcrn, bcro, bcrp, bcrq, bcrr, bcrs, bcrt, bcru, bcrv, bcs, bcsc, bcsd,
bcse, bcsf, bcsg, bcsh, bcsi, bcsj, bcsl, bcsm, bcsn, bcso, bcsp, bcsq, bcsr, bcst, bcsv, bct, bctc, bctd, bcte,
bctf, bctg, bcth, bcti, bctj, bctl, bctm, bctn, bctp, bctq, bctr, bcts, bctt, bctu, bctv, bcu, bcuc, bcue, bcuf,
bcup, bcv, bcvc, bcvd, bcve, bcvf, bcvg, bcvh, bcvi, bcvk, bcvl, bcvm, bcvn, bcvo, bcvp, bcvq, bcvr,
bcvs, bcvt, bcvu, bcvv, bdbe, bdbf, bdbg, bdbh, bdbi, bdbj, bdbk, bdbl, bdbm, bdbn, bdbo, bdbp, bdbq,
bdbr, bdbs, bdbt, bdbu, bdbv, bdc, bdcc, bdcd, bdce, bdcf, bdcg, bdch, bdci, bdcj, bdck, bdcl, bdcm,
bdcn, bdco, bdcp, bdcq, bdcr, bdct, bdcv, bddc, bddd, bdde, bddf, bddg, bddh, bddi, bddj, bddk, bddl,
bddm, bddn, bddo, bddp, bddq, bddr, bddt, bddv, bde, bdec, bded, bdee, bdef, bdeg, bdeh, bdei, bdej,
bdek, bdel, bdem, bden, bdeo, bdep, bdeq, bder, bdes, bdet, bdeu, bdev, bdf, bdfc, bdfd, bdfe, bdff, bdfg,
bdfh, bdfi, bdfj, bdfk, bdfl, bdfm, bdfn, bdfp, bdfq, bdfr, bdfs, bdft, bdfu, bdfv, bdgc, bdgd, bdge, bdgf,
bdgg, bdgh, bdgi, bdgj, bdgk, bdgl, bdgm, bdgn, bdgo, bdgp, bdgq, bdgr, bdgs, bdgt, bdgu, bdgv, bdh,
bdhc, bdhd, bdhe, bdhf, bdhg, bdhh, bdhi, bdhj, bdhk, bdhl, bdhm, bdhn, bdho, bdhp, bdhq, bdhr, bdhs,
bdht, bdhv, bdi, bdic, bdid, bdie, bdif, bdig, bdih, bdii, bdik, bdil, bdim, bdin, bdio, bdip, bdiq, bdir, bdis,
bdit, bdiv, bdj, bdjc, bdje, bdjf, bdjg, bdjh, bdji, bdjj, bdjk, bdjm, bdjp, bdjq, bdjr, bdjs, bdjt, bdju, bdk,
bdkc, bdkd, bdke, bdkf, bdkg, bdkh, bdki, bdkj, bdkk, bdkl, bdkm, bdkn, bdkp, bdkq, bdkr, bdks, bdkt,
bdkv, bdl, bdlc, bdld, bdle, bdlf, bdlg, bdlh, bdli, bdlj, bdlk, bdll, bdlm, bdln, bdlp, bdlq, bdlr, bdls, bdlt,
bdlu, bdlv, bdmc, bdme, bdmf, bdmg, bdmb, bdmj, bdml, bdmn, bdmo, bdmq, bdmr, bdms, bdmt, bdmv,
bdn, bdnc, bdnd, bdnf, bdng, bdnh, bdnj, bdnm, bdnr, bdns, bdnt, bdnu, bdnv, bdo, bdoc, bdod, bdoe,
bdof, bdog, bdoj, bdok, bdon, bdoq, bdor, bdot, bdou, bdov, bdp, bdpc, bdpd, bdpe, bdpf, bdpg, bdph,
bdpi, bdpj, bdpk, bdpl, bdpn, bdpo, bdpp, bdpq, bdpr, bdpt, bdpu, bdpv, bdq, bdqc, bdqd, bdqe, bdqf,
bdqg, bdqh, bdqi, bdqi, bdqk, bdql, bdqm, bdqn, bdqo, bdqp, bdqq, bdqr, bdqt, bdqu, bdqv, bdr, bdrc,
bdrd, bdre, bdrf, bdrg, bdrh, bdri, bdrj, bdrk, bdrl, bdrm, bdrn, bdro, bdrp, bdrq, bdrr, bdrt, bdru, bdrv,
bds, bdsd, bdse, bdsf, bdsg, bdsk, bdsl, bdsn, bdso, bdsq, bdsr, bdst, bdsv, bdt, bdtc, bdtd, bdte, bdtf,
bdtg, bdth, bdti, bdtj, bdtk, bdtl, bdtm, bdtn, bdto, bdtp, bdtq, bdtr, bdts, bdtt, bdtu, bdtv, bdu, bdue, bduj,
bduk, bdul, bdun, bdup, bduq, bdur, bdut, bduv, bdv, bdvc, bdvd, bdve, bdvf, bdvg, bdvh, bdvi, bdvj,
bdvk, bdvl, bdvm, bdvn, bdvo, bdvp, bdvq, bdvr, bdvt, bdvu, bdvv, bebh, bebi, bebj, bebk, bebl, bebm,
bebn, bebo, bebp, bebq, bebr, bebs, bebt, bebu, bebv, bec, becd, bech, beci, becj, beck, becl, becm, becn,
beco, becp, becq, becr, becs, bect, becv, bedc, bede, bedf, bedg, bedh, bedi, bedj, bedk, bedl, bedm, bedn,
bedo, bedp, bedq, bedr, beds, bedt, bedu, bedv, beec, beed, beeh, beei, beej, beek, beel, beem, been, beeo,
beep, beeq, beer, bees, beet, beeu, beev, bef, befc, befd, beff, befh, befi, befj, befk, befl, befm, befn, befo,
befp, befq, befr, befs, beft, befu, befv, begc, begd, begh, begi, begj, begk, begl, begm, begn, bego, begp,
begq, begr, begs, begt, begu, begv, beh, behc, behd, behe, behf, behg, behh, behi, behj, behk, behl, behm,
behn, beho, behp, behq, behr, behs, beht, behu, behv, bei, beic, beid, beie, beif, beig, beih, beii, beij,
beik, beil, beim, bein, beio, beip, beiq, beir, beis, beit, beiv, bej, bejc, bejd, beje, bejf, bejg, bejh, beji,
bejj, bejm, bejp, bejq, bejr, bejs, bejt, beju, bejv, bek, bekc, bekd, beke, bekf, bekg, bekh, beki, bekj,
bekk, bekl, bekm, bekn, beko, bekp, bekq, bekr, beks, bekt, beku, bekv, bel, belc, beld, bele, belf, belg,
belh, beli, belj, belk, bell, belm, beln, belo, belp, belq, belr, bels, belt, belv, bemc, bemd, beme, bemf,
bemg, bemh, bemi, bemj, beml, bemm, bemn, bemo, bemp, bemq, bemr, bemt, bemv, ben, benc, bend,
bene, benf, beng, benh, beni, benj, benk, benl, benm, benp, benq, benr, bens, bent, benu, benv, beo, beoc,
beod, beoe, beof, beog, beoh, beoi, beoj, beok, beol, beom, beon, beop, beoq, beor, beos, beot, beov, bep,
bepc, bepd, bepe, bepf, bepg, beph, bepi, bepj, bepk, bepl, bepm, bepn, bepo, bepp, bepq, bepr, beps,
bept, bepu, bepv, beq, beqc, beqd, beqe, beqf, beqg, beqh, beqi, beqj, beqk, beql, beqm, beqn, beqo,
beqp, beqq, beqr, beqs, beqt, bequ, beqv, ber, berc, berd, bere, berf, berg, berh, beri, berj, berk, berl,
berm, bern, bero, berp, berg, berr, bers, bert, beru, berv, bes, besc, besd, bese, besf, besg, besh, besi, besj,
besk, besl, besm, besn, beso, besp, besq, besr, bess, best, besu, besv, bet, betc, betd, bete, betf, betg, beth,
beti, betj, betk, betl, betm, betn, beto, betp, betq, betr, bett, betv, beu, beuc, beud, beue, beuf, beug, beuh,
beui, beuj, beuk, beul, beun, beuo, beup, beuq, beur, beut, beuv, bev, bevc, bevd, beve, bevf, bevg, bevh,
bevi, bevj, bevk, bevl, bevm, bevn, bevo, bevp, bevq, bevr, bevs, bevt, bevv, bfbh, bfbi, bfbj, bfbk, bfbl,
bfbm, bfbn, bfbo, bfbp, bbq, bfbr, bfbs, bfbt, bfbu, bfbv, bfc, bfcd, bfch, bfci, bfcj, bfck, bfcl, bfcm,
bfcn, bfcp, bfcq, bfcr, bfcs, bfct, bfcu, bfcv, bfd, bfdc, bfdd, bfde, bfdf, bfdg, bfdh, bfdi, bfdj, bfdk, bfdl,
bfdm, bfdn, bfdo, bfdp, bfdq, bfdr, bfds, bfdt, bfdv, bfe, bfec, bfed, bfeh, bfei, bfej, bfek, bfel, bfem,
bfen, bfeo, bfep, bfeq, bfer, bfes, bfet, bfeu, bfev, bff, bffc, bffd, bffe, bfff, bffg, bffh, bffi, bffj, bffk, bffl,
bffm, bffo, bffp, bffq, bffr, bffs, bfft, bffu, bffv, bfg, bfgc, bfgd, bfgf, bfgh, bfgi, bfgj, bfgk, bfgl, bfgm,
bfgn, bfgo, bfgp, bfgq, bfgr, bfgs, bfgt, bfgu, bfgv, bfh, bfhc, bfhd, bfhe, bfhf, bfhg, bfhh, bfhi, bfhk,
bfhl, bfhm, bfhn, bfho, bfhp, bfhq, bfhr, bfhs, bfht, bfhv, bfi, bfic, bfid, bfie, bfif, bfig, bfih, bfii, bfik,
bfil, bfim, bfin, bfio, bfip, bfiq, bfir, bfis, bfit, bfiv, bfj, bfje, bfjd, bfje, bfjf, bfjg, bfjh, bfji, bfjk, bfjm,
bfjp, bfjq, bfjr, bfjs, bfjt, bfju, bfjv, bfk, bfkc, bfkd, bfke, bfkf, bfkg, bfkh, bfki, bfkj, bfkk, bfkl, bfkm,
bfkn, bfkp, bfkq, bfkr, bfks, bfkt, bfku, bfkv, bfl, bflc, bfld, bfle, bflf, bflg, bflh, bfli, bflj, bflk, bfll, bflm,
bfln, bflp, bflq, bflr, bfls, bflt, bflu, bflv, bfm, bfmc, bfmd, bfme, bfmf, bfmg, bfmh, bfmi, bfmj, bfml,
bfmm, bfmo, bfmp, bfmq, bfmr, bfms, bfmt, bfmv, bfn, bfnf, bfnj, bfnr, bfnt, bfnu, bfo, bfoe, bfoj, bfp,
bfpc, bfpd, bfpe, bfpf, bfpg, bfph, bfpi, bfpj, bfpk, bfpl, bfpm, bfpn, bfpo, bfpp, bfpq, bfpr, bfpt, bfpu,
bfpv, bfq, bfqc, bfqd, bfqe, bfqf, bfqg, bfqh, bfqi, bfqj, bfqk, bfql, bfqm, bfqn, bfqo, bfqp, bfqq, bfqr,
bfqt, bfqu, bfqv, bfr, bfrc, bfrd, bfre, bfrf, bfrg, bfrh, bfri, bfrj, bfrk, bfrl, bfrm, bfm, bfro, bfrp, bfrq, bfrr,
bfrs, bfrt, bfru, bfrv, bfs, bfsc, bfsd, bfse, bfsf, bfsg, bfsh, bfsi, bfsk, bfsl, bfsm, bfsn, bfso, bfsp, bfsq,
bfsr, bfst, bfsu, bfsv, bft, bftc, bftd, bfte, bftf, bftg, bfth, bfti, bftj, bftk, bfil, bftm, bfin, bfto, bftp, bftq,
bftr, bfts, bftt, bftu, bftv, bfu, bfuc, bfud, bfue, bfuf, bfug, bfuh, bfui, bfuj, bfuk, bful, bfun, bfuo, bfuq,
bfur, bfut, bfuv, bfv, bfvc, bfvd, bfve, bfvf, bfvg, bfvh, bfvi, bfvk, bfvl, bfvm, bfvn, bfvo, bfvp, bfvq,
bfvr, bfvt, bfvv, bgbh, bgbj, bgbk, bgbl, bgbm, bgbn, bgbo, bgbp, bgbq, bgbr, bgbs, bgbt, bgbu, bgbv,
bgcd, bgch, bgci, bgcj, bgck, bgcl, bgcm, bgcn, bgcp, bgcq, bgcr, bgcs, bgct, bgcu, bgcv, bgd, bgdc,
bgdd, bgde, bgdf, bgdg, bgdh, bgdi, bgdj, bgdk, bgdl, bgdm, bgdn, bgdo, bgdp, bgdq, bgdr, bgds, bgdt,
bgdu, bgdv, bgec, bged, bgeh, bgei, bgej, bgek, bgel, bgem, bgen, bgeo, bgep, bgeq, bger, bges, bget,
bgeu, bgev, bgf, bgfc, bgfd, bgff, bgfh, bgfi, bgfj, bgfk, bgfl, bgfm, bgfn, bgfo, bgfp, bgfq, bgfr, bgfs,
bgft, bgfu, bgfv, bgg, bggc, bggd, bggh, bggj, bggk, bggl, bggm, bggn, bggo, bggp, bggq, bggr, bggs,
bggt, bggu, bggv, bgh, bghc, bghd, bghe, bghf, bghg, bghh, bghi, bghj, bghk, bghl, bghm, bghn, bgho,
bghp, bghq, bghr, bghs, bght, bghu, bghv, bgi, bgic, bgid, bgie, bgif, bgih, bgik, bgil, bgim, bgin, bgio,
bgip, bgiq, bgir, bgis, bgit, bgiv, bgj, bgjc, bgjd, bgje, bgjf, bgjg, bgjh, bgji, bgij, bgjk, bgjm, bgjp, bgjq,
bgjr, bgjs, bgjt, bgju, bgjv, bgk, bgkc, bgkd, bgke, bgkf, bgkg, bgkh, bgki, bgkj, bgkk, bgkl, bgkm, bgkn,
bgkp, bgkq, bgkr, bgks, bgkt, bgku, bgkv, bgl, bglc, bgld, bgle, bglf, bglg, bglh, bgli, bglj, bglk, bgll,
bglm, bgln, bglp, bglq, bglr, bgls, bglt, bglu, bglv, bgm, bgmc, bgmd, bgme, bgmf, bgmg, bgmh, bgmi,
bgmj, bgmk, bgml, bgmm, bgmn, bgmo, bgmp, bgmq, bgmr, bgms, bgmt, bgmv, bgn, bgnc, bgnd, bgne,
bgnf, bgnh, bgnj, bgnk, bgnm, bgnp, bgnq, bgnr, bgns, bgnt, bgnu, bgnv, bgo, bgoc, bgod, bgoe, bgof,
bgog, bgoh, bgoi, bgoj, bgok, bgol, bgom, bgop, bgoq, bgor, bgos, bgot, bgou, bgov, bgp, bgpc, bgpd,
bgpe, bgpf, bgpg, bgph, bgpi, bgpj, bgpk, bgpl, bgpm, bgpn, bgpo, bgpp, bgpq, bgpr, bgps, bgpt, bgpu,
bgpv, bgq, bgqc, bgqd, bgqe, bgqf, bgqg, bgqh, bgqi, bgqj, bgqk, bgql, bgqm, bgqn, bgqo, bgqp, bgqq,
bgqr, bgqs, bgqt, bgqu, bgqv, bgr, bgrc, bgrd, bgre, bgrf, bgrg, bgrh, bgri, bgrj, bgrk, bgrl, bgrm, bgrn,
bgro, bgrp, bgrq, bgrr, bgrs, bgrt, bgru, bgrv, bgs, bgsc, bgsd, bgse, bgsf, bgsg, bgsh, bgsi, bgsk, bgsl,
bgsm, bgsn, bgso, bgsp, bgsq, bgsr, bgss, bgst, bgsu, bgsv, bgt, bgtc, bgtd, bgte, bgtf, bgtg, bgth, bgti,
bgtj, bgtk, bgtl, bgtm, bgtn, bgto, bgtp, bgtq, bgtr, bgts, bgtt, bgtu, bgtv, bgu, bguc, bgud, bgue, bguf,
bgug, bguh, bgui, bguj, bguk, bgul, bgum, bgun, bguo, bgup, bguq, bgur, bgus, bgut, bguu, bguv, bgv,
bgvc, bgvd, bgve, bgvf, bgvg, bgvh, bgvi, bgvj, bgvk, bgvl, bgvm, bgvn, bgvo, bgvp, bgvq, bgvr, bgvs,
bgvt, bgvu, bgvv, bhbh, bhbi, bhbj, bhbk, bhbl, bhbm, bhbn, bhbo, bhbp, bhbq, bhbr, bhbs, bhbt, bhbu,
bhbv, bhc, bhcc, bhcd, bhce, bhcf, bhcg, bhch, bhci, bhcj, bhck, bhcl, bhcm, bhcn, bhcp, bhcq, bhcr,
bhcs, bhct, bhcv, bhd, bhdc, bhdd, bhde, bhdf, bhdg, bhdh, bhdi, bhdj, bhdk, bhdl, bhdm, bhdn, bhdo,
bhdp, bhdq, bhdr, bhds, bhdt, bhdu, bhdv, bhe, bhec, bhed, bhee, bhef, bheg, bheh, bhei, bhek, bhel,
bhem, bhen, bhep, bheq, bher, bhes, bhet, bheu, bhev, bhf, bhfc, bhfd, bhfe, bhff, bhfg, bhfh, bhfi, bhfj,
bhfm, bhfp, bhfq, bhfr, bhfs, bhft, bhfu, bhfv, bhg, bhgc, bhgd, bhge, bhgf, bhgg, bhgh, bhgi, bhgj, bhgk,
bhgl, bhgm, bhgn, bhgp, bhgq, bhgr, bhgs, bhgt, bhgu, bhgv, bhh, bhhc, bhhd, bhhe, bhhf, bhhg, bhhh,
bhhi, bhhk, bhhl, bhhm, bhho, bhhp, bhhq, bhhr, bhhs, bhht, bhhu, bhhv, bhi, bhic, bhid, bhie, bhif, bhig,
bhih, bhii, bhik, bhil, bhim, bhin, bhio, bhip, bhiq, bhir, bhis, bhit, bhiv, bhj, bhjf, bhjh, bhjj, bhjs, bhjt,
bhk, bhkc, bhkd, bhke, bhkf, bhkg, bhkh, bhki, bhkj, bhkk, bhkl, bhkm, bhkn, bhkp, bhkq, bhkr, bhks,
bhkt, bhku, bhkv, bhl, bhlc, bhld, bhle, bhlf, bhlg, bhlh, bhli, bhlj, bhlk, bhll, bhlm, bhln, bhlp, bhlq, bhlr,
bhls, bhlt, bhlu, bhlv, bhm, bhmc, bhmd, bhme, bhmf, bhmg, bhmi, bhmj, bhml, bhmp, bhmq, bhmr,
bhn, bhnc, bhnd, bhne, bhnf, bhng, bhnh, bhni, bhnj, bhnm, bhnp, bhnr, bhns, bhnt, bhnu, bhnv, bho,
bhoc, bhod, bhoc, bhof, bhog, bhoh, bhoi, bhoj, bhol, bhom, bhop, bhoq, bhor, bhos, bhot, bhou, bhov,
bhp, bhpc, bhpd, bhpe, bhpf, bhpg, bhph, bhpi, bhpj, bhpk, bhpl, bhpm, bhpn, bhpo, bhpp, bhpq, bhpr,
bhps, bhpt, bhpu, bhpv, bhq, bhqc, bhqd, bhqe, bhqf, bhqg, bhqh, bhqi, bhqj, bhqk, bhql, bhqm, bhqn,
bhqp, bhqq, bhqr, bhqs, bhqt, bhqu, bhqv, bhr, bhrc, bhrd, bhre, bhrf, bhrg, bhrh, bhri, bhrj, bhrk, bhrl,
bhrm, bhrn, bhro, bhrp, bbrq, bhrr, bhrs, bhrt, bhru, bhrv, bhs, bhsd, bhse, bhsf, bhsg, bhsh, bhsi, bhsl,
bhsm, bhso, bhsp, bhsq, bhsr, bhss, bhst, bhsv, bht, bhtc, bhtd, bhte, bhtf, bhtg, bhth, bhti, bhtj, bhtl,
bhtm, bhtn, bhto, bhtp, bhtq, bhtr, bhts, bhtt, bhtu, bhtv, bhu, bhue, bhuf, bhug, bhui, bhuj, bhum, bhur,
bhut, bhuv, bhv, bhvc, bhvd, bhve, bhvf, bhvg, bhvh, bhvi, bhvk, bhvl, bhvm, bhvn, bhvo, bhvp, bhvq,
bhvr, bhvs, bhvt, bhvu, bhvv, bibj, bibk, bibl, bibm, bibn, bibo, bibp, bibq, bibr, bibs, bibt, bibu, bibv,
bic, bicc, bice, bicf, bicg, bici, bicj, bick, bicl, bicn, bicp, bicq, bicr, bict, bicv, bid, bidc, bidd, bide, bidf,
bidg, bidh, bidi, bidj, bidk, bidl, bidm, bido, bidp, bidq, bidr, bids, bidt, bidu, bidv, bie, biec, bied, biee,
bief, bieg, bieh, biei, biej, biek, biel, biem, bien, biep, bieq, bier, bies, biet, bieu, biev, bif, bifc, bife, biff,
bifg, bifh, bifi, bifj, bifm, bifq, bifr, bifs, bift, bifu, big, bigc, bigd, bige, bigf, bigh, bigj, bigk, bigl, bigm,
bign, bigp, bigq, bigr, bigs, bigt, bigu, bigv, bih, bihc, bihd, bihe, bihf, bihg, bihh, bihi, bihj, bihl, bihm,
bihn, biho, bihp, bihq, bihr, biht, bibu, bihv, bii, biic, biid, biie, biif, biik, biil, biim, biin, biio, biip, biiq,
biir, biit, biiv, bij, bijf, bijj, bik, bikc, bikd, bike, bikf, bikg, bikh, biki, bikj, bikk, bikl, bikm, bikn, bikp,
bikq, bikr, biks, bikt, biku, bikv, bil, bilc, bild, bile, bilf, bilg, bilh, bili, bilj, bilk, bill, bilm, biln, bilp,
bilq, bilr, bilt, bilu, bilv, bim, bimc, bimd, bime, bimf, bimg, bimh, bimi, bimj, biml, bimm, bimq, bimr,
bimt, bin, binc, bine, binf, bing, binh, binj, binm, binp, binr, bins, bint, binu, binv, bio, bioc, biod, bioe,
biof, biog, bioh, bioi, bioj, biok, biol, biom, bion, biop, bioq, bior, biot, biou, biov, bip, bipc, bipd, bipe,
bipf, bipg, biph, bipi, bipj, bipk, bipl, bipm, bipn, bipo, bipp, bipq, bipr, bips, bipt, bipu, bipv, biq, biqc,
biqd, biqe, biqf, biqg, biqh, biqi, biqj, biqk, biql, biqm, biqn, biqp, biqq, biqr, biqs, biqt, biqu, biqv, bir,
birc, bird, bire, birf, birg, birh, biri, birj, birk, birl, birm, birn, biro, birp, birq, birr, birs, birt, biru, birv,
bis, bisc, bisd, bise, bisf, bisg, bish, bisi, bisl, bism, biso, bisq, bisr, bist, bisu, bisv, bit, bitc, bitd, bite,
bitf, bitg, bith, biti, bitj, bitl, bitm, bitp, bitq, bitr, bitt, bitu, bitv, biu, biuf, biuj, biv, bivc, bivd, bive, bivf,
bivg, bivh, bivi, bivk, bivl, bivm, bivn, bivp, bivq, bivr, bivs, bivt, bivv, bjbj, bjbk, bjbl, bjbm, bjbn, bjbo,
bjbp, bjbq, bjbr, bjbt, bjbu, bjbv, bjc, bjcd, bjce, bjcf, bjcg, bjch, bjcj, bjck, bjcm, bjcn, bjcp, bjcq, bjcr,
bjct, bjcv, bjd, bjdd, bjde, bjdf, bjdg, bjdh, bjdj, bjdk, bjdl, bjdn, bjdq, bjdr, bjdt, bjdv, bje, bjed, bjee,
bjef, bjeg, bjej, bjek, bjel, bjem, bjen, bjeo, bjeq, bjer, bjes, bjet, bjev, bjf, bjfc, bjfd, bjfe, bjff, bjfg, bjfh,
bjfi, bjfj, bjfk, bjfl, bjfm, bjfp, bjfq, bjfr, bjfs, bjft, bjfu, bjfv, bjg, bjgd, bjge, bjgf, bjgg, bjgj, bjgl, bjgn,
bjgo, bjgq, bjgr, bjgt, bjgv, bjh, bjhd, bjhe, bjhf, bjhg, bjhj, bjhk, bjhl, bjhm, bjhn, bjho, bjhq, bjhr, bjhs,
bjht, bjhv, bji, bjid, bjie, bjig, bjik, bjil, bjin, bjip, bjiq, bjir, bjis, bjit, bjiv, bjjc, bjjd, bjje, bijf, bijg, bjjh,
bjji, bjjj, bjjm, bjjp, bjjq, bjjr, bjjs, bjjt, bjju, bjjv, bjk, bjkd, bjke, bjkj, bjkk, bjkq, bjkr, bjkt, bjkv, bjl,
bjld, bjle, bjlk, bjln, bjlq, bjlv, bjm, bjmd, bjme, bjmf, bjmg, bjml, bjmn, bjmo, bjmq, bjmr, bjmt, bjmv,
bjn, bjnf, bjo, bjp, bjpd, bjpe, bjpf, bjpg, bjph, bjpj, bjpk, bjpl, bjpm, bjpn, bjpo, bjpp, bjpq, bjpr, bjpt,
bjpv, bjq, bjqd, bjqe, bjqf, bjqg, bjqi, bjqk, bjql, bjqn, bjqq, bjqr, bjqt, bjqv, bjr, bjrc, bjrd, bjre, bjrf, bjrg,
bjrh, bjri, bjrj, bjrk, bjrl, bjrm, bjrn, bjro, bjrp, bjrq, bjrr, bjrt, bjrv, bjs, bjsd, bjse, bjsf, bjsg, bjsh, bjsk,
bjsl, bjsm, bjsn, bjso, bjsq, bjsr, bjst, bjsv, bjt, bjte, bjtd, bjte, bjtf, bjtg, bjth, bjti, bjtj, bjtk, bjtl, bjtm,
bjtn, bjtp, bjtq, bjtr, bjtt, bjtv, bjuc, bjud, bjue, bjuf, bjug, bjuh, bjui, bjuj, bjuk, bjul, bjum, bjun, bjup,
bjuq, bjur, bjut, bjuv, bjv, bjvd, bjve, bjvf, bjvg, bjvh, bjvk, bjvl, bjvm, bjvn, bjvo, bjvp, bjvq, bjvr, bjvt,
bjvv, bkbk, bkbl, bkbm, bkbn, bkbo, bkbp, bkbq, bkbr, bkbs, bkbt, bkbu, bkbv, bkc, bkcc, bkcd, bkce,
bkcf, bkcg, bkch, bkci, bkck, bkcl, bkcm, bkcn, bkco, bkcp, bkcq, bkcr, bkcs, bkct, bkcv, bkd, bkdc,
bkdd, bkde, bkdf, bkdg, bkdh, bkdj, bkdk, bkdl, bkdm, bkdn, bkdo, bkdp, bkdq, bkdr, bkds, bkdt, bkdv,
bke, bkec, bked, bkee, bkef, bkeg, bkeh, bkei, bkej, bkek, bkel, bkem, bken, bkeo, bkep, bkeq, bker,
bkes, bket, bkeu, bkev, bkf, bkfc, bkfd, bkfe, bkff, bkfg, bkfh, bkfi, bkfj, bkfk, bkfl, bkfm, bkfp, bkfq,
bkfr, bkfs, bkft, bkfu, bkfv, bkg, bkgc, bkgd, bkge, bkgf, bkgg, bkgh, bkgi, bkgj, bkgk, bkgl, bkgm, bkgn,
bkgo, bkgp, bkgq, bkgr, bkgs, bkgt, bkgu, bkgv, bkh, bkhc, bkhd, bkhe, bkhf, bkhg, bkhh, bkhi, bkhj,
bkhk, bkhl, bkhm, bkhn, bkho, bkhp, bkhq, bkhr, bkhs, bkht, bkhv, bki, bkic, bkid, bkie, bkif, bkig, bkih,
bkii, bkik, bkil, bkim, bkin, bkio, bkip, bkiq, bkir, bkis, bkit, bkiv, bkj, bkjc, bkjd, bkjf, bkjg, bkjh, bkji,
bkjj, bkjk, bkjm, bkjp, bkjq, bkjr, bkjt, bkjv, bkk, bkkd, bkke, bkkf, bkkg, bkkh, bkki, bkkj, bkkk, bkkl,
bkkm, bkkn, bkkp, bkkq, bkkr, bkkt, bkku, bkkv, bkl, bkld, bkle, bklf, bklg, bklh, bklj, bklk, bkll, bklm,
bkln, bklo, bklp, bklq, bklr, bklt, bklv, bkm, bkmc, bkmd, bkme, bkmf, bkmg, bkmh, bkmj, bkmk, bkml,
bkmn, bkmo, bkmp, bkmq, bkmr, bkmt, bkn, bknc, bknf, bknj, bknm, bknt, bko, bkoe, bkof, bkoj, bkoq,
bkor, bkp, bkpc, bkpd, bkpe, bkpf, bkpg, bkph, bkpi, bkpj, bkpk, bkpl, bkpn, bkpo, bkpp, bkpq, bkpr,
bkpt, bkpu, bkpv, bkq, bkqc, bkqd, bkqe, bkqf, bkqg, bkqh, bkqi, bkqj, bkqk, bkql, bkqm, bkqn, bkqo,
bkqp, bkqq, bkqr, bkqt, bkqu, bkqv, bkr, bkrc, bkrd, bkre, bkrf, bkrg, bkrh, bkri, bkrj, bkrk, bkrl, bkrm,
bkrn, bkro, bkrp, bkrq, bkrr, bkrt, bkru, bkrv, bks, bksc, bksd, bkse, bksf, bksg, bksk, bksl, bksn, bksp,
bksq, bksr, bkst, bksu, bksv, bkt, bktc, bktd, bkte, bktf, bktg, bkth, bkti, bktj, bktk, bktl, bktm, bktn, bkto,
bktp, bktq, bktr, bkts, bktt, bktu, bktv, bku, bkud, bkue, bkuf, bkug, bkuh, bkuj, bkul, bkun, bkuo, bkup,
bkuq, bkur, bkut, bkuv, bkv, bkvc, bkvd, bkve, bkvf, bkvg, bkvh, bkvi, bkvj, bkvk, bkvl, bkvm, bkvn,
bkvo, bkvp, bkvq, bkvr, bkvs, bkvt, bkvu, bkvv, blbl, blbm, blbn, blbo, blbp, blbq, blbr, blbs, blbt, blbu,
blbv, blc, blcd, blce, blcf, blcg, blci, blck, blcl, blcm, blcn, blco, blcp, blcq, blcr, blcs, blct, blcv, bld,
bldc, bldd, blde, bldf, bldg, bldh, bldj, bldk, bldl, bldm, bldn, bldo, bldp, bldq, bldr, blds, bldt, bldv, ble,
blec, bled, blee, blef, bleg, bleh, blei, blej, blek, blel, blem, blen, bleo, blep, bleq, bler, bles, blet, bleu,
blev, blf, blfc, blfd, blfe, biff, blfg, blfh, blfi, blfj, blfk, blfl, blfm, blfn, blfo, blfp, blfq, blfr, blfs, blft,
blfu, blfv, blg, blgc, blgd, blge, blgf, blgg, blgh, blgi, blgj, blgk, blgl, blgm, blgn, blgo, blgp, blgq, blgr,
blgs, blgt, blgu, blgv, blh, blhc, blhd, blhe, blhf, blhg, blhh, blhi, blhk, blhl, blhm, blhn, blho, blhp, blhq,
blhr, blht, blhv, bli, blic, blid, blie, blif, blig, blih, blii, blik, blil, blim, blin, blio, blip, bliq, blir, blis, blit,
bliv, blj, bljc, bljd, blje, bljf, bljg, bljh, blji, bljj, bljk, bljl, bljm, bljn, bljp, bljq, bljr, bljs, bljt, blju, bljv,
blk, blkc, blkd, blke, blkf, blkg, blkh, blki, blkk, blkl, blkm, blkn, blko, blkp, blkq, blkr, blkt, blku, blkv,
bll, bllc, blld, blle, bllf, bllg, bllh, blli, blij, bllk, blll, bllm, blln, bllo, bllp, bllq, bllr, bllt, bllu, bllv, blm,
blmc, blmd, blme, blmf, blmg, blmh, blmj, blmk, blml, blmm, blmo, blmp, blmq, blmr, blmt, blmv, bln,
blnc, blnd, blne, blnf, blng, blnh, blni, blnj, blnk, blnm, blnn, blnp, blng, blnr, blnt, blnu, blnv, blo, blod,
bloe, blof, bloj, blok, blol, blon, bloq, blor, blot, blov, blp, blpc, blpd, blpe, blpf, blpg, blph, blpi, blpj,
blpk, blpl, blpm, blpn, blpo, blpp, blpq, blpr, blpt, blpv, blq, blqc, blqd, blqe, blqf, blqg, blqh, blqi, blqj,
blqk, blql, blqm, blqn, blqo, blqp, blqq, blqr, blqt, blqu, blqv, blr, blrc, blrd, blre, blrf, blrg, blrh, blri,
blrj, blrk, blrl, blrm, blrn, blro, blrp, blrq, blrr, blrs, blrt, blru, blrv, bls, blsc, blsd, blse, blsf, blsg, blsk,
blsl, blsn, blso, blsp, blsq, blsr, blst, blsv, blt, bltc, bltd, blte, bltf, bltg, blth, blti, bltj, bltk, bltl, bltm, bltn,
blto, bltp, bltq, bltr, bltt, bltu, bltv, blu, blue, bluf, blug, bluh, blul, blun, bluo, blup, bluq, blur, blut, bluv,
blv, blvc, blvd, blve, blvf, blvg, blvh, blvi, blvj, blvk, blvl, blvm, blvn, blvo, blvp, blvq, blvr, blvs, blvt,
blvv, bmbm, bmbp, bmbq, bmbr, bmbs, bmbt, bmbu, bmbv, bmc, bmcc, bmcd, bmce, bmcf, bmcg, bmch,
bmci, bmcj, bmcl, bmcm, bmcn, bmcp, bmcq, bmcr, bmcs, bmct, bmcu, bmcv, bmd, bmdc, bmde, bmdf,
bmdg, bmdh, bmdi, bmdm, bmdp, bmdr, bmds, bmdt, bmdu, bmdv, bme, bmec, bmed, bmee, bmef,
bmeg, bmeh, bmei, bmek, bmem, bmep, bmeq, bmer, bmes, bmet, bmev, bmf, bmfc, bmfd, bmfe, bmff,
bmfg, bmfh, bmfi, bmfj, bmfk, bmfm, bmfp, bmfq, bmfr, bmfs, bmft, bmfu, bmfv, bmg, bmgc, bmgd,
bmge, bmgf, bmgg, bmgh, bmgi, bmgk, bmgm, bmgp, bmgq, bmgr, bmgs, bmgt, bmgu, bmgv, bmh,
bmhf, bmhg, bmhh, bmhi, bmhk, bmhm, bmhp, bmhq, bmbr, bmht, bmhv, bmi, bmic, bmid, bmie, bmig,
bmih, bmii, bmik, bmim, bmip, bmiq, bmir, bmit, bmiv, bmj, bmjc, bmjf, bmjg, bmjh, bmjj, bmjm, bmjr,
bmjs, bmjt, bmju, bmk, bmkf, bmkg, bmkj, bmkm, bml, bmlc, bmld, bmle, bmlf, bmlg, bmlh, bmli,
bmlk, bmll, bmlm, bmlp, bmlq, bmlr, bmls, bmlt, bmlu, bmlv, bmm, bmmc, bmmf, bmn, bmnf, bmnh,
bmnm, bmns, bmo, bmoc, bmod, bmoe, bmof, bmog, bmoh, bmoi, bmoj, bmol, bmom, bmop, bmoq,
bmor, bmos, bmot, bmou, bmov, bmp, bmpc, bmpe, bmpf, bmpg, bmph, bmpi, bmpj, bmpn, bmpp,
bmpq, bmpr, bmps, bmpt, bmpu, bmpv, bmq, bmqc, bmqd, bmqg, bmqh, bmqj, bmqm, bmqs, bmqt,
bmr, bmrc, bmrd, bmre, bmrf, bmrg, bmrh, bmri, bmrj, bmrk, bmrm, bmrn, bmrp, bmrq, bmrr, bmrs,
bmrt, bmru, bmrv, bms, bmsg, bmsh, bmsi, bmsq, bmsr, bmst, bmsv, bmt, bmte, bmtf, bmtg, bmth, bmtj,
bmtm, bmtr, bmts, bmtt, bmtu, bmu, bmv, bmvc, bmvd, bmve, bmvg, bmvh, bmvm, bmvq, bmvr, bmvs,
bmvt, bmvu, bmvv, bnbn, bnbo, bnbp, bnbq, bnbr, bnbt, bnbu, bnbv, bnc, bncd, bnce, bncf, bncg, bnch,
bncj, bnck, bncl, bncn, bncp, bncq, bncr, bnct, bncv, bnd, bndd, bnde, bndg, bndj, bndk, bndl, bndn,
bndq, bndt, bndv, bne, bned, bnee, bnef, bneg, bnej, bnek, bnel, bnen, bneo, bnep, bneq, bner, bnes, bnet,
bnev, bnf, bnfc, bnfd, bnfe, bnff, bnfg, bnfh, bnfi, bnfj, bnik, bnfl, bnfm, bnfn, bnfo, bnfp, bnfq, bnfr,
bnfs, bnft, bnfu, bnfv, bng, bngd, bnge, bngf, bngg, bngj, bngl, bngn, bngo, bngp, bngq, bngr, bngt, bngv,
bnh, bnhd, bnhe, bnhf, bnhg, bnhj, bnhk, bnhl, bnhn, bnho, bnhp, bnhq, bnhr, bnht, bnhv, bni, bnid, bnie,
bnif, bnig, bnik, bnil, bnin, bnio, bnip, bniq, bnir, bnit, bniv, bnj, bnjc, bnjd, bnje, bnjf, bnjg, bnjh, bnji,
bnjj, bnjk, bnjm, bnjn, bnjp, bnjq, bnjr, bnjs, bnjt, bnju, bnjv, bnk, bnke, bnkg, bnkk, bnkl, bnkn, bnkq,
bnkr, bnkt, bnkv, bnl, bnle, bnln, bnlq, bnlv, bnm, bnmd, bnme, bnmf, bnmg, bnmj, bnmk, bnml, bnmo,
bnmp, bnmq, bnmr, bnmt, bnmv, bnn, bno, bnp, bnpd, bnpe, bnpf, bnpg, bnpj, bnpk, bnpl, bnpn, bnpo,
bnpp, bnpq, bnpr, bnpt, bnpv, bnq, bnqd, bnqe, bnqf, bnqg, bnqj, bnqk, bnql, bnqn, bnqq, bnqr, bnqt,
bnqv, bnr, bnrd, bnre, bnrf, bnrg, bnrh, bnrj, bnrk, bnrl, bnr, bnro, bnrp, bnrq, bnrr, bnrt, bnrv, bns,
bnsd, bnse, bnsf, bnsg, bnsk, bnsl, bnsn, bnso, bnsq, bnsr, bnst, bnsv, bnt, bntd, bnte, bntf, bntg, bnth,
bnti, bntj, bntk, bntl, bntn, bnto, bntp, bntq, bntr, bntt, bntv, bnud, bnue, bnuf, bnug, bnuh, bnuk, bnul,
bnun, bnuo, bnuq, bnur, bnut, bnuv, bnv, bnvd, bnve, bnvf, bnvg, bnvj, bnvk, bnvl, bnvn, bnvo, bnvp,
bnvq, bnvr, bnvt, bnvv, bobo, bobp, bobq, bobr, bobt, bobu, bobv, boc, bocd, boce, bocf, bocg, bock,
bocl, bocm, bocn, boco, bocp, bocq, bocr, boct, bocv, bod, bodd, bode, bodf, bodg, bodj, bodk, bodl,
bodn, bodo, bodp, bodq, bodr, bodt, bodv, boe, boec, boed, boee, boef, boeg, boeh, boei, boej, boek,
boel, boem, boen, boeo, boep, boeq, boer, boes, boet, boeu, boev, bof, bofc, bofd, bofe, boff, bofg, bofh,
bofi, bofk, bofl, bofm, bofn, bofo, bofp, bofq, bofr, bofs, boft, bofu, bofv, bog, bogc, bogd, boge, bogf,
bogg, bogh, bogi, bogj, bogk, bogl, bogm, bogn, bogp, bogq, bogr, bogt, bogu, bogv, boh, bohd, bohe,
bohf, bobg, bohh, bohk, bohl, bohn, boho, bohp, bohq, bohr, boht, bohv, boi, boid, boie, boif, boig, boii,
boik, boil, boim, boio, boip, boiq, boir, bois, boit, boiv, boj, bojc, bojd, boje, bojf, bojg, bojh, boji, bojj,
bojk, bojl, bojp, bojq, bojr, bojs, bojt, boju, bojv, bok, bokd, boke, bokf, bokg, bokh, boki, bokk, bokl,
bokn, boko, bokp, bokq, bokr, bokt, bokv, bol, bold, bole, bolf, bolg, bolk, boll, boln, bolo, bolp, bolq,
bolr, bolt, bolv, bom, bome, bomq, bomr, bomv, bon, bone, bonf, bong, boni, bonj, bonk, bonq, bonr,
bont, bonv, boo, bop, bopd, bope, bopf, bopg, boph, bopk, bopl, bopm, bopn, bopo, bopp, bopq, bopr,
bopt, bopv, boq, boqd, boqe, boqf, boqg, boqh, boqj, boqk, boql, boqn, boqo, boqp, boqq, boqr, boqt,
boqv, bor, bord, bore, borf, borg, borh, borj, bork, borl, borm, born, boro, borp, borq, borr, bort, borv,
bos, bosc, bose, bosi, bosl, boso, bosq, bosr, bosv, bot, botd, bote, botf, botg, both, botj, botk, botl, botm,
botn, boto, botp, botq, botr, bott, botv, bou, boud, boue, bouf, boug, boul, bouo, boup, bouq, bour, bout,
bouv, bov, bovd, bove, bovf, bovg, bovh, bovi, bovk, bovl, bovm, bovn, bovo, bovp, bovq, bovr, bovt,
bovv, bpbp, bpbq, bpbr, bpbs, bpbt, bpbu, bpbv, bpc, bpcc, bpcd, bpcf, bpcg, bpch, bpci, bpck, bpcl,
bpcm, bpcn, bpco, bpcp, bpcq, bpcr, bpcs, bpct, bpcu, bpcv, bpd, bpdc, bpdd, bpde, bpdf, bpdg, bpdh,
bpdi, bpdj, bpdk, bpdl, bpdm, bpdn, bpdp, bpdq, bpdr, bpds, bpdt, bpdv, bpe, bpec, bped, bpee, bpef,
bpeg, bpeh, bpei, bpej, bpek, bpel, bpem, bpen, bpeo, bpep, bpeq, bper, bpes, bpet, bpeu, bpev, bpf, bpfc,
bpfd, bpfe, bpff, bpfg, bpfh, bpfi, bpfj, bpfk, bpfl, bpfm, bpfp, bpfq, bpfr, bpfs, bpft, bpfu, bpfv, bpgc,
bpgd, bpge, bpgf, bpgg, bpgh, bpgi, bpgj, bpgk, bpgl, bpgm, bpgn, bpgo, bpgp, bpgq, bpgr, bpgs, bpgt,
bpgu, bpgv, bph, bphc, bphd, bphe, bphf, bphg, bphh, bphi, bphk, bphl, bphm, bphn, bpho, bphp, bphq,
bphr, bphs, bpht, bphv, bpi, bpic, bpid, bpie, bpif, bpig, bpih, bpii, bpil, bpim, bpin, bpio, bpip, bpiq,
bpir, bpis, bpit, bpiu, bpiv, bpj, bpjc, bpjd, bpjf, bpjg, bpjh, bpji, bpjj, bpjm, bpjp, bpjr, bpjs, bpjt, bpju,
bpjv, bpk, bpkc, bpkd, bpke, bpkg, bpkh, bpki, bpkk, bpkl, bpkm, bpkp, bpkq, bpkr, bpks, bpkt, bpku,
bpkv, bpl, bplc, bpld, bple, bplf, bplg, bplb, bpli, bplj, bplk, bpll, bplm, bpln, bplo, bplp, bplq, bplr, bpls,
bplt, bplu, bplv, bpm, bpmc, bpme, bpmf, bpmg, bpmn, bpmo, bpmp, bpmq, bpmr, bpmt, bpmu, bpmv,
bpn, bpnc, bpnd, bpne, bpnf, bpng, bpnh, bpni, bpnj, bpnm, bpnp, bpnq, bpnr, bpns, bpnt, bpnu, bpnv,
bpo, bpoc, bpod, bpoe, bpof, bpog, bpoh, bpoi, bpoj, bpok, bpol, bpom, bpon, bpop, bpoq, bpor, bpos,
bpot, bpou, bpov, bpp, bppd, bppe, bppf, bppg, bpph, bppi, bppk, bppl, bppm, bppn, bppo, bppp, bppq,
bppr, bppt, bppu, bppv, bpq, bpqc, bpqd, bpqe, bpqf, bpqg, bpqh, bpqi, bpqi, bpqk, bpql, bpqm, bpqn,
bpqo, bpqp, bpqq, bpqr, bpqs, bpqt, bpqu, bpqv, bpr, bprc, bprd, bpre, bprf, bprg, bprh, bpri, bprj, bprk,
bprl, bprm, bprn, bpro, bprp, bprq, bprr, bprs, bprt, bpru, bprv, bps, bpsc, bpsd, bpse, bpsg, bpsi, bpsl,
bpso, bpsq, bpsr, bpst, bpsv, bpt, bptc, bptd, bpte, bptf, bptg, bpth, bpti, bptj, bptk, bptl, bptm, bptn, bpto,
bptp, bptq, bptr, bpts, bptt, bptu, bptv, bpu, bpue, bpug, bpuh, bpul, bpuo, bpup, bpuq, bpur, bput, bpuv,
bpv, bpvc, bpvd, bpve, bpvf, bpvg, bpvh, bpvi, bpvj, bpvk, bpvl, bpvm, bpvn, bpvo, bpvp, bpvq, bpvr,
bpvs, bpvt, bpvu, bpvv, bqbq, bqbr, bqbs, bqbt, bqbu, bqbv, bqc, bqcc, bqcd, bqce, bqcf, bqcg, bqch,
bqci, bqcj, bqck, bqcl, bqcm, bqcn, bqco, bqcp, bqcq, bqcr, bqcs, bqct, bqcv, bqd, bqdc, bqdd, bqde,
bqdf, bqdg, bqdh, bqdi, bqdj, bqdk, bqdl, bqdm, bqdn, bqdo, bqdp, bqdq, bqdr, bqds, bqdt, bqdv, bqe,
bqec, bqed, bqee, bqef, bqeg, bqeh, bqei, bqej, bqek, bqel, bqem, bqen, bqeo, bqep, bqeq, bqer, bqes,
bqet, bqeu, bqev, bqf, bqfc, bqfd, bqfe, bqff, bqfg, bqfh, bqfi, bqfj, bqfk, bqfl, bqfm, bqfn, bqfo, bqfp,
bqfq, bqfr, bqfs, bqft, bqfu, bqfv, bqg, bqgc, bqgd, bqge, bqgf, bqgg, bqgh, bqgi, bqgj, bqgk, bqgl, bqgm,
bqgn, bqgo, bqgp, bqgq, bqgr, bqgs, bqgt, bqgu, bqgv, bqh, bqhc, bqhd, bqhe, bqhf, bqhg, bqhh, bqhi,
bqhj, bqhk, bqhl, bqhm, bqhn, bqho, qbhp, bqhq, bqhr, bqhs, bqht, bqhv, bqi, bqic, bqid, bqie, bqif, bqig,
bqih, bqii, bqik, bqil, bqim, bqin, bqio, bqip, bqiq, bqir, bqis, bqit, bqiv, bqj, bqjc, bqjd, bqje, bqjf, bqjg,
bqjh, bqji, bqjj, bqjk, bqjl, bqjm, bqjn, bqjp, bqjq, bqjr, bqjs, bqjt, bqju, bqjv, bqk, bqkc, bqkd, bqke,
bqkf, bqkg, bqkh, bqki, bqkk, bqkl, bqkm, bqkn, bqkp, bqkq, bqkr, bqks, bqkt, bqku, bqkv, bql, bqlc,
bqld, bqle, bqlf, bqlg, bqlh, bqli, bqlj, bqlk, bqll, bqlm, bqln, bqlo, bqlp, bqlq, bqlr, bqls, bqlt, bqlu, bqlv,
bqm, bqmc, bqmd, bqme, bqmf, bqmg, bqmh, bqmi, bqmk, bqml, bqmo, bqmp, bqmq, bqmr, bqmt,
bqmv, bqn, bqnc, bqnd, bqne, bqnf, bqng, bqnh, bqni, bqnj, bqnk, bqnl, bqnm, bqnp, bqnq, bqnr, bqns,
bqnt, bqnu, bqnv, bqo, bqod, bqoe, bqof, bqog, bqoi, bqoj, bqok, bqol, bqom, bqon, bqop, bqoq, bqor,
bqot, bqov, bqp, bqpc, bqpd, bqpe, bqpf, bqpg, bqpi, bqpj, bqpk, bqpl, bqpn, bqpo, bqpp, bqpq, bqpr,
bqps, bqpt, bqpu, bqpv, bqq, bqqc, bqqd, bqqe, bqqf, bqqg, bqqh, bqqi, bqqj, bqqk, bqql, bqqm, bqqn,
bqqo, bqqp, bqqq, bqqr, bqqs, bqqt, bqqu, bqqv, bqr, bqro, bqrd, bqre, bqrf, bqrg, bqrh, bqri, bqrj, bqrk,
bqrl, bqrm, bqrn, bqro, bqrp, bqrq, bqrr, bqrs, bqrt, bqru, bqrv, bqs, bqsc, bqsd, bqse, bqsf, bqsg, bqsh,
bqsi, bqsl, bqsm, bqso, bqsq, bqsr, bqst, bqsv, bqt, bqtc, bqtd, bqte, bqtf, bqtg, bqth, bqti, bqtj, bqtk, bqtl,
bqtm, bqtn, bqto, bqtp, bqtq, bqtr, bqts, bqtt, bqtu, bqtv, bqu, bque, bqud, bque, bquf, bqug, bquh, bquk,
bqul, bqun, bquo, bqup, bquq, bqur, bqut, bquv, bqv, bqvc, bqvd, bqve, bqvf, bqvg, bqvh, bqvi, bqvj,
bqvk, bqvl, bqvm, bqvn, bqvo, bqvp, bqvq, bqvr, bqvt, bqvu, bqvv, brbr, brbs, brbt, brbu, brbv, brc, brcc,
brcd, brce, brcf, brcg, brch, brci, broj, brck, brcl, brcm, brcn, brco, brcp, brcq, brcr, brcs, brct, brcu, brcv,
brd, brdc, brdd, brde, brdf, brdg, brdh, brdj, brdk, brdl, brdm, brdn, brdp, brdq, brdr, brds, brdt, brdv,
bre, brec, bred, bree, bref, breg, breh, brei, brej, brek, brel, brem, bren, breo, brep, breq, brer, bres, bret,
breu, brev, brf, brfc, brfd, brfe, brff, brfg, brfh, brfi, brfj, brfk, brfl, brfm, brfn, brfo, brfp, brfq, brfr, brfs,
brft, brfu, brfv, brg, brgc, brgd, brge, brgf, brgg, brgh, brgi, brgj, brgk, brgl, brgm, brgn, brgo, brgp, brgq,
brgr, brgs, brgt, brgu, brgv, brh, brhc, brhd, brhe, brhf, brbg, brhh, brhi, brhj, brhk, brhl, brhm, brhn,
brho, brhp, brhq, brhr, brhs, brht, brhv, bri, bric, brid, brie, brif, brig, brih, brii, brik, bril, brim, brin, brio,
brip, briq, brir, bris, brit, briv, brj, brjc, brjd, brje, brjf, brjg, brjh, brji, brij, brjk, brjl, brjm, brjp, brjq,
brjr, bris, brjt, brju, brjv, brk, brkc, brkd, brke, brkf, brkg, brkh, brki, brkj, brkk, brkl, brkm, brkn, brkp,
brkq, brkr, brks, brkt, brku, brkv, brl, brlc, brld, brle, brlf, brlg, brlh, brli, brlj, brlk, brll, brlm, brln, brlp,
brlq, brlr, brls, brlt, brlu, brlv, brm, brmc, brmd, brme, brmf, brmg, brmh, brmk, brml, brmm, brmo,
brmp, brmq, brmr, brmt, brmv, brn, brnc, brnd, brne, brnf, brng, brnh, brni, brnj, brnk, brnm, brnp, brnq,
brnr, brus, brnt, brnu, brnv, bro, broc, brod, broe, brof, brog, broh, broi, broj, brol, brom, bron, brop,
broq, bror, bros, brot, brou, brov, brp, brpc, brpd, brpe, brpf, brpg, brph, brpi, brpj, brpk, brpl, brpm,
brpn, brpo, brpp, brpq, brpr, brps, brpt, brpu, brpv, brq, brqc, brqd, brqe, brqf, brqg, brqh, brqi, brqj,
brqk, brql, brqm, brqn, brqo, brqp, brqq, brqr, brqs, brqt, brqu, brqv, brr, brrc, brrd, brre, brrf, brrg, brrh,
brri, brrj, brrk, brrl, brrm, brrn, brro, brrp, brrq, brrr, brrs, brrt, brru, brrv, brs, brsc, brsd, brse, brsf, brsg,
brsi, brsl, brsm, brsn, brso, brsg, brsr, brss, brst, brsu, brsv, brt, brtc, brtd, brte, brtf, brtg, brth, brti, brtj,
brtk, brtl, brtm, brtn, brto, brtp, brtq, brtr, brts, brtt, brtu, brtv, bru, bruc, brud, brue, bruf, brug, bruh,
bruk, brul, brun, bruo, brup, brug, brur, brut, bruv, brv, brve, brvd, brve, brvf, brvg, brvh, brvi, brvj, brvk,
brvl, brvm, brvn, brvo, brvp, brvq, brvr, brvs, brvt, brvu, brvv, bsbs, bsbt, bsbu, bsbv, bsc, bscc, bscd,
bsce, bscf, bscg, bsci, bsck, bscl, bscp, bscq, bscr, bscs, bsct, bscv, bsd, bsdc, bsde, bsdf, bsdg, bsdh,
bsdk, bsdm, bsdp, bsdr, bsdt, bsdu, bse, bsec, bsed, bsee, bsef, bseg, bseh, bsei, bsek, bsel, bsem, bsen,
bsep, bseq, bser, bses, bset, bseu, bsev, bsf, bsfc, bsfd, bsff, bsfg, bsfh, bsfi, bsfk, bsfl, bsfm, bsfp, bsfq,
bsfr, bsft, bsfu, bsfv, bsg, bsgc, bsgd, bsge, bsgf, bsgg, bsgh, bsgi, bsgk, bsgl, bsgm, bsgp, bsgq, bsgr,
bsgs, bsgt, bsgu, bsgv, bsh, bshd, bshe, bshf, bshg, bshh, bshi, bshk, bshl, bshm, bshp, bshq, bshr, bshs,
bsht, bsi, bsic, bsid, bsie, bsig, bsih, bsii, bsik, bsil, bsim, bsip, bsiq, bsir, bsis, bsit, bsiv, bsj, bsjf, bsk,
bskc, bskd, bskf, bskg, bskh, bski, bskk, bskm, bskp, bskq, bskr, bsks, bskt, bsku, bskv, bsl, bslc, bsld,
bsle, bslf, bslg, bslh, bsli, bslk, bsll, bslm, bslp, bslg, bslr, bsls, bslt, bslu, bslv, bsm, bsmc, bsme, bsmf,
bsmg, bsmi, bsmq, bsmr, bsmt, bsn, bsnd, bsnf, bsng, bsnh, bsnj, bsnm, bsnr, bsns, bsnt, bsnu, bso, bsoc,
bsod, bsoe, bsof, bsog, bsoi, bsok, bsol, bsom, bsop, bsoq, bsor, bsos, bsot, bsou, bsov, bsp, bspc, bspe,
bspf, bspg, bsph, bspi, bspj, bspm, bspq, bspr, bsps, bspt, bspu, bspv, bsq, bsqc, bsqd, bsqe, bsqf, bsqg,
bsqh, bsqi, bsqj, bsqk, bsql, bsqm, bsqn, bsqp, bsqq, bsqr, bsqs, bsqt, bsqu, bsqv, bsr, bsrc, bsrd, bsre,
bsrf, bsrg, bsrh, bsri, bsrj, bsrm, bsrp, bsrq, bsrr, bsrt, bsru, bsrv, bss, bssc, bssp, bssq, bssr, bsst, bssu,
bssv, bst, bstc, bste, bstf, bstg, bsth, bsti, bstj, bstm, bstp, bstr, bsts, bstt, bstu, bsu, bsuh, bsuj, bsup, bsur,
bsut, bsv, bsvc, bsvd, bsve, bsvf, bsvg, bsvh, bsvi, bsvk, bsvm, bsvn, bsvp, bsvq, bsvr, bsvs, bsvt, bsvu,
bsvv, btbt, btbu, btbv, btc, btcc, btcd, btce, btcf, btcg, btch, btci, btcj, btck, btcl, btcm, btcn, btco, btcp,
btcq, btcr, btcs, btct, btcv, btd, btdc, btdd, btde, btdf, btdg, btdh, btdi, btdj, btdk, btdl, btdm, btdn, btdp,
btdq, btdr, btds, btdt, btdv, bte, btec, bted, btee, btef, bteg, bteh, btei, btej, btek, btel, btem, bten, bteo,
btep, bteq, bter, btes, btet, bteu, btev, btf, btfc, btfd, btfe, btff, btfg, btfh, btfi, btfj, btfk, btfl, btfm, btfn,
btfo, btfp, btfq, btfr, btfs, btft, btfu, btfv, btg, btgc, btgd, btge, btgf, btgg, btgh, btgi, btgj, btgk, btgl, btgm,
btgn, btgo, btgp, btgq, btgr, btgs, btgt, btgu, btgv, bth, bthc, bthd, bthe, bthf, bthg, bthh, bthi, bthj, bthk,
bthl, bthm, bthn, btho, bthp, bthq, bthr, bths, btht, bthv, bti, btic, btid, btie, btif, btig, btih, btii, btik, btil,
btim, btin, btio, btip, btiq, btir, btis, btit, btiv, btj, btjc, btjd, btje, btjf, btjg, btjh, btji, btjj, btjk, btjl, btjm,
btjn, btjp, btjq, btjr, btjs, btjt, btju, btjv, btk, btkc, btkd, btke, btkf, btkg, btkh, btki, btkj, btkk, btkl, btkm,
btkn, btkp, btkq, btkr, btks, btkt, btku, btkv, btl, btlc, btld, btle, btlf, btlg, btlh, btli, btlj, btlk, btll, btlm,
btln, btlp, btlq, btlr, btls, btlt, btlu, btlv, btm, btmc, btmd, btme, btmf, btmg, btmh, btmk, btml, btmm,
btmo, btmp, btmq, btmr, btms, btmt, btmv, btn, btnc, btnd, btne, btnf, btng, btnh, btnj, btnm, btnp, btnr,
btns, btnt, btnu, btnv, bto, btod, btoe, btof, btog, btoh, btoi, btoj, btom, bton, btop, btoq, btor, btos, btot,
btou, btov, btp, btpc, btpd, btpe, btpf, btpg, btph, btpi, btpj, btpk, btpl, btpm, btpn, btpo, btpq, btpr, btpt,
btpu, btpv, btq, btqc, btqd, btqe, btqf, btqg, btqh, btqi, btqj, btqk, btql, btqm, btqn, btqo, btqp, btqq, btqr,
btqs, btqt, btqu, btqv, btr, btrc, btrd, btre, btrf, btrg, btrh, btri, btrj, btrk, btrl, btrm, btrn, btro, btrp, btrq,
btrr, btrs, btrt, btru, btrv, bts, btsc, btsd, btse, btsf, btsg, btsi, btsl, btsm, btsn, btso, btsq, btsr, btss, btst,
btsu, btsv, btt, bttc, bttd, btte, bttf, bttg, btth, btti, bttj, bttk, bttl, bttm, bttn, btto, bttp, bttq, bttr, btts, bttt,
bttu, bttv, btu, btuc, btud, btue, btuf, btug, btuh, btuk, btul, btum, btun, btuo, btup, btuq, btur, but, btuv,
btv, btvc, btvd, btve, btvf, btvg, btvh, btvi, btvj, btvk, btvl, btvm, btvn, btvo, btvp, btvq, btvr, btvs, btvt,
btvu, btvv, bubu, bubv, buc, bucc, bucd, bucg, buch, buci, bucl, bucm, bucp, bucq, bucr, bucs, buct, bud,
budc, budd, budf, budg, budh, budp, budq, budr, buds, budt, budv, bue, buec, bued, buee, buef, bueg,
bueh, buei, buej, buek, buel, buem, buep, bueq, buer, bues, buet, bueu, buev, buf, bufc, bufd, bufe, buff,
bufg, bufh, bufi, bufl, bufm, bufp, bufq, bufr, bufs, buft, bufv, bug, bugc, bugd, buge, bugf, bugg, bugh,
bugi, bugj, bugk, bugl, bugm, bugp, bugq, bugr, bugs, bugt, bugu, bugv, buh, buhf, buhg, buhk, buhl,
buhm, buhp, buhq, buhr, buhs, buht, bui, buic, buid, buif, buig, buil, buim, buip, buir, buis, buit, buiv,
buj, bujc, bujd, buje, bujf, bujg, bujh, bujj, bujp, bujr, bujs, bujt, buju, bujv, buk, buke, bukg, bukh, buki,
bukk, bukl, bukm, bukp, bukq, bukr, buks, bukt, bukv, bul, bulc, buld, bule, bulf, bulg, bulh, buli, bulk,
bull, bulm, bulp, bulq, bulr, bult, bulu, bulv, bum, bumc, bumd, bumf, bumg, bumh, bumm, bumr, bun,
bunf, bung, bunh, bunj, bunm, bunr, bunt, bunu, buo, buoe, buoj, buoq, buov, bup, bupc, bupd, bupe,
bupg, buph, bupi, bupj, bupl, bupm, bupq, bupr, bupt, bupv, buq, buqc, buqd, buqe, buqf, buqg, buqh,
buqi, buqj, buqk, buql, buqm, buqn, buqp, buqq, buqr, buqt, buqv, bur, burc, burd, bure, burf, burg, burh,
buri, burj, burl, burm, burp, burq, burr, burs, burt, burv, bus, busi, busl, but, butc, butd, bute, butf, butg,
buth, buti, butj, butl, butm, butp, butq, butr, buts, butt, butv, buu, buuc, buur, buut, buuv, buv, buvc,
buvd, buve, buvf, buvg, buvh, buvi, buvj, buvk, buvl, buvm, buvn, buvp, buvq, buvr, buvs, buvt, buvv,
bvbv, bvc, bvcc, bvcd, bvce, bvcf, bvcg, bvch, bvci, bvcj, bvck, bvcl, bvcm, bvcn, bvco, bvcp, bvcq,
bvcr, bvct, bvcu, bvcv, bvd, bvdc, bvdd, bvde, bvdf, bvdg, bvdh, bvdi, bvdj, bvdk, bvdl, bvdm, bvdn,
bvdo, bvdp, bvdq, bvdr, bvds, bvdt, bvdu, bvdv, bve, bvec, bved, bvee, bvef, bveg, bveh, bvei, bvej,
bvek, bvel, bvem, bven, bveo, bvep, bveq, bver, bves, bvet, bveu, bvev, bvf, bvfc, bvfd, bvfe, bvff, bvfg,
bvfh, bvfi, bvfj, bvfk, bvfl, bvfm, bvfn, bvfo, bvfp, bvfq, bvfr, bvft, bvfu, bvfv, bvg, bvgc, bvgd, bvge,
bvgf, bvgg, bvgh, bvgi, bvgj, bvgk, bvgl, bvgm, bvgn, bvgo, bvgp, bvgq, bvgr, bvgs, bvgt, bvgu, bvgv,
bvh, bvhc, bvhd, bvhe, bvhf, bvhg, bvhh, bvhi, bvhj, bvhk, bvhl, bvhm, bvhn, bvho, bvhp, bvhq, bvhr,
bvhs, bvht, bvhu, bvhv, bvi, bvic, bvid, bvie, bvif, bvig, bvih, bvii, bvik, bvil, bvim, bvin, bvio, bvip,
bviq, bvir, bvis, bvit, bviv, bvj, bvjf, bvjj, bvjm, bvjr, bvjs, bvjt, bvju, bvk, bvkc, bvkd, bvke, bvkf, bvkg,
bvkh, bvki, bvkj, bvkk, bvkl, bvkm, bvkn, bvkp, bvkq, bvkr, bvks, bvkt, bvku, bvkv, bvl, bvlc, bvld,
bvle, bvlf, bvlg, bvlh, bvli, bvlj, bvlk, bvll, bvlm, bvln, bvlo, bvlp, bvlq, bvlr, bvls, bvlt, bvlu, bvlv, bvm,
bvmc, bvmd, bvme, bvmf, bvmg, bvmh, bvmj, bvmk, bvml, bvmn, bvmo, bvmp, bvmq, bvmr, bvmt,
bvmv, bvn, bvnc, bvnd, bvne, bvnf, bvng, bvnh, bvni, bvnj, bvnk, bvnl, bvnm, bvnp, bvnq, bvnr, bvns,
bvnt, bvnu, bvnv, bvo, bvoc, bvod, bvoe, bvof, bvog, bvoh, bvoi, bvoj, bvok, bvol, bvom, bvon, bvop,
bvoq, bvor, bvos, bvot, bvou, bvov, bvp, bvpc, bvpd, bvpe, bvpf, bvpg, bvph, bvpi, bvpj, bvpk, bvpl,
bvpm, bvpn, bvpo, bvpp, bvpq, bvpr, bvps, bvpt, bvpu, bvpv, bvq, bvqc, bvqd, bvqe, bvqf, bvqg, bvqh,
bvqi, bvqj, bvqk, bvql, bvqm, bvqn, bvqo, bvqp, bvqq, bvqr, bvqs, bvqt, bvqu, bvqv, bvr, bvrc, bvrd,
bvre, bvrf, bvrg, bvrh, bvri, bvrj, bvrk, bvrl, bvrm, bvrn, bvro, bvrp, bvrq, bvrr, bvrs, bvrt, bvru, bvrv,
bvs, bvsc, bvsd, bvse, bvsf, bvsg, bvsh, bvsi, bvsj, bvsk, bvsl, bvsm, bvsn, bvso, bvsp, bvsq, bvsr, bvss,
bvst, bvsv, bvt, bvtc, bvtd, bvte, bvtf, butg, bvth, bvti, bvtj, bvtk, bvtl, bvtm, bvtn, bvto, bvtp, bvtq, bvtr,
bvts, bvtt, bvtu, bvtv, bvu, bvuc, bvud, bvue, bvuf, bvug, bvuj, bvuk, bvul, bvun, bvuo, bvup, bvuq, bvur,
bvut, bvuv, bvv, bvvc, bvvd, bvve, bvvf, bvvg, bvvh, bvvi, bvvj, bvvk, bvvl, bvvm, bvvn, bvvo, bvvp,
bvvq, bvvr, bvvs, bvvt, bvvu, bvvv, ccc, cccd, ccci, cccp, cccq, cccr, ccd, ccdd, ccde, ccdf, ccdg, ccdq,
ccdr, ccdt, cce, ccee, ccef, cceg, cceh, ccel, ccem, ccen, ccep, cceq, ccer, cces, ccet, ccev, ccf, ccfd, ccfe,
ccff, ccfg, ccfh, ccfi, ccfj, ccfk, ccfm, ccfp, ccfq, ccfr, ccfs, ccft, ccfu, ccfv, ccg, ccgd, ccge, ccgf, ccgg,
ccgh, ccgi, ccgl, ccgp, ccgq, ccgr, ccgt, ccgu, ccgv, cch, cchd, cchf, cchg, cchi, cchp, cchq, cchr, cchs,
ccht, cci, ccie, ccii, ccip, cciq, ccir, ccit, cciv, ccj, ccjf, ccjh, cck, cckf, cckh, cckj, ccks, cckt, ccl, ccle,
cclf, cclg, ccli, cclj, cclk, cclq, cclr, ccls, cclt, cclv, ccm, ccmd, ccme, ccmf, ccmg, ccmi, ccmp, ccmq,
ccmr, ccn, ccnf, cco, ccof, ccoj, ccp, ccpd, ccpe, ccpf, ccpg, ccpi, ccpj, ccpm, ccpq, ccpr, ccps, ccpt, ccq,
ccqd, ccqe, ccqf, ccqg, ccqi, ccqi, ccqm, ccqn, ccqq, ccqr, ccqs, ccqt, ccqv, ccr, ccrd, ccre, ccrf, ccrg,
ccrj, ccrl, ccrm, ccrp, ccrq, ccrr, ccrs, ccrt, ccru, ccrv, ccs, ccsf, ccsg, ccsr, ccst, ccsv, cct, ccte, cctf, ccth,
ccti, cctj, cctl, cctp, cctq, cctr, ccts, cctt, cctu, cctv, ccu, ccur, ccut, ccv, ccvd, ccve, ccvf, ccvg, ccvh,
ccvi, ccvk, ccvm, ccvp, ccvq, ccvr, ccvs, ccvt, ccvv, cdcd, cdce, cdcf, cdcg, cdck, cdcl, cdcm, cdcn, cdco,
cdcp, cdcq, cdcr, cdcs, cdct, cdcv, cddd, cdde, cddg, cddh, cddi, cddl, cddm, cddq, cddr, cddt, cde, cded,
cdee, cdef, cdeg, cdej, cdel, cden, cdeo, cdeq, cder, cdet, cdev, cdf, cdfd, cdfe, cdff, cdfg, cdfh, cdfj, cdfk,
cdfl, cdfq, cdfr, cdfs, cdft, cdfu, cdfv, cdg, cdgd, cdge, cdgf, cdgg, cdgh, cdgi, cdgj, cdgk, cdgl, cdgm,
cdgo, cdgq, cdgr, cdgs, cdgt, cdgu, cdgv, cdh, cdhd, cdhe, cdhf, cdhg, cdhh, cdhi, cdhk, cdhl, cdhm,
cdho, cdhq, cdhr, cdhs, cdht, cdi, cdid, cdie, cdif, cdig, cdih, cdik, cdil, cdim, cdip, cdiq, cdir, cdit, cdiv,
cdj, cdjf, cdjj, cdjr, cdjt, cdju, cdk, cdkd, cdke, cdki, cdkl, cdl, cdle, cdlf, cdlg, cdlh, cdlj, cdln, cdlp, cdlq,
cdlr, cdlt, cdlv, cdm, cdmd, cdme, cdmg, cdmh, cdmi, cdml, cdmm, cdmo, cdmp, cdmq, cdn, cdnf, cdnh,
cdnj, cdnr, cdo, cdoe, cdog, cdoj, cdp, cdpd, cdpe, cdpf, cdpg, cdph, cdpl, cdpm, cdpn, cdpq, cdpr, cdpt,
cdpv, cdq, cdqd, cdqe, cdqf, cdqg, cdqh, cdqi, cdqj, cdql, cdqm, cdqn, cdqq, cdqr, cdqt, cdqv, cdr, cdrd,
cdre, cdrf, cdrg, cdrh, cdri, cdrj, cdrk, cdrl, cdrm, cdrq, cdrr, cdrt, cdru, cdrv, cds, cdse, cdsf, cdsg, cdsl,
cdsq, cdsr, cdsv, cdt, cdtd, cdte, cdtf, cdtg, cdth, cdti, cdtj, cdtl, cdtm, cdto, cdtp, cdtq, cdtr, cdtt, cdtu,
cdtv, cdu, cdue, cduf, cdul, cduq, cdur, cdut, cduv, cdv, cdvd, cdve, cdvf, cdvg, cdvh, cdvi, cdvl, cdvm,
cdvn, cdvo, cdvp, cdvq, cdvr, cdvt, cdvv, ceci, ceck, cecl, cecm, cecn, ceco, cecp, cecq, cecr, cect, cecv,
cedd, cede, cedf, cedg, cedh, cedi, cedj, cedk, cedl, cedm, cedn, cedo, cedp, cedq, cedr, cedt, cedv, cee,
ceed, ceeh, ceei, ceej, ceek, ceel, ceem, ceen, ceeo, ceep, ceeq, ceer, cees, ceet, ceeu, ceev, cef, cefd, ceff,
cefh, cefi, cefj, cefk, cefl, cefm, cefn, cefo, cefp, cefq, cefr, cefs, ceft, cefv, ceg, cegd, cegh, cegi, cegj,
cegk, cegl, cegm, cegn, cego, cegp, cegq, cegr, cegs, cegt, cegu, cegv, ceh, cehd, cehe, cehf, cehg, cehh,
cehi, cehk, cehl, cehm, cehn, ceho, cehq, cehr, cehs, ceht, cehv, cei, ceid, ceie, ceif, ceig, ceii, ceik, ceil,
ceim, cein, ceio, ceiq, ceir, ceis, ceit, ceiv, cej, cejf, cejj, cejq, cejr, cejs, cejt, cek, cekd, ceke, cekf, cekg,
cekh, ceki, cekj, cekk, cekl, cekn, ceko, cekq, cekr, ceks, cekt, cekv, cel, celd, cele, celf, celg, celh, celi,
celj, celk, cell, celm, celn, celo, celq, celr, cels, celt, celv, cem, cemd, ceme, cemf, cemg, cemj, ceml,
cemn, cemo, cemq, cemr, cemt, cemv, cen, cend, cene, cenf, ceni, cenj, cenk, cenq, cenr, cens, cent,
cenv, ceo, ceoe, ceof, ceoi, ceoj, cep, cepd, cepe, cepf, cepg, ceph, cepi, cepj, cepk, cepl, cepm, cepn,
cepo, cepq, cepr, ceps, cept, cepv, ceq, ceqd, ceqe, ceqf, ceqg, ceqh, ceqi, ceqi, ceqk, ceql, ceqm, ceqn,
ceqo, ceqq, ceqr, ceqs, ceqt, ceqv, cer, cerd, cere, cerf, cerg, cerh, ceri, cerj, cerk, cerl, cerm, cern, cero,
cerp, cerq, cerr, cers, cert, cerv, ces, cese, cesf, cesg, cesi, cesk, cesl, cesn, ceso, cesq, cesr, cess, cest,
cesv, cet, cetd, cete, cetf, cetg, ceth, ceti, cetj, cetk, cetl, cetm, cetn, ceto, cetp, cetq, cetr, cets, cett, cetv,
ceu, ceud, ceue, ceuf, ceug, ceuh, ceui, ceuk, ceul, ceum, ceun, ceuq, ceur, ceut, ceuv, cev, cevd, ceve,
cevf, cevg, cevh, cevk, cevl, cevm, cevn, cevo, cevp, cevq, cevr, cevs, cevt, cevv, cfci, cfcj, cfck, cfcl,
cfcm, cfcn, cfcp, cfcq, cfcr, cfct, cfcv, cfd, cfdd, cfde, cfdf, cfdg, cfdh, cfdi, cfdl, cfdm, cfdn, cfdq, cfdr,
cfdt, cfdv, cfe, cfed, cfef, cfeh, cfei, cfej, cfek, cfel, cfem, cfen, cfeo, cfeq, cfer, cfet, cfev, cff, cffd, cffe,
cfff, cffg, cffh, cffi, cffj, cffk, cffm, cffp, cffq, cffr, cfft, cffu, cffv, cfg, cfgd, cfgf, cfgh, cfgi, cfgj, cfgk,
cfgl, cfgm, cfgn, cfgo, cfgq, cfgr, cfgs, cfgt, cfgv, cfh, cfhd, cfhe, cfhf, cfhg, cfhh, cfhi, cfhj, cfhk, cfhl,
cfhm, cfhn, cfho, cfhq, cfhr, cfhs, cfht, cfhv, cfi, cfid, cfie, cfif, cfig, cfih, cfik, cfil, cfim, cfin, cfip, cfiq,
cfir, cfis, cfit, cfiv, cfj, cfjf, cfjg, cfjh, cfji, cfjj, cfjk, cfjr, cfjt, cfjv, cfk, cfke, cfkf, cfkg, cfkh, cfki, cfkj,
cfkl, cfkm, cfkr, cfkt, cfkv, cfl, cfld, cfle, cflg, cflh, cflk, cfln, cflq, cflr, cflt, cflv, cfm, cfmd, cfme, cfmf,
cfmg, cfmi, cfmj, cfmk, cfml, cfmm, cfmn, cfmo, cfmq, cfmr, cfmt, cfmv, cfn, cfnf, cfnj, cfo, cfp, cfpd,
cfpe, cfpf, cfpg, cfph, cfpi, cfpj, cfpk, cfpl, cfpm, cfpn, cfpo, cfpq, cfpr, cfpt, cfpv, cfq, cfqd, cfqe, cfqf,
cfqg, cfqh, cfqi, cfqj, cfqk, cfql, cfqm, cfqn, cfqq, cfqr, cfqt, cfqu, cfqv, cfr, cfrd, cfre, cfrf, cfrg, cfrh,
cfri, cfrj, cfrk, cfrl, cfrm, cfm, cfrq, cfrr, cfrs, cfrt, cfrv, cfs, cfsd, cfse, cfsg, cfsh, cfsi, cfsk, cfsl, cfsm,
cfsn, cfso, cfsq, cfsr, cfst, cfsv, cft, cftd, cfte, cftf, cftg, cfth, cfti, cftj, cftk, cftl, cftm, cftn, cftq, cftr, cfts,
cftt, cftv, cfu, cfud, cfue, cfuf, cfug, cfuh, cfui, cfuj, cful, cfum, cfuq, cfur, cfus, cfut, cfuv, cfv, cfvd,
cfve, cfvf, cfvg, cfvh, cfvi, cfvk, cfvl, cfvm, cfvn, cfvo, cfvq, cfvr, cfvt, cfvv, cgch, cgci, cgck, cgel,
cgem, cgen, cgco, cgep, cgoq, cger, cget, cgev, cgd, cgdd, cgde, cgdf, cgdg, cgdh, cgdi, cgdj, cgdk, cgdl,
cgdm, cgdn, cgdo, cgdp, cgdq, cgdr, cgds, cgdt, cgdv, cge, cged, cgeh, cgei, cgej, cgek, cgel, cgem, cgen,
cgeo, cgep, cgeq, cger, cges, cget, cgeu, cgev, cgf, cgfd, cgff, cgfh, cgfi, cgfj, cgfk, cgfl, cgfn, cgfp, cgfq,
cgfr, cgfs, cgft, cgfu, cgfv, cgg, cggd, cggh, cggi, cggj, cggk, cggl, cggm, cggn, cggo, cggp, cggq, cggr,
cggs, cggt, cggu, cggv, cgh, cghd, cghe, cghf, cghg, cghh, cghi, cghj, cghk, cghl, cghm, cghn, cgho, cghq,
cghr, cghs, cght, cghv, cgi, cgid, cgie, cgif, cgig, cgih, cgii, cgik, cgil, cgim, cgin, cgio, cgip, cgiq, cgir,
cgis, cgit, cgiv, cgj, cgjf, cgji, cgjj, cgjk, cgjq, cgjr, cgjs, cgjt, cgju, cgjv, cgk, cgkd, cgke, cgkf, cgkg,
cgki, cgkj, cgkk, cgkl, cgkm, cgkn, cgkq, cgkr, cgkt, cgku, cgkv, cgl, cgld, cgle, cglf, cglg, cglh, cgli,
cglj, cglk, cgll, cglm, cgln, cglo, cglp, cglq, cglr, cglt, cglv, cgm, cgmd, cgme, cgmf, cgmg, cgmh, cgmi,
cgmj, cgml, cgmm, cgmo, cgmq, cgmr, cgmv, cgn, cgnf, cgnj, cgnk, cgnr, cgnt, cgo, cgoe, cgoj, cgp,
cgpd, cgpe, cgpf, cgpg, cgph, cgpi, cgpj, cgpk, cgpl, cgpm, cgpn, cgpo, cgpq, cgpr, cgpt, cgpu, cgpv,
cgq, cgqd, cgqe, cgqf, cgqg, cgqh, cgqi, cgqj, cgqk, cgql, cgqm, cgqn, cgqo, cgqp, cgqq, cgqr, cgqt, cgqu,
cgqv, cgr, cgrd, cgre, cgrf, cgrg, cgrh, cgri, cgrj, cgrk, cgrl, cgrm, cgrn, cgro, cgrp, cgrq, cgrr, cgrs, cgrt,
cgrv, cgs, cgse, cgsf, cgsg, cgsi, cgsl, cgsm, cgsn, cgso, cgsp, cgsq, ccgsr, cgst, cgsv, cgt, cgtd, cgte, cgtf,
cgtg, cgth, cgti, cgtj, cgtk, cgtl, cgtm, cgtn, cgto, cgtp, cgtq, cgtr, cgts, cgtt, cgtu, cgtv, cgu, cgud, cgue,
cguf, cgug, cgui, cguk, cgul, cgum, cguq, cgur, cgut, cguv, cgv, cgvd, cgve, cgvf, cgvg, cgvh, cgvi, cgvk,
cgvl, cgvm, cgvn, cgvo, cgvp, cgvq, cgvr, cgvt, cgvv, chch, chci, chcl, chcp, chcr, chcs, chct, chd, chdd,
chdf, chdg, chdh, chdi, chdl, chdm, chdp, chdr, chdt, che, chee, chef, cheg, chej, chel, chem, chep, cheq,
cher, chet, chev, chf, chff, chfg, chfj, chfm, chfp, chfq, chfr, chfs, chft, chfu, chfv, chg, chge, chgf, chgg,
chgh, chgi, chgj, chgk, chgl, chgm, chgn, chgp, chgq, chgr, chgs, chgt, chgu, chgv, chh, chhd, chhe, chhf,
chhg, chhh, chhk, chhl, chhq, chhr, chhv, chi, chid, chif, chig, chih, chim, chip, chiq, chir, chis, chiv, chj,
chk, chkf, chkg, chkh, chki, chkq, chks, chkt, chl, chle, chlf, chlg, chli, chlj, chlm, chln, chlp, chlq, chlr,
chls, chlt, chlv, chm, chme, chmf, chmg, chmi, chmm, chn, chnf, chnj, cho, choe, chof, choj, chos, chp,
chpd, chpe, chpg, chph, chpi, chpm, chpr, chpt, chpu, chq, chqd, chqe, chqf, chqg, chqi, chqj, chqm,
chqq, chqr, chqt, chqv, chr, chrd, chre, chrf, chrg, chrh, chri, chrj, chrl, chrm, chrq, chrr, chrs, chrt, chrv,
chs, chsg, chsi, chsp, chsq, chsr, chsv, cht, chte, chtf, chtg, chth, chti, chtj, chtm, chtq, chtr, chts, chtt,
chtv, chu, chue, chui, chur, chv, chvd, chve, chvg, chvh, chvi, chvm, chvq, chvr, chvt, chvv, cici, cicl,
cicm, cicp, cicq, cicr, cid, cidd, cide, cidf, cidg, cidh, cidi, cidj, cidk, cidm, cidp, cidq, cidr, cidt, cidv,
cie, cied, ciee, cief, cieg, cieh, ciei, ciek, ciel, ciep, cieq, cier, cies, ciet, ciev, cif, ciff, cifj, cifs, cift, cifu,
cig, cige, cigf, cigg, cigh, cigi, cigk, cigm, cigp, cigq, cigr, cigs, cigt, cigu, cigv, cih, cihe, cihg, cihi,
cihk, cihm, cihp, cihq, cihr, ciht, cihv, cii, ciid, ciie, ciig, ciii, ciil, ciim, ciip, ciiq, ciir, ciiv, cij, cik, cikd,
cike, cikf, cikg, cikh, ciki, cikj, cikm, cikp, ciks, cikt, cil, cild, cile, cilf, cilg, cilh, cili, cilj, cill, cilm, cilp,
cilq, cilr, cils, cilt, cilv, cim, cime, cimf, cimg, cimi, cin, cinf, cinh, cinr, cins, cio, cioe, ciof, cioh, cios,
ciov, cip, cipd, cipe, cipf, cipg, cipi, cipj, cipm, cipn, cipr, cips, cipt, cipv, ciq, ciqd, ciqe, ciqf, ciqg, ciqh,
ciqi, ciqj, ciqk, ciqm, ciqn, ciqq, ciqr, ciqs, ciqt, ciqu, ciqv, cir, cird, cire, cirf, cirg, cirh, ciri, cirj, cirm,
cirp, cirq, cirr, cirs, cirt, cirv, cis, cisd, cise, cisf, cisg, cisi, cism, cisn, cisq, cisr, cist, cisv, cit, citd, cite,
citf, citg, cith, citi, citj, citm, citp, citr, cits, citt, citv, ciu, ciuf, ciuv, civ, civd, cive, civg, civh, civi, civl,
civm, civn, civp, civq, civr, civs, civt, civv, cjcj, cjck, cjcn, cjcq, cjcr, cjct, cjcv, cjd, cjde, cjdc, cje, cjed,
cjee, cjek, cjen, cjeq, cjer, cjet, cjev, cjf, cjfd, cjfe, cjff, cjfg, cjfh, cjfl, cjfm, cjfq, cjfr, cjfs, cjft, cjfv, cjg,
cjgd, cjge, cjgf, cjgg, cjgj, cjgl, cjgn, cjgq, cigr, cjgt, cjgv, cjh, cihd, cjhe, cjhg, cjhj, cjhl, cjhn, cjhq, cjhr,
cjht, cjhv, cji, cjid, cjie, cjik, cjin, cjiq, cjir, cjit, cjiv, cjjf, cjjs, cjjt, cjk, cjke, cjl, cjm, cjmd, cjme, cjmg,
cjmj, cjml, cjmn, cjmq, cjmr, cjmt, cjmv, cjn, cjp, cjpd, cjpe, cjpg, cjpk, cjpl, cjpn, cjpq, cjpr, cjpt, cjpv,
cjq, cjqe, cjqq, cjr, cjrd, cjre, cjrf, cjrg, cjrh, cjri, cjrj, cjrl, cjrm, cjmn, cjrt, cjrv, cjs, cjsd, cjse, cjsg, cjsl,
cisn, cjsq, cjsr, cist, cjsv, cjt, cjtd, cjte, cjtf, cjtg, cjth, cjti, cjtj, cjtl, cjtm, cjtn, cjtq, cjtr, cjtt, cjtv, cjud,
cjue, cjug, cjuh, cjui, cjuj, cjul, cjuv, cjv, cjvd, cjve, cjvg, cjvh, cjvk, cjvl, cjvm, cjvn, cjvq, cjvr, cjvt,
cjvv, ckck, ckcl, ckcn, ckcp, ckcq, ckcr, ckct, ckcv, ckd, ckde, ckdg, ckdj, ckdl, ckdn, ckdo, ckdp, ckdq,
ckdr, ckdt, ckdv, cke, cked, ckee, ckef, ckeg, ckeh, ckej, ckek, ckel, cken, ckeo, ckeq, cker, cket, ckeu,
ckev, ckf, ckfd, ckff, ckfg, ckfh, ckfp, ckfr, ckfs, ckft, ckfu, ckg, ckgd, ckge, ckgf, ckgg, ckgj, ckgk, ckgl,
ckgo, ckgp, ckgq, ckgr, ckgt, ckgu, ckgv, ckh, ckhd, ckhe, ckhf, ckhg, ckhi, ckhk, ckhl, ckhm, ckhn,
ckho, ckhq, ckhr, ckht, ckhv, cki, ckid, ckie, ckif, ckig, ckih, ckik, ckil, ckim, ckin, ckio, ckip, ckiq, ckir,
ckis, ckit, ckiv, ckj, ckjd, ckjf, ckjg, ckjr, ckjt, ckju, ckk, ckke, ckkf, ckkg, ckkk, ckkl, ckkq, ckkr, ckkt,
ckkv, ckl, ckle, cklf, cklg, cklh, cklj, ckln, cklp, cklq, cklr, cklt, cklv, ckm, ckmd, ckme, ckmf, ckmg,
ckmj, ckmo, ckmr, ckn, cknf, cknj, cko, ckp, ckpd, ckpe, ckpf, ckpg, ckph, ckpi, ckpj, ckpk, ckpl, ckpm,
ckpn, ckpo, ckpq, ckpr, ckpt, ckpv, ckq, ckqd, ckqe, ckqf, ckqg, ckqh, ckqi, ckqj, ckqk, ckql, ckqm, ckqn,
ckqo, ckqq, ckqr, ckqt, ckqv, ckr, ckrd, ckre, ckrf, ckrg, ckri, ckrj, ckrk, ckrl, ckrm, ckrn, ckro, ckrp,
ckrq, ckrr, ckrs, ckrt, ckrv, cks, cksd, ckse, cksg, ckso, cksq, cksr, ckst, ckt, cktd, ckte, cktf, cktg, ckth,
ckti, cktj, cktk, cktl, cktm, cktn, ckto, cktp, cktq, cktr, ckts, cktt, cktv, cku, ckue, ckuf, ckug, ckul, ckuo,
ckuq, ckur, ckut, ckuv, ckv, ckvd, ckve, ckvf, ckvg, ckvh, ckvi, ckvk, ckvl, ckvm, ckvn, ckvo, ckvq,
ckvr, ckvt, ckvv, clcl, clcm, clco, clcq, clcr, clcs, clct, clcv, cld, cldd, clde, cldf, cldg, cldh, cldk, cldl,
cldq, cldr, clds, cldt, cldv, cle, cled, clee, clef, cleg, cleh, clei, clej, clek, clel, clem, clen, cleo, clep, cleq,
cler, cles, clet, cleu, clev, clf, clfd, clfe, clff, clfg, clfh, clfi, clfk, clfl, clfm, clfp, clfq, clfr, clfs, clft, clfu,
clfv, clg, clgd, clge, clgf, clgg, clgh, clgi, clgj, clgk, clgl, clgm, clgn, clgo, clgp, clgq, clgr, clgs, clgt,
clgu, clgv, clh, clhd, clhe, clhf, clhg, clhh, clhi, clhk, clhl, clhm, clho, clhp, clhq, clhr, clhs, clhv, cli, clid,
clie, clif, clig, clii, clik, clil, clim, clio, clip, cliq, clir, clis, clit, cliv, clj, cljf, cljg, clji, cljj, cljk, cljq, cljr,
cljt, clju, cljv, clk, clkd, clke, clkg, clkh, clki, clkl, clkp, clkq, clkr, clkt, clkv, cll, clld, clle, cllf, cllg, cllh,
clli, cllj, clik, clll, cllm, clln, cllo, cllq, cllr, cllt, cllv, clm, clmd, clme, clmg, clmh, clmi, clmk, clml,
clmm, clmo, clmp, clmr, clmv, cln, clnf, clni, clnj, clnq, clns, clo, cloe, clog, cloj, clon, clp, clpd, clpe,
clpf, clpg, clph, clpi, clpk, clpl, clpm, clpn, clpo, clpp, clpq, clpr, clps, clpt, clpv, clq, clqd, clqe, clqf,
clqg, clqh, clqi, clqj, clqk, clql, clqm, clqn, clqo, clqq, clqr, clqt, clqu, clqv, clr, clrd, clre, clrf, clrg, clri,
clrj, clrk, clrl, clrm, clrn, clro, clrp, clrq, clrr, clrt, clrv, cls, clse, clsg, clsi, clsl, clsm, clso, clsp, clsq, clsr,
clst, clt, cltd, clte, cltf, cltg, clth, clti, cltj, cltk, cltl, clim, clto, cltp, cltq, cltr, clts, cltt, cltv, clu, clud, clue,
cluf, clug, clul, cluq, chur, clut, cluv, clv, clvd, clve, clvf, clvg, clvh, clvi, clvj, clvk, clvl, clvm, clvn,
clvo, clvp, clvq, clvr, clvs, clvt, clvu, clvv, cmcm, cmcp, cmcq, cmcr, cmcv, cmd, cmdg, cmdh, cmdj,
cmdm, cmdr, cmdt, cme, cmef, cmeg, cmem, cmep, cmeq, cmer, cmes, cmet, cmev, cmf, cmfe, cmff,
cmfg, cmfh, cmfi, cmfj, cmfm, cmfp, cmfq, cmfr, cmfs, cmft, cmfu, cmfv, cmg, cmgf, cmgg, cmgh,
cmgm, cmgp, cmgr, cmgs, cmgt, cmgv, cmh, cmhg, cmht, cmi, cmie, cmii, cmiq, cmj, cmjf, cmjj, cmk,
cmkf, cmkj, cml, cmle, cmlf, cmlg, cmlh, cmlm, cmlp, cmlt, cmlv, cmm, cmmf, cmn, cmnf, cmo, cmp,
cmpe, cmpf, cmpg, cmpj, cmpm, cmpn, cmpr, cmpt, cmpu, cmpv, cmq, cmqf, cmqm, cmqr, cmqt, cmqu,
cmr, cmrf, cmrj, cmrm, cmrr, cmrs, cmrt, cms, cmsg, cmsm, cmt, cmtf, cmtj, cmtt, cmu, cmv, cmvr,
cmvt, cnon, cncp, cncq, cncr, cncv, cnd, cnde, cndg, cndj, cndl, cndn, cndq, cne, cnee, chef, cneg, cnej,
cnel, cnen, cneo, cner, cnet, cnev, cnf, cnfd, cnfe, cnff, cnfg, cnfh, cnfj, cnfk, cnfl, cnfp, cnfq, cnfr, cnft,
cnfv, cng, cngd, cnge, cngf, cngg, cngj, cngk, cngl, cngn, cngo, cngq, cngr, cngt, cnh, cnhd, cnhe, cnhf,
cnhg, cnhk, cnhl, cnhn, cnho, cnhq, cnhr, cnht, cnhv, cni, cnid, cnie, cnig, cnik, cnin, cniq, cnit, cniv,
cnj, cnjf, cnjj, cnjq, cnjr, cnjs, cnjt, cnk, cnke, cnkg, cnkl, cnkn, cnl, cnle, cnm, cnmd, cnme, cnmf, cnmg,
cnmj, cnmk, cnml, cnmo, cnmq, cnmr, cnmt, cnmv, cnn, cno, cnp, cnpd, cnpe, cnpf, cnpg, cnpl, cnpn,
cnpo, cnpq, cnpr, cnpt, cnpv, cnq, cnqe, cnqg, cnqi, cnqk, cnql, cnqn, cnqq, cnqr, cnqt, cnqv, cnr, cnrd,
cnre, cnrf, cnrg, cnrh, cnrj, cnrk, cnrl, cnrm, cnrn, cnrq, cnrr, cnrt, cnrv, cns, cnsd, cnse, cnsf, cnsg, cnsk,
cnso, cnsq, cnsr, cnst, cnsv, cnt, cntd, cnte, cntf, cntg, cnth, cntj, cntk, cntl, cntm, cntn, cnto, cntq, cntr,
cntt, cntv, cnud, cnue, cnuf, cnug, cnuh, cnuk, cnul, cnum, cnun, cnur, cnut, cnuv, cnv, cnvd, cnve, cnvf,
cnvg, cnvk, cnvl, cnvn, cnvq, cnvr, cnvt, cnvv, coco, cocq, cocs, cocv, cod, code, codg, codk, codl, codq,
codr, codv, coe, coed, coee, coef, coeg, coeh, coei, coek, coel, coem, coen, coeo, coep, coeq, coer, coes,
coet, coeu, coev, cof, cofd, cofe, cofg, cofi, cofk, cofl, cofo, cofp, cofq, cofr, cofs, coft, cofu, cofv, cog,
cogd, coge, cogi, cogk, cogl, cogq, cogr, cogt, cogv, coh, cohd, cohe, cohg, cohh, cohl, cohm, coho,
cohq, cohr, cohv, coi, coid, coie, coif, coig, coik, coil, coio, coiq, coir, cois, coiv, coj, cojd, coje, cojg,
coji, cojk, cojp, cojq, cojr, cojs, cojt, cojv, cok, cokd, coke, cokg, cokl, cokq, cokt, cokv, col, cold, cole,
colg, colk, coll, coln, colo, colq, colr, colt, colv, com, comd, comg, comq, con, cone, conr, cont, coo,
cop, copd, cope, copf, copg, copl, copo, copq, copr, copv, coq, coqd, coqe, coqf, coqg, coqj, coqk, coql,
coqn, coqo, coqq, coqr, coqt, coqv, cor, cord, core, corf, corg, cork, corl, coro, corq, corr, cort, corv, cos,
cosg, cosi, cosl, cosq, cosv, cot, cotd, cote, cotf, cotg, cotk, cotl, coto, cotp, cotq, cotr, cott, cotv, cou,
coud, coue, coug, coul, couq, cour, couv, cov, covd, cove, covf, covg, covk, covl, covn, covo, covq, covr,
covt, covv, cpcp, cpcq, cpcr, cpct, cpcv, cpd, cpdd, cpde, cpdf, cpdg, cpdh, cpdj, cpdk, cpdn, cpdp, cpdr,
cpds, cpdt, cpdv, cpe, cpee, cpef, cpeg, cpeh, cpei, cpej, cpek, cpel, cpem, cpen, cpeo, cpep, cpeq, cper,
cpes, cpet, cpeu, cpev, cpf, cpff, cpfg, cpfh, cpfj, cpfm, cpfp, cpfq, cpfr, cpft, cpfu, cpg, cpgd, cpge, cpgf,
cpgg, cpgh, cpgi, cpgj, cpgk, cpgl, cpgm, cpgn, cpgp, cpgq, cpgr, cpgs, cpgt, cpgu, cpgv, cph, cphd, cphf,
cphg, cphk, cphl, cphm, cpho, cphp, cphq, cphr, cpht, cpi, cpif, cpig, cpih, cpil, cpim, cpio, cpip, cpiq,
cpir, cpit, cpiv, cpj, cpjd, cpjj, cpjr, cpjt, cpju, cpk, cpkd, cpke, cpkf, cpkg, cpkh, cpkk, cpkm, cpkn, cpkp,
cpkr, cpkt, cpku, cpl, cpld, cple, cplf, cplg, cplh, cpli, cplk, cpll, cplm, cpln, cplp, cplq, cplr, cpls, cplt,
cplu, cplv, cpm, cpme, cpmg, cpmp, cpmq, cpn, cpnf, cpnj, cpnr, cpnt, cpo, cpoe, cpof, cpog, cpoi, cpoj,
cpol, cpon, cpoq, cpor, cpot, cpp, cppe, cppg, cppi, cppl, cppm, cppn, cppo, cppp, cppq, cppr, cppt, cppv,
cpq, cpqd, cpqe, cpqf, cpqg, cpqh, cpqi, cpqi, cpqk, cpql, cpqm, cpqn, cpqo, cpqq, cpqr, cpqs, cpqt, cpqu,
cpqv, cpr, cpre, cprf, cprg, cprh, cpri, cprj, cprk, cprl, cprm, cprn, cpro, cprp, cprq, cprr, cprs, cprt, cpru,
cprv, cps, cpse, cpsg, cpsl, cpsq, cpsr, cpt, cptd, cpte, cptf, cptg, cpth, cpti, cptj, cptk, cptl, cptn, cpto,
cptp, cptq, cptr, cpts, cptt, cptu, cptv, cpu, cpud, cpue, cpuf, cpug, cpul, cpuo, cpup, cpuq, cpur, cput,
cpuv, cpv, cpvd, cpve, cpvf, cpvg, cpvh, cpvi, cpvj, cpvk, cpvl, cpvm, cpvn, cpvo, cpvp, cpvq, cpvr,
cpvs, cpvt, cpvu, cpvv, cqcq, cqcr, cqcs, cqct, cqcv, cqd, cqdd, cqde, cqdf, cqdg, cqdh, cqdj, cqdk, cqdl,
cqdn, cqdo, cqdp, cqdq, cqdr, cqds, cqdt, cqdv, cqe, cqed, cqee, cqef, cqeg, cqeh, cqei, cqej, cqek, cqel,
cqem, cqen, cqeo, cqep, cqeq, cqer, cqes, cqet, cqeu, cqev, cqf, cqfd, cqfe, cqff, cqfg, cqfh, cqfi, cqfj,
cqfk, cqfl, cqfm, cqfn, cqfo, cqfp, cqfq, cqfr, cqfs, cqft, cqfu, cqfv, cqg, cqgd, cqge, cqgf, cqgg, cqgh,
cqgi, cqgj, cqgk, cqgl, cqgm, cqgn, cqgo, cqgp, cqgq, cqgr, cqgs, cqgt, cqgu, cqgv, cqh, cqhd, cqhe, cqhf,
cqhg, cqhj, cqhk, cqhl, cqhn, cqho, cqhp, cqhq, cqhr, cqht, cqhv, cqi, cqid, cqie, cqif, cqig, cqih, cqui,
cqik, cqil, cqim, cqio, cqip, cqiq, cqir, cqis, cqit, cqiv, cqj, cqid, cqje, cqjf, cqig, cqjh, cqji, cqij, cqik,
cqjl, cqjm, cqjp, cqjq, cqjr, cqjs, cqjt, cqju, cqjv, cqk, cqke, cqkf, cqkg, cqkh, cqki, cqkk, cqkl, cqkm,
cqkn, cqkp, cqkq, cqkr, cqks, cqkt, cqkv, cql, cqld, cqle, cqlf, cqlg, cqlh, cqli, cqlj, cqll, cqlm, cqln, cqlo,
cqlp, cqlq, cqlr, cqls, cqlt, cqlv, cqm, cqmd, cqme, cqmg, cqmh, cqmi, cqml, cqmo, cqmp, cqmq, cqmr,
cqn, cqnf, cqnj, cqnq, cqor, cqns, cqnt, cqnu, cqnv, cqo, cqod, cqoe, cqof, cqoj, cqok, cqon, cqp, cqpd,
cqpe, cqpf, cqpg, cqph, cqpi, cqpl, cqpm, cqpn, cqpo, cqpp, cqpq, cqpr, cqpt, cqpv, cqq, cqqd, cqqe, cqqf,
cqqg, cqqh, cqqi, cqqi, cqqk, cqql, cqqm, cqqn, cqqo, cqqq, cqqr, cqqs, cqqt, cqqv, cqr, cqrd, cqre, cqrf,
cqrg, cqrh, cqri, cqrj, cqrk, cqrl, cqrm, cqrn, cqro, cqrp, cqrq, cqrr, cqrs, cqrt, cqrv, cqs, cqsd, cqse, cqsg,
cqsh, cqsj, cqsl, cqsm, cqso, cqsq, cqsr, cqst, cqsv, cqt, cqtd, cqte, cqtf, cqtg, cqth, cqti, cqtj, cqtk, cqtl,
cqtm, cqtn, cqto, cqtp, cqtq, cqtr, cqts, cqtt, cqtv, cqu, cqud, cque, cquf, cqug, cqui, cquj, cqul, cqum,
cquo, cqup, cquq, cqur, cqut, cquv, cqv, cqvd, cqve, cqvf, cqvg, cqvh, cqvi, cqvk, cqvl, cqvm, cqvn,
cqvo, cqvq, cqvr, cqvs, cqvt, cqvv, crcr, crct, crcu, crcv, crd, crdd, crde, crdf, crdg, crdj, crdk, crdl, crdn,
crdp, crdq, crdr, crdt, crdv, cre, cred, cree, cref, creg, creh, crei, crej, crek, crel, crem, cren, creo, crep,
creq, crer, cres, cret, creu, crev, crf, crfd, crfe, crff, crfg, crfh, crfi, crfj, crfk, crfl, crfm, crfp, crfq, crfr,
crfs, crft, crfu, crfv, crg, crgd, crge, crof, crgg, crgh, crgi, crgj, crgk, crgl, crgm, crgn, crgo, crgp, crgq,
crgr, crgs, crgt, crgu, crgv, crh, crhd, crhe, crhf, crhg, crhh, crhk, crhl, crhm, crhn, crho, crhp, crhq, crhr,
crht, crhv, cri, crid, crie, crif, crig, crih, crii, crik, cril, crim, crin, crio, crip, criq, crir, cris, crit, criv, crj,
crjd, crje, crjf, crjg, crjh, crji, crjj, crjk, crjm, crjp, crjq, crjr, crjs, crjt, crju, crjv, crk, crkd, crke, crkg,
crkh, crki, crkk, crkl, crkn, crkp, crkq, crkr, crks, crkt, crku, crkv, crl, crld, crle, crlf, crlg, crlh, crli, crlj,
crlk, crll, crlm, crln, crlo, crlp, crlq, crlr, crls, crlt, crlv, crm, crmd, crme, crmf, crmg, crml, crmm, crmo,
crmp, crmq, crmr, crmv, crn, crnf, crni, crnj, crnq, crnr, crns, crnt, cro, croe, crof, crog, croj, crp, crpd,
crpe, crpf, crpg, crph, crpi, crpj, crpl, crpm, crpn, crpo, crpp, crpq, crpr, crpt, crpv, crq, crqd, crqe, crqf,
crqg, crqi, crqj, crqk, crql, crqm, crqn, crqo, crqq, crqr, crqt, crqu, crqv, crr, crrd, crre, crrf, crrg, crrh,
crri, crrj, crrk, crrl, crrm, crrn, crro, crrp, crrq, crrr, crrs, crrt, crru, crrv, crs, crse, crsg, crsi, crsk, crsl,
crsm, crsn, crso, crsq, crsr, crss, crst, crsv, crt, crtd, crte, crtf, crtg, crth, crti, crtj, crtk, crtl, crtm, crtn,
crto, crtp, crtq, crtr, crts, crtt, crtu, crtv, cru, crud, crue, cruf, crug, crui, crul, crum, cruq, crur, crus, crut,
cruv, crv, crvd, crve, crvf, crvg, crvh, crvi, crvj, crvk, crvl, crvm, crvn, crvo, crvp, crvq, crvr, crvs, crvt,
crvu, crvv, cscs, csct, csd, cse, csee, csef, cseg, cseh, csej, csen, csep, cset, cseu, csf, csfg, csfk, csfm,
csfr, csft, csg, csge, csgf, csgg, cgh, csgm, csgp, csgr, csgt, csgu, csh, cshe, cshg, cshr, csht, cshu, csi,
csim, csin, csip, csit, csj, csjf, csk, cskq, cskr, cskt, csl, csld, csle, cslf, cslg, cslj, cslk, cslm, csln, cslp,
cslr, csls, cslt, cslv, csm, csme, csn, csnf, csnt, cso, csoe, csof, csog, csoj, cson, csop, csot, csp, cspe,
csph, cspi, cspt, cspv, csq, csqe, csqf, csqg, csqj, csqm, csqq, csqr, csqs, csqt, csr, csre, csrf, csrh, csrj,
csrm, csrr, csrs, csrt, csru, css, csse, cssr, cst, cstd, cstf, cstg, csth, cstj, cstr, cstt, cstu, csu, csur, csut, csv,
csvd, csve, csvf, csvg, csvh, csvm, csvq, csvr, csvt, ctct, ctcv, ctd, ctde, ctdf, ctdn, ctdp, ctdq, ctdr, ctdv,
cte, cted, ctee, ctef, cteg, ctei, ctej, ctel, ctem, cten, cteo, ctep, cteq, cter, ctes, ctet, ctev, ctf, ctfd, ctfe,
ctff, ctfg, ctfh, ctfi, ctfj, ctfk, ctfl, ctfm, ctfp, ctfq, ctfr, ctfs, ctft, ctfu, ctfv, ctg, ctgd, ctge, ctgf, ctgg, ctgh,
ctgj, ctgk, ctgl, ctgm, ctgn, ctgo, ctgp, ctgq, ctgr, ctgs, ctgt, ctgv, cth, cthd, cthe, cthf, cthg, cthh, cthi,
cthk, cthl, cthm, ctho, cthq, cthr, ctht, cthv, cti, ctid, ctie, ctif, ctig, ctih, ctik, ctil, ctim, ctin, ctip, ctiq,
ctir, ctis, ctit, ctiv, ctj, ctjd, ctjf, ctjg, ctjh, ctjk, ctjp, ctjq, ctjr, ctjs, ctjt, ctju, ctjv, ctk, ctke, ctkf, ctkh,
ctki, ctkn, ctkp, ctkr, ctks, ctkt, ctkv, ctl, ctld, ctle, ctlf, ctlg, ctlh, ctli, ctlj, ctlk, ctlm, ctln, ctlp, ctlq, ctlr,
ctls, ctlt, ctlv, ctm, ctmd, ctme, ctmg, ctml, ctmo, ctmq, ctmr, ctmt, ctn, ctnf, ctnj, cto, ctp, ctpd, ctpe,
ctpf, ctpg, ctph, ctpi, ctpl, ctpm, ctpn, ctpo, ctpq, ctpr, ctpt, ctpv, ctq, ctqd, ctqe, ctqf, ctqg, ctqh, ctqi,
ctqj, ctqk, ctql, ctqm, ctqn, ctqp, ctqq, ctqr, ctqs, ctqt, ctqv, ctr, ctrd, ctre, ctrf, ctrg, ctrh, ctri, ctrj, ctrk,
ctrl, ctrm, ctrn, ctrq, ctrr, ctrs, ctrt, ctru, ctrv, cts, ctsd, ctse, ctsg, ctsi, ctsm, ctso, ctsq, ctss, ctst, ctsv, ctt,
cttd, ctte, cttf, cttg, ctth, ctti, cttj, cttk, cttl, cttm, cttn, ctto, cttq, cttr, ctts, cttt, cttu, cttv, ctu, ctue, ctuf,
ctug, ctuh, ctui, ctul, ctum, ctuo, ctuq, ctur, ctus, ctut, ctuv, ctv, ctvd, ctve, ctvf, ctvg, ctvh, ctvi, ctvj,
ctvk, ctvl, ctvm, ctvn, ctvo, ctvp, ctvq, ctvr, ctvt, ctvv, cud, cudr, cue, cues, cuev, cuf, cufd, cufm, cug,
cuh, cui, cuj, cujh, cujv, cuk, cul, culg, culh, culi, culm, culs, cum, cun, cuo, cup, cupe, cupg, cuph, cupi,
cupm, cupv, cuq, cuqe, cuqm, cuqv, cur, cure, curf, curm, curs, cus, cut, cutd, cute, cutf, cutm, cuts, cutv,
cuu, cuv, cuve, cuvm, cuvv, cvcv, cvd, cvdd, cvde, cvdf, cvdg, cvdh, cvdi, cvdj, cvdk, cvdl, cvdn, cvdo,
cvdp, cvdq, cvdr, cvds, cvdt, cvdv, cve, cved, cvee, cvef, cveg, cveh, cvei, cvej, cvek, cvel, cvem, cven,
cveo, cvep, cveq, cver, cves, cvet, cveu, cvev, cvf, cvfd, cvfe, cvff, cvfg, cvfj, cvfl, cvfm, cvfn, cvfp,
cvfq, cvfr, cvft, cvg, cvgd, cvge, cvgf, cvgg, cvgh, cvgi, cvgj, cvgk, cvgl, cvgm, cvgn, cvgo, cvgp, cvgq,
cvgr, cvgs, cvgt, cvgu, cvgv, cvh, cvhd, cvhe, cvhf, cvhg, cvhh, chi, cvhk, cvhl, cvhm, cvhn, cvho,
cvhp, cvhq, cvhr, cvht, cvi, cvid, cvie, cvif, cvig, cvih, cvii, cvik, cvil, cvim, cvin, cvio, cvip, cviq, cvir,
cvis, cvit, cviv, cvj, cvjt, cvju, cvk, cvkd, cvke, cvkf, cvkg, cvkh, cvki, cvkk, cvkl, cvkn, cvko, cvkp,
cvkq, cukr, cvks, cvkt, cvku, cvkv, cvl, cvld, cvle, cvlf, cvlg, cvlh, cvli, cvlj, cvlk, cvll, cvlm, cvln, cvlo,
cvlp, cvlq, cvlr, cvls, cvlt, cvlv, cvm, cvme, cvmf, cvmg, cvmh, cvmi, cvmj, cvmk, cvml, cvmn, como,
cvmp, cvmq, cvmr, cvmt, cvn, cvnf, cvng, cvnh, cvnj, cvnq, cvnr, cvns, cvnt, cvnu, cvnv, cvo, cvoe,
cvof, cvog, cvon, cvoq, cvot, cvov, cvp, cvpd, cvpe, cvpf, cvpg, cvpi, cvpj, cvpk, cvpl, cvpm, cvpn, cvpo,
cvpp, cvpq, cvpr, cvpt, cvpv, cvq, cvqd, cvqe, cvqf, cvqg, cvqh, cvqi, cvqj, cvqk, cvql, cvqm, cvqn, cvqo,
cvqp, cvqq, cvqr, cvqs, cvqt, cvqv, cvr, cvrd, cvre, cvrf, cvrg, cvrh, cvri, cvrj, cvrk, cvrl, cvrm, cvm,
cvro, cvrp, cvrq, cvrr, cvrs, cvrt, cvru, cvrv, cvs, cvsd, cvse, cvsg, cvsk, cvsl, cvsm, cvsn, cvso, cvsq,
cvsr, cvst, cvsv, cvt, cvtd, cvte, cvtf, cvtg, cvth, cvti, cvtj, cvtk, cvtl, cvtm, cvtn, cvto, cvtp, cvtq, cvtr,
cvts, cvtt, cvtu, cvtv, cvu, cvue, cvul, cvuo, cvup, cvuq, cvur, cvut, cvv, cvvd, cvve, cvvf, cvvg, cvvh,
cvvi, cvvj, cvvk, cvvl, cvvm, cvvn, cvvo, cvvp, cvvq, cvvr, cvvt, cvvv, dddf, dddh, dddj, dddl, dddn,
dddo, dddp, dddq, dddr, ddds, dddt, dddu, dddv, ddee, ddef, ddeg, ddeh, ddei, ddej, ddek, ddel, dden,
ddep, ddeq, dder, ddes, ddet, ddeu, ddev, ddf, ddff, ddfg, ddfh, ddfi, ddfj, ddfk, ddfl, ddfp, ddfq, ddfr,
ddfs, ddft, ddfv, ddge, ddgf, ddgh, ddgi, ddgj, ddgk, ddgl, ddgp, ddgq, ddgr, ddgs, ddgt, ddgu, ddgv, ddh,
ddhe, ddhf, ddhg, ddhi, ddhk, ddhl, ddhp, ddhq, ddhr, ddht, ddhv, ddi, ddie, ddig, ddih, ddii, ddil, ddio,
ddip, ddiq, ddir, ddit, ddiv, ddjf, ddjh, ddji, ddjj, ddjp, ddjq, ddjr, ddjs, ddjt, ddju, ddjv, ddke, ddkf, ddkg,
ddkh, ddkj, ddkp, ddkq, ddkr, ddks, ddkt, ddku, ddkv, ddl, ddle, ddlf, ddlg, ddlh, ddlj, ddlk, ddll, ddln,
ddlo, ddlp, ddlq, ddlr, ddls, ddlt, ddlu, ddlv, ddme, ddmf, ddmg, ddmj, ddml, ddn, ddnf, ddni, ddnj, ddnk,
ddnr, dnt, ddnu, ddo, ddoe, ddof, ddog, ddok, ddol, ddon, ddoq, ddor, ddot, ddov, ddp, ddpe, ddpf, ddpg,
ddpi, ddpj, ddpk, ddpl, ddpn, ddpp, ddpr, ddpt, ddpv, ddq, ddqe, ddqf, ddqg, ddqj, ddqk, ddql, ddqn,
ddqp, ddqq, ddqr, ddqs, ddqt, ddqv, ddr, ddre, ddrf, ddrg, ddrh, ddri, ddrj, ddrk, ddrl, ddrm, ddrn, ddrp,
ddrq, ddrr, ddrt, ddru, ddrv, dds, ddse, ddsf, ddsg, ddsk, ddsl, ddst, ddsv, ddt, ddte, ddtf, ddtg, ddti, ddtj,
ddtk, ddtl, ddtp, ddtq, ddtr, ddtt, ddtv, ddu, dduk, dduq, ddv, ddve, ddvf, ddvg, ddvh, ddvk, ddvl, ddvn,
ddvp, ddvq, ddvr, ddvt, ddvu, ddvv, dedh, dedi, dedj, dedk, dedl, dedn, dedo, dedp, dedq, dedt, dedv,
deee, deeh, deei, deej, deek, deel, deem, deen, deeo, deep, deeq, deer, dees, deet, deeu, deev, def, defh,
defi, defj, defk, defl, defm, defn, defo, defp, defq, defr, deft, defu, defv, degh, degi, degj, degk, degl,
degm, degn, dego, degp, degq, degr, degs, degt, degv, deh, dehe, dehf, dehg, dehh, dehi, dehk, dehl,
dehm, dehn, deho, dehq, dehr, deht, dehv, dei, deie, deig, deih, deii, deik, deil, dein, deio, deip, deiq,
deir, deit, deiv, dej, dejf, dejh, deji, dejj, dejk, dejm, dejp, dejq, dejr, dejt, dejv, dek, deke, dekf, dekg,
dekh, deki, dekj, dekk, dekl, dekm, dekn, deko, dekp, dekq, dekr, dekt, dekv, del, dele, delf, delg, delh,
deli, delj, delk, dell, deln, delo, delp, delq, delr, dels, delt, delv, dem, deme, demf, demg, deml, demn,
demo, demv, den, dene, denf, denh, deni, denj, denp, denq, denr, dens, dent, denu, denv, deo, deoe, deoh,
deoi, deol, deoq, deov, dep, depe, depf, depg, deph, depi, depj, depk, depl, depm, depn, depo, depp, depq,
depr, dept, depv, deq, deqe, deqf, deqg, deqh, deqi, deqj, deqk, deql, deqm, deqn, deqo, deqp, deqq, deqr,
deqt, deqv, der, dere, derf, derg, derh, deri, derj, derk, derl, derm, dern, dero, derp, derq, derr, dert, derv,
des, dese, desf, desg, desl, desm, desn, deso, dest, desv, det, dete, detf, detg, deth, deti, detj, detk, detl,
detm, detn, deto, detp, detq, detr, dett, detv, deu, deue, deuf, deug, deuh, deuj, deuk, deul, deun, deuo,
deup, deuq, deut, deuv, dev, deve, devf, devg, devh, devi, devk, devl, devm, devn, devo, devp, devq,
devr, devs, devt, devv, dfdh, dfdi, dfdk, dfdl, dfdn, dfdp, dfdq, dfdr, dfdt, dfdv, dfe, dfef, dfeh, dfei, dfej,
dfek, dfel, dfem, dfen, dfeo, dfep, dfeq, dfer, dfes, dfet, dfeu, dfev, dff, dffe, dffh, dffi, dffj, dffk, dffl,
dffm, dffp, dffq, dffr, dffs, dfft, dffu, dffv, dfg, dfgh, dfgi, dfgj, digk, dfgl, dfgm, dfgp, dfgq, dfgr, dfgs,
dfgt, dfgu, dfgv, dfh, dfhe, dfhf, dfhg, dfhh, dfhi, dfhk, dfhl, dfhm, dfho, dfhp, dfhq, dfhr, dfhs, dfht,
dfhv, dfi, dfie, dfig, dfih, dfii, dfik, dfil, dfim, dfio, dfip, dfiq, dfir, dfis, dfiv, dfj, dfje, dfjh, dfji, dfjj,
dfjk, dfjl, dfjm, dfjp, dfjq, dfjr, dfjt, dfju, dfjv, dfk, dfke, dfkf, dfkg, dfkh, dfki, dfkj, dfkk, dfkl, dfkm,
dfkp, dfkq, dfkr, dikt, dikv, dfl, dfle, dflf, dflg, dflh, dfli, dflj, dflk, dfll, dflm, dfln, dflp, dflq, dflr, dflt,
dflv, dfm, dfme, dfmf, dfmg, dfmh, dfmi, dfmj, dfml, dfn, dfnf, dfnh, dfnj, dfnp, dfnr, dfnt, dfnu, dfnv,
dfo, dfoe, dfoq, dfor, dfot, dfou, dfov, dfp, dfpe, dfpf, dfpg, dfph, dfpi, dfpj, dfpk, dfpl, dfpm, dfpn, dfpo,
dfpp, dfpq, dfpr, dfpt, dfpv, dfq, dfqe, dfqf, dfqg, dfqh, dfqi, dfqj, dfqk, dfql, dfqm, dfqn, dfqo, dfqp,
dfqq, dfqr, dfqs, dfqt, dfqv, dfr, dfre, dfrf, dfrg, dfrh, dfri, dfrj, dirk, dfrl, dfrm, dfro, dfrp, dfrq, dfrr, dfrt,
dfru, dfrv, dfs, dfse, dfsf, dfsg, dfsi, dfsl, dfsq, dfst, dft, dfte, dftf, dftg, dfth, dfti, dftj, dftk, dftl, dftm,
dfto, dftp, dftq, dftr, dfts, dftt, dftu, dftv, dfu, dfue, dfuf, dfug, dfuh, dfui, dful, dfuq, dfuv, dfv, dfve,
dfvf, dfvg, dfvh, dfvi, dfvk, dfvl, dfvn, dfvo, dfvp, dfvq, dfvr, dfvs, dfvt, dfvv, dgdh, dgdi, dgdk, dgdl,
dgdm, dgdp, dgdq, dgdr, dgds, dgdt, dgdv, dgeh, dgei, dgej, dgek, dgel, dgem, dgen, dgeo, dgep, dgeq,
dger, dges, dget, dgeu, dgev, dgf, dgfh, dgfi, dgfj, dgfk, dgfl, dgfm, dgfp, dgfq, dgfr, dgfs, dgft, dgfu,
dgfv, dggh, dggi, dggj, dggk, dggl, dggm, dggo, dggp, dggq, dggr, dggs, dggt, dggu, dggv, dgh, dghe,
dghf, dghg, dghh, dghi, dghk, dghl, dghm, dghn, dgho, dghp, dghq, dghr, dght, dghv, dgi, dgie, dgig,
dgih, dgii, dgik, dgil, dgin, dgio, dgip, dgiq, dgir, dgis, dgit, dgiv, dgj, dgjh, dgji, dgjj, dgjk, dgjl, dgjm,
dgjp, dgjq, dgjr, dgjt, dgjv, dgk, dgke, dgkf, dgkg, dgkh, dgki, dgkj, dgkk, dgkl, dgko, dgkp, dgkq, dgkr,
dgkt, dgkv, dgl, dgle, dglf, dglg, dglh, dgli, dglj, dglk, dgll, dgln, dglo, dglp, dglq, dglr, dgls, dglt, dglu,
dglv, dgm, dgme, dgmf, dgmg, dgml, dgmo, dgmr, dgn, dgnf, dgng, dgnh, dgni, dgnj, dgnk, dgnl, dgnm,
dgnp, dgnq, dgnr, dgnt, dgnu, dgnv, dgo, dgoe, dgoh, dgoj, dgol, dgop, dgoq, dgor, dgot, dgou, dgov,
dgp, dgpe, dgpf, dgpg, dgph, dgpi, dgpk, dgpl, dgpm, dgpn, dgpo, dgpp, dgpq, dgpr, dgps, dgpt, dgpu,
dgpv, dgq, dgqe, dgqf, dgqg, dgqh, dgqi, dgqj, dgqk, dgql, dgqm, dgqn, dgqo, dgqp, dgqq, dgqr, dgqt,
dgqu, dgqv, dgr, dgre, dgrf, dgrg, dgrh, dgri, dgrj, dgrk, dgrl, dgrm, dgro, dgrp, dgrq, dgrr, dgrt, dgrv,
dgs, dgse, dgsf, dgsg, dgsh, dgsq, dgsr, dgst, dgsv, dgt, dgte, dgtf, dgtg, dgth, dgti, dgtj, dgtk, dgtl, dgtm,
dgto, dgtp, dgtq, dgtr, dgts, dgtt, dgtu, dgtv, dgu, dgue, dguf, dgug, dguh, dguj, dguk, dgul, dguo, dguq,
dgur, dgut, dguv, dgv, dgve, dgvf, dgvg, dgvh, dgvi, dgvk, dgvl, dgvm, dgvn, dgvo, dgvp, dgvq, dgvr,
dgvt, dgvv, dhdh, dhdi, dhdm, dhdp, dhdq, dhdr, dhds, dhdt, dhdv, dhe, dhee, dhef, dheg, dheh, dhei,
dhek, dhel, dhem, dheo, dhep, dheq, dher, dhes, dhet, dheu, dhev, dhf, dhfe, dhff, dhfg, dhfh, dhfi, dhfj,
dhfm, dhfp, dhfq, dhfr, dhfs, dhft, dhfv, dhg, dhge, dhgf, dhgg, dhgh, dhgi, dhgj, dhgk, dhgl, dhgm, dhgp,
dhgq, dhgr, dhgs, dhgt, dhgu, dhgv, dhh, dhhe, dhhf, dhhg, dhhi, dhhp, dhhq, dhhr, dhhv, dhi, dhie, dhig,
dhih, dhii, dhil, dhip, dhiq, dhir, dhit, dhiv, dhj, dhjf, dhig, dhjh, dhjp, dhjt, dhk, dhkf, dhkg, dhkh, dhki,
dhkm, dhkp, dhkq, dhks, dhkv, dhl, dhle, dhlf, dhlg, dhlh, dhli, dhlj, dhlk, dhll, dhlm, dhlo, dhlp, dhlq,
dhlr, dhls, dhlt, dhlv, dhm, dhme, dhmf, dhmg, dhml, dhn, dhne, dhnf, dhng, dhnh, dhni, dhnm, dhnp,
dhnr, dhns, dhnt, dhnv, dho, dhoe, dhog, dhoh, dhoi, dhol, dhop, dhoq, dhor, dhot, dhov, dhp, dhpe, dhpg,
dhph, dhpl, dhpp, dhpq, dhpu, dhq, dhqe, dhqf, dhqg, dhqh, dhqi, dhqj, dhqk, dhql, dhqm, dhqp, dhqq,
dhqr, dhqs, dhqt, dhqv, dhr, dhre, dhrf, dhrg, dhrh, dhri, dhrj, dhrl, dhrm, dhrp, dhrq, dhrr, dhrt, dhru,
dhrv, dhs, dht, dhte, dhtf, dhtg, dhth, dhti, dhtj, dhtk, dhtl, dhtm, dhtp, dhtq, dhtr, dhts, dhtt, dhtu, dhtv,
dhu, dhuv, dhv, dhve, dhvg, dhvh, dhvi, dhvl, dhvm, dhvn, dhvp, dhvq, dhvr, dhvs, dhvt, dhvv, didi,
didm, didp, didq, didr, didt, didv, die, diee, dief, dieg, dieh, diei, diek, diem, diep, dieq, dier, dies, diet,
dieu, diev, dif, dife, diff, difg, difh, difi, difm, difp, difq, dift, difv, dig, dige, digf, digg, digh, digi, digk,
digl, digm, digp, digq, digr, digs, digt, digv, dih, dihf, dihg, dihi, dii, diig, diiv, dij, dik, dike, dikf, dikg,
dikh, diki, dikj, dikm, dikp, dikr, dikv, dil, dile, dilf, dilg, dilh, dili, dilj, dilk, dill, dilp, dilq, dilr, dilt,
dilv, dim, dimf, dimg, dimh, dimi, din, dine, dinf, ding, dinh, dinm, dinp, ding, dinr, dins, dint, dinu,
dinv, dio, dioe, diof, diog, dioi, dioj, diok, diol, diop, dioq, dior, diot, diov, dip, dipe, dipf, dipg, diph,
dipi, dipj, dipl, dipn, dipr, dipt, diq, diqe, diqf, diqg, diqh, diqi, diqj, diqk, diql, diqq, diqr, diqs, diqt,
diqu, diqv, dir, dire, dirf, dirg, dirh, diri, dirj, dirl, dirp, dirq, dirr, dirs, dirt, diru, dirv, dis, disg, dish, disr,
dit, dite, ditf, ditg, dith, diti, ditj, ditp, ditq, ditr, ditt, ditu, ditv, diu, diuf, div, dive, divg, divh, divi, divk,
divm, divp, divq, divr, divs, divt, divv, djdj, djdk, djdl, didp, didq, djdt, didv, dje, djee, djef, djeg, djek,
djel, djen, djeo, djeq, djet, djev, djf, djfe, djff, djfg, djfh, djfi, djfk, djfl, djfm, djfp, djfq, djfr, djfs, djft,
djfv, djg, djge, djgf, djgg, djgj, djgl, djgo, djgp, digq, djgr, djgv, djh, djhe, djhg, djhl, djho, djhq, djhr,
djht, djhv, dji, djie, djig, djik, djil, djin, djip, djiq, djir, djit, djiv, djje, djjf, djjg, djjh, djji, djjl, djjp, djjq,
djjr, djjs, djjt, djjv, djk, djke, djki, djkj, djkp, djkq, djkr, djkt, djkv, djl, djlq, djlv, djm, djme, djmg, djml,
djmr, din, djo, djp, djpe, djpf, djpg, djph, djpi, djpj, djpk, djpl, djpn, djpo, djpq, djpr, djpt, djpv, djq, djqe,
djqf, djqg, djqi, djqj, djqk, djql, djqn, diqo, djqp, djqq, djqr, djqt, djqv, djr, djre, djrf, djrg, djrh, djri, djrj,
djrk, djrl, djrn, djro, djrp, djrq, djrr, djrt, djrv, djs, djse, djsg, djsn, djso, djsq, djst, djsv, djt, djte, djtf,
djtg, djth, djti, djtj, djtk, djtl, djtn, djto, djtp, djtq, djtr, djtt, djtv, dju, djuf, djug, djuh, djui, djuk, djul,
djuq, djur, djut, djuv, djv, djve, djvf, djvg, djvk, djvl, djvn, djvo, djvp, djvq, djvr, djvt, djvv, dkdl, dkdn,
dkdo, dkdp, dkdq, dkdr, dkds, dkdt, dkdv, dke, dkee, dkef, dkeg, dkeh, dkej, dkek, dkel, dken, dkeo,
dkep, dkeq, dker, dkes, dket, dkeu, dkev, dkf, dkff, dkfi, dkfj, dkfk, dkfp, dkfq, dkfr, dkfs, dkft, dkfu,
dkfv, dkg, dkge, dkgf, dkgg, dkgh, dkgi, dkgj, dkgk, dkgl, dkgp, dkgq, dkgr, dkgt, dkgu, dkgv, dkh, dkhe,
dkhg, dkhi, dkhk, dkhl, dkho, dkhq, dkhr, dkht, dkhv, dki, dkie, dkig, dkih, dkii, dkik, dkil, dkin, dkio,
dkip, dkiq, dkir, dkit, dkiv, dkj, dkjf, dkjg, dkjl, dkjp, dkjq, dkjr, dkjs, dkjt, dkjv, dkke, dkkf, dkkg, dkkh,
dkkj, dkkl, dkko, dkkp, dkkq, dkkr, dkkt, dkkv, dkl, dkle, dklf, dklg, dkli, dklj, dklk, dkll, dkln, dklo,
dklp, dklq, dklr, dklt, dklv, dkm, dkme, dkmg, dkmj, dkn, dknf, dknh, dknj, dknq, dknr, dknt, dknu, dko,
dkoe, dkof, dkog, dkok, dkoq, dkot, dkp, dkpe, dkpf, dkpg, dkpk, dkpl, dkpn, dkpo, dkpq, dkpr, dkpt,
dkpu, dkpv, dkq, dkqe, dkqf, dkqg, dkqj, dkqk, dkql, dkqn, dkqo, dkqp, dkqq, dkqr, dkqt, dkqv, dkr,
dkre, dkrf, dkrg, dkri, dkrj, dkrk, dkrl, dkrn, dkro, dkrp, dkrq, dkrr, dkrt, dkrv, dks, dkse, dksf, dksg, dkt,
dkte, dktf, dktg, dkth, dkti, dktj, dktk, dktl, dkto, dktp, dktq, dktr, dktt, dktv, dku, dkuq, dkv, dkve, dkvf,
dkvg, dkvk, dkvl, dkvn, dkvo, dkvp, dkvq, dkvr, dkvt, dkvu, dkvv, dldl, dldn, dldp, dldq, dldv, dle, dlee,
dlef, dleg, dleh, dlei, dlej, dlek, dlel, dlem, dlen, dleo, dlep, dleq, dler, dlet, dleu, dlev, dlf, dlfe, dlff, dlfg,
dlfh, dlfi, dlfj, dlfk, dlfl, dlfp, dlfq, dlfr, dlfs, dlft, dlfu, dlfv, dlg, dlge, dlgf, dlgg, dlgh, dlgi, dlgj, dlgk,
dlgl, dlgn, dlgp, dlgq, dlgr, dlgt, dlgv, dlh, dlhe, dlhg, dlhk, dlhl, dlhm, dlho, dlhq, dlhr, dlhv, dli, dlie,
dlig, dlik, dlil, dlin, dlip, dliq, dliv, dlj, dlje, dljg, dljh, dlji, dljj, dljl, dljp, dljq, dljr, dljs, dljt, dljv, dlk,
dlke, dlkf, dlkg, dlkh, dlki, dlkj, dlkk, dlkl, dlkp, dlkq, dlkr, dlkt, dlkv, dll, dlle, dllf, dllg, dllh, dlli, dllj,
dllk, dlll, dlln, dllo, dllp, dllq, dllr, dllt, dllv, dlm, dlme, dlmf, dlmg, dlml, dln, dlne, dlnf, dlng, dlnh,
dlni, dlnj, dlnk, dlni, dlnm, dlnp, dlnq, dlnr, dlnt, dlnv, dlo, dloe, dlof, dlog, dloj, dlok, dlp, dlpe, dlpf,
dlpg, dlpj, dlpk, dlpl, dlpn, dlpo, dlpp, dlpq, dlpt, dlpv, dlq, dlqe, dlqf, dlqg, dlqh, dlqi, dlqj, dlqk, dlql,
dlqm, dlqn, dlqo, dlqp, dlqg, dlqr, dlqt, dlqv, dlr, dlre, dlrf, dlrg, dlrh, dlri, dlrj, dlrk, dlrl, dlrn, dlro, dlrp,
dlrq, dlrr, dlrt, dlrv, dls, dlse, dlsf, dlsg, dlsk, dlsl, dlsv, dlt, dlte, dltf, dltg, dlth, dlti, dltj, dltk, dltl, dltn,
dlto, dltp, dltq, dltr, dltt, dltv, dlu, dlue, dlug, dlv, dlve, dlvf, dlvg, dlvh, dlvj, dlvk, dlvl, dlvn, dlvo, dlvp,
dlvq, dlvr, dlvt, dlvv, dmdm, dmdp, dmdq, dmee, dmef, dmeg, dmeh, dmei, dmek, dmem, dmep, dmeq,
dmer, dmes, dmet, dmev, dmf, dmfe, dmff, dmfg, dmfh, dmfi, dmfk, dmfl, dmfm, dmfp, dmfq, dmfr,
dmft, dmfu, dmfv, dmge, dmgf, dmgg, dmgh, dmgi, dmgk, dmgm, dmgp, dmgq, dmgr, dmgs, dmgt,
dmgv, dmh, dmhh, dmhi, dmhp, dmhr, dmht, dmi, dmih, dmii, dmip, dmiq, dmj, dmjf, dmjh, dmjk, dmjp,
dmjq, dmjr, dmjt, dmju, dmjv, dmk, dmkf, dmkg, dml, dmle, dmlf, dmlg, dmli, dmlp, dmlq, dmlr, dmlt,
dmlu, dmlv, dmn, dmng, dmnh, dmni, dmnt, dmo, dmoe, dmoi, dmol, dmop, dmov, dmp, dmpe, dmpf,
dmpg, dmph, dmpm, dmpp, dmpr, dmpt, dmpu, dmpv, dmq, dmqe, dmqf, dmqg, dmqm, dmqp, dmqr,
dmqt, dmqu, dmqv, dmr, dmre, dmrf, dmrg, dmrh, dmrm, dmrp, dmrt, dmrv, dms, dmt, dmte, dmtf,
dmtg, dmth, dmtm, dmtp, dmts, dmtt, dmtu, dmtv, dmu, dmv, dmve, dmvg, dmvh, dmvm, dmvp, dmvq,
dmvr, dmvt, dmvu, dmvv, dndn, dndv, dne, dnee, dnef, dneg, dnej, dnek, dnel, dnen, dneo, dneq, dner,
dnet, dnev, dnf, dnfe, dnff, dnfg, dnfh, dnfi, dnfj, dnfk, dnfl, dnfm, dnfp, dnfq, dnfr, dnfs, dnft, dnfu,
dnfv, dng, dnge, dngf, dngg, dngj, dngl, dngo, dngq, dngr, dngv, dnh, dnhe, dnhf, dnhg, dnhi, dnhk, dnhl,
dnhn, dnho, dnhq, dnhr, dnht, dnhv, dni, dnie, dnig, dnik, dnil, dnin, dnio, dniq, dnir, dnit, dniv, dnj, dnjf,
dnjg, dnjh, dnji, dnjj, dnjk, dnjp, dnjq, dnjr, dnjt, dnjv, dnk, dnke, dnkj, dnkl, dnkn, dnkq, dnkv, dnl,
dnln, dnlq, dnlv, dnm, dnme, dnmg, dnml, dnmp, dnmr, dnmv, dnn, dno, dnp, dnpe, dnpf, dnpg, dnpk,
dnpl, dnpn, dnpo, dnpp, dnpq, dnpr, dnpt, dnpv, dnq, dnqe, dnqf, dnqg, dnqi, dnqj, dnqk, dnql, dnqn,
dnqo, dnqp, dnqq, dnqr, dnqt, dnqv, dnr, dnre, dnrf, dnrg, dnrj, dnrk, dnrl, dnrn, dnro, dnrp, dnrq, dnrr,
dnrt, dnrv, dns, dnse, dnsf, dnsg, dnsk, dnsn, dnsq, dnsv, dnt, dnte, dntf, dntg, dnth, dntj, dntk, dntl, dntn,
dnto, dntp, dntq, dntr, dntt, dntv, dnu, dnue, dnuf, dnug, dnuk, dnul, dnun, dnuo, dnup, dnuq, dnur, dnut,
dnuv, dnv, dnve, dnvf, dnvg, dnvk, dnvl, dnvn, dnvo, dnvp, dnvq, dnvr, dnvt, dnvv, dodq, dodr, dods,
doe, doee, doef, doeg, doeh, doei, doej, doek, doel, doeo, doeq, doer, doet, doev, dof, dofe, doff, dofg,
dofi, dofl, dofq, dofv, dog, doge, dogf, dogg, dogh, dogi, dogk, dogl, dogv, doh, dohe, dohg, dohl, doho,
dohv, doi, doie, doig, doik, doip, doiq, doiv, doj, dojf, dojg, dojh, doji, dojq, dojr, dojt, dojv, dok, doke,
dokf, dokg, dokl, dokn, dol, dole, dolf, dolg, dolj, dolk, doll, doln, dolo, dolv, dom, dome, don, donf,
donj, donq, doo, dop, dope, dopf, dopg, dopk, dopl, dopo, dopv, doq, doqe, doqf, doqg, doqj, doqk, doql,
doqn, doqo, doqp, doqq, doqr, doqt, doqv, dor, dore, dorf, dorg, dorh, dorj, dork, dorl, dorq, dorr, dorv,
dos, dot, dote, dotf, dotg, doth, dotj, dotk, dotl, dotp, dotq, dotr, dott, dotv, dou, doue, douk, dov, dove,
dovf, dovg, dovk, dovl, dovn, dovo, dovq, dovr, dovv, dpdp, dpdq, dpdr, dpdt, dpdu, dpdv, dpe, dpee,
dpef, dpeg, dpeh, dpei, dpej, dpek, dpel, dpem, dpen, dpeo, dpep, dpeq, dper, dpes, dpet, dpeu, dpev, dpf,
dpfe, dpff, dpfg, dpfh, dpfi, dpfk, dpfl, dpfp, dpfq, dpfr, dpfs, dpft, dpfu, dpfv, dpg, dpge, dpgf, dpgg,
dpgh, dpgi, dpgk, dpgl, dpgm, dpgo, dpgp, dpgq, dpgr, dpgs, dpgt, dpgu, dpgv, dph, dphe, dphg, dphi,
dphl, dphm, dpho, dphq, dphr, dphv, dpi, dpie, dpih, dpii, dpim, dpip, dpiq, dpir, dpit, dpiv, dpj, dpjf,
dpjh, dpji, dpjj, dpjp, dpjq, dpjr, dpjs, dpjt, dpju, dpjv, dpk, dpke, dpkg, dpkh, dpkk, dpkl, dpkm, dpkp,
dpkq, dpkr, dpks, dpkt, dpkv, dpl, dple, dplf, dplg, dplh, dpli, dplj, dplk, dpll, dplm, dplo, dplp, dplq,
dplr, dpls, dplt, dplu, dplv, dpm, dpmv, dpn, dpne, dpnf, dpng, dpnh, dpni, dpnj, dpnk, dpnm, dpnp, dpnq,
dpnr, dpns, dpnt, dpnu, dpnv, dpo, dpoe, dpof, dpog, dpoi, dpoj, dpok, dpol, dpon, dpop, dpoq, dpor,
dpot, dpou, dpov, dpp, dppe, dppf, dppg, dppk, dppl, dppn, dppo, dppp, dppq, dppr, dppt, dppu, dppv,
dpq, dpqe, dpqf, dpqg, dpqh, dpqj, dpqk, dpql, dpqm, dpqn, dpqo, dpqp, dpqq, dpqr, dpqs, dpqt, dpqu,
dpqv, dpr, dpre, dprf, dprg, dprh, dpri, dprj, dprk, dprl, dprm, dprp, dprq, dprr, dprs, dprt, dpru, dprv,
dps, dpsq, dpsr, dpt, dpte, dptf, dptg, dpth, dpti, dptj, dptk, dptl, dptm, dptp, dptq, dptr, dpts, dptt, dptu,
dptv, dpu, dpue, dpv, dpve, dpvf, dpvg, dpvh, dpvi, dpvk, dpvl, dpvm, dpvn, dpvo, dpvp, dpvq, dpvr,
dpvt, dpvu, dpvv, dqdq, dqdr, dqds, dqdt, dqdv, dqe, dqee, dqef, dqeg, dqeh, dqei, dqej, dqek, dqel, dqem,
dqen, dqeo, dqep, dqeq, dqer, dqes, dqet, dqeu, dqev, dqf, dqfe, dqff, dqfg, dqfh, dqfi, dqfj, dqfk, dqfl,
dqfn, dqfp, dqfq, dqfr, dqfs, dqft, dqfu, dqfv, dqg, dqge, dqgf, dqgg, dqgh, dqgi, dqgj, dqgk, dqgl, dqgm,
dqgn, dqgo, dqgp, dqgq, dqgr, dqgs, dqgt, dqgu, dqgv, dqh, dqhe, dqhf, dqhg, dqhi, dqhk, dqhl, dqho,
dqhp, dqhq, dqhr, dqhv, dqi, dqie, dqig, dqih, dqii, dqik, dqil, dqio, dqip, dqiq, dqir, dqit, dqiv, dqj, dqje,
dqjf, dqjg, dqjh, dqji, dqjj, dqjk, dqjl, dqjp, dqjq, dqjr, dqjs, dqjt, dqju, dqjv, dqk, dqke, dqkf, dqkg, dqkh,
dqki, dqkk, dqkl, dqkm, dqkn, dqkp, dqkq, dqkr, dqks, dqkt, dqku, dqkv, dql, dqle, dqlf, dqlg, dqlh, dqli,
dqlj, dqlk, dqll, dqln, dqlo, dqlp, dqlq, dqlr, dqls, dqlt, dqlu, dqlv, dqm, dqme, dqmg, dqmp, dqmv, dqn,
dqne, dqnf, dqng, dqnh, dqni, dqnj, dqnk, dqnm, dqnp, dqnq, dqnr, dqnt, dqnu, dqnv, dqo, dqoe, dqof,
dqog, dqoh, dqoi, dqoj, dqok, dqol, dqon, dqoq, dqor, dqos, dqot, dqov, dqp, dqpe, dqpf, dqpg, dqpk,
dqpl, dqpn, dqpo, dqpp, dqpq, dqpr, dqpt, dqpu, dqpv, dqq, dqqe, dqqf, dqqg, dqqh, dqqi, dqqi, dqqk,
dqql, dqqn, dqqo, dqqp, dqqq, dqqr, dqqs, dqqt, dqqu, dqqv, dqr, dqre, dqrf, dqrg, dqrh, dqri, dqrj, dqrk,
dqrl, dqrn, dqro, dqrp, dqrq, dqrr, dqrs, dqrt, dqru, dqrv, dqs, dqse, dqsg, dqsi, dqt, dqte, dqtf, dqtg, dqth,
dqti, dqti, dqtk, dqtl, dqtm, dqtn, dqto, dqtp, dqtq, dqtr, dqts, dqtt, dqtv, dqu, dque, dqug, dqul, dquo,
dquq, dqur, dquv, dqv, dqve, dqvf, dqvg, dqvh, dqvi, dqvj, dqvk, dqvl, dqvn, dqvo, dqvp, dqvq, dqvr,
dqvt, dqvu, dqvv, drdr, drds, drdt, drdv, dre, dree, dref, dreg, dreh, drei, drej, drek, drel, drem, dren, dreo,
drep, dreq, drer, dres, dret, dreu, drev, drf, drfe, drff, drfg, drfh, drfi, drfj, drfk, drfl, drfm, drfp, drfq, drfr,
drfs, drft, drfu, drfv, drg, drge, drgf, drgg, drgh, drgi, drgj, drgk, drgl, drgm, drgo, drgp, drgq, drgr, drgs,
drgt, drgu, drgv, drh, drhe, drhg, drhh, drhi, drhk, drhl, drho, drhp, drhq, drhr, drht, drhv, dri, drie, drig,
drih, drii, drik, dril, drim, drio, drip, driq, drir, drit, driv, drj, drje, drjf, drjg, drjh, drji, drjj, drjk, drjl,
drjm, drjp, drjq, drjr, drjs, drjt, drju, drjv, drk, drke, drkg, drkh, drki, drkk, drkl, drkm, drkp, drkq, drkr,
drks, drkt, drkv, drl, drle, drlf, drlg, drlh, drli, drlj, drlk, drll, drlm, drln, drlo, drlp, drlq, drlr, drls, drlt,
drlu, drlv, drm, drme, drmg, drml, drmp, drn, drne, drnf, drng, drnh, drnj, drnk, drnm, drnp, drnq, drnr,
drns, drnt, drnu, drnv, dro, droe, drof, drog, droh, droi, droj, drok, drol, dron, drop, droq, dror, dros, drot,
drou, drov, drp, drpe, drpf, drpg, drpi, drpj, drpk, drpl, drpn, drpo, drpp, drpq, drpr, drps, drpt, drpu, drpv,
drq, drqe, drqf, drqg, drqh, drqi, drqj, drqk, drql, drqm, drqn, drqo, drqp, drqq, drqr, drqt, drqu, drqv, drr,
drre, drrf, drrg, drrh, drri, drrj, derk, drrl, drrm, drro, drrp, drrq, drrr, drrt, drru, drrv, drs, drse, drsg, drsk,
drsl, drsq, drsv, drt, drte, drtf, drtg, drth, drti, drtj, drtk, drtl, drtm, drto, drtp, drtq, drtr, drts, drtt, drtu,
drtv, dru, drue, druf, drug, druk, drul, drv, drve, drvf, drvg, drvh, drvi, drvk, drvl, drvn, drvo, drvp, drvq,
drvr, drvt, drvu, drvv, dsds, dsdt, dsdu, dsdv, dse, dsee, dsef, dseg, dseh, dsei, dsek, dsel, dsem, dsep,
dseq, dser, dses, dset, dseu, dsev, dsf, dsfe, dsfg, dsfk, dsfl, dsfq, dsfr, dsfs, dsft, dsfv, dsg, dsge, dsgg,
dsgh, dsgk, dsgl, dsgm, dsgp, dsgq, dsgr, dsgt, dsgu, dsgv, dsh, dshe, dshl, dshp, dshq, dsht, dsi, dsie,
dsil, dsip, dsiq, dsir, dsiv, dsj, dsjh, dsjr, dsjt, dsju, dsk, dskg, dskk, dskp, dskq, dskr, dskt, dsl, dsle, dslf,
dslg, dslh, dsli, dslk, dsll, dslm, dslp, dslq, dslr, dslt, dslu, dslv, dsm, dsn, dsne, dsnf, dsnh, dsni, dsnr,
dsnt, dso, dsoe, dsof, dsog, dsoh, dsoi, dsok, dsol, dsop, dsoq, dsor, dsot, dsov, dsp, dspl, dspp, dspr,
dspt, dspv, dsq, dsqe, dsqf, dsqg, dsqh, dsqi, dsqk, dsql, dsqm, dsqp, dsqq, dsqr, dsqs, dsqt, dsqu, dsqv,
dsr, dsre, dsrh, dsri, dsrl, dsrp, dsrq, dsrr, dsrt, dsrv, dss, dst, dste, dstg, dstk, dstl, dstm, dstp, dstq, dstr,
dstt, dstv, dsu, dsv, dsve, dsvf, dsvg, dsvh, dsvl, dsvm, dsvn, dsvp, dsvq, dsvr, dsvt, dsvu, dsvv, dtdt,
dtdv, dte, dtee, dtef, dteg, dteh, dtei, dtej, dtek, dtel, dtem, dten, dteo, dtep, dteq, dter, dtes, dtet, dteu,
dtev, dtf, dtfe, dtff, dtfg, dtfh, dtfi, dtfj, dtfk, dtfl, dtfm, dtfp, dtfq, dtfr, dtfs, dtft, dtfu, dtfv, dtg, dtge,
dtgf, dtgg, dtgh, dtgi, dtgj, dtgk, dtgl, dtgm, dtgo, dtgp, dtgq, dtgr, dtgs, dtgt, dtgu, dtgv, dth, dthe, dthf,
dthg, dthh, dthi, dihk, dthl, dtho, dthp, dthq, dthr, dthv, dti, dtie, dtig, dtih, dtii, dtik, dtim, dtin, dtip, dtiq,
dtir, dtit, dtiv, dtj, dtje, dtjf, dtjg, dtjh, dtji, dtjk, dtjl, dtjm, dtjp, dtjq, dtjr, dtjs, dtjt, dtju, dtjv, dtk, dtke,
dtkg, dtkh, dtki, dtkj, dtkk, dtkl, dtkm, dtkp, dtkq, dtkr, dtks, dtkt, dtku, dtkv, dtl, dtle, dtlf, dtlg, dtlh,
dtli, dtlj, dtlk, dtll, dtlm, dtln, dtlp, dtlq, dtlr, dtls, dtlt, dtlu, dtlv, dtm, dtme, dtmg, dtmr, dtn, dtne, dtnf,
dtng, dtnh, dtnj, dtnm, dtnp, dtnq, dtnr, dtns, dtnt, dtnu, dtnv, dto, dtoe, dtof, dtog, dtoh, dtoi, dtoj, dtok,
dtol, dton, dtoq, dtor, dtos, dtot, dtou, dtov, dtp, dtpe, dtpf, dtpg, dtph, dtpi, dtpj, dtpk, dtpl, dtpn, dtpo,
dtpp, dtpq, dtpr, dtpt, dtpu, dtpv, dtq, dtqe, dtqf, dtqg, dtqh, dtqi, dtqj, dtqk, dtql, dtqm, dtqn, dtqo, dtqp,
dtqq, dtqr, dtqs, dtqt, dtqu, dtqv, dtr, dtre, dtrf, dtrg, dtrh, dtri, dtrj, dtrk, dtrl, dtrm, dtrn, dtro, dtrp, dtrq,
dtrr, dtrs, dtrt, dtru, dtrv, dts, dtse, dtsf, dtsg, dtsh, dtsp, dtsq, dtsv, dtt, dtte, dttf, dttg, dtth, dtti, dttj, dttk,
dttl, dttm, dttn, dtto, dttp, dttq, dttr, dtts, ditt, dttu, dttv, dtu, dtue, dtuf, dtug, dtuh, dtuk, dtuv, dtv, dtve,
dtvf, dtvg, dtvh, dtvi, dtvj, dtvk, dtvl, dtvn, dtvo, dtvp, dtvq, dtvr, dtvt, dtvu, dtvv, dudv, due, duee, duef,
dueg, dueh, duei, duel, duem, duep, dueq, duer, duet, duev, duf, dufh, dufi, dufp, dufq, dufv, dug, duge,
dugi, dugm, dugp, dugq, dugs, dugv, duh, dui, duj, duje, dujg, dujp, dujv, duk, duke, duki, dukp, dukq,
dukr, dukv, dul, dule, dulf, dulg, dulh, duli, dulm, dulp, dulq, dulr, duls, dulv, dum, dun, dune, dung,
dunh, dunp, dunr, dunt, dunv, duo, duof, duoq, duov, dup, dupf, dupg, dupv, duq, duqe, duqf, duqg, duqh,
duqi, duqj, duql, duqq, duqv, dur, dure, durf, durg, durh, duri, durm, durq, durv, dus, dut, dute, dutf, dutg,
duth, duti, dutm, dutp, dutq, dutv, duu, duv, duvf, duvi, duvl, duvt, duvv, dvdv, dve, dvee, dvef, dveg,
dveh, dvei, dvej, dvek, dvel, dvem, dven, dveo, dvep, dveq, dver, dves, dvet, dveu, dvev, dvf, dvfe, dvff,
dvfg, dvfh, dvfi, dvfj, dvfk, dvfl, dvfo, dvfp, dvfq, dvfr, dvfs, dvft, dvfv, dvg, dvge, dvgf, dvgg, dvgh,
dvgi, dvgj, dvgk, dvgl, dvgm, dvgn, dvgo, dvgp, dvgq, dvgr, dvgs, dvgt, dvgu, dvgv, dvh, dvhe, dvhg,
dvhh, dvhi, dvhk, dvhl, dvho, dvhp, dvhq, dvhr, dvht, dvhv, dvi, dvie, dvig, dvih, dvii, dvik, dvil, dvim,
dvin, dvip, dviq, dvir, dvit, dviv, dvj, dvjf, dvji, dvjj, dvk, dvke, dvkf, dvkg, dvkh, dvkk, dvkl, dvkm,
dvkp, dvkq, dvkr, dvks, dvkt, dvku, dvkv, dvl, dvle, dvlf, dvlg, dvlh, dvli, dvlj, dvlk, dvll, dvlm, dvln,
dvlo, dvlp, dvlq, dvlr, dvls, dvlt, dvlu, dvlv, dvm, dvme, dvmf, dvmg, dvmi, dvmj, dvmo, dvn, dvne,
dvnf, dvng, dvnh, dvni, dvnj, dvnk, dvnm, dvnp, dvnq, dvnr, dvns, dvnt, dvnu, dvnv, dvo, dvoe, dvof,
dvog, dvoh, dvoi, dvoj, dvok, dvol, dvon, dvop, dvoq, dvor, dvot, dvou, dvov, dvp, dvpe, dvpf, dvpg,
dvpk, dvpl, dvpn, dvpo, dvpp, dvpq, dvpr, dvpt, dvpu, dvpv, dvq, dvqe, dvqf, dvqg, dvqh, dvqi, dvqj,
dvqk, dvql, dvqm, dvqn, dvqo, dvqp, dvqq, dvqr, dvqs, dvqt, dvqu, dvqv, dvr, dvre, dvrf, dvrg, dvrh,
dvri, dvrj, dvrk, dvrl, dvrm, dvrn, dvro, dvrp, dvrq, dvrr, dvrs, dvrt, dvru, dvrv, dvs, dvse, dvsg, dvsl,
dvso, dvsq, dvst, dvsv, dvt, dvte, dvtf, dvtg, dvth, dvti, dvtj, dvtk, dvtl, dvtm, dvto, dvtp, dvtq, dvtr, dvts,
dvtt, dvtu, dvtv, dvu, dvue, dvuf, dvug, dvuj, dvul, dvup, dvuv, dvv, dvve, dvvf, dvvg, dvvh, dvvi, dvvk,
dvvl, dvvm, dvvn, dvvo, dvvp, dvvq, dvvr, dvvs, dvvt, dvvu, dvvv, eeeh, eeei, eeej, eeek, eeem, eeeo,
eeep, eeeq, eeer, eees, eeet, eeeu, eeev, eeff, eefh, eefi, eefj, eefk, eefl, eefm, eefn, eefo, eefp, eefq, eefr,
eefs, eeft, eefu, eefv, eegh, eegi, eegj, eegk, eegm, eegn, eego, eegp, eegq, eegr, eegs, eegt, eegu, eegv,
eeh, eehf, eehg, eehh, eehi, eehj, eehk, eehl, eehn, eeho, eehp, eehq, eehr, eeht, eehv, eei, eeif, eeig, eeih,
eeii, eeij, eeik, eeil, eeim, eein, eeio, eeip, eeiq, eeir, eeis, eeit, eeiv, eej, eejf, eejg, eejh, eeji, eejj, eejk,
eejl, eejm, eejp, eejq, eejr, eejs, eejt, eeju, eejv, eek, eekf, eekg, eekh, eeki, eekj, eekk, eekl, eekm, eekn,
eeko, eekp, eekg, eekr, eeks, eekt, eeku, eekv, eel, eelf, eelh, eeli, eelj, eelk, eell, eelm, eeln, eelo, eelp,
eelq, eelr, eels, eelt, eelv, eem, eemf, eemg, eemi, eemj, eemk, eeml, eemm, eemn, eemo, eemp, eemq,
eemr, eems, eemt, eemv, eenf, eeng, eenh, eeni, eenj, eenk, eenl, eenm, eenp, eenq, eenr, eens, eent,
eenu, eenv, eeo, eeof, eeog, eeoh, eeoi, eeoj, eeok, eeol, eeom, eeon, eeoo, eeop, eeoq, eeor, eeot, eeou,
eeov, eep, eepf, eepg, eeph, eepi, eepj, eepk, eepl, eepm, eepn, eepo, eepp, eepq, eepr, eeps, eept, eepu,
eepv, eeq, eeqf, eeqg, eeqh, eeqi, eeqi, eeqk, eeql, eeqm, eeqn, eego, eeqp, eeqq, eeqr, eeqs, eeqt, eequ,
eeqv, eer, eerf, eerg, eerh, eeri, eerj, eerk, eerl, eerm, eern, eero, eerp, eerq, eerr, eers, eert, eeru, eerv,
ees, eesf, eesg, eesh, eesi, eesj, eesk, eesl, eesm, eesn, eeso, eesp, eesq, eesr, eest, eesv, eet, eetf, eetg,
eeth, eeti, eetj, eetk, eetl, eetm, eetn, eeto, eetp, eetq, eetr, eets, eett, eetu, eetv, eeu, eeuf, eeug, eeuh,
eeui, eeuk, eeul, eeun, eeuo, eeup, eeuq, eeur, eeut, eeuv, eev, eevf, eevg, eevh, eevi, eevj, eevk, eevl,
eevm, eevn, eevo, eevp, eevq, eevr, eevs, eevt, eevu, eevv, efef, efeh, efei, efej, efek, efel, efem, efen,
efeo, efep, efeq, efer, efes, efet, efeu, efev, efff, effg, effh, effi, effj, effk, effl, effm, effo, effp, effq, effr,
effs, efft, effu, effv, efgf, efgh, efgi, efgj, efgk, efgl, efgm, efgn, efgo, efgp, efgq, efgr, efgs, efgt, efgu,
efgv, efh, efhf, efhg, efhh, efhi, efhk, efhl, efho, efhp, efhq, efhr, efht, efhu, efhv, efi, efif, efig, efih, efii,
efik, efil, efim, efin, efio, efip, efiq, efir, efis, efit, efiv, efj, efjf, efjg, efjh, efji, efjj, efjk, efjl, efjm, efjp,
efjq, efjr, efjs, efjt, efju, efjv, efk, efkf, efkg, efkh, efki, efkj, efkk, efkl, efkm, efkn, efko, efkp, ofkq,
efkr, efks, efkt, efku, efkv, efl, eflf, eflg, eflh, efli, eflj, eflk, efll, efln, eflo, eflp, eflq, eflr, efls, eflt, eflu,
eflv, efin, efmf, efmg, efmh, efmi, efmj, efmk, efml, efmo, efmp, efmr, efmt, efmv, efn, efnf, efng, efnh,
efni, efnj, efnk, efnm, efnp, efnq, efnr, efns, efnt, efnu, efnv, efo, efof, efog, efoh, efoi, efoj, efok, efol,
efon, efop, efoq, efor, efos, efot, efou, efov, efp, efpf, efpg, efph, efpi, efpj, efpk, efpl, efpm, efpn, efpo,
efpp, ofpq, efpr, efpt, efpu, efpv, efq, ofqf, efqg, efqh, efqi, efqj, efqk, efql, efqm, efqn, efqo, efqp, efqq,
efqr, efqs, efqt, efqu, efqv, efr, efrf, efrg, efrh, efri, efrj, efrk, efrl, efrm, efm, efro, efrp, efrq, efrr, efrs,
efrt, efru, efrv, efs, efsf, efsg, efsh, efsk, efsl, efso, efsp, efsq, efsr, efst, efsv, eft, eftf, eftg, efth, efti, eftj,
eftk, eftl, eftm, eftn, efto, eftp, eftq, eftr, efts, eftt, eftu, eftv, efu, efuf, efug, efuk, eful, efuo, efup, efuq,
efur, efut, efuv, efv, efvf, efvg, efvh, efvi, efvj, efvk, efvl, efvm, efvn, efvo, efvp, efvq, efvr, efvs, efvt,
efvu, efvv, egeh, egei, egej, egek, egem, egen, egeo, egep, egeq, eger, eges, eget, egeu, egev, egff, egfh,
egfi, egfj, egfk, egfl, egfm, egfn, egfo, egfp, egfq, egfr, egfs, egft, egfu, egfv, eggh, eggi, eggi, eggk,
eggm, eggn, eggo, eggp, eggq, eggr, eggs, eggt, eggu, eggv, egh, eghf, eghg, eghh, eghi, eghk, eghl,
eghm, eghn, egho, eghp, eghq, eghr, eghs, eght, eghv, egi, egif, egig, egih, egii, egik, egil, egim, egin,
egio, egip, egiq, egir, egis, egit, egiu, egiv, egj, egjf, egjg, egjh, egji, egjj, egjk, egjl, egjm, egjp, egjq,
egjr, egjs, egjt, egju, egjv, egk, egkf, egkg, egkh, egki, egkj, egkk, egkl, egkm, egkn, egko, egkp, egkq,
egkr, egks, egkt, egku, egkv, egl, eglf, eglh, egli, eglj, eglk, egll, eglm, egln, eglo, eglp, eglq, eglr, egls,
eglt, eglu, eglv, egm, egmf, egmg, egmj, egmk, egml, egmo, egmp, egmq, egmr, egmt, egmv, egn, egnf,
egnh, egni, egnj, egnk, egnm, egnp, egnq, egnr, egns, egnt, egnu, egnv, ego, egof, egog, egoh, egoi, egoj,
egok, egol, egom, egon, egop, egoq, egor, egot, egou, egov, egp, egpf, egpg, egph, egpi, egpj, egpk, egpl,
egpm, egpn, egpo, egpp, egpq, egpr, egpt, egpu, egpv, egq, egqf, egqg, egqh, egqi, egqi, egqk, egql,
egqm, egqn, egqo, egqp, egqq, egqr, egqs, egqt, egqu, egqv, egr, egrf, egrg, egrh, egri, egrj, egrk, egrl,
egrm, egrn, egro, egrp, egrq, egrr, egrs, egrt, egru, egrv, egs, egsf, egsg, egsi, egsk, egsl, egsm, egsn,
egso, egsp, egsq, egsr, egst, egsv, egt, egtf, egtg, egth, egti, egtj, egtk, egtl, egtm, egtn, egto, egtp, egtq,
egtr, egts, egtt, egtu, egtv, egu, eguf, egug, eguh, eguk, egul, egun, eguo, egup, eguq, egur, egut, eguv,
egv, egvf, egvg, egvh, egvi, egvj, egvk, egvl, egvm, egvn, egvo, egvp, egvq, egvr, egvs, egvt, egvu, egvv,
eheh, ehei, ehej, ehek, ehel, ehem, ehen, ehep, eheq, eher, ehes, ehet, eheu, ehev, ehf, ehff, ehfg, ehfh,
ehfi, ehfk, ehfl, ehfm, ehfp, ehfq, ehfr, ehfs, ehft, ehfv, ehg, ehgf, ehgg, ehgh, ehgi, ehgk, ehgl, ehgm,
ehgo, ehgp, ehgq, ehgr, ehgs, ehgt, ehgu, ehgv, ehh, ehhf, ehhg, ehhh, ehhi, ehhk, ehhm, ehho, ehhp,
ehhq, ehhr, ehht, ehhv, ehi, ehif, ehig, ehih, ehii, ehik, ehil, ehim, ehio, ehip, ehiq, ehir, ehis, ehit, ehiv,
ehj, ehjf, ehjg, ehjh, ehji, ehjm, ehjp, ehjq, ehjr, ehjs, ehjt, ehju, ehjv, ehk, ehkg, ehkh, ehki, ehkk, ehkl,
ehkm, ehko, ehkp, ehkq, ehkr, ehks, ehkt, ehku, ehkv, ehl, ehlf, ehlg, ehlh, ehli, ehlj, ehlk, ehll, ehln,
ehlo, ehlp, ehlq, ehlr, ehls, ehlt, ehlu, ehlv, ehm, ehmf, ehmg, ehml, ehmp, ehmq, ehmr, ehmv, ehn, ehnf,
ehng, ehnh, ehni, ehnj, ehnk, ehnl, ehnm, ehnp, ehnq, ehnr, ehns, ehnt, ehnu, ehnv, eho, ehof, ehog, ehoh,
ehoi, ehoj, ehok, ehol, ehom, ehon, ehop, ehoq, ehor, ehos, ehot, ehou, ehov, ehp, ehpg, ehph, ehpi, ehpk,
ehpl, ehpm, ehpn, ehpo, ehpp, ehpq, ehpr, ehps, ehpt, ehpu, ehpv, ehq, ehqf, ehqg, ehqh, ehqi, ehqj, ehqk,
ehql, ehqm, ehqn, ehqo, ehqp, ehqq, ehqr, ehqs, ehqt, ehqu, ehqv, ehr, ehrf, ehrg, ehrh, ehri, ehrj, ehrk,
ehrl, ehrm, ehro, ehrp, ehrq, ehrr, ehrs, ehrt, ehru, ehrv, ehs, ehsg, ehsm, ehsq, ehsv, eht, ehtf, ehtg, ehth,
ehti, ehtj, ehtk, ehtl, ehtm, ehto, ehtp, ehtq, ehtr, ehts, ehtt, ehtu, ehtv, ehu, ehuf, ehuh, ehup, ehuq, ehuv,
ehv, ehvf, ehvg, ehvh, ehvi, ehvk, ehvl, ehvm, ehvn, ehvo, ehvp, ehvq, ehvr, ehvs, ehvt, ehvu, ehvv, eiei,
eiej, eiek, eiel, eiem, eien, eieo, eiep, eieq, eier, eies, eiet, eieu, eiev, eif, eiff, eifg, eifh, eifi, eifk, eifl,
eifp, eifq, eifr, eifs, eift, eifu, eifv, eig, eigf, eigg, eigh, eigi, eigk, eigl, eigm, eign, eigo, eigp, eigq, eigr,
eigs, eigt, eigu, eigv, eih, eihg, eihh, eihk, eihl, eihm, eiho, eihp, eihq, eihr, eihs, eiht, eihv, eii, eiff, eiig,
eiii, eiik, eiil, eiio, eiip, eiiq, eiir, eiit, eiiv, eij, eijf, eijg, eijh, eijm, eijp, eijs, eijt, eiju, eik, eikf, eikg,
eikh, eiki, eikk, eikl, eikm, eiko, eikp, eikq, eikr, eiks, eikt, eiku, eikv, eil, eilf, eilg, eilh, eili, eilk, eill,
eilm, eiln, eilo, eilp, eilq, eilr, eils, eilt, eilu, eilv, eim, eimf, eimr, ein, einf, eing, einh, eini, einj, eink,
einl, einm, einp, einq, einr, eins, eint, einu, einv, eio, eiof, eiog, eioh, eioi, eioj, eiok, eiol, eiom, eion,
eiop, eioq, eior, eios, eiot, eiou, eiov, eip, eipf, eipg, eiph, eipk, eipl, eipm, eipn, eipo, eipp, eipq, eipr,
eips, eipt, eipu, eipv, eiq, eiqf, eiqg, eiqh, eiqi, eiqj, eiqk, eiql, eiqm, eiqn, eiqo, eiqp, eiqq, eiqr, eiqs,
eiqt, eiqu, eiqv, eir, eirf, eirg, eirh, eiri, eirj, eirk, eirl, eirm, eiro, eirp, eirq, eirr, eirs, eirt, eiru, eirv, eis,
eisf, eish, eisl, eism, eiso, eisp, eisq, eisr, eiss, eist, eisv, eit, eitf, eitg, eith, eiti, eitj, eitk, eitl, eitm, eito,
eitp, eitq, eitr, eits, eitt, eitu, eitv, eiu, eiuq, eiut, eiuv, eiv, eivf, eivg, eivh, eivi, eivk, eivl, eivm, eivn,
eivo, eivp, eivq, eivr, eivs, eivt, eivu, eivv, ejek, ejel, ejen, ejeo, ejep, ejeq, ejer, ejes, ejet, ejeu, ejev, ejf,
ejff, ejfg, ejfh, ejfi, ejfj, ejfk, ejfl, ejfm, ejfn, ejfo, ejfp, ejfq, ejfr, ejfs, ejft, ejfu, ejfv, ejg, ejgf, ejgg, ejgi,
ejgk, ejgl, ejgn, ejgo, ejgp, ejgq, ejgr, ejgt, ejgu, ejgv, ejh, ejhf, ejhg, ejhh, ejhi, ejhk, ejhl, ejhn, ejho,
ejhp, ejhq, ejhr, ejht, ejhv, eji, ejif, ejig, ejih, ejii, ejik, ejil, ejin, ejio, ejip, ejiq, ejir, ejis, ejit, ejiv, ejj,
ejjf, ejjg, ejjh, ejji, ejjk, ejjl, ejjm, ejjp, ejjq, ejjr, ojjs, ejjt, ejjv, ejk, ejkf, ejkg, ejkh, ejkk, ejkl, ejkn, ejko,
ejkp, ejkq, ejkr, ejkt, ejkv, ejl, ejlf, ejlg, ejlk, ejll, ejln, ejlq, ejlr, ejlt, ejlv, ejm, ejmg, ejmj, ejmp, ejmr,
ejmt, ejmv, ejn, ejnf, ejnj, ejnu, ejo, ejp, ejpf, ejpg, ejph, ejpk, ejpl, ejpn, ejpo, ejpp, ejpq, ejpr, ejpt, ejpv,
ejq, ejqf, ejqg, ejqh, ejqj, ejqk, ejql, ejqn, ejqo, ejqp, ejqq, ejqr, ejqt, ejqv, ejr, ejrf, ejrg, ejrh, ejrj, ejrk,
ejrl, ejrn, ejro, ejrp, ejrq, ejrr, ejrt, ejrv, ejs, ejsg, ejsk, ejsl, ejso, ejsp, ejsq, ejsr, ejst, ejsv, ejt, ejtf, ejtg,
ejth, ejti, ejtj, ejtk, ejtl, ejtn, ejto, ejtp, ejtq, ejtr, ejtt, ejtv, eju, ejuf, ejug, ejuk, ejul, ejun, ejuo, ejup, ejuq,
ejur, ejut, ejuv, ejv, ejvf, ejvg, ejvh, ejvk, ejvl, ejvn, ejvo, ejvp, ejvq, ejvr, ejvt, ejvv, ekek, ekel, ekem,
eken, ekeo, ekep, ekeq, eker, ekes, eket, ekeu, ekev, ekf, ekff, ekfg, ekfh, ekfi, ekfj, ekfk, ekfl, ekfm,
ekfp, ekfq, ekfr, ekfs, ekft, ekfu, ekfv, ekg, ekgf, ekgg, ekgh, ekgi, ekgj, ekgk, ekgl, ekgm, ekgn, ekgp,
ekgq, ekgr, ekgs, ekgt, ekgu, ekgv, ekh, ekhf, ekhg, ekhh, ekhi, ekhj, ekhk, ekhl, ekhn, ekho, ekhp, ekhq,
ekhr, ekht, ekhv, eki, ekif, ekig, ekih, ekii, ekik, ekil, ekim, ekin, ekio, ekip, ekiq, ekir, ekis, ekit, ekiv,
ekj, ekjf, ekjg, ekjh, ekji, ekjl, ekjp, ekjq, ekjr, ekjs, ekjt, ekjv, ekk, ekkf, ekkg, ekkh, ekki, ekkk, ekkl,
ekkm, ekkn, ekko, ekkp, ekkq, ekkr, ekks, ekkt, ekku, ekkv, ekl, eklf, eklg, eklh, ekli, eklj, eklk, ekll,
eklm, ekln, eklo, eklp, eklq, eklr, ekls, eklt, eklu, eklv, ekm, ekmf, ekmg, ekmj, ekmn, ekmo, ekmq,
ekmv, ekn, eknf, ekng, eknh, ekni, eknj, eknk, eknm, eknp, eknq, eknr, ekns, eknt, eknu, eknv, eko, ekof,
ekog, ekoh, ekoi, ekok, ekol, ekop, ekoq, ekor, ekos, ekot, ekou, ekov, ekp, ekpf, ekpg, ekph, ekpi, ekpj,
ekpk, ekpl, ekpn, ekpo, ekpp, ekpq, ekpr, ekps, ekpt, ekpv, ekq, ekqf, ekqg, ekqh, ekqi, ekqi, ekqk, ekql,
ekqm, ekqn, ekqo, ekqp, ekqq, ekqr, ekqs, ekqt, ekqu, ekqv, ekr, ekrf, ekrg, ekrh, ekri, ekrj, ekrk, ekrl,
ekrm, ekrn, ekro, ekrp, ekrq, ekrr, ekrs, ekrt, ekru, ekrv, eks, eksg, eksk, eksn, ekso, eksq, ekst, eksv, ekt,
ektf, ektg, ekth, ekti, ektj, ektk, ektl, ektm, ektn, ekto, ektp, ektq, ektr, ektt, ektu, ektv, eku, ekuf, ekug,
ekuk, ekul, ekun, ekuo, ekuq, ekur, ekut, ekuv, ekv, ekvf, ekvg, ekvh, ekvi, ekvk, ekvl, ekvm, ekvn, ekvo,
ekvp, ekvq, ekvr, ekvs, ekvt, ekvu, ekvv, elel, elem, elen, eleo, elep, eleq, eler, eles, elet, eleu, elev, elf,
elff, elfg, elfh, elfi, elfj, elfk, elfl, elfn, elfo, elfp, elfq, elfr, elfs, elft, elfu, elfv, elg, elgf, elgh, elgi, elgi,
elgk, elgl, elgn, elgo, elgp, elgq, elgr, elgs, elgt, elgu, elgv, elh, elhf, elhg, elhh, elhi, elhk, elhl, elhn,
elho, elhp, elhq, elhr, elht, elhu, elhv, eli, elif, elig, elih, elii, elik, elil, elin, elio, elip, eliq, elir, elit, eliu,
eliv, elj, eljf, eljg, eljh, eljj, eljl, eljp, eljq, eljr, eljs, eljt, eljv, elk, elkf, elkg, elkh, elki, elkk, elkl, elkn,
elko, elkp, elkq, elkr, elks, elkt, elku, elkv, ell, ellf, ellg, ellh, elli, ellj, ellk, elll, elln, ello, ellp, ellq, ellr,
ells, ellt, ellu, ellv, elm, elmf, elmg, elmj, elmk, elmo, elmp, elmq, elmr, elmt, elmv, eln, elnf, elng, elnh,
elni, elnj, elnk, elnl, elnm, elnp, elnq, elnr, elns, elnt, elnu, elnv, elo, elof, elog, eloh, eloi, eloj, elok, elol,
elon, elop, eloq, elor, elot, elou, elov, elp, elpf, elpg, elpj, elpk, elpl, elpn, elpo, elpp, elpq, elpr, elps,
elpt, elpv, elq, elqf, elqg, elqh, elqi, elqi, elqk, elql, elqm, elqn, elqo, elqp, elqq, elqr, elqs, elqt, elqv, elr,
elrf, elrg, elrh, elri, elrj, elrk, elrl, elrn, elro, elrp, elrq, elrr, elrs, elrt, elrv, els, elsf, elsg, elsn, elso, elsp,
elsq, elsr, elst, elsv, elt, eltf, eltg, elth, elti, eltj, eltk, eltl, eltm, eltn, elto, eltp, eltq, eltr, elts, eltt, eltv, elu,
eluk, elul, elup, eluq, elur, elut, eluv, elv, elvf, elvg, elvh, elvi, elvj, elvk, elvl, elvm, elvn, elvo, elvp,
elvq, elvr, elvs, elvt, elvu, elvv, emem, emep, emeq, emer, emes, emet, emeu, emev, emf, emff, emfg,
emfh, emfi, emfj, emfk, emfl, emfm, emfp, emfq, emfr, emfs, emft, emfu, emfv, emg, emgf, emgg, emgh,
emgi, emgk, emgl, emgm, emgp, emgq, emgr, emgs, emgt, emgu, emgv, emh, emhg, emhh, emhm,
emhq, emhs, emhv, emi, emig, emih, emii, emim, emip, emiq, emir, emit, emiv, emj, emjf, emjg, emjh,
emji, emjj, emjm, emjp, emjq, emjr, emjs, emjt, emju, emjv, emk, emkg, emkh, emki, emkm, emkp,
emkq, emkr, emks, emkt, emku, emkv, eml, emlf, emlg, emlh, emli, emlk, emll, emlm, emlp, emlq, emlr,
emls, emlt, emlv, emm, emn, emnf, emng, emnh, emni, emnm, emnp, emnq, emnr, emns, emnt, emnu,
emnv, emo, emog, emoh, emoi, emok, emol, emom, emop, emoq, emor, emot, emov, emp, empg, emph,
empi, empk, empl, empm, empp, empq, empr, emps, empt, empu, empv, emq, emqf, emqg, emqh, emqi,
emqj, emql, emqm, emqq, emqr, emqs, emqt, emqu, emqv, emr, emrf, emrg, emrh, emri, emrk, emrl,
emrm, emrp, emrq, emrr, emrs, emrt, emru, emrv, ems, emsh, emsm, emsp, emsv, emt, emtf, emtg, emth,
emti, emtm, emtp, emtq, emtr, emts, emtt, emtu, emtv, emu, emup, emuq, emuv, emv, emvf, emvg,
emvh, emvi, emvk, emvl, emvm, emvn, emvp, emvq, emvr, emvs, emvt, emvu, emvv, eneo, enep, eneq,
ener, enes, enet, eneu, enev, enf, enff, enfg, enfh, enfi, enfj, enfk, enfl, enfm, enfo, enfp, enfq, enfr, enfs,
enft, enfu, enfv, eng, engf, engg, engh, engi, engj, engk, engl, engm, engn, engo, engp, engq, engr, engs,
engt, engu, engv, enh, enhf, enhg, enhi, enhj, enhk, enhl, enhn, enho, enhp, enhq, enhr, enht, enhv, eni,
enif, enig, enih, enik, enil, enin, enio, enip, eniq, enir, enis, enit, eniv, enj, enjf, enjg, enjh, enji, enjj, enjk,
enjl, enjp, enjq, enjr, enjs, enjt, enju, enjv, enk, enkf, enkg, enkh, enki, enkk, enkl, enkn, enkp, enkq,
enkr, enkt, enkv, enl, enlh, enlk, enll, enlq, enlv, enm, enmf, enmg, enmk, enml, enmp, enmr, enmt,
enmv, ennf, enni, ennj, ennp, ennq, ennr, enns, ennt, ennu, ennv, eno, enp, enpf, enpg, enph, enpi, enpj,
enpk, enpl, enpn, enpo, enpp, enpq, enpr, enpt, enpv, enq, enqf, enqg, engh, enqi, enqj, enqk, enql, enqn,
enqo, enqp, enqq, enqr, enqt, enqv, enr, enrf, enrg, enrh, enri, enrj, enrk, enrl, enrn, enro, enrp, enrq, enrr,
enrt, enrv, ens, ensf, ensg, ensi, ensj, ensk, ensl, ensn, enso, ensq, ensr, enst, ensv, ent, entf, entg, enth,
enti, entj, entk, entl, entn, ento, entp, entq, entr, entt, entv, enu, enuf, enug, enuh, enuj, enul, enun, enuo,
enuq, enur, enut, enuv, env, envf, envg, envh, envi, envj, envk, envl, envn, envo, envp, envq, envr, envt,
envv, eoeo, eoep, eoeq, eoer, eoes, eoet, eoev, eof, eoff, eofg, eofj, eofk, eofl, eofn, eofo, eofp, eofq, eofr,
eoft, eofu, eofv, eog, eogf, eogg, eogh, eogj, eogk, eogl, eogn, eogo, eogp, eogq, eogr, eogt, eogv, eoh,
eohg, eohk, eohl, eohn, eoho, eohp, eohq, eohr, eoht, eohv, eoi, eoif, eoig, eoii, eoik, eoil, eoio, eoip,
eoiq, eoir, eoit, eoiv, eoj, eojf, eojg, eojh, eojj, eojk, eojl, eojp, eojq, eojr, eojt, eojv, eok, eokf, eokg,
eokk, eokl, eokn, eokq, eokr, eokt, eokv, eol, eolf, eolg, eolj, eolk, eoll, eoln, eolo, eolq, eolr, eolt, eolv,
eom, eomf, eomq, eomr, eon, eonf, eong, eoni, eonj, eonk, eonl, eonn, eonp, eonq, eonr, eont, eonv, eoo,
eoog, eoon, eop, eopg, eopk, eopl, eopn, eopo, eopq, eopr, eopt, eopv, eoq, eoqf, eoqg, eoqh, eoqj, eoqk,
eoql, eoqn, eoqo, eoqp, eoqq, eoqr, eoqt, eoqv, eor, eorf, eorg, eorj, eork, eorl, eorn, eoro, eorp, eorq,
eorr, eort, eorv, eos, eosi, eosl, eosp, eosq, eosv, eot, eotf, eotg, eotj, eotk, eotl, eotn, eoto, eotp, eotq,
eotr, eott, eotv, eou, eoug, eouk, eoul, eouo, eour, eout, eouv, eov, eovf, eovg, eovj, eovk, eovl, eovn,
eovo, eovp, eovq, eovr, eovt, eovv, epep, epeq, eper, epes, epet, epeu, epev, epf, epff, epfg, epfh, epfi,
epfk, epfl, epfm, epfp, epfq, epfr, epfs, epft, epfu, epfv, epg, epgf, epgg, epgh, epgi, epgk, epgl, epgm,
epgn, epgo, epgp, epgq, epgr, epgs, epgt, epgu, epgv, eph, ephf, ephg, ephh, ephi, ephk, ephl, ephm,
ephn, epho, ephp, ephq, ephr, epht, ephv, epi, epig, epih, epii, epik, epil, epim, epin, epio, epip, epiq,
epir, epis, epit, epiv, epj, epjf, epjg, epjh, epji, epjk, epjl, epjm, epjp, epjq, epjr, epjs, epjt, epju, epjv, epk,
epkg, epkh, epki, epkk, epkl, epkm, epkn, epko, epkp, epkq, epkr, epks, epkt, epku, epkv, epl, eplf, eplg,
eplh, epli, eplk, epll, eplm, eplo, eplp, eplq, eplr, epls, eplt, eplu, eplv, epm, epmf, epmi, epmn, epmp,
epmq, epmr, epmt, epmv, epn, epnf, epng, epnh, epni, epnj, epnk, epnl, epnm, epnp, epnq, epnr, epns,
epnt, epnu, epnv, epo, epof, epog, epoh, epoi, epoj, epok, epol, epom, epoo, epop, epoq, epor, epos, epot,
epou, epov, epp, eppg, epph, eppi, eppk, eppl, eppm, eppn, eppo, eppp, eppq, eppr, epps, eppt, eppu,
eppv, epq, epqf, epqg, epqh, epqi, epqi, epqk, epql, epqm, epqn, epqo, epqp, epqq, epqr, epqs, epqt, epqu,
epqv, epr, eprf, eprg, eprh, epri, eprj, eprk, eprl, eprm, eprn, epro, eprp, eprq, eprr, eprs, eprt, epru, eprv,
eps, epsg, epsi, epsl, epsn, epso, epsp, epsq, epsr, epst, epsv, ept, eptf, eptg, epth, epti, eptj, eptk, eptl,
eptm, eptn, epto, eptp, eptq, eptr, epts, eptt, eptu, eptv, epu, epug, epul, epun, epuo, epup, epuq, epur,
epus, eput, epuv, epv, epvf, epvg, epvh, epvi, epvj, epvk, epvl, epvm, epvn, epvo, epvp, epvq, epvr, epvs,
epvt, epvu, epvv, eqeq, eqer, eqes, eqet, eqeu, eqev, eqf, eqff, eqfg, eqfh, eqfi, eqfj, eqfk, eqfl, eqfm,
eqfn, eqfo, eqfp, eqfq, eqfr, eqfs, eqft, eqfu, eqfv, eqg, eqgf, eqgg, eqgh, eqgi, eqgj, eqgk, eqgl, eqgm,
eqgn, eqgo, eqgp, eqgq, eqgr, eqgs, eqgt, eqgu, eqgv, eqh, eqhf, eqhg, eqhh, eqhi, eghj, eqhk, eqhl, eqhm,
eqhn, eqho, eqhp, eqhq, eqhr, eqhs, eqht, eqhv, eqi, eqif, eqig, eqih, eqii, eqik, eqil, eqim, eqin, egio,
eqip, eqiq, eqir, eqis, eqit, eqiv, eqj, eqjf, eqjg, eqjh, eqji, eqjj, eqjk, eqjl, eqjm, eqjo, eqjp, eqjq, eqjr,
eqjs, eqjt, eqju, eqjv, eqk, eqkf, eqkg, eqkh, eqki, eqkk, eqkl, eqkm, eqkn, eqko, eqkp, eqkq, eqkr, eqks,
eqkt, eqku, eqkv, eql, eqlf, eqlg, eqlh, eqli, eqlj, eqlk, eqll, eqlm, eqln, eqlo, eqlp, eqlq, eqlr, eqls, eqlt,
eqlu, eqlv, eqm, eqmf, eqmg, eqmh, eqmi, eqmj, eqmk, eqml, eqmm, eqmn, eqmp, eqmq, eqmr, eqms,
eqmt, eqmv, eqn, eqnf, eqng, eqnh, eqni, eqnj, eqnk, eqnl, eqnm, eqnp, eqnq, eqnr, eqns, eqnt, eqnu,
eqnv, eqo, eqof, eqog, eqoh, eqoi, eqoj, eqok, eqol, eqom, eqon, eqoo, eqop, eqoq, eqor, eqos, eqot, eqou,
eqov, eqp, eqpf, eqpg, eqph, eqpi, eqpk, eqpl, eqpm, eqpn, eqpo, eqpp, eqpq, eqpr, eqps, eqpt, eqpu,
eqpv, eqq, eqqf, eqqg, eqqh, eqqi, eqqi, eqqk, eqql, eqqm, eqqn, eqqo, eqqp, eqqq, eqqr, eqqs, eqqt, eqqu,
eqqv, eqr, eqrf, eqrg, eqrh, eqri, eqrj, eqrk, eqrl, eqrm, eqrn, eqro, eqrp, eqrq, eqrr, eqrs, eqrt, eqru, eqrv,
eqs, eqsg, eqsh, eqsi, eqsk, eqsl, eqsn, eqsp, eqsq, eqsr, eqst, eqsv, eqt, eqtf, eqtg, eqth, eqti, eqtj, eqtk,
eqtl, eqtm, eqtn, eqto, eqtp, eqtq, eqtr, eqts, eqtt, eqtu, eqtv, equ, equg, equk, equl, equn, equp, equq,
equr, equs, equt, equv, eqv, eqvf, eqvg, eqvh, eqvi, eqvj, eqvk, eqvl, eqvm, eqvn, eqvo, eqvp, eqvq, eqvr,
eqvs, eqvt, eqvu, eqvv, erer, eres, eret, ereu, erev, erf, erff, erfg, erfh, erfi, erfj, erfk, erfl, erfm, erfn, erfo,
erfp, erfq, erfr, erfs, erft, erfu, erfv, erg, ergf, ergg, ergh, ergi, ergj, ergk, ergl, ergm, ergn, ergo, ergp,
ergq, ergr, ergs, ergt, ergu, ergv, erh, erhf, erhg, erhh, erhi, erhk, erhl, erhm, erhn, erho, erhp, erhq, erhr,
erhs, erht, erhv, eri, erif, erig, erih, erii, erik, eril, erim, erin, erio, erip, eriq, erir, eris, erit, eriv, erj, erjf,
erjg, erjh, erji, erjj, erjk, erjl, erjm, erjp, erjq, erjr, erjs, erjt, erju, erjv, erk, erkf, erkg, erkh, erki, erkk,
erkl, erkm, erkn, erkp, erkq, erkr, erks, erkt, erku, erkv, erl, erlf, erlg, erlh, erli, erlj, erlk, erll, erlm, erln,
erlo, erlp, erlq, erlr, erls, erlt, erlu, erlv, erm, ermf, ermg, ermh, ermi, ermj, erml, ermp, ermq, ermr, ermt,
ermv, ern, ernf, erng, ernh, erni, ernj, ernk, ernl, ernm, ernp, ernq, ernr, erns, ernt, ernu, ernv, ero, erof,
erog, eroh, eroi, eroj, erok, erol, erom, eron, erop, eroq, eror, eros, erot, erou, erov, erp, erpf, erpg, erph,
erpi, erpk, erpl, erpm, erpn, erpo, erpp, erpq, erpr, erps, erpt, erpu, erpv, erq, erqf, erqg, erqh, erqi, erqj,
erqk, erql, erqm, erqn, erqo, erqp, erqq, erqr, erqs, erqt, erqu, erqv, err, errf, errg, errh, erri, errj, errk,
errl, errm, errn, erro, errp, errq, errr, errs, errt, erru, errv, ers, ersf, ersg, ersk, ersl, ersn, erso, ersp, ersq,
erst, ersv, ert, ertf, ertg, erth, erti, ertj, ertk, ertl, ertm, ertn, erto, ertp, ertq, ertr, erts, ertt, ertu, ertv, eru,
erug, eruk, erul, eruo, erup, eruq, erur, erut, eruv, erv, ervf, ervg, ervh, ervi, ervj, ervk, ervl, ervm, ervn,
ervo, ervp, ervq, ervr, ervs, ervt, ervu, ervv, eses, eset, eseu, esev, esf, esff, esfg, esfh, esfi, esfk, esfl,
esfp, esfq, esfr, esfs, esft, esfv, esgf, esgg, esgh, esgi, esgk, esgl, esgm, esgp, esgq, esgr, esgs, esgt, esgu,
esgv, esh, eshf, eshg, eshh, eshi, eshl, eshp, eshq, eshv, esi, esig, esih, esil, esim, esio, esip, esiq, esir, esj,
esjf, esig, esjh, esjj, esjm, esjp, esjr, esjt, esjv, esk, eskg, eskh, eski, eskl, eskp, eskq, eskr, eskt, esku,
eskv, esl, eslf, eslg, eslh, esli, eslj, eslk, esll, eslm, eslp, eslq, eslr, esls, eslt, eslv, esm, esmp, esn, esnf,
esnh, esnj, esnk, esnm, esnp, esnq, esnr, esns, esnt, esnu, esnv, eso, esof, esog, esoh, esoj, esok, esol,
esom, eson, esop, esoq, esor, esot, esov, esp, espg, esph, espi, espl, espp, espq, espr, espt, espv, esq, esqf,
esqg, esqh, esqi, esqj, esqk, esql, esqm, esqp, esqq, esqr, esqt, esqu, esqv, esr, esrf, esrg, esrh, esri, esrj,
esrk, esrl, esrm, esrp, esrq, esrr, esrs, esrt, esru, esrv, ess, essl, essr, esst, est, estf, estg, esth, esti, estj,
estk, estl, estm, estp, estq, estr, ests, estt, estu, estv, esu, esuv, esv, esvf, esvg, esvh, esvi, esvk, esvl, esvn,
esvp, esvq, esvr, esvt, esvv, etet, eteu, etev, etf, etff, etfg, etfh, etfi, etfj, etfk, etfl, etfm, etfn, etfo, etfp,
etfq, etfr, etfs, etft, etfu, etfv, etg, etgf, etgg, etgh, etgi, etgj, etgk, etgl, etgm, etgn, etgo, etgp, etgq, etgr,
etgs, etgt, etgu, etgv, eth, ethf, ethg, ethh, ethi, ethk, ethl, ethm, ethn, etho, ethp, ethq, ethr, eths, etht,
ethv, eti, etif, etig, etih, etii, etik, etil, etim, etin, etio, etip, etiq, etir, etis, etit, etiv, etj, etjf, etjg, etjh, etji,
etjj, etjk, etjl, etjm, etjn, etjp, etjq, etjr, etjs, etjt, etju, etjv, etk, etkf, etkg, etkh, etki, etkk, etkl, etkm,
etkn, etko, etkp, etkq, etkr, etks, etkt, etku, etkv, etl, etlf, etlg, etlh, etli, etlj, etlk, etll, etlm, etln, etlo,
etlp, etlq, etlr, etls, etlt, etlu, etlv, etm, etmf, etmg, etmh, etmi, etmj, etml, etmm, etmo, etmp, etmq, etmr,
etmt, etmv, etn, etnf, etng, etnh, etni, etnj, etnk, etnl, etnm, etnp, etnq, etnr, etns, etnt, etnu, etnv, eto,
etof, etog, etoh, etoi, etoj, etok, etol, etom, eton, etop, etoq, etor, etos, etot, etou, etov, etp, etpf, etpg,
etph, etpi, etpj, etpk, etpl, etpm, etpn, etpo, etpp, etpq, etpr, etps, etpt, etpu, etpv, etq, etqf, etqg, etqh,
etqi, etqi, etqk, etql, etqm, etqn, etqo, etqp, etqq, etqr, etqs, etqt, etqu, etqv, etr, etrf, etrg, etrh, etri, etrj,
etrk, etrl, etrm, etrn, etro, etrp, etrq, etrr, etrs, etrt, etru, etrv, ets, etsg, etsh, etsi, etsk, etsl, etsm, etsn,
etsp, etsq, etsr, etst, etsv, ett, ettf, ettg, etth, etti, ettj, ettk, ettl, ettm, ettn, etto, ettp, ettq, ettr, etts, ettt,
ettu, ettv, etu, etuf, etug, etuk, etul, etuo, etup, etuq, etur, etut, etuv, etv, etvf, etvg, etvh, etvi, etvj, etvk,
etvl, etvm, etvn, etvo, etvp, etvq, etvr, etvs, etvt, etvu, etvv, eueu, euev, euf, euff, eufg, eufh, eufi, eufk,
eufl, eufm, eufp, eufq, eufr, eufs, euft, eufu, eufv, eug, eugf, eugg, eugh, eugi, eugk, eugl, eugm, eugo,
eugp, eugq, eugr, eugs, eugt, eugu, eugv, euh, euhf, euhg, euhi, euhk, euhl, euho, euhq, euhr, euhv, eui,
enig, euii, euil, euip, euiq, euir, euit, euiv, euj, eujf, eujg, eujh, eujk, eujl, eujp, eujq, eujr, eujs, eujt, eujv,
euk, eukf, eukg, eukh, euki, eukk, eukl, eukp, eukq, eukr, eukt, eukv, eul, eulf, eulg, eulh, euli, eulj, eulk,
eull, euln, eulo, eulp, eulq, eulr, euls, eult, eulu, eulv, eum, eumh, eumi, eun, eunf, eung, eunh, euni,
eunj, eunk, eunm, eunp, eunq, eunr, eunt, eunu, eunv, euo, euof, euog, euoj, euok, euol, euon, euop, euoq,
euor, euot, euou, euov, eup, eupg, eupk, eupl, eupo, eupq, eupr, eupt, eupv, euq, euqf, euqg, eugh, euqi,
euqj, euqk, euql, euqn, enqo, euqp, euqq, euqr, euqt, euqu, euqv, eur, eurf, eurg, eurh, euri, eurj, eurk,
eurl, euro, eurp, eurq, eurr, eurt, euru, eurv, eus, eut, eutf, eutg, euth, euti, eutj, eutk, eutl, eutm, euto,
eutp, eutq, eutr, eutt, eutu, eutv, euu, euv, euvf, euvg, euvh, euvi, euvj, euvk, euvl, euvn, euvo, euvq,
euvr, euvt, euvu, euvv, evev, evf, evff, evfg, evfh, evfi, evfj, evfk, evfl, evfm, evfp, evfq, evfr, evfs, evft,
evfu, evfv, evg, evgf, evgg, evgh, evgi, evgj, evgk, evgl, evgm, evgn, evgo, evgp, evgq, evgr, evgs, evgt,
evgu, evgv, evh, evhf, evhg, evhh, evhi, evhj, evhk, evhl, evhm, evhn, evho, evhp, evhq, evhr, evht, evhv,
evi, evif, evig, evih, evii, evik, evil, evim, evin, evio, evip, eviq, evir, evis, evit, eviv, evj, evjf, evjg, evjh,
evji, evjm, evjp, evjq, evjr, evjs, evjt, evju, evjv, evk, evkf, evkg, evkh, evki, evkk, evkl, evkm, evkn,
evko, evkp, evkq, evkr, evks, evkt, evku, evkv, evl, evlf, evlg, evlh, evli, evlj, evlk, evll, evlm, evln, evlo,
evlp, evlq, evlr, evls, evlt, evlu, evlv, evm, evmf, evmg, evmh, evmi, evmj, evmk, evmm, evmn, evmp,
evmq, evmr, evmt, evmv, evn, evnf, evng, evnh, evni, evnj, evnk, evnl, evnm, evnn, evnp, evnq, evnr,
evns, evnt, evnu, evnv, evo, evof, evog, evoh, evoi, evoj, evok, evol, evom, evon, evoo, evop, evoq, evor,
evos, evot, evou, evov, evp, evpf, evpg, evph, evpi, evpk, evpl, evpm, evpn, evpo, evpp, evpq, evpr,
evps, evpt, evpu, evpv, evq, evqf, evqg, evqh, evqi, evqj, evqk, evql, evqm, evqn, evqo, evqp, evqq, evqr,
evqs, evqt, evqu, evqv, evr, evrf, evrg, evrh, evri, evrj, evrk, evrl, evrm, evrn, evro, evrp, evrq, evrr, evrs,
evrt, evru, evrv, evs, evsf, evsg, evsh, evsk, evsl, evsn, evso, evsp, evsq, evsr, evss, evst, evsv, evt, evtf,
evtg, evth, evti, evtj, evtk, evtl, evtm, evtn, evto, evtp, evtq, evtr, evts, evtt, evtu, evtv, evu, evuf, evug,
evup, evuq, evur, evus, evv, evvf, evvg, evvh, evvi, evvk, evvl, evvm, evvn, evvo, evvp, evvq, evvr,
evvs, evvt, evvu, evvv, fffh, fffi, fffj, fffk, fffm, fffn, fffo, fffp, fffq, fffr, fffs, ffft, fffu, fffv, ffgh, ffgi,
ffgj, ffgk, ffgm, ffgn, ffgo, ffgp, ffgq, ffgr, ffgs, ffgt, ffgu, ffgv, ffh, ffhg, ffhh, ffhi, ffhj, ffhk, ffhl, ffhm,
ffhn, ffho, ffhp, ffhq, ffhr, ffht, ffhu, ffhv, ffi, ffig, ffih, ffii, ffik, ffil, ffim, ffin, ffio, ffip, ffiq, ffir, ffis,
ffit, ffiv, ffj, ffjg, ffjh, ffji, ffjj, ffjk, ffjm, ffjn, ffjp, ffjq, ffjr, ffjs, ffjt, ffju, ffjv, ffk, ffkg, ffkh, ffki, ffkj,
ffkk, ffkl, ffkn, ffkp, ffkq, ffkr, ffkt, ffkv, ffl, fflh, ffli, fflj, fflk, ffll, film, filn, fflp, ffiq, fflr, ffls, fflt,
fflv, ffm, ffmg, ffmh, ffmi, fimj, ffmk, ffml, ffmm, ffmo, ffmp, ffmq, ffmr, fims, ffmt, ffmv, ffn, ffng,
ffnh, ffnj, ffns, ffo, ffoj, ffp, ffpg, ffph, ffpi, ffpj, ffpk, ffpl, ffpm, ffpn, ffpo, ffpp, ffpq, ffpr, ffps, ffpt,
ffpv, ffq, ffqg, ffqh, ffqi, ffqj, ffqk, ffql, ffqm, ffqn, ffqo, ffqp, ffqq, ffqr, ffqs, ffqt, ffqu, ffqv, ffr, ffrg,
ffrh, ffri, ffrj, ffrk, ffrl, ffrm, ffrn, ffro, ffrp, ffrq, ffrr, ffrs, ffrt, ffrv, ffs, ffsg, ffsh, ffsi, ffsk, ffsl, ffsm,
ffsn, ffso, ffsq, ffsr, ffst, ffsv, fft, fftg, ffth, ffti, fftj, fftk, fftl, fftm, fftn, ffto, fftp, fftq, fftr, ffts, fftt, fftu,
fftv, ffu, ffug, ffuh, ffui, ffuj, ffuk, fful, ffum, ffun, ffuo, ffup, ffuq, ffur, ffut, ffuv, ffv, ffvg, ffvh, ffvi,
ffvj, ffvk, ffvl, ffvm, ffvn, ffvo, ffvp, ffvq, ffvr, ffvs, ffvt, ffvv, fgfh, fgfi, fgfj, fgfk, fgfm, fgfn, fgfo,
fgfp, fgfq, fgfr, fgfs, fgft, fgfu, fgfv, fggh, fggi, fggj, fggk, fggm, fggn, fggo, fggp, fggq, fggr, fggs, fggt,
fggu, fggv, fgh, fghg, fghh, fghi, fghj, fghl, fghm, fghn, fgho, fghp, fghq, fghr, fght, fghv, fgi, fgig, fgih,
fgij, fgik, fgil, fgim, fgin, fgio, fgip, fgiq, fgir, fgis, fgit, fgiu, fgiv, fgj, fgjg, fgjh, fgji, fgij, fgjk, fgjm,
fgjn, fgip, fgjq, fgjr, fgjs, fgjt, fgju, fgjv, fgk, fgkg, fgkh, fgki, fgkk, fgkl, fgkm, fgkn, fgko, fgkp, fgkq,
fgkr, fgkt, fgku, fgkv, fgl, fglh, fgli, fglj, fglk, fgll, fglm, fgln, fglp, fglq, fglr, fgls, fglt, fglu, fglv, fgm,
fgmg, fgmj, fgmk, fgml, fgmn, fgmo, fgmp, fgmq, fgmr, fgmt, fgn, fgng, fgnh, fgnj, fgnm, fgnn, fgnp,
fgnq, fgnr, fgnt, fgnu, fgnv, fgo, fgog, fgoh, fgoi, fgoj, fgol, fgom, fgon, fgop, fgoq, fgor, fgot, fgov, fgp,
fgpg, fgph, fgpj, fgpl, fgpm, fgpn, fgpo, fgpp, fgpq, fgpr, fgpt, fgpv, fgq, fgqg, fgqh, fgqi, fgqj, fgqk,
fgql, fgqn, fgqo, fgqp, fgqq, fgqr, fgqt, fgqu, fgqv, fgr, fgrg, fgrh, fgri, fgrj, fgrk, fgrl, fgrm, fgrn, fgro,
fgrp, fgrq, fgrr, fgrt, fgru, fgrv, fgs, fgsg, fgsj, fgsl, fgsn, fgso, fgsq, fgsr, fgst, fgt, fgtg, fgth, fgti, fgtj,
fgtk, fgtl, fgtm, fgtn, fgto, fgtp, fgtq, fgtr, fgtt, fgtu, fgtv, fgu, fgug, fguh, fguj, fgul, fgun, fguo, fguq,
fgur, fgut, fguv, fgv, fgvg, fgvh, fgvj, fgvk, fgvl, fgvm, fgvn, fgvo, fgvp, fgvq, fgvr, fgvs, fgvt, fgvu,
fgvv, fhfh, fhfi, fhfj, fhfk, fhfm, fhfn, fhfp, fhfq, fhfr, fhfs, fhft, fhfu, fhfv, fhg, fhgg, fhgh, fhgi, fhgj,
fhgk, fhgl, fhgm, fhgn, fhgo, fhgp, fhgq, fhgr, fhgs, fhgt, fhgu, fhgv, fhh, fhhg, fhhh, fhhm, fhhn, fhhp,
fhhq, fhhr, fhht, fhhu, fhi, fhig, fhih, fhii, fhik, fhil, fhim, fhin, fhio, fhip, fhiq, fhir, fhit, fhiu, fhj, fhjg,
fhjj, fhjm, fhjr, fhjt, fhk, fhkg, fhkh, fhkk, fhkl, fhkm, fhkn, fhkp, fhkq, fhkr, fhkt, fhku, fhkv, fhl, fhlg,
fhlh, fhli, fhij, fhik, fhil, fhim, fhin, fhip, fhiq, fhir, fhis, fhit, fhlu, fhlv, fhm, fhmg, fhmj, fhmn, fhmp,
fhmr, fhmt, fhmu, fhn, fhng, fhnh, fhni, fhnj, fhnk, fhnm, fhnp, fhnq, fhnr, fhns, fhnt, fhnu, fhnv, fho,
fhog, fhoi, fhoj, fhok, fhol, fhon, fhop, fhoq, fhor, fhot, fhov, fhp, fhpg, fhph, fhpj, fhpl, fhpm, fhpn,
fhpo, fhpq, fhpr, fhpt, fhpv, fhq, fhqg, fhqh, fhqi, fhqj, fhqk, fhql, fhqm, fhqn, fhqo, fhqp, fhqq, fhqr,
fhqt, fhqu, fhqv, fhr, fhrg, fhrh, fhri, fhrj, fhrk, fhrl, fhrm, fhrn, fhro, fhrp, fhrq, fhrr, fhrs, fhrt, fhru, fhrv,
fhs, fhsg, fhsj, fhsl, fhsm, fhsn, fhso, fhsq, fhsr, fhst, fhsv, fht, fhtg, fhth, fhtj, fhtk, fhtl, fhtm, fhtn, fhto,
fhtp, fhtq, fhtr, fhtt, fhtu, fhtv, fhu, fhug, fhuj, fhun, fhur, fhut, fhuv, fhv, fhvg, fhvh, fhvk, fhvl, fhvm,
fhvn, fhvo, fhvp, fhvq, fhvr, fhvt, fhvu, fhvv, fifi, fifj, fifq, fifr, fift, fifu, fifv, fig, figg, figh, figi, figj,
figk, figl, figm, fign, figp, figq, figr, figs, figt, figu, figv, fih, fihg, fihh, fihl, fihm, fihq, fihr, fiht, fii, fiig,
fiil, fiio, fiiq, fiir, fiit, fiiu, fij, fik, fikg, fikh, fiki, fikj, fikl, fikm, fikn, fikp, fikq, fikr, fiks, fikt, fiku, fikv,
fil, filg, filh, fili, filj, filk, fill, filn, filo, filp, filq, filr, filt, filu, filv, fim, fimg, fimj, fimt, fin, fing, finh,
finj, finm, finp, finr, fins, fint, finv, fio, fiog, fioh, fioi, fioj, fiok, fiol, fion, fioq, fior, fiot, fiov, fip, fipg,
fiph, fipi, fipj, fipk, fipl, fipm, fipn, fipo, fipq, fipr, fipt, fipv, fiq, fiqg, fiqh, fiqi, fiqi, fiqk, fiql, fiqm,
fiqn, fiqo, fiqp, fiqq, fiqr, fiqt, fiqu, fiqv, fir, firg, firh, firi, firj, firk, firl, firm, firn, firo, firp, firq, firr,
firs, firt, firu, firv, fis, fisg, fish, fisi, fisl, fiso, fisq, fisr, fist, fisv, fit, fitg, fith, fiti, fitj, fitk, fitl, fitn, fito,
fitp, fitq, fitr, fitt, fitu, fitv, fiu, fiuq, fiv, fivg, fivh, fivi, fivj, fivk, fivl, fivm, fivn, fivo, fivp, fivq, fivr,
fivs, fivt, fivu, fivv, fjfj, fjfl, fjfm, fjfn, fjfp, fjfq, fjfr, fjft, fjfu, fjfv, fjg, fjgg, fjgl, fjgm, fjgn, fjgo, fjgq,
fjgr, fjgt, figv, fjh, fjhg, fjhi, fjhj, fjhl, fjhm, fjhn, fjho, fjhq, fjhr, fjht, fjhv, fji, fjig, fjik, fjil, fjim, fjin,
fjio, fjiq, fjir, fjis, fjit, fjiv, fjj, fjjg, fjjh, fjjp, fjjq, fjjr, fjjs, fjjt, fjju, fjjv, fjk, fjkg, fjki, fjkl, fjkn, fikr, fjkt,
fjkv, fjl, fjlg, fjlj, fjlk, fjln, filq, fjlv, fjm, fjmg, fjml, fjmm, fjmn, fjmo, fjmq, fjmr, fjmt, fjn, fjnj, fjo, fjp,
fjpg, fjpi, fjpj, fjpk, fjpl, fjpm, fjpn, fjpo, fjpq, fjpr, fjpt, fjpv, fjq, fjqg, fjqi, fjqj, fjqk, fjql, fjqm, fjqn,
fjqq, fjqr, fjqt, fjqv, fjr, fjrg, fjrh, fjri, fjrj, fjrk, fjrl, fjrm, fjrn, fjro, fjrp, fjrq, fjrr, fjrt, fjrv, fjs, fjsg, fjsi,
fjsm, fjsq, fjsr, fjst, fjsv, fjt, fjtg, fjth, fjti, fjtj, fjtk, fjtl, fjtm, fjtn, fjto, fjtq, fjtr, fjtt, fjtv, fjug, fjui, fjuj,
fjuk, fjul, fjun, fjuq, fjur, fjut, fjuv, fjv, fjvg, fjvh, fjvi, fjvj, fjvk, fjvl, fjvm, fjvn, fjvo, fjvq, fjvr, fjvt,
fjvv, fkfk, fkfl, fkfm, fkfn, fkfo, fkfp, fkfq, fkfr, fkft, fkfu, fkfv, fkg, fkgg, fkgh, fkgi, fkgj, fkgk, fkgl,
fkgm, fkgn, fkgo, fkgp, fkgq, fkgr, fkgs, fkgt, fkgu, fkgv, fkh, fkhg, fkhj, fkhl, fkhm, fkhn, fkho, fkhq,
fkhr, fkhs, fkht, fkhv, fki, fkig, fkih, fkij, fkik, fkil, fkim, fkin, fkio, fkip, fkiq, fkir, fkis, fkit, fkiv, fkj,
fkjg, fkjk, fkjm, fkjp, fkjq, fkjr, fkjt, fkjv, fkk, fkkg, fkkj, fkkk, fkkl, fkkn, fkkp, fkkq, fkkr, fkkt, fkkv,
fkl, fklg, fkli, fklk, fkll, fkin, fklo, fklp, fklq, fklr, fkls, fklt, fklv, fkm, fkmg, fkmk, fkml, fkmn, fkmo,
fkmp, fkmq, fkmr, fkmt, fkn, fknh, fknj, fknp, fknq, fknr, fknt, fknu, fknv, fko, fkoq, fkor, fkot, fkp,
fkpg, fkpj, fkpl, fkpn, fkpo, fkpq, fkpr, fkpt, fkpv, fkq, fkqg, fkqh, fkqi, fkqk, fkql, fkqn, fkqo, fkqp,
fkqq, fkqr, fkqt, fkqv, fkr, fkrg, fkrh, fkrj, fkrl, fkrn, fkro, fkrp, fkrq, fkrr, fkrt, fkrv, fks, fksj, fksn, fksq,
fksr, fkst, fkt, fktg, fkth, fkti, fktj, fktl, fktn, fkto, fktp, fktq, fktr, fktt, fktv, fku, fkug, fkuo, fkuq, fkur,
fkut, fkuv, fkv, fkvg, fkvh, fkvj, fkvk, fkvl, fkvm, fkvn, fkvo, fkvp, fkvg, fkvr, fkvs, fkvt, fkvv, flfl, flfm,
flfn, flfo, flfp, flfq, flfr, flfs, flft, flfu, flfv, flg, flgh, flgi, flgj, flgk, flgl, flgm, flgn, flgo, flgp, flgq, flgr,
flgs, flgt, flgu, flgv, flh, flhg, flhj, flhl, flhn, flho, flhq, flhr, flht, fli, flig, flij, flik, flil, flim, flin, flio, flip,
fliq, flir, flis, flit, fliv, flj, fljg, fljk, fljl, fljm, fljn, fljp, fljq, fljr, fljt, fljv, flk, flkg, flkh, flki, flkj, flkk,
flkl, flkm, flkn, flko, flkp, flkq, flkr, flkt, flku, flkv, fll, fllg, fllh, fllj, fllk, flll, flln, fllo, fllp, fllq, fllr, fllt,
fllv, flm, flml, flmq, flmr, flmt, flmv, fln, flng, flnh, flni, flnj, flnk, flnn, flnp, flnq, flnr, flnt, flnu, flnv,
flo, flol, floq, flov, flp, flpg, flph, flpj, flpl, flpn, flpo, flpp, flpq, flpr, flpt, flpv, flq, flqg, flqh, flqi, flqj,
flqk, flql, flqm, flqn, flqo, flqp, flqq, flqr, flqt, flqv, flr, flrg, flrh, flri, flrj, flrl, flrn, flro, flrp, flrq, flrr,
flrt, firv, fls, flsl, flsn, flso, flsq, flsr, flst, flsv, flt, fltg, flth, flti, fltj, fltk, fltl, fltn, flto, fltp, fltq, fltr, fltt,
fltv, flu, flug, fluj, flul, fluo, flug, flur, flut, fluv, flv, flvg, flvh, flvi, flvj, flvk, flvl, flvm, flvn, flvo, flvp,
flvq, flvr, flvs, flvt, flvu, flvv, fmfm, fmfn, fmfp, fmfq, fmfr, fmfs, fmft, fmfu, fmfv, fmg, fmgg, fmgh,
fmgi, fmgj, fmgk, fmgm, fmgn, fmgp, fmgq, fmgr, fmgs, fmgt, fmgu, fmgv, fmh, fmhg, fmhh, fmhi,
fmhm, fmhn, fmhr, fmht, fmi, fmig, fmih, fmim, fmir, fmit, fmj, fmjg, fmjh, fmjj, fmjp, fmjq, fmjr, fmjt,
fmju, fmjv, fmk, fmkt, fmku, fml, fmlg, fmlh, fmli, fmlj, fmll, fmlm, fmln, fmlp, fmlq, fmlr, fmls, fmlt,
fmlv, fmm, fmn, fmng, fmnm, fmnt, fmo, fmog, fmoj, fmom, fmon, fmop, fmoq, fmor, fmos, fmot, fmou,
fmov, fmp, fmpg, fmph, fmpn, fmpp, fmpq, fmpr, fmpt, fmpv, fmq, fmqg, fmqh, fmqr, fmqt, fmqu, fmr,
fmrg, fmrh, fmrj, fmrk, fmrl, fmrm, fmrn, fmrq, fmrr, fmrt, fmru, fmrv, fms, fmsl, fmsn, fmsr, fmt, fmtg,
fmth, fmtj, fmtm, fmtr, fmtt, fmtu, fmu, fmv, fmvp, fmvq, fmvr, fmvt, fmvu, fmvv, fnfn, fnfo, fnfp, fnfq,
fnfr, fnfs, fnft, fnfu, fnfv, fng, fngg, fngn, fngq, fngr, fngt, fnh, fnhg, fnhj, fnhl, fnhn, fnho, fnhq, fnhr,
fnht, fni, fnil, fniq, fniv, fnj, fnjg, fnji, fnjj, fnjk, fnjl, fnjn, fnjp, fnjq, fnjr, fnjt, fnju, fnjv, fnk, fnkq, fnkr,
fnkv, fnl, fnlq, fnlv, fnm, fnmg, fnmo, fnmq, fnmr, fnmt, fnn, fno, fnp, fnpg, fnpl, fnpn, fnpo, fnpq, fnpr,
fnpt, fnpv, fnq, fnqg, fnql, fnqo, fnqq, fnqr, fnqv, fnr, fnrg, fnrj, fnrl, forn, fnro, fnrp, fnrq, fnrr, fnrt, fnrv,
fns, fnsg, fnsj, fnsl, fnsn, fnso, fnsq, fnsr, fnst, fnt, fntg, fntj, fntl, fntn, fnto, fntp, fntq, fntr, fntt, fntv,
fnug, fnuj, fnul, fnun, fnuo, fnuq, fnur, fnut, fnuv, fnv, fnvg, fnvl, fnvn, fnvo, fnvq, fnvr, fnvv, fofo, fofq,
fofr, foft, fofv, fog, fogl, fogn, fogq, fogr, fogv, foh, foho, fohq, fohr, foht, foi, foig, foin, foio, foiq, foir,
foj, fojg, fojk, fojl, fojm, fojn, fojq, fojr, fojt, fok, fokl, fokn, fokr, fokv, fol, folq, folv, fom, fomq, fon,
fonn, fonq, foo, fop, fopl, fopq, fopr, fopt, fopv, foq, foqg, foqj, fogl, foqn, fogo, foqq, fogr, foqt, fogv,
for, forg, forj, forl, forn, foro, forq, forr, fort, forv, fos, fosl, foso, fosq, fosr, fot, fotg, fotj, fotl, fotn, foto,
fotq, fotr, fott, fotv, fou, foul, fouo, fouq, four, fout, fouv, fov, fovg, fovj, fovl, fovn, fovo, fovq, fovr,
fovt, fovv, fpfp, fpfq, fpfr, fpfs, fpft, fpfu, fpfv, fpg, fpgg, fpgh, fpgi, fpgj, fpgk, fpgl, fpgm, fpgn, fpgo,
fpgp, fpgq, fpgr, fpgs, fpgt, fpgu, fpgv, fph, fphg, fphh, fphj, fphk, fphl, fphm, fphn, fpho, fphp, fphq,
fphr, fpht, fphv, fpi, fpig, fpih, fpij, fpik, fpil, fpim, fpin, fpio, fpip, fpiq, fpir, fpis, fpit, fpiu, fpj, fpjg,
fpjh, fpjj, fpjk, fpjm, fpjp, fpjq, fpjr, fpjt, fpju, fpjv, fpk, fpkg, fpkh, fpki, fpkk, fpkl, fpkm, fpkn, fpkp,
fpkq, fpkr, fpks, fpkt, fpku, fpkv, fpl, fplg, fplh, fpli, fplj, fplk, fpll, fplm, fpln, fplo, fplp, fplq, fplr, fpls,
fplt, fplu, fplv, fpm, fpmg, fpmo, fpmp, fpmr, fpmt, fpn, fpng, fpnh, fpni, fpnj, fpnk, fpnm, fpnn, fpnp,
fpnq, fpnr, fpns, fpnt, fpnu, fpnv, fpo, fpog, fpoi, fpoj, fpok, fpol, fpom, fpon, fpop, fpoq, fpor, fpot,
fpou, fpov, fpp, fppg, fppk, fppl, fppn, fppo, fppp, fppq, fppr, fpps, fppt, fppu, fppv, fpq, fpqg, fpqh, fpqi,
fpqj, fpqk, fpql, fpqm, fpqn, fpqo, fpqp, fpqq, fpqr, fpqt, fpqu, fpqv, fpr, fprg, fprh, fpri, fprj, fprk, fprl,
fprm, fprn, fpro, fprp, fprq, fprr, fprs, fprt, fpru, fprv, fps, fpsr, fpst, fpsu, fpsv, fpt, fptg, fpth, fpti, fptj,
fptk, fptl, fptm, fptn, fpto, fptp, fptq, fptr, fpts, fptt, fptu, fptv, fpu, fpul, fpun, fpup, fpuq, fpur, fput, fpuv,
fpv, fpvg, fpvh, fpvi, fpvj, fpvk, fpvl, fpvm, fpvn, fpvo, fpvp, fpvq, fpvr, fpvt, fpvu, fpvv, fqfq, fqfr, fqfs,
fqft, fqfu, fqfv, fqg, fqgg, fqgh, fqgi, fqgj, fqgk, fqgl, fqgm, fqgn, fqgo, fqgp, fqgq, fqgr, fqgs, fqgt, fqgu,
fqgv, fqh, fqhg, fqhh, fqhi, fqhj, fqhl, fqhm, fqhn, fqho, fqhq, fqhr, fqht, fqhv, fqi, fqig, fqih, fqii, fqij,
fqik, fqil, fqim, fqin, fqio, fqip, fqiq, fqir, fqis, fqit, fqiv, fqj, fqjg, fqjh, fqji, fqij, fqjk, fqjl, fqjm, fqjn,
fqjo, fqjp, fqjq, fqjr, fqjs, fqjt, fqju, fqjv, fqk, fqkg, fqkh, fqki, fqkj, fqkk, fqkl, fqkm, fqkn, fqko, fqkp,
fqkq, fqkr, fqks, fqkt, fqku, fqkv, fql, fqlg, fqlh, fqli, fqlj, fqlk, fqll, fqlm, fqln, fqlo, fqlp, fqlq, fqlr, fqls,
fqlt, fqlu, fqlv, fqm, fqmg, fqml, fqmn, fqmq, fqmr, fqmt, fqn, fqng, fqnh, fqni, fqnj, fqnk, fqnm, fqnn,
fqnp, fqnq, fqnr, fqns, fqnt, fqnu, fqnv, fqo, fqog, fqok, fqol, fqon, fqop, fqoq, fqor, fqot, fqov, fqp, fqpg,
fqpj, fqpk, fqpl, fqpn, fqpo, fqpp, fqpq, fqpr, fqpt, fqpv, fqq, fqqg, fqqh, fqqj, fqqk, fqql, fqqm, fqqn,
fqqo, fqqp, fqqq, fqqr, fqqt, fqqu, fqqv, fqr, fqrg, fqrh, fqri, fqrj, fqrk, fqrl, fqrm, fqrn, fqro, fqrp, fqrq,
fqrr, fqrt, fqru, fqrv, fqs, fqsg, fqsl, fqsn, fqso, fqsq, fqsr, fqst, fqt, fqtg, fqth, fqti, fqtj, fqtk, fqtl, fqtm,
fqtn, fqto, fqtp, fqtq, fqtr, fqtt, fqtu, fqtv, fqu, fqug, fqul, fqun, fquo, fqup, fquq, fqur, fqut, fquv, fqv,
fqvg, fqvh, fqvi, fqvj, fqvk, fqvl, fqvm, fqvn, fqvo, fqvp, fqvq, fqvr, fqvt, fqvu, fqvv, frfr, frfs, frft, frfu,
frfv, frg, frgg, frgh, frgi, frgj, frgk, frgl, frgm, frgn, frgo, frgp, frgq, frgr, frgs, frgt, frgu, frgv, frh, frhg,
frhh, frhi, frhj, frhk, frhl, frhm, frhn, frho, frhp, frhq, frhr, frhs, frht, frhv, fri, frig, frih, frii, frij, frik, fril,
frim, frin, frio, frip, friq, frir, fris, frit, friv, frj, frjg, frjh, frji, frjj, frjk, frjm, frjn, frjp, frjq, frjr, frjs, frjt,
frju, frjv, frk, frkg, frkh, frki, frkk, frkl, frkm, frkn, frkp, frkq, frkr, frks, frkt, frku, frkv, frl, frlg, frlh, frli,
frlj, frlk, frll, frlm, frln, frlo, frlp, frlq, frlr, frls, frlt, frlu, frlv, frm, frmg, frmh, frmi, frmk, frml, frmm,
frmo, frmp, frmq, frmr, frmt, frmv, frn, frng, frnh, frni, frnj, frnk, frnm, frnn, frnp, frnq, frnr, frns, frnt,
frnu, frnv, fro, frog, froj, frok, frol, fron, frop, froq, fror, frot, frou, frov, frp, frpg, frpj, frpl, frpn, frpo,
frpp, frpq, frpr, frpt, frpu, frpv, frq, frqg, frqh, frqi, frqi, frqk, frql, frqm, frqn, frqo, frqp, frqq, frqr, frqs,
frqt, frqu, frqv, frr, frrg, frrh, frri, frrj, frrk, frrl, firm, frrn, frro, frrp, frrq, frrr, frrs, frrt, frru, frrv, frs,
frsg, frsi, frsj, frsl, frsn, frso, frsq, frsr, frst, frt, frtg, frth, frti, frtj, frtk, frtl, frtm, frtn, frto, frtp, frtq, frtr,
frts, frtt, frtu, frtv, fru, frug, fruj, frul, frun, fruo, frup, fruq, frur, frut, fruv, frv, frvg, frvh, frvi, frvj, frvk,
frvl, frvm, frvn, frvo, frvp, frvq, frvr, frvs, frvt, frvu, frvv, fsft, fsfv, fsg, fsgg, fsgh, fsgi, fsgj, fsgk, fsgl,
fsgm, fsgn, fsgp, fsgq, fsgr, fsgs, fsgt, fsgu, fsgv, fsh, fshg, fshl, fshm, fshn, fshq, fshr, fsht, fshv, fsi,
fsig, fsih, fsik, fsil, fsim, fsin, fsio, fsiq, fsir, fsit, fsj, fsjj, fsk, fskg, fskk, fskm, fskn, fskp, fskq, fskr, fskt,
fskv, fsl, fslg, fslh, fsli, fslk, fsll, fsln, fslo, fslp, fslq, fslr, fslt, fslv, fsm, fsmq, fsn, fsng, fsnh, fsnj, fsnp,
fsnr, fsns, fsnt, fsnu, fso, fsog, fsoh, fsoi, fsoj, fson, fsoq, fsor, fsot, fsov, fsp, fspg, fspn, fspq, fspr, fspt,
fspv, fsq, fsqg, fsqj, fsqn, fsqp, fsqq, fsqr, fsqt, fsqu, fsqv, fsr, fsrg, fsrh, fsrj, fsrl, fsrm, fsrn, fsrp, fsrq,
fsrr, fsrt, fsrv, fss, fssr, fsst, fst, fstg, fsth, fstj, fstk, fstl, fstm, fstn, fstp, fstq, fstr, fsts, fstt, fstu, fstv, fsu,
fsuh, fsuk, fsup, fsuq, fsur, fsut, fsv, fsvg, fsvi, fsvj, fsvl, fsvm, fsvn, fsvp, fsvq, fsvr, fsvt, fsvu, fsvv, ftft,
ftfu, ftfv, ftg, ftgg, ftgh, ftgi, ftgj, ftgk, ftgl, ftgm, ftgn, ftgo, ftgp, ftgq, ftgr, ftgs, ftgt, ftgu, ftgv, fth, fthg,
fthh, fthi, fthj, fthk, fthl, fthm, fthn, ftho, fthp, fthq, fthr, fths, ftht, fthv, fti, ftig, ftih, ftii, ftij, ftik, ftil,
ftim, ftin, ftio, flip, ftiq, ftir, ftis, ftit, ftiv, ftj, ftjg, ftjh, ftji, ftjj, ftjk, ftjm, ftjn, ftjp, ftjq, ftjr, ftjs, ftjt, ftju,
ftjv, ftk, ftkg, ftkh, ftki, ftkk, ftkl, ftkm, ftkn, ftkp, ftkq, ftkr, ftks, ftkt, ftkv, ftl, ftlg, ftlh, ftli, ftlj, ftlk,
ftll, ftlm, ftln, ftlo, ftlp, ftlq, ftlr, ftls, ftlt, ftlu, ftlv, ftm, ftmg, ftmh, ftmi, fimk, ftml, ftmm, ftmo, ftmp,
ftmq, ftmr, ftmt, ftn, ftng, ftnh, ftnj, ftnm, ftnp, ftnq, ftnr, ftns, ftnt, ftnu, ftnv, fto, ftog, ftoi, ftoj, ftok,
ftol, fton, ftop, ftoq, ftor, ftot, ftov, ftp, ftpg, ftpj, ftpk, ftpl, ftpn, ftpo, ftpp, ftpq, ftpr, ftpt, ftpv, ftq, ftqg,
ftqh, ftqi, ftqj, ftqk, ftql, ftqm, ftqn, ftqo, ftqp, ftqq, ftqr, ftqs, ftqt, ftqv, ftr, ftrg, ftrh, ftri, ftrj, ftrk, ftrl,
ftrm, ftrn, ftro, ftrp, ftrq, ftrr, ftrs, ftrt, ftru, ftrv, fts, ftsg, ftsi, ftsl, ftsm, ftsn, ftsq, ftsr, ftst, ftt, fttg, ftth,
ftti, fttj, fttk, fttl, fttm, fttn, ftto, fttp, fttq, fttr, ftts, fttt, fttu, fttv, ftu, ftug, ftuj, ftul, ftun, ftuo, ftup, ftuq,
ftur, ftut, ftuv, ftv, ftvg, ftvh, ftvi, ftvj, ftvk, ftvl, ftvm, ftvn, ftvo, ftvp, ftvq, ftvr, ftvs, ftvt, ftvv, fufu, fug,
fugg, fugh, fugi, fugj, fugk, fugl, fugm, fugn, fugp, fugq, fugr, fugs, fugt, fuh, fuhg, fuhh, fuhk, fuhl,
fuhq, fuhr, fuht, fui, fuig, fuih, fuik, fuil, fuim, fuip, fuiq, fuir, fuit, fuj, fujg, fujh, fujk, fujm, fujp, fujr,
fujs, fujt, fuk, fukg, fukk, fukl, fukm, fukn, fukq, fukr, fukt, ful, fulg, fulh, fuli, fulj, full, fuln, fulo, fulq,
fulr, fult, fum, fumr, fun, fung, funr, funs, funt, fuo, fuog, fuoi, fuoj, fuon, fuot, fup, fupl, fupn, fupq,
fupr, fupt, fuq, fugg, fuqi, fuqj, fugl, fuqm, fuqn, fuqq, fuqr, fuqs, fuqt, fur, furg, furh, furi, furj, furl,
furn, furq, furr, furt, furv, fus, fust, fut, futg, futh, futi, futj, futl, futm, futn, futq, futr, futt, fuu, fuuq, fuut,
fuv, fuvg, fuvi, fuvk, fuvl, fuvm, fuvn, fuvo, fuvq, fuvr, fuvs, fuvt, fvfv, fvg, fvgg, fvgh, fvgi, fvgj, fvgk,
fvgl, fvgm, fvgn, fvgo, fvgp, fvgq, fvgr, fvgs, fvgt, fvgu, fvgv, fvh, fvhg, fvhh, fvhi, fvhj, fvhl, fvhm,
fvhn, fvho, fvhp, fvhq, fvhr, fvht, fvhv, fvi, fvig, fvih, fvij, fvik, fvil, fvim, fvin, fvio, fvip, fviq, fvir, fvit,
fviv, fvj, fvjg, fvjj, fvjp, fvjr, fvjt, fvju, fvjv, fvk, fvkg, fvkh, fvki, fvkj, fvkk, fvkl, fvkm, fvkn, fvko,
fvkp, fvkq, fvkr, fvks, fvkt, fvku, fvkv, fvl, fvlg, fvlh, fvli, fvlj, fvlk, fvll, fvlm, fvln, fvlo, fvlp, fvlq, fvlr,
fvls, fvlt, fvlu, fvlv, fvm, fvmg, fvmj, fvmk, fvml, fvmn, fvmo, fvmq, fvmr, fvmt, fvn, fvng, fvnh, fvni,
fvnj, fvnk, fvnm, fvnn, fvnp, fvnq, fvnr, fvns, fvnt, fvnu, fvnv, fvo, fvog, fvoj, fvok, fvol, fvon, fvop,
fvoq, fvor, fvot, fvou, fvov, fvp, fvpg, fvph, fvpj, fvpk, fvpl, fvpm, fvpn, fvpo, fvpp, fvpq, fvpr, fvps,
fvpt, fvpu, fvpv, fvq, fvqg, fvqh, fvqi, fvqj, fvqk, fvql, fvqm, fvqn, fvqo, fvqp, fvqq, fvqr, fvqs, fvqt,
fvqu, fvqv, fvr, fvrg, fvrh, fvri, fvrj, fvrk, fvrl, fvrm, fvrn, fvro, fvrp, fvrq, fvrr, fvrs, fvrt, fvru, fvrv, fvs,
fvsg, fvsj, fvsl, fvsn, fvsq, fvsr, fvst, fvsu, fvsv, fvt, fvtg, fvth, fvti, fvtj, fvtk, fvtl, fvtm, fvtn, fvto, fvtp,
fvtq, fvtr, fvts, fvtt, fvtu, fvtv, fvu, fvug, fvun, fvup, fvuq, fvur, fvut, fvuv, fvv, fvvg, fvvh, fvvi, fvvj,
fvvk, fvvl, fvvm, fvvn, fvvo, fvvp, fvvq, fvvr, fvvs, fvvt, fvvu, fvvv, gggj, gggk, gggm, gggn, gggo,
gggp, gggq, gggr, gggs, gggt, gggu, gggv, gghi, gghj, gghk, gghl, gghm, gghn, ggho, gghp, gghq, gghr,
gghs, gght, gghv, ggi, ggih, ggik, ggil, ggim, ggin, ggio, ggip, ggiq, ggir, ggis, ggit, ggiv, ggj, ggjh, ggji,
ggij, ggjk, ggjm, ggjp, ggjq, ggjr, ggjs, ggit, ggju, ggjv, ggk, ggkh, ggki, ggkj, ggkk, ggkl, ggkm, ggkn,
ggkp, ggkq, ggkr, ggks, ggkt, ggku, ggkv, ggl, gglh, ggli, gglj, gglk, ggll, gglm, ggln, gglo, gglp, gglq,
gglr, ggls, gglt, gglu, gglv, ggmh, ggmj, ggmk, ggml, ggmm, ggmn, ggmo, ggmq, ggmr, ggms, ggmt,
ggmv, ggn, ggnh, ggni, ggnj, ggnk, ggnm, ggnp, ggnq, ggnr, ggns, ggnt, ggnu, ggnv, ggo, ggoh, ggoi,
ggoj, ggok, ggol, ggom, ggop, ggoq, ggor, ggos, ggot, ggou, ggov, ggp, ggph, ggpi, ggpj, ggpk, ggpl,
ggpn, ggpo, ggpp, ggpq, ggpr, ggps, ggpt, ggpu, ggpv, ggq, ggqh, ggqi, ggqj, ggqk, ggql, ggqm, ggqn,
ggqo, ggqp, ggqq, ggqr, ggqs, ggqt, ggqu, ggqv, ggr, ggrh, ggri, ggrj, ggrk, ggrl, ggrm, ggrn, ggro, ggrp,
ggrq, ggrr, ggrs, ggrt, ggru, ggrv, ggs, ggsh, ggsi, ggsj, ggsk, ggsl, ggsn, ggso, ggsp, ggsq, ggsr, ggst,
ggsu, ggsv, ggt, ggth, ggti, ggtj, ggtk, ggtl, ggtm, ggtn, ggto, ggtp, ggtq, ggtr, ggts, ggtt, ggtu, ggtv, ggu,
gguh, ggui, gguj, gguk, ggul, ggum, ggun, gguo, ggup, gguq, ggur, ggut, gguv, ggv, ggvh, ggvi, ggvj,
ggvk, ggvl, ggvm, ggvn, ggvo, ggvp, ggvq, ggvr, ggvs, ggvt, ggvu, ggvv, ghgi, ghgk, ghgl, ghgm, ghgo,
ghgp, ghgq, ghor, ghgs, ghgt, ghgu, ghgv, ghhi, ghhk, ghhl, ghhm, ghhp, ghhq, ghhr, ghhs, ghht, ghhv,
ghi, ghih, ghii, ghik, ghil, ghim, ghip, ghiq, ghir, ghis, ghit, ghiv, ghj, ghjh, ghjm, ghjp, ghjr, ghjs, ghjt,
ghju, ghk, ghkh, ghki, ghkk, ghkl, ghkm, ghkp, ghkq, ghkr, ghkt, ghku, ghkv, ghl, ghlh, ghli, ghlj, ghlk,
ghll, ghlm, ghln, ghlo, ghlp, ghlq, ghlr, ghls, ghlt, ghlv, ghm, ghmh, ghmi, ghmk, ghml, ghmm, ghmq,
ghmr, ghmv, ghn, ghnh, ghni, ghnj, ghnk, ghnm, ghnp, ghnq, ghnr, ghns, ghnt, ghnu, ghnv, gho, ghoh,
ghoi, ghoj, ghok, ghol, ghom, ghon, ghop, ghoq, ghor, ghos, ghot, ghou, ghov, ghp, ghph, ghpi, ghpk,
ghpl, ghpm, ghpo, ghpp, ghpq, ghpr, ghps, ghpt, ghpu, ghpv, ghq, ghqh, ghqi, ghqj, ghqk, ghql, ghqm,
ghqn, ghqo, ghqp, ghqq, ghqr, ghqs, ghqt, ghqu, ghqv, ghr, ghrh, ghri, ghrk, ghrl, ghrm, ghr, ghro, ghrp,
ghrq, ghrr, ghrs, ghrt, ghru, ghrv, ghs, ghsh, ghsi, ghsk, ghsl, ghsp, ghsq, ghsr, ghsv, ght, ghth, ghti, ghtj,
ghtk, ghtl, ghtm, ghtp, ghtq, ghtr, ghts, ghtt, ghtu, ghtv, ghu, ghuh, ghup, ghuq, ghur, ghut, ghuv, ghv,
ghvh, ghvi, ghvk, ghvl, ghvm, ghvn, ghvo, ghvp, ghvq, ghvr, ghvs, ghvt, ghvu, ghvv, gigj, gigk, gigl,
gigm, gign, gigo, gigp, gigq, gigr, gigs, gigt, gigu, gigv, gih, gihh, gihi, gihk, gihl, gihm, giho, gihp, gihq,
gihr, gihs, giht, gihv, gii, giih, giil, giio, giiq, giir, giiv, gij, gijs, gik, gikh, giki, gikj, gikk, gikl, gikm,
gikp, gikq, gikr, giks, gikt, giku, gikv, gil, gilh, gili, gilj, gilk, gill, gilm, giln, gilo, gilp, gilq, gilr, gilt,
gilv, gim, gimh, gimi, giml, gimr, gimv, gin, ginh, gini, ginj, gink, ginm, ginp, ginq, ginr, gins, gint, ginu,
ginv, gio, gioh, gioi, gioj, giok, giol, giom, giop, gioq, gior, giot, giov, gip, giph, gipi, gipj, gipk, gipl,
gipm, gipn, gipo, gipp, gipq, gipr, gips, gipt, gipu, gipv, giq, giqh, giqi, giqj, giqk, giql, giqm, giqn, giqo,
giqp, giqq, giqr, giqs, giqt, giqu, giqv, gir, girh, giri, girj, girk, girl, girm, giro, girp, girq, girr, girs, girt,
giru, girv, gis, gish, gisl, gism, giso, gisp, gisq, gisr, giss, gist, gisv, git, gith, giti, gitj, gitk, gitl, gitm,
gitp, gitq, gitr, gits, gitt, gitu, gitv, giu, giup, giv, givh, givi, givk, givl, givm, givn, givo, givp, givq, givr,
givs, givt, givu, givv, gjgl, gjgo, gjgq, gjgr, gjgs, gjgt, gjgv, gjh, gjhi, gjhk, gjhl, gjhin, gjho, gjhq, gjhr,
gjht, gjhv, gji, gjik, gjil, gjin, gjio, gjip, gjiq, gjir, gjit, gjiv, gjj, gjjh, gjji, gjjj, gjjk, gjjm, gjjp, gjjq, gjjr,
gjjs, gjjt, gjju, gjjv, gjk, gjkh, gjki, gjkk, gjkl, gjkn, gjkq, gjkr, gjkt, gjkv, gjl, gjlk, gjln, gjlq, gjlt, gjlv,
gjm, gjmj, gjml, gjmo, gjmp, gjmq, gjmr, gjmt, gjn, gjnj, gjo, gjp, gjph, gjpi, gjpj, gjpk, gjpl, gjpn, gjpo,
gjpp, gjpq, gjpr, gjpt, gjpv, gjq, gjqh, gjqi, gjqj, gjqk, gjql, gjqn, gjqo, gjqp, gjqq, gjqr, gjqt, gjqv, gjr,
gjrh, gjri, gjrj, gjrk, girl, gjrn, gjro, gjrp, gjrq, gjrr, gjrt, gjrv, gjs, gjsh, gjsk, gjsl, gjsn, gjso, gjsq, gjsr,
gjst, gjsv, gjt, gjth, gjti, gjtj, gjtk, gjtl, gjtn, gjto, gjtp, gjtq, gjtr, gjtt, gjtv, gjuh, gjui, gjuk, gjul, gjun,
gjup, gjuq, gjur, gjut, gjuv, gjv, gjvh, gjvk, gjvl, gjvn, gjvo, gjvp, gjvq, gjvr, gjvt, gjvv, gkgk, gkgl, gkgm,
gkgn, gkgp, gkgq, gkgr, gkgs, gkgt, gkgu, gkgv, gkh, gkhh, gkhi, gkhk, gkhl, gkhm, gkhn, gkho, gkhp,
gkhq, gkhr, gkhs, gkht, gkhv, gki, gkih, gkii, gkik, gkil, gkim, gkio, gkip, gkiq, gkir, gkis, gkit, gkiv, gkj,
gkjh, gkji, gkjp, gkjq, gkjr, gkjs, gkjt, gkjv, gkk, gkkh, gkki, gkkk, gkkl, gkkm, gkko, gkkp, gkkq, gkkr,
gkkt, gkkv, gkl, gklh, gkli, gklj, gklk, gkll, gklm, gkln, gklo, gklp, gklq, gklr, gkls, gklt, gklu, gklv, gkm,
gkmh, gkmk, gkml, gkmn, gkmp, gkmq, gkmr, gkms, gkmt, gkmv, gkn, gknh, gkni, gknj, gknm, gknp,
gknq, gknr, gkns, gknt, gknu, gknv, gko, gkoi, gkoj, gkok, gkol, gkon, gkop, gkoq, gkor, gkot, gkov,
gkp, gkph, gkpi, gkpk, gkpl, gkpn, gkpo, gkpp, gkpq, gkpr, gkpt, gkpv, gkq, gkqh, gkqi, gkqj, gkqk,
gkql, gkqn, gkqo, gkqp, gkqq, gkqr, gkqt, gkqu, gkqv, gkr, gkrh, gkri, gkrj, gkrk, gkrl, gkrm, gkrn, gkro,
gkrp, gkrq, gkrr, gkrs, gkrt, gkru, gkrv, gks, gksl, gksn, gkso, gksq, gksr, gkst, gksv, gkt, gkth, gkti, gktj,
gktk, gktl, gktm, gktn, gkto, gktp, gktq, gktr, gkts, gktt, gktu, gktv, gku, gkuh, gkui, gkul, gkun, gkuo,
gkuq, gkut, gkuv, gkv, gkvh, gkvi, gkvk, gkvl, gkvm, gkvn, gkvo, gkvp, gkvq, gkvr, gkvs, gkvt, gkvv,
glgl, glgm, glgn, glgp, glgq, glgr, glgs, glgt, glgu, glgv, glh, glhh, glhi, glhk, glhl, glhn, glho, glhp, glhq,
glhr, glht, glhv, gli, glik, glil, glin, glio, glip, gliq, glir, glis, glit, gliv, glj, gljh, glji, gljj, gljk, gljl, gljp,
gljq, gljr, gljs, gljt, glju, gljv, glk, glkh, glki, glkk, glkl, glkm, glkn, glkp, glkq, glkr, glks, glkt, glku,
glkv, gll, gllh, glli, gllk, glll, glln, gllo, gllp, gllq, gllr, gllt, gllv, glm, glmh, glmk, glml, glmq, glmr,
glmv, gln, glnh, glni, glnj, glnk, glnl, glnm, glnp, glnq, glnr, glns, glnt, glnu, glnv, glo, gloh, gloi, gloj,
glok, glol, glop, gloq, glor, glot, glov, glp, glph, glpi, glpj, glpk, glpl, glpn, glpo, glpp, glpq, glpr, glpt,
glpv, glq, glqh, glqi, glqj, glqk, glql, glqm, glqn, glqo, glqp, glqq, glqr, glqs, glqt, glqv, glr, glrh, glri,
glrj, glrk, glrl, glrm, glrn, glro, glrp, glrq, glrr, glrt, glru, glrv, gls, glsh, glsi, glsl, glsn, glsq, glsr, glsv,
glt, glth, glti, gltj, gltk, gltl, gltm, gltn, glto, gltp, gltq, gltr, glts, gltt, gltu, gltv, glu, gluh, gluj, gluk, glul,
gluq, gluv, glv, glvh, glvi, glvj, glvk, glvl, glvn, glvo, glvp, glvq, glvr, glvs, glvt, glvu, glvv, gmgm,
gmgp, gmgq, gmgr, gmgs, gmgt, gmgu, gmgv, gmh, gmhk, gmhl, gmhm, gmhp, gmhq, gmhr, gmht,
gmhv, gmi, gmih, gmik, gmil, gmim, gmiq, gmir, gmit, gmj, gmjh, gmji, gmjm, gmjp, gmjq, gmjr, gmjs,
gmjt, gmju, gmjv, gmk, gmkh, gmki, gmkm, gmkr, gmkt, gmku, gml, gmlh, gmli, gmlk, gmll, gmlm,
gmlp, gmlq, gmlr, gmls, gmlt, gmlu, gmlv, gmm, gmmh, gmmm, gmn, gmnh, gmni, gmnm, gmnt, gmo,
gmoh, gmoi, gmok, gmol, gmom, gmop, gmoq, gmor, gmos, gmot, gmou, gmov, gmp, gmph, gmpk,
gmpm, gmpp, gmpq, gmpr, gmps, gmpt, gmpv, gmq, gmqh, gmqi, gmqj, gmqm, gmqp, gmqr, gmqs,
gmqt, gmqu, gmr, gmrh, gmri, gmrk, gmrl, gmrm, gmrp, gmrq, gmrr, gmrs, gmrt, gmru, gmrv, gms,
gmsh, gmsl, gmt, gmth, gmtj, gmtm, gmtp, gmtr, gmts, gmtt, gmtu, gmu, gmv, gmvh, gmvi, gmvm,
gmvp, gmvq, gmvr, gmvs, gmvt, gmvu, gmvv, gngo, gngp, gngq, gngr, gngt, gngv, gnh, gnhj, gnhl,
gnhn, gnho, gnhp, gnhq, gnhr, gnht, gnhv, gni, gnik, gnil, gnin, gnio, gnip, gniq, gnir, gnit, gniv, gnj,
gnjh, gnji, gnjj, gnjk, gnjl, gnjm, gnjp, gnjq, gnjr, gnjs, gnjt, gnju, gnjv, gnk, gnkh, gnki, gnkk, gnkl,
gnkq, gnkr, gnkt, gnkv, gnl, gnlk, gnlq, gnlv, gnm, gnmk, gnml, gnmn, gnmo, gnmp, gnmq, gnmr, gnmt,
gnmv, gnn, gno, gnp, gnph, gnpk, gnpl, gnpn, gnpo, gnpq, gnpr, gnpt, gnpv, gnq, gnqh, gnqj, gnqk, gnql,
gnqn, gnqo, gnqp, gnqq, gnqr, gnqt, gnqv, gnr, gnrh, gnri, gnrj, gnrk, gnrl, gnrn, gnro, gnrp, gnrq, gnrr,
gnrt, gnrv, gns, gnsh, gnsl, gnsn, gnso, gnsq, gnsr, gnst, gnsv, gnt, gnth, gnti, gntj, gntk, gntl, gntn, gnto,
gntp, gntq, gntr, gntt, gntv, gnu, gnuh, gnui, gnuk, gnul, gnuo, gnug, gnur, gnut, gnuv, gnv, gnvh, gnvk,
gnvl, gnvn, gnvo, gnvp, gnvq, gnvr, gnvt, gnvv, gogo, gogp, gogg, gogr, gogt, gogv, goh, gohj, gohk,
gohn, goho, gohq, gohr, goht, gohv, goi, goij, goik, goil, goin, goip, goiq, goir, goit, goiv, goj, goji, gojj,
gojl, gojp, gojq, gojr, gojv, gok, gokh, gokn, gokp, gokq, gokv, gol, golk, goln, golq, golr, golt, golv,
gom, gomj, goml, gomo, gomr, gon, gonj, gong, gonv, goo, gop, gopj, gopl, gopn, gopq, gopr, gopt, goq,
goqh, goqj, goqk, goql, goqn, goqo, goqp, goqq, goqr, goqt, goqv, gor, gorh, gorj, gorl, gorn, goro, gorq,
gorr, gort, gorv, gos, gosj, gosn, gosr, gosv, got, goth, gotj, gotk, gotl, gotn, goto, gotp, gotq, gotr, gott,
gotv, gou, goul, gouv, gov, govh, govj, govk, govl, govn, govo, govp, govq, govr, govt, govv, gpgp,
gpgq, gpgr, gpgs, gpgt, gpgu, gpgv, gph, gphh, gphi, gphk, gphl, gphm, gphn, gpho, gphp, gphq, gphr,
gphs, gpht, gphv, gpi, gpih, gpii, gpik, gpil, gpim, gpio, gpip, gpiq, gpir, gpis, gpit, gpiv, gpj, gpjh, gpjj,
gpjm, gpjp, gpjq, gpjr, gpjs, gpjt, gpju, gpjv, gpk, gpkh, gpki, gpkk, gpkl, gpkm, gpkn, gpkp, gpkq, gpkr,
gpks, gpkt, gpku, gpkv, gpl, gplh, gpli, gplk, gpll, gplm, gplo, gplp, gplq, gplr, gpls, gplt, gplu, gplv,
gpm, gpmj, gpmk, gpmn, gpmp, gpmq, gpmr, gpms, gpmt, gpmv, gpn, gpnh, gpni, gpnj, gpnk, gpnl,
gpnm, gpnp, gpnq, gpnr, gpns, gpnt, gpnu, gpnv, gpo, gpoh, gpoi, gpoj, gpok, gpol, gpom, gpon, gpop,
gpoq, gpor, gpos, gpot, gpou, gpov, gpp, gpph, gppi, gppk, gppl, gppm, gppn, gppp, gppq, gppr, gpps,
gppt, gppu, gppv, gpq, gpqh, gpqi, gpqj, gpqk, gpql, gpqm, gpqn, gpqo, gpqp, gpqq, gpqr, gpqs, gpqt,
gpqu, gpqv, gpr, gprh, gpri, gprj, gprk, gprl, gprm, gprn, gpro, gprp, gprq, gprr, gprs, gprt, gpru, gprv,
gps, gpsk, gpsq, gpsr, gpst, gpsv, gpt, gpth, gpti, gptj, gptk, gptl, gptm, gptn, gpto, gptp, gptq, gptr, gpts,
gptt, gptu, gptv, gpu, gpuh, gpui, gpun, gpup, gpuq, gpur, gput, gpuv, gpv, gpvh, gpvi, gpvj, gpvk, gpvl,
gpvm, gpvn, gpvo, gpvp, gpvq, gpvr, gpvs, gpvt, gpvu, gpvv, gqgq, gqgr, gqgs, gqgt, gqgu, gqgv, gqh,
gqhh, gqhi, gqhj, gqhk, gqhl, gqhm, gqhn, gqho, gqhp, gqhq, gqhr, gqhs, gqht, gqhv, gqi, gqih, gqii,
gqik, gqil, gqim, gqin, gqio, gqip, gqiq, gqir, gqis, gqit, gqiv, gqj, gqjh, gqji, gqij, gqjk, gqjl, gqjm, gqjp,
gqjq, gqjr, gqjs, gqjt, gqju, gqjv, gqk, gqkh, gqki, gqkk, gqkl, gqkm, gqkn, gqko, gqkp, gqkq, gqkr, gqks,
gqkt, gqku, gqkv, gql, gqlh, gqli, gqlj, gqlk, gqll, gqlm, gqln, gqlo, gqlp, gqlq, gqlr, gqls, gqlt, gqlu, gqlv,
gqm, gqmh, gqmj, gqmk, gqml, gqmn, gqmp, gqmq, gqmr, gqms, gqmt, gqmv, gqn, gqnh, gqni, gqnj,
gqnk, gqnl, gqnm, gqnp, gqng, gqnr, gqns, gqnt, gqnu, gqnv, gqo, gqoh, gqoi, gqoj, gqok, gqol, gqon,
gqop, gqoq, gqor, gqot, gqou, gqov, gqp, gqph, gqpi, gqpk, gqpl, gqpm, gqpn, gqpo, gqpp, gqpq, gqpr,
gqpt, gqpv, gqq, gqqh, gqqi, gqqj, gqqk, gqql, gqqm, gqqn, gqqo, gqqp, gqqq, gqqr, gqqs, gqqt, gqqu,
gqqv, gqr, gqrh, gqri, gqrj, gqrk, gqrl, gqrm, gqrn, gqro, gqrp, gqrq, gqrr, gqrs, gqrt, gqru, gqrv, gqs,
gqsh, gqsi, gqsk, gqsl, gqsn, gqso, gqsq, gqsr, gqst, gqsv, gqt, gqth, gqti, gqtj, gqtk, gqtl, gqtm, gqtn,
gqto, gqtp, gqtq, gqtr, gqts, gqtt, gqtu, gqtv, gqu, gquh, gqui, gquk, gqul, gquo, gqup, gquq, gqur, gqus,
gqut, gquv, gqv, gqvh, gqvi, gqvj, gqvk, gqvl, gqvm, gqvn, gqvo, gqvp, gqvq, gqvr, gqvs, gqvt, gqvu,
gqvv, grgr, grgs, grgt, grgu, grgv, grh, grhh, grhi, grhk, grhl, grhm, grhn, grho, grhp, grhq, grhr, grhs,
grht, grhv, gri, grih, grii, grik, gril, grim, grin, grio, grip, griq, grir, gris, grit, griv, grj, grjh, grji, grjj,
grjk, grjl, grjm, grjp, grjq, grjr, grjs, grjt, grju, grjv, grk, grkh, grki, grkk, grkl, grkm, grkn, grkp, grkq,
grkr, grks, grkt, grku, grkv, grl, grlh, grli, grlj, grlk, grll, grlm, grln, grlo, grlp, grlq, grlr, grls, grlt, grlu,
grlv, grm, grmj, grmk, grml, grmn, grmp, grmq, grmr, grms, grmt, grmv, grn, grnh, grni, grnj, grnk,
grnm, grnp, grnq, grnr, grns, grnt, grnu, grnv, gro, groh, groi, groj, grok, grol, gron, grop, groq, gror,
gros, grot, grou, grov, grp, grph, grpi, grpj, grpk, grpl, grpm, grpn, grpo, grpp, grpq, grpr, grps, grpt,
grpu, grpv, grq, grqh, grqi, grqj, grqk, grql, grqm, grqn, grqo, grqp, grqq, grqr, grqs, grqt, grqu, grqv, grr,
grrh, grri, grrj, grrk, grrl, grrm, grrn, grro, grrp, grrq, grrr, grrs, grrt, grru, grrv, grs, grsi, grsl, grsn, grso,
grsq, grsr, grst, grsv, grt, grth, grti, grtj, grtk, grtl, grim, grtn, grto, grtp, grtq, grtr, grts, grtt, grtu, grtv,
gru, gruh, grui, gruk, grul, grun, gruo, grup, gruq, grur, grut, gruv, grv, grvh, grvi, grvk, grvl, grvm, grvn,
grvo, grvp, grvq, grvr, grvs, grvt, grvu, grvv, gsgs, gsgt, gsgu, gsgv, gsh, gshk, gshl, gshm, gshp, gshq,
gshr, gshs, gsht, gsi, gsii, gsik, gsil, gsim, gsip, gsiq, gsir, gsit, gsiv, gsj, gsjh, gsjj, gsjm, gsjp, gsjs, gsk,
gskh, gski, gskm, gskp, gskq, gskr, gsks, gskt, gsku, gskv, gsl, gslh, gsli, gslk, gsll, gslm, gslp, gslq, gslr,
gsls, gslt, gslv, gsm, gsmq, gsmv, gsn, gsnh, gsnj, gsnm, gsnp, gsnr, gsns, gsnt, gsnu, gsnv, gso, gsoi,
gsoj, gsok, gsol, gsom, gsop, gsoq, gsor, gsos, gsot, gsou, gsov, gsp, gsph, gspi, gspp, gspq, gspr, gspt,
gspv, gsq, gsqh, gsqi, gsqj, gsqk, gsql, gsqm, gsqp, gsqq, gsqr, gsqs, gsqt, gsqu, gsqv, gsr, gsrh, gsri, gsrj,
gsrl, gsrm, gsrp, gsrq, gsrr, gsrs, gsrt, gsru, gsrv, gss, gssr, gsst, gst, gsth, gsti, gstj, gstl, gstm, gstp, gstq,
gstr, gsts, gstt, gstu, gstv, gsu, gsum, gsv, gsvh, gsvi, gsvk, gsvl, gsvm, gsvp, gsvq, gsvr, gsvs, gsvt, gsvu,
gsvv, gtgt, gtgu, gtgv, gth, gthh, gthi, gthk, gthl, gthm, gthn, gtho, gthp, gthq, gthr, gths, gtht, gthv, gti,
gtih, gtii, gtik, gtil, gtim, gtin, gtio, gtip, gtiq, gtir, gtis, gtit, gtiv, gtj, gtjh, gtji, gtjj, gtjk, gtjl, gtjm, gtjp,
gtjq, gtjr, gtjs, gtjt, gtju, gtjv, gtk, gtkh, gtki, gtkk, gtkl, gtkm, gtkn, gtkp, gtkq, gtkr, gtks, gtkt, gtkv, gtl,
gtlh, gtli, gtlj, gtlk, gtll, gtlm, gtln, gtlo, gtlp, gtlq, gtlr, gtls, gtlt, gtlu, gtlv, gtm, gtmh, gtmj, gtmk, gtml,
gtmn, gtmo, gtmp, gtmq, gtmr, gtmt, gtmv, gtn, gtnh, gtni, gtnj, gtnk, gtnm, gtnp, gtng, gtnr, gtns, gtnt,
gtnu, gtnv, gto, gtoh, gtoi, gtoj, gtok, gtol, gton, gtop, gtoq, gtor, gtos, gtot, gtou, gtov, gtp, gtph, gtpi,
gtpk, gtpl, gtpm, gtpn, gtpo, gtpp, gtpq, gtpr, gtpt, gtpv, gtq, gtqh, gtqi, gtqj, gtqk, gtql, gtqm, gtqn, gtqo,
gtqp, gtqq, gtqr, gtqs, gtqt, gtqu, gtqv, gtr, gtrh, gtri, gtrj, gtrk, gtrl, gtrm, gtr, gtro, gtrp, gtrq, gtrr, gtrs,
gtrt, gtru, gtrv, gts, gtsi, gtsl, gtsn, gtso, gtsq, gtsr, gtst, gtsv, gtt, gtth, gtti, gttj, gtik, gitl, gttm, gitn, gtto,
gttp, gttq, gttr, gtts, gttt, gttu, gttv, gtu, gtuh, gtui, gtuk, gtul, gtun, gtuo, gtup, gtuq, gtur, gtut, gtuv, gtv,
gtvh, gtvi, gtvk, gtvl, gtvm, gtvn, gtvo, gtvp, gtvq, gtvr, gtvs, gtvt, gtvu, gtvv, gugu, gugv, guh, guhh,
guhi, guhk, guhl, guhq, guhr, guht, guhv, gui, guih, guii, guil, guim, guip, guir, guit, guiv, guj, gujh, guji,
gujm, gujp, gujq, gujr, gujs, gujt, gujv, guk, gukh, guki, gukk, gukl, gukp, gukq, gukr, gukt, gukv, gul,
gulh, guli, gulj, gulk, gull, gulm, gulo, gulp, gulq, gulr, guls, gult, gulu, gulv, gum, gun, gunh, guni, gunj,
gunp, gunq, gunr, guns, gunt, gunu, gunv, guo, guoi, guoj, guok, guon, guoq, guor, guot, guov, gup, gupi,
gupl, gupn, gupq, gupr, gupt, gupv, guq, guqh, guqi, guqi, guqk, guql, guqn, guqp, guqq, guqr, guqt,
guqv, gur, gurh, guri, gurj, gurk, gurl, gurm, gurp, gurq, gurr, gurt, guru, gurv, gus, gut, guth, guti, gutj,
gutk, gutl, gutp, gutq, gutr, gutt, gutu, gutv, guu, guv, guvh, guvi, guvk, guvl, guvn, guvo, guvp, guvq,
guvr, guvt, guvv, gvgv, gvh, gvhh, gvhi, gvhj, gvhk, gvhl, gvhm, gvhn, gvho, gvhp, gvhq, gvhr, gvhs,
gvht, gvhv, gvi, gvih, gvii, gvik, gvil, gvim, gvin, gvio, gvip, gviq, gvir, gvis, gvit, gviv, gvj, gvjh, gvjj,
gvjm, gvjp, gvjr, gvjs, gvjt, gvju, gvjv, gvk, gvkh, gvki, gvkk, gvkl, gvkm, gvkn, gvko, gvkp, gvkq, gvkr,
gvks, gvkt, gvku, gvkv, gvl, gvlh, gvli, gvlj, gvlk, gvll, gvlm, gvln, gvlo, gvlp, gvlq, gvlr, gvls, gvlt, gvlu,
gvlv, gvm, gvmh, gvmi, gvmj, gvmk, gvmn, gvmp, gvmq, gvmr, gvmt, gvmv, gvn, gvnh, gvni, gvnj,
gvnk, gvnl, gvnm, gvnp, gvnq, gvnr, gvns, gvnt, gvnu, gvnv, gvo, gvoh, gvoi, gvoj, gvok, gvol, gvom,
gvon, gvop, gvoq, gvor, gvos, gvot, gvou, gvov, gvp, gvph, gvpi, gvpk, gvpl, gvpm, gvpn, gvpo, gvpp,
gvpq, gvpr, gvps, gvpt, gvpu, gvpv, gvq, gvqh, gvqi, gvqj, gvqk, gvql, gvqm, gvqn, gvqo, gvqp, gvqq,
gvqr, gvqs, gvqt, gvqu, gvqv, gvr, gvrh, gvri, gvrj, gvrk, gvrl, gvrm, gvrn, gvro, gvrp, gvrq, gvrr, gvrs,
gvrt, gvru, gvrv, gvs, gvsh, gvsi, gvsk, gvsl, gvsn, gvso, gvsp, gvsq, gvsr, gvst, gvsv, gvt, gvth, gvti, gvtj,
gvtk, gvtl, gvtm, gvtn, gvto, gvtp, gvtq, gvtr, gvts, gvtt, gvtu, gvtv, gvu, gvuh, gvul, gvuo, gvuq, gvur,
gvut, gvuv, gvv, gvvh, gvvi, gvvj, gvvk, gvvl, gvvm, gvvn, gvvo, gvvp, gvvq, gvvr, gvvs, gvvt, gvvu,
gvvv, hhhi, hhhj, hhhk, hhhl, hhhm, hhhp, hhhq, hhhr, hhhs, hhhv, hhi, hhik, hhil, hhim, hhin, hhio, hhiq,
hhir, hhis, hhiv, hhj, hhjt, hhk, hhki, hhkk, hhkq, hhkr, hhkt, hhkv, hhl, hhli, hhlj, hhlk, hhll, hhlp, hhlq,
hhlr, hhlt, hhlv, hhm, hhmk, hhmn, hhmo, hhmp, hhmq, hhmr, hhmv, hhn, hhnj, hho, hhoi, hhor, hhot,
hhp, hhpl, hhpo, hhpq, hhpr, hhpt, hhpv, hhq, hhqi, hhqj, hhqk, hhql, hhqm, hhqn, hhqp, hhqq, hhqr,
hhqt, hhqv, hhr, hhri, hhrj, hhrk, hhrl, hhrp, hhrq, hhrr, hhrt, hhru, hhrv, hhs, hhsp, hhsq, hhsv, hht, hhti,
hhtj, hhtk, hhtl, hhtp, hhtq, hhtr, hhtt, hhtu, hhtv, hhu, hhuj, hhup, hhuv, hhv, hhvi, hhvk, hhvl, hhvm,
hhvn, hhvq, hhvr, hhvt, hhvv, hihi, hihk, hihl, hihm, hihg, hihr, hii, hiii, hiik, hiil, hiin, hiio, hiig, hiir,
hiiv, hij, hik, hiki, hikk, hikl, hikp, hikt, hikv, hil, hili, hilj, hilk, hill, hilm, hiln, hilp, hilq, hilr, hilt, hilv,
him, himk, hin, hinj, hinp, hinq, hinr, hint, hinu, hinv, hio, hioj, hiok, hiol, hioq, hior, hiot, hip, hipi, hipk,
hipl, hipo, hipr, hipv, hiq, hiqi, hiqj, hiqk, hiql, hiqm, hiqn, hiqp, hiqq, hiqr, hiqt, hiqu, hiqv, hir, hiri,
hirj, hirk, hirl, hirn, hirp, hirq, hirr, hirt, hirv, his, hit, hiti, hitj, hitl, hitp, hitq, hitr, hitt, hitv, hiu, hiuj,
hiv, hivi, hivk, hivl, hivm, hivp, hivq, hivr, hivs, hivt, hivv, hjhn, hjhq, hjhr, hjht, hji, hjin, hij, hjjj, hjjq,
hjjr, hjjt, hjk, hjl, hjm, hjml, hjmo, hjmp, hjmr, hjn, hjo, hjp, hjpk, hjpl, hjpn, hjpq, hjpv, hjq, hjql, hjr,
hjrj, hjrk, hjrl, hjrn, hjs, hjsl, hjsr, hjsv, hjt, hjtj, hjtk, hjtl, hjtn, hjto, hjtq, hjtt, hjul, hjun, hjv, hjvk, hjvl,
hjvn, hjvq, hjvv, hkhk, hkhl, hkhm, hkhn, hkho, hkhp, hkhq, hkhr, hkhs, hkht, hkhv, hki, hkik, hkil, hkin,
hkio, hkip, hkiq, hkir, hkis, hkit, hkiv, hkj, hkji, hkjr, hkjt, hkk, hkkk, hkkq, hkkr, hkkt, hkku, hkkv, hkl,
hklj, hklk, hkll, hklm, hkln, hklq, hklr, hklt, hklv, hkm, hkmj, hkmk, hkmn, hkmo, hkmp, hkmq, hkmr,
hkms, hkmt, hkmv, hkn, hknj, hko, hkoj, hkp, hkpk, hkpl, hkpn, hkpo, hkpq, hkpr, hkpt, hkpv, hkq, hkqj,
hkqk, hkql, hkqn, hkqo, hkqp, hkqq, hkqr, hkqt, hkqv, hkr, hkrj, hkrk, hkrl, hkrn, hkro, hkrq, hkrr, hkrt,
hkru, hkrv, hks, hksl, hksn, hkso, hksq, hksr, hkss, hkst, hkt, hkti, hktj, hktk, hktl, hktn, hkto, hktp, hktq,
hktr, hktt, hktu, hktv, hku, hkuj, hkuk, hkul, hkun, hkuq, hkuv, hkv, hkvj, hkvk, hkvl, hkvn, hkvo, hkvq,
hkvr, hkvs, hkvt, hkvv, hlhl, hlhn, hlho, hlhp, hlhq, hlhr, hlht, hlhv, hli, hlik, hlil, hlin, hlio, hlip, hliq,
hlir, hlit, hliv, hlj, hlji, hljj, hljk, hljq, hljr, hljs, hljt, hlju, hljv, hlk, hlki, hlkk, hlkl, hlkn, hlko, hlkp, hlkq,
hlkr, hlkt, hlkv, hll, hlli, hllj, hllk, hlll, hlln, hllo, hllp, hllq, hllr, hllt, hllu, hllv, hlm, hlmj, hlml, hlmp,
hlmq, hlmr, hlmv, hln, hlni, hlnj, hlnr, hlnt, hlo, hlon, hloq, hlp, hlpi, hlpj, hlpk, hlpl, hlpn, hlpo, hlpp,
hlpq, hlpr, hlpt, hlpv, hlq, hlqi, hlqi, hlqk, hlql, hlqm, hlqn, hlqo, hlqp, hlqq, hlqr, hlqt, hlqv, hlr, hlri,
hlrj, hlrk, hlrl, hlrn, hlro, hlrp, hlrq, hlrr, hlrs, hlrt, hlrv, hls, hlsi, hlsl, hlsn, hlsq, hlsr, hlst, hlsv, hlt, hlti,
hltj, hltk, hltl, hltn, hlto, hltp, hltq, hltr, hlts, hltt, hltu, hltv, hlu, hluj, hluk, hlul, hluq, hlur, hlut, hluv,
hlv, hlvi, hlvj, hlvk, hlvl, hlvn, hlvo, hlvp, hlvq, hlvr, hlvt, hlvv, hmhm, hmhq, hmhr, hmi, hmii, hmik,
hmil, hmim, hmip, hmiq, hmir, hmit, hmj, hmjr, hmjt, hmju, hmk, hmkm, hml, hmll, hmlm, hmlr, hmlv,
hmm, hmmm, hmmt, hmn, hmo, hmoi, hmp, hmpi, hmq, hmqm, hmqp, hmqs, hmqu, hmr, hmri, hmrm,
hmrp, hmrr, hmrt, hmrv, hms, hmt, hmu, hmv, hmvm, hnhn, hnho, hnhp, hnhq, hnhr, hnht, hnhv, hni,
hnik, hnil, hnin, hniq, hnir, hnit, hniv, hnj, hnji, hnjk, hnjp, hnjq, hnjr, hnjs, hnjt, hnju, hnjv, hnk, hnkn,
hnkv, hnl, hnm, hnmj, hnmk, hnml, hnmn, hnmp, hnmq, hnmr, hnmt, hnmv, hnn, hno, hnp, hnpj, hnpk,
hnpl, hnpn, hnpq, hnpr, hnpt, hnpv, hnq, hnqk, hnql, hnqn, hnqq, hnqt, hnqv, hnr, hnrj, hnrk, hnrl, hnrn,
hnro, hnrq, hnrr, hnrt, hnrv, hns, hnsk, hnsl, hnsn, hnsq, hnsr, hnst, hnsv, hnt, hnti, hntj, hntk, hntl, hntn,
hnto, hntq, hntr, hntt, hntv, hnuk, hnul, hnun, hnuo, hnuq, hnur, hnut, hnuv, hnv, hnvj, hnvk, hnvl, hnvn,
hnvp, hnvq, hnvr, hnvt, hnvv, hoho, hohp, hohq, hohr, hoht, hohv, hoi, hoik, hoil, hoin, hoio, hoip, hoiq,
hoir, hoit, hoiv, hoj, hojj, hojk, hojp, hojq, hojr, hojt, hojv, hok, hokk, hokl, hokn, hoko, hokq, hokr, hokt,
hokv, hol, holj, holk, holl, holn, holo, holp, holq, holr, holt, holv, hom, homl, homo, homp, homq, homr,
homv, hon, honj, hong, honr, hont, honv, hoo, hop, hopj, hopl, hopn, hopo, hopq, hopr, hopt, hopv, hoq,
hoqj, hoqk, hoql, hoqn, hoqo, hoqp, hoqq, hoqr, hoqt, hoqv, hor, horj, hork, horl, horn, horo, horp, horq,
horr, hort, horv, hos, hosl, hosr, host, hosv, hot, hoti, hotj, hotk, hotl, hotn, hoto, hotp, hotq, hotr, hott,
hotv, hou, houj, houl, houo, houq, hour, hout, hov, hovj, hovk, hovl, hovn, hovo, hovp, hovq, hovr, hovt,
hovv, hphp, hphq, hphr, hphs, hpht, hphv, hpi, hpii, hpil, hpin, hpio, hpip, hpiq, hpir, hpiv, hpj, hpjj,
hpjp, hpjr, hpjs, hpjt, hpk, hpkk, hpkn, hpkp, hpkq, hpkr, hpks, hpkt, hpku, hpkv, hpl, hplk, hpll, hplp,
hplq, hplr, hplt, hplu, hplv, hpm, hpmk, hpmo, hpmp, hpmq, hpmr, hpmt, hpmv, hpn, hpni, hpnj, hpo,
hpoj, hpon, hpor, hpot, hppk, hppl, hppo, hppp, hppq, hppr, hppt, hppv, hpqj, hpqk, hpql, hpqm, hpqn,
hpqo, hpqp, hpqq, hpqr, hpqs, hpqt, hpqu, hpqv, hpr, hprj, hprk, hprl, hprn, hpro, hprp, hprq, hprr, hprt,
hpru, hprv, hps, hpsi, hpsr, hpst, hpsv, hpt, hpti, hptj, hptk, hptl, hptm, hptn, hpto, hptp, hptq, hptr, hpts,
hptt, hptu, hptv, hpu, hpul, hpuo, hpuq, hpur, hpus, hpuv, hpv, hpvi, hpvk, hpvl, hpvm, hpvn, hpvo, hpvp,
hpvq, hpvr, hpvs, hpvt, hpvu, hpvv, hqhq, hqhr, hqhs, hqht, hqhv, hqi, hqii, hqik, hqil, hqim, hqin, hqio,
hqip, hqiq, hqir, hqis, hqit, hqiv, hqj, hqji, hqjj, hqjk, hqjl, hqjp, hqjq, hqjr, hqjs, hqjt, hqjv, hqk, hqki,
hqkk, hqkl, hqkn, hqkp, hqkq, hqkr, hqkt, hqkv, hql, hqli, hqlk, hqll, hqlm, hqln, hqlo, hqlp, hqlq, hqlr,
hqls, hqlt, hqlv, hqm, hqmk, hqml, hqmn, hqmo, hqmp, hqmq, hqmr, hqms, hqmt, hqmv, hqn, hqni, hqnj,
hqnk, hqnp, hqnq, hqr, hqnt, hqnu, hqnv, hqo, hqoj, hqok, hqol, hqon, hqop, hqoq, hqot, hqou, hqov,
hqp, hqpi, hqpj, hqpk, hqpl, hqpm, hqpn, hqpo, hqpp, hqpq, hqpr, hqpt, hqpv, hqq, hqqi, hqqj, hqqk,
hqql, hqqn, hqqo, hqqp, hqqq, hqqr, hqqs, hqqt, hqqv, hqr, hqri, hqrj, hqrk, hqrl, hqrm, hqrn, hqro, hqrp,
hqrq, hqrr, hqrs, hqrt, hqru, hqrv, hqs, hqsi, hqsj, hqsl, hqsn, hqso, hqsp, hqsq, hqsr, hqst, hqsv, hqt, hqti,
hqtj, hqtk, hqtl, hqtm, hqtn, hqto, hqtp, hqtq, hqtr, hqtt, hqtu, hqtv, hqu, hqui, hquk, hqul, hquo, hquq,
hqur, hqut, hquv, hqv, hqvi, hqvj, hqvk, hqvl, hqvm, hqvn, hqvo, hqvp, hqvq, hqvr, hqvt, hqvv, hrhr,
hrhs, hrht, hrhv, hri, hrii, hrik, hril, hrim, hrin, hrio, hrip, hriq, hrir, hris, hrit, hriv, hrj, hrji, hrjj, hrjk,
hrjp, hrjq, hrjr, hrjs, hrjt, hrju, hrjv, hrk, hrkk, hrkl, hrkn, hrkp, hrkq, hrkr, hrkt, hrku, hrkv, hrl, hrlj, hrlk,
hrll, hrlm, hrln, hrlo, hrlp, hrlq, hrlr, hrlt, hrlv, hrm, hrmj, hrmk, hrml, hrmn, hrmo, hrmp, hrmq, hrmr,
hrmt, hrmv, hrn, hrnj, hrnk, hrnq, hrnr, hrt, hro, hroj, hron, hrot, hrp, hrpi, hrpj, hrpk, hrpl, hrpm, hrpn,
hrpo, hrpp, hrpq, hrpr, hrps, hrpt, hrpv, hrq, hrqi, hrqj, hrqk, hrql, hrqm, hrqn, hrqo, hrqp, hrqq, hrqr,
hrqs, hrqt, hrqu, hrqv, hrr, hrri, hrrj, hrrk, hrrl, hrrm, hrrn, hrro, hrrp, hrrq, hrrr, hrrs, hrrt, hrru, hrrv, hrs,
hrsi, hrsl, hrsn, hrso, hrsp, hrsq, hrsr, hrst, hrsu, hrsv, hrt, hrti, hrtj, hrtk, hrtl, hrtm, hrtn, hrto, hrtp, hrtq,
hrtr, hrts, hrtt, hrtu, hrtv, hru, hruk, hrul, hrun, hruo, hrup, hruq, hrur, hrut, hruv, hrv, hrvi, hrvj, hrvk,
hrvl, hrvm, hrvn, hrvo, hrvp, hrvq, hrvr, hrvs, hrvt, hrvu, hrvv, hshs, hsht, hshv, hsi, hsj, hsk, hski, hsl,
hsli, hslq, hslr, hsls, hslt, hslu, hslv, hsm, hsmi, hsmp, hsmt, hsmu, hsmv, hsn, hso, hsoj, hsor, hsot, hsp,
hspq, hsq, hsqi, hsqj, hsql, hsqp, hsqt, hsqu, hsqv, hsr, hsri, hsrj, hsr, hsrt, hsru, hsrv, hss, hssq, hsst, hst,
hsti, hstr, hstt, hstv, hsu, hsv, hsvp, hsvr, hsvt, hsvv, htht, hthv, hti, htii, htik, htil, htin, htip, htiq, htir,
htit, htiv, htj, htji, htjj, htjk, htjp, htjq, htjr, htjs, htjt, htju, htjv, htk, htkp, htkq, htkr, htkt, htkv, htl, htli,
htlj, htlk, htlm, htlp, htlq, htlr, htlt, htlv, htm, htmk, html, htmn, htmp, htmq, htmr, htmt, htmv, htn, hto,
htoj, htos, htp, htpi, htpj, htpk, htpl, htpm, htpn, htpo, htpq, htpr, htpt, htpv, htq, htqi, htqj, htqk, htql,
htqn, htqo, htqp, htqq, htqr, htqs, htqt, htqu, htqv, htr, htri, htrj, htrk, htrl, htrm, htrn, htro, htrp, htrq, htrr,
htrs, htrt, htru, htrv, hts, htsi, htsl, htsn, htso, htsp, htsq, htsr, htst, htsv, htt, htti, httj, httk, httl, httm, httn,
htto, http, httq, httr, htts, httt, httu, httv, htu, htuk, htul, htun, htuo, htup, htuq, htur, htut, htuv, htv, htvi,
htvj, htvk, htvl, htvm, htvn, htvo, htvp, htvq, htvr, htvs, htvt, htvu, htvv, hui, huj, hujp, hujv, huk, hul,
huli, hulv, hum, hun, huo, hup, hupv, huq, huqi, huqj, huql, huqv, hur, huri, hurl, hurm, hurv, hus, husl,
hut, huti, hutl, hutm, hutv, huu, huv, huvi, huvk, huvl, huvv, hvhv, hvi, hvis, hvik, hvil, hvin, hvio, hvip,
hviq, hvir, hvis, hvit, hviv, hvj, hvjj, hvk, hvkk, hvkl, hvkn, hvko, hvkp, hvkq, hvkr, hvkt, hvkv, hvl,
hvlk, hvll, hvln, hvlp, hvlq, hvlr, hvls, hvlt, hvlv, hvm, hvmi, hvmj, hvmk, hvml, hvmn, hvmp, hvmq,
hvmr, hvms, hvmt, hvmv, hvn, hvni, hvnj, hvnk, hvnp, hvnq, hvnr, hvnt, hvnu, hvnv, hvo, hvoj, hvol,
hvon, hvop, hvoq, hvor, hvot, hvou, hvov, hvp, hvpi, hvpj, hvpk, hvpl, hvpm, hvpn, hvpo, hvpp, hvpq,
hvpr, hvps, hvpt, hvpv, hvq, hvqj, hvqk, hvql, hvqm, hvqn, hvqo, hvqp, hvqq, hvqr, hvqs, hvqt, hvqv,
hvr, hvri, hvrj, hvrk, hvrl, hvrm, hvrn, hvro, hvrp, hvrq, hvrr, hvrs, hvrt, hvru, hvrv, hvs, hvsi, hvsj, hvsk,
hvsl, hvsn, hvso, hvsq, hvsr, hvst, hvsv, hvt, hvtj, hvtk, hvtl, hvtm, hvtn, hvto, hvtp, hvtq, hvtr, hvts, hvtt,
hvtu, hvtv, hvu, hvuo, hvup, hvuq, hvuv, hvv, hvvi, hvvk, hvvl, hvvm, hvvn, hvvo, hvvp, hvvq, hvvr,
hvvs, hvvt, hvvv, iii, iiil, iiio, iiip, iiiq, iiir, iij, iik, iikp, iikq, iikr, iikt, iiku, iikv, iil, iilj, iilk, iill, iilm,
iiln, iilo, iilp, iilq, iilr, iils, iilt, iilv, iim, iin, iinj, iinr, iint, iinu, iio, iioj, iios, iiot, iip, iipl, iipm, iipo, iipp,
iipq, iipr, iips, iipt, iipv, iiq, iiqj, iiqk, iiql, iiqm, iiqo, iiqp, iiqq, iiqr, iiqs, iiqt, iiqu, iiqv, iir, iirj, iirl, iirn,
iirp, iirq, iirr, iirs, iirt, iirv, iis, iisq, iit, iitm, iitp, iitq, iitr, iits, iitt, iitv, iiu, iiup, iiuq, iius, iiuv, iiv, iivk,
iivl, iivm, iivo, iivp, iivq, iivr, iivs, iivt, iivv, ijj, ijjj, ijk, ijl, ijm, ijmo, ijn, ijo, ijp, ijq, ijr, ijs, ijt, ijtl, ijul,
ijv, ikik, ikil, ikim, ikin, ikio, ikip, ikiq, ikir, ikit, ikiv, ikj, ikjk, ikjp, ikjq, ikjr, ikjt, ikjv, ikk, ikkj, ikkl,
ikkn, ikkq, ikkr, ikkt, ikkv, ikl, iklj, iklk, ikln, iklq, iklr, iklt, iklv, ikm, ikmj, ikmk, ikml, ikmp, ikmr,
ikmt, ikmv, ikn, iknj, iko, ikp, ikpj, ikpk, ikpl, ikpn, ikpo, ikpp, ikpq, ikpr, ikpt, ikpv, ikq, ikqj, ikqk, ikql,
ikqn, ikqo, ikqq, ikqr, ikqt, ikqv, ikr, ikrj, ikrk, ikrl, ikrn, ikro, ikrp, ikrq, ikrr, ikrt, ikrv, iks, iksk, iksl,
ikso, iksr, ikst, ikt, iktj, iktk, iktl, iktn, ikto, iktp, iktq, iktr, iktt, iktv, iku, ikuj, ikuk, ikul, ikun, ikuo, ikup,
ikuq, ikur, ikut, ikuv, ikv, ikvj, ikvk, ikvl, ikvn, ikvo, ikvp, ikvq, ikvr, ikvs, ikvt, ikvv, ilil, ilim, ilio, ilip,
iliq, ilir, ilis, ilit, iliv, ilj, iljk, iljp, iljq, iljr, iljt, iljv, ilk, ilkk, ilkl, ilkn, ilkp, ilkq, ilkr, ilkt, ilkv, ill, illj,
illk, illl, illn, illo, illq, illr, ills, illt, illv, ilm, ilmk, ilml, ilmo, ilmp, ilmq, ilmr, iln, ilnj, ilnq, ilnr, ilnt, ilo,
iloj, ilok, ilol, ilon, ilp, ilpk, ilpl, ilpn, ilpo, ilpp, ilpq, ilpr, ilpt, ilpv, ilq, ilqi, ilqk, ilql, ilqn, ilqo, ilqq,
ilqr, ilqt, ilqv, ilr, ilrj, ilrk, ilrl, ilrn, ilro, ilrp, ilrq, ilrr, ilrs, ilrt, ilrv, ils, ilsl, ilso, ilsq, ilsr, ilst, ilsv, ilt,
iltj, iltk, iltl, iltn, ilto, iltp, iltq, iltr, iltt, iltv, ilu, ilul, iluo, ilup, iluq, iluv, ilv, ilvj, ilvk, ilvl, ilvn, ilvo,
ilvp, ilvq, ilvr, ilvt, ilvv, imim, imip, imiq, imir, imit, imiv, imj, imjj, imk, iml, imlm, imlp, imlq, imlr,
imls, imlt, imlv, imm, imn, imns, imo, imp, impt, imq, imqm, imqp, imqq, imqr, imqt, imqv, imr, imrp,
imrq, imrr, imrs, imrt, imrv, ims, imsq, imsr, imst, imsv, imt, imtp, imtq, imtr, imts, imtt, imtv, imu,
imup, imut, imuv, imv, imvm, imvp, imvr, imvs, inin, inir, init, iniv, inj, injj, injq, injr, injt, injv, ink,
inkk, inkl, inkn, inkv, inl, inlv, inm, inmj, inmk, inml, inmn, inmo, inmq, inmr, inmt, inmv, inn, ino, inp,
inpj, inpk, inpl, inpn, inpo, inpp, inpq, inpr, inpt, inpv, inq, inqj, inqk, inql, inqn, inqr, inqt, inqv, inr, inrj,
inrk, inrl, inrn, inro, inrq, inrr, inrt, inrv, ins, insk, insl, insn, inso, insp, insq, insr, inst, insv, int, intj, intk,
intl, intn, into, intq, intr, intt, intv, inu, inuj, inuk, inul, inuq, inur, inut, inuv, inv, invj, invk, invl, invn,
invo, invp, invq, invr, invt, invv, ioio, ioip, ioiq, ioir, ioit, ioiv, ioj, iojk, iojl, iojp, iojq, iojr, iojs, iojt,
iojv, iok, iokk, iokl, iokn, ioko, iokq, iokt, iokv, iol, iolk, ioll, ioln, iolo, iolq, iolr, iolt, iolv, iom, iomq,
ion, ionj, iont, ioo, iop, iopl, iopn, iopo, iopp, iopq, iopr, iopt, iopv, ioq, ioqj, ioqk, ioql, ioqn, ioqo, ioqp,
ioqq, ioqr, ioqt, ioqv, ior, iorj, iork, iorl, ioro, iorq, iorr, iort, iorv, ios, iosl, iosq, iot, iotj, iotk, iotl, ioto,
iotp, iotq, iotr, iott, iotv, iou, iouk, ioul, iouo, iouq, iour, iout, iouv, iov, iovk, iovl, iovn, iovo, iovq, iovr,
iovt, iovv, ipip, ipiq, ipir, ipis, ipit, ipiv, ipj, ipjj, ipjk, ipjp, ipjq, ipjr, ipjs, ipjt, ipjv, ipk, ipkk, ipkl, ipkn,
ipkp, ipkq, ipkr, ipks, ipkt, ipkv, ipl, iplj, iplk, ipll, iplm, ipln, iplo, iplp, iplq, iplr, ipls, iplt, iplu, iplv,
ipm, ipmk, ipmo, ipmp, ipmq, ipmr, ipmt, ipmv, ipn, ipnj, ipnk, ipnp, ipnq, ipnr, ipnt, ipnu, ipo, ipoj,
ipol, ipon, ipoq, ipor, ipos, ipot, ippk, ippl, ippn, ippo, ippp, ippq, ippr, ippt, ippv, ipqj, ipqk, ipql, ipqm,
ipqn, ipqo, ipqp, ipqq, ipqr, ipqs, ipqt, ipqu, ipqv, ipr, iprj, iprk, iprl, iprm, iprn, ipro, iprp, iprq, iprr, iprt,
ipru, iprv, ips, ipsl, ipsn, ipsv, ipt, iptj, iptk, iptl, iptn, ipto, iptp, iptq, iptr, ipts, iptt, iptv, ipu, ipul, ipuo,
ipuq, ipur, iput, ipuv, ipv, ipvj, ipvk, ipvl, ipvm, ipvn, ipvo, ipvp, ipvq, ipvr, ipvs, ipvt, ipvu, ipvv, iqiq,
iqir, iqis, iqit, iqiv, iqj, iqjj, iqjk, iqjl, iqjp, iqjq, iqjr, iqjs, iqjt, iqjv, iqk, iqkk, iqkl, iqkn, iqkq, iqkr, iqks,
iqkt, iqkv, iql, iqlj, iqlk, iqll, iqln, iqlo, iqlp, iqlq, iqlr, iqlt, iqlv, iqm, iqmk, iqml, iqmn, iqmo, iqmp,
iqmq, iqmr, iqmt, iqmv, iqn, iqnj, iqnk, iqnn, iqnq, iqnr, iqnt, iqo, iqoj, iqol, iqon, iqos, iqov, iqpj, iqpk,
iqpl, iqpm, iqpn, iqpo, iqpq, iqpr, iqpt, iqpv, iqqj, iqqk, iqql, iqqm, iqqn, iqqo, iqqp, iqqq, iqqr, iqqs, iqqt,
iqqv, iqr, iqrj, iqrk, iqrl, iqrn, iqro, iqrp, iqrq, iqrr, iqrs, iqrt, iqrv, iqs, iqsl, iqso, iqst, iqsv, iqt, iqtj, iqtk,
iqtl, iqtn, iqto, iqtp, iqtq, iqtr, iqts, iqtt, iqtv, iqu, iquk, iqul, iqun, iquo, iqup, iquq, iqur, iqus, iqut, iquv,
iqv, iqvj, iqvk, iqvl, iqvn, iqvo, iqvp, iqvq, iqvr, iqvs, iqvt, iqvv, irir, iris, irit, iriv, irj, irjj, irjk, irjp, irjq,
irjr, irjs, irjt, irju, irjv, irk, irkk, irkn, irkq, irkr, irkt, irkv, irl, irlj, irlk, irll, irlm, irln, irlp, irlq, irlr, irls,
irlt, irlu, irlv, irm, irmj, irmk, irml, irmn, irmo, irmp, irmq, irmr, irmt, irmv, irn, irnj, irnq, irnr, irnt, irnv,
iro, iroj, iron, iros, irp, irpj, irpk, irpl, irpm, irpn, irpo, irpq, irpr, irps, irpt, irpv, irq, irqj, irqk, irql, irqm,
irqn, irqo, irqp, irqq, irqr, irqs, irqt, irqv, irr, irrj, irrk, irrl, irrm, irrn, irro, irrp, irrq, irrr, irrs, irrt, irrv, irs,
irsk, irsl, irsn, irso, irsq, irsr, irss, irst, irsu, irsv, irt, irtj, irtk, irtl, irtm, irtn, irto, irtp, irtq, irtr, irts, irtt,
irtu, irtv, iru, iruk, irul, irun, iruo, irup, iruq, irur, irut, iruv, irv, irvj, irvk, irvl, irvn, irvo, irvp, irvq, irvr,
irvs, irvt, irvv, isis, isit, isj, isk, iskr, iskt, isl, islk, isll, islp, islq, islr, islt, islv, ism, ismr, isn, isnr, isnt,
isnu, iso, isoj, isok, isol, ison, isoq, isor, isot, isp, ispn, isq, isqj, isqk, isqq, isqr, isqs, isqt, isqv, isr, isrj,
isrk, isrq, isrr, isrt, iss, isso, ist, istj, istk, istl, istr, ists, istt, istu, isu, isuj, isuk, isur, isut, isv, isvk, isvn,
isvq, isvr, isvt, isvv, itit, itiv, itj, itjk, itjp, itjq, itjr, itjs, itjt, itju, itjv, itk, itkk, itkn, itkq, itkr, itks, itkt,
itkv, itl, itlj, itlm, itlp, itlq, itlr, itls, itlt, itlv, itm, itmk, itml, itmo, itmp, itmq, itmr, itmt, itmv, itn, itnj,
ito, itoj, itp, itpj, itpk, itpl, itpm, itpn, itpo, itpq, itpr, itps, itpt, itpv, itq, itqj, itqk, itql, itqm, itqn, itqp,
itqq, itqr, itqs, itqt, itqv, itr, itrj, itrk, itrl, itrm, itrn, itro, itrp, itrq, itrr, itrs, itrt, itrv, its, itsl, itsn, itso, itsq,
itsr, itst, itsv, itt, ittj, ittk, ittl, ittm, ittn, itto, ittp, ittq, ittr, itts, ittt, ittv, itu, ituk, itul, ituo, itup, ituq, itur,
itut, ituv, itv, itvj, itvk, itvl, itvm, itvn, itvo, itvp, itvq, itvr, itvs, itvt, itvv, iuj, iujp, iujq, iujr, iujt, iujv,
iuk, iuks, iul, ium, iun, iuo, iup, iupm, iupv, iuq, iuqs, iuqv, iur, iurs, ius, iut, iutq, iuts, iutv, iuu, iuv,
iuvs, iuvv, iviv, ivj, ivjj, ivk, ivkj, ivkk, ivkl, ivkn, ivko, ivkp, ivkq, ivkr, ivks, ivkt, ivku, ivkv, ivl, ivlj,
ivlk, ivll, ivln, ivlo, ivlp, ivlq, ivlr, ivls, ivlt, ivlu, ivlv, ivm, ivmj, ivmk, ivmn, ivmo, ivmp, ivmq, ivmr,
ivmt, ivmv, ivn, ivnj, ivnk, ivnp, ivnq, ivnr, ivnt, ivnu, ivnv, ivo, ivoj, ivon, ivoq, ivos, ivp, ivpj, ivpk,
ivpl, ivpm, ivpn, ivpo, ivpp, ivpq, ivpr, ivps, ivpt, ivpv, ivq, ivqj, ivqk, ivql, ivqm, ivqn, ivqo, ivqp, ivqq,
ivqr, ivqs, ivqt, ivqv, ivr, ivrj, ivrk, ivrl, ivrm, ivrn, ivro, ivrp, ivrq, ivrr, ivrs, ivrt, ivrv, ivs, ivsj, ivsk,
ivsl, ivsn, ivso, ivsp, ivsq, ivsr, ivss, ivst, ivsv, ivt, ivtj, ivtk, ivtl, ivtm, ivtn, ivto, ivtp, ivtq, ivtr, ivts, ivtt,
ivtv, ivu, ivul, ivuo, ivuq, ivur, ivuv, ivv, ivvj, ivvk, ivvl, ivvm, ivvn, ivvo, ivvp, ivvq, ivvr, ivvs, ivvt,
ivvu, ivvv, jjjj, jjjm, jjjp, jjjr, jjjt, jjjv, jjk, jjkp, jjkq, jjkr, jjkv, jjl, jjlq, jjlv, jjm, jjml, jjmn, jjmo, jjmq,
jjmr, jjmt, jjn, jjo, jjp, jjpl, jjpn, jjpo, jjpq, jjpr, jjpt, jjpv, jjq, jjql, jjqn, jjqp, jjqq, jjqr, jjqt, jjqv, jjr, jjrl,
jjrn, jjro, jjrp, jjrq, jjrr, jjrt, jjrv, jjsn, jjso, jjsq, jjsr, jjst, jjt, jjtl, jjto, jjto, jjtp, jjtq, jjtr, jjtt, jjtv, jjul, jjun,
jjuq, jjur, jjut, jjuv, jjv, jjvl, jjvn, jjvo, jjvp, jjvq, jjvr, jjvt, jjvv, jkjk, jkjl, jkjm, jkjn, jkjp, jkjq, jkjr, jkjt,
jkk, jkkn, jkko, jkkq, jkkr, jkkt, jkkv, jkl, jkll, jkln, jklo, jklp, jklq, jklr, jklt, jklv, jkm, jkmp, jkmq, jkn,
jknr, jko, jkp, jkpn, jkpo, jkpq, jkpr, jkpt, jkq, jkql, jkqn, jkqo, jkqq, jkqr, jkqt, jkqv, jkr, jkrl, jkrn, jkro,
jkrq, jkrr, jkrt, jks, jkt, jktl, jktn, jkto, jktq, jktr, jktt, jktv, jku, jkuo, jkuq, jkut, jkv, jkvl, jkvm, jkvn, jkvo,
jkvq, jkvr, jkvt, jkvv, jljl, jljn, jljq, jljr, jljt, jlk, jlkl, jlkn, jlko, jlkq, jlkr, jlkt, jll, jlll, jlln, jllo, jllq, jlm,
jlmq, jln, jlnl, jlnn, jlnq, jlnr, jlnt, jlo, jlp, jlpo, jlpq, jlpr, jlpt, jlq, jlql, jlqn, jlqo, jlqp, jlqq, jlqr, jlqt, jlqv,
jlr, jlrl, jlrn, jlro, jlrq, jlrr, jlrt, jls, jlt, jltl, jltn, jlto, jltq, jltr, jltt, jlu, jluq, jlv, jlvl, jlvn, jlvo, jlvp, jlvq,
jlvr, jlvt, jmjm, jmjq, jmjr, jmjt, jmk, jml, jmlq, jmlr, jmlt, jmlu, jmlv, jmm, jmn, jmnt, jmo, jmop, jmor,
jmos, jmot, jmp, jmpq, jmpr, jmpt, jmpv, jmq, jmqm, jmqr, jmr, jmrm, jmrn, jmrp, jmrq, jmrr, jmrt, jmrv,
jms, jmt, jmtm, jmtr, jmu, jmv, jmvm, jmvq, jmvr, jmvv, jnjn, jnjq, jnjr, jnjt, jnk, jnl, jnm, jnn, jno, jnp,
jnpq, jnq, jnr, jnrq, jns, jnso, jnsq, jnsr, jnt, jnto, jntq, jntt, jnuo, jnuq, jnv, jojq, jok, jol, jom, jon, joo,
jop, joq, jor, jos, jot, jou, jov, jpjp, jpjq, jpjr, jpjt, jpju, jpk, jpkk, jpkl, jpkm, jpkn, jpko, jpkp, jpkq, jpkr,
jpkt, jpku, jpkv, jpl, jplk, jpll, jplm, jpln, jplo, jplp, jplq, jplr, jpls, jplt, jplu, jplv, jpm, jpmr, jpmt, jpn,
jpnm, jpnn, jpnp, jpnq, jpnr, jpnt, jpnu, jpnv, jpo, jpol, jpon, jpop, jpoq, jpor, jpot, jpov, jpp, jppk, jppn,
jppo, jppq, jppr, jppt, jpq, jpql, jpqm, jpqn, jpqo, jpqp, jpqq, jpqr, jpqt, jpqv, jpr, jprk, jprl, jprm, jprn,
jpro, jprp, jprq, jprr, jprt, jpru, jprv, jps, jpsn, jpt, jptk, jptl, jptm, jptn, jpto, jptp, jptq, jptr, jptt, jptv, jpu,
jpuq, jpv, jpvk, jpvl, jpvm, jpvn, jpvo, jpvp, jpvq, jpvr, jpvt, jpvu, jpvv, jqjq, jqjr, jqjt, jqk, jqkk, jqkl,
jqkm, jqkn, jqko, jqkp, jqkq, jqkr, jqkt, jqku, jqkv, jql, jqll, jqlm, jqln, jqlo, jqlp, jqlq, jqlr, jqlt, jqlu, jqlv,
jqm, jqmq, jqn, jqnl, jqnm, jqnn, jqnp, jqnq, jqnr, jqnt, jqnv, jqo, jqol, jqon, jqoq, jqor, jqot, jqov, jqp,
jqpl, jqpn, jqpo, jqpq, jqpr, jqpt, jqpv, jqq, jqql, jqqn, jqqo, jqqp, jqqq, jqqr, jqqt, jqqv, jqr, jqrl, jqrn,
jqro, jqrp, jqrq, jqrr, jqrt, jqru, jqrv, jqs, jqt, jqtl, jqtm, jqtn, jqto, jqtp, jqtq, jqtr, jqtt, jqtv, jqu, jquq, jqur,
jqv, jqvl, jqvn, jqvo, jqvq, jqvr, jqvt, jqvu, jqvv, jrjr, jrjt, jrju, jrjv, jrk, jrkk, jrkl, jrkm, jrkn, jrkp, jrkq,
jrkr, jrkt, jrkv, jrl, jrlk, jrll, jrlm, jrln, jrlo, jrlp, jrlq, jrlr, jrls, jrlt, jrlu, jrlv, jrm, jrmp, jrmq, jrmr, jrmt, jrn,
jrnm, jrnp, jrnq, jrnr, jrnt, jrnv, jro, jrol, jron, jroq, jror, jrot, jrov, jrp, jrpl, jrpn, jrpo, jrpq, jrpr, jrpt, jrpv,
jrq, jrql, jrqn, jrqo, jrqp, jrqq, jrqr, jrqt, jrqv, jrr, jrrk, jrrl, jrrn, jrro, jrrp, jrrq, jrrr, jrrt, jrrv, jrs, jrsr, jrt,
jrtl, jrtm, jrtn, jrto, jrtp, jrtq, jrtr, jrtt, jrtv, jru, jruo, jruq, jrur, jrut, jrv, jrvk, jrvl, jrvm, jrvn, jrvo, jrvq,
jrvr, jrvt, jrvu, jrvv, jsk, jskk, jskl, jskp, jskq, jskr, jskt, jsl, jslk, jsll, jslo, jslp, jslq, jslr, jslt, jslv, jsm, jsn,
jsnk, jsnm, jsnp, jsnq, jsnr, jsnt, jsnu, jso, jsop, jsoq, jsor, jsot, jsou, jsov, jsp, jspq, jspr, jspt, jspv, jsq,
jsql, jsqp, jsqq, jsqr, jsqt, jsqv, jsr, jsrl, jsrn, jsrp, jsrq, jsrr, jsrt, jsrv, jss, jst, jstk, jstl, jstm, jstp, jstq, jstr,
jstt, jstv, jsu, jsuq, jsut, jsv, jsvk, jsvl, jsvm, jsvn, jsvp, jsvq, jsvr, jsvt, jsvv, jtjt, jtju, jtjv, jtk, jtkl, jtkm,
jtkn, jtkp, jtkq, jtkr, jtkt, jtku, jtkv, jtl, jtlk, jtll, jtlm, jtln, jtlo, jtlp, jtlq, jtlr, jtls, jtlt, jtlu, jtlv, jtm, jtmp,
jtmq, jtmr, jtmt, jtn, jtnm, jtnp, jtnr, jtnv, jto, jton, jtop, jtoq, jtor, jtov, jtp, jtpl, jtpn, jtpo, jtpq, jtpr, jtpt,
jtpv, jtq, jtql, jtqn, jtqo, jtqp, jtqq, jtqr, jtqt, jtqu, jtqv, jtr, jtrl, jtrn, jtro, jtrp, jtrq, jtrr, jtrs, jtrt, jtru, jtrv,
jts, jtsq, jtsr, jtt, jttl, jttm, jttn, jtto, jttp, jttq, jttr, jttt, jttu, jttv, jtu, jtuo, jtuq, jtur, jtut, jtv, jtvk, jtvl, jtvm,
jtvn, jtvo, jtvq, jtvr, jtvt, jtvu, jtvv, juju, juk, jukm, jukp, jukq, jukr, jukt, jukv, jul, julm, julp, julq, julr,
juls, julv, jum, junm, junp, junr, junt, junv, juoq, juor, juov, jup, jupn, jupq, juq, juql, juqn, juqq, juqr,
juqt, juqv, jur, jurl, jurq, jurr, jurt, jus, jut, jutp, jutq, jutr, jutt, jutv, juur, juut, juv, juvk, juvl, juvn, juvq,
juvr, juvt, jvk, jvkk, jvkl, jvkm, jvkn, jvko, jvkp, jvkq, jvkr, jvkt, jvku, jvkv, jvl, jvll, jvlm, jvln, jvlo,
jvlp, jvlq, jvlr, jvls, jvlt, jvlu, jvlv, jvm, jvmq, jvn, jvnm, jvnn, jvnp, jvnq, jvnr, jvnt, jvnu, jvnv, jvo, jvok,
jvol, jvon, jvop, jvoq, jvor, jvot, jvov, jvp, jvpn, jvpo, jvpq, jvpr, jvpt, jvpv, jvq, jvql, jvqm, jvqn, jvqo,
jvqp, jvqq, jvqr, jvqt, jvqv, jvr, jvrk, jvrl, jvrm, jvm, jvro, jvrp, jvrq, jvrr, jvrt, jvru, jvrv, jvs, jvsq, jvst,
jvt, jvtk, jvtl, jvtm, jvtn, jvto, jvtp, jvtq, jvtr, jvtt, jvtv, jvu, jvuq, jvv, jvvk, jvvl, jvvm, jvvn, jvvo, jvvp,
jvvq, jvvr, jvvt, jvvu, jvvv, kkkl, kkkn, kkkp, kkkq, kkkr, kkkt, kkkv, kkl, kkll, kkln, kklo, kklp, kklq,
kklr, kklt, kklu, kklv, kkm, kkmp, kkmv, kkn, kknp, kknq, kknr, kknt, kknu, kknv, kko, kkoq, kkor, kkot,
kkov, kkp, kkpl, kkpn, kkpo, kkpp, kkpq, kkpr, kkpt, kkpv, kkq, kkql, kkqn, kkqo, kkqp, kkqq, kkqr,
kkqt, kkqv, kkr, kkrl, kkrn, kkro, kkrp, kkrq, kkrr, kkrt, kkru, kkrv, kks, kksv, kkt, kktl, kkto, kktp, kktq,
kktr, kktt, kktv, kku, kkuq, kkuv, kkv, kkvl, kkvn, kkvo, kkvp, kkvq, kkvr, kkvt, kkvv, klkl, klkn, klko,
klkp, klkq, klkr, klks, klkt, klku, klkv, kll, klll, klln, kllo, kllp, kllq, kllr, kllt, kllv, klm, klml, klmp, klmq,
klmv, kln, klnl, klnp, klnq, klnr, klns, klnt, klnu, klnv, klo, klol, klon, kloq, klot, klov, klp, klpl, klpn,
klpo, klpp, klpq, klpr, klpt, klpv, klq, klql, klqm, klqn, klqo, klqp, klqq, klqr, klqs, klqt, klqv, klr, klrl,
klm, klro, klrp, klrq, klrr, klrs, klrt, klru, klrv, kls, klt, kltl, kltn, klto, kltp, kltq, kltr, klts, kltt, kltv, klu,
kluq, kluv, klv, klvl, klvn, klvo, klvp, klvq, klvr, klvs, klvt, klvu, klvv, kmkm, kml, kmll, kmlm, kmlp,
kmlq, kmlr, kmlv, kmm, kmn, kmnm, kmo, kmol, kmov, kmp, kmpm, kmpp, kmpq, kmpr, kmpt, kmpv,
kmq, kmqm, kmqq, kmqr, kmqs, kmqt, kmqu, kmr, kmrl, kmrm, kmrp, kmrq, kmrr, kmrs, kmrt, kmru,
kmrv, kms, kmsm, kmsq, kmt, kmtm, kmtp, kmtr, kmtt, kmtu, kmu, kmv, kmvm, kmvq, kmvr, kmvt,
kmvu, kmvv, knkn, knko, knkq, knkt, knkv, knl, knlq, knm, knmp, knmr, knmv, knn, kno, knp, knpl,
knpn, knpo, knpq, knpr, knpt, knpv, knq, knql, knqn, knqo, knqq, knqr, knqt, knr, knrl, knrn, knro, knrp,
knrq, knrr, knrt, knrv, kns, knsq, knsr, knsv, knt, kntl, kntn, knto, kntp, kntq, kntr, kntt, kntv, knu, knul,
knuo, knup, knuq, knur, knut, knv, knvl, knvn, knvo, knvq, knvr, knvt, knvv, kokv, kol, koln, kom, kon,
kong, konv, koo, kop, koq, koql, koqn, koqo, koqp, koqq, koqr, koqt, koqv, kor, korl, koro, korq, korv,
kos, kot, kotl, koto, kotp, kotq, kotv, kou, kouo, kov, kovl, kovn, kovo, kovq, kovv, kpkp, kpkq, kpkr,
kpks, kpkt, kpku, kpkv, kpl, kpll, kplp, kplq, kplr, kpls, kplt, kplu, kplv, kpm, kpmv, kpn, kpnm, kpnp,
kpnq, kpnr, kpns, kpnt, kpnu, kpnv, kpo, kpol, kpom, kpon, kpop, kpoq, kpor, kpos, kpot, kpou, kpov,
kppl, kppo, kppp, kppq, kppr, kppt, kppv, kpql, kpqm, kpqn, kpqo, kpqp, kpqq, kpqr, kpqs, kpqt, kpqu,
kpqv, kpr, kprl, kpro, kprp, kprq, kprr, kprs, kprt, kpru, kprv, kps, kpst, kpsv, kpt, kptl, kptm, kpto, kptp,
kptq, kptr, kpts, kptt, kptu, kptv, kpu, kpuq, kpus, kpuv, kpvl, kpvn, kpvo, kpvp, kpvq, kpvr, kpvs, kpvt,
kpvu, kpvv, kqkq, kqkr, kqks, kqkt, kqku, kqkv, kql, kqll, kqln, kqlo, kqlp, kqlq, kqlr, kqls, kqlt, kqlu,
kqlv, kqm, kqmp, kqmq, kqmv, kqn, kqnl, kqnp, kqnq, kqnr, kqns, kqnt, kqnu, kqnv, kqo, kqol, kqon,
kqoo, kqop, kqoq, kqor, kqot, kqou, kqov, kqpl, kqpn, kqpo, kqpp, kqpq, kqpr, kqps, kqpt, kqpv, kqql,
kqqn, kqqo, kqqp, kqqq, kqqr, kqqs, kqqt, kqqv, kqr, kqrl, kqrn, kqro, kqrp, kqrq, kqrr, kqrs, kqrt, kqru,
kqrv, kqs, kqsl, kqsq, kqsv, kqt, kqtl, kqtn, kqto, kqtp, kqtq, kqtr, kqts, kqtt, kqtu, kqtv, kqu, kquo, kqus,
kquv, kqvl, kqvn, kqvo, kqvp, kqvq, kqvr, kqvt, kqvu, kqvv, krkr, krks, krkt, krkv, krl, krll, krlm, krln,
krlo, krlp, krlq, krlr, krls, krlt, krlu, krlv, krm, krmp, krmv, krn, krnm, krnp, krnq, krnr, krns, krnt, krnu,
krnv, kro, krol, krom, kron, krop, kroq, kror, krot, krou, krov, krp, krpl, krpn, krpo, krpp, krpq, krpr,
krps, krpt, krpv, krq, krql, krqm, krqn, krqo, krqp, krqq, krqr, krqt, krqu, krqv, krr, krrl, krrm, krrn, krro,
krrp, krrq, krrr, krrs, krrt, krru, krrv, krs, krsq, krsr, krsv, krt, krtl, krtm, krto, krtp, krtq, krtr, krts, krtt,
krtu, krtv, kru, krul, kruo, kruq, kruv, krv, krvl, krvn, krvo, krvp, krvq, krvr, krvt, krvu, krvv, ksks, kskt,
ksku, kskv, ksl, ksll, kslm, kslp, kslq, kslr, ksls, kslt, kslv, ksm, ksn, ksnm, ksnp, ksnq, ksnr, ksnt, ksnv,
kso, ksol, ksom, ksop, ksoq, ksot, ksp, kspq, kspr, kspt, kspv, ksq, ksql, ksqp, ksqq, ksqr, ksqt, ksqv, ksr,
ksrl, ksrp, ksrq, ksrr, ksrs, ksrt, ksru, ksrv, kss, kst, kstl, kstp, kstq, kstr, ksts, kstt, kstu, kstv, ksu, ksuv,
ksv, ksvl, ksvm, ksvp, ksvq, ksvr, ksvt, ksvv, ktkt, ktku, ktkv, ktl, ktll, ktlm, ktln, ktlo, ktlp, ktlq, ktlr,
ktls, ktlt, ktlu, ktlv, ktm, ktmp, ktms, ktmv, ktn, ktnm, ktnp, ktnq, ktnr, ktns, ktnt, ktnu, ktnv, kto, ktol,
kton, ktop, ktoq, ktor, ktot, ktou, ktov, ktp, ktpl, ktpn, ktpo, ktpp, ktpq, ktpr, ktpt, ktpv, ktq, ktql, ktqm,
ktqn, ktqo, ktqp, ktqq, ktqr, ktqt, ktqu, ktqv, ktr, ktrl, ktrm, ktrn, ktro, ktrp, ktrq, ktrr, ktrs, ktrt, ktru, ktrv,
kts, ktsq, ktsr, ktsv, ktt, kttl, kttm, kttn, ktto, kttp, kttq, kttr, ktts, kttt, kttu, kttv, ktu, ktuq, ktuv, ktv, ktvl,
ktvn, ktvo, ktvp, ktvq, ktvr, ktvt, ktvu, ktvv, kuku, kukv, kul, kull, kulm, kulq, kulr, kult, kulu, kulv,
kum, kun, kunm, kung, kunr, kunv, kuo, kuoq, kuor, kuov, kup, kupr, kupt, kupv, kuq, kuql, kuqp, kuqq,
kuqr, kuqt, kuqv, kur, kurl, kurq, kurr, kurt, kuru, kurv, kus, kut, kutl, kutm, kutq, kutr, kuts, kutt, kutu,
kutv, kuu, kuuv, kuv, kuvl, kuvp, kuvq, kuvr, kuvt, kuvu, kuvv, kvkv, kvl, kvll, kvln, kvlo, kvlp, kvlq,
kvlr, kvls, kvlt, kvlu, kvlv, kvm, kvmv, kvn, kvnl, kvnp, kvnq, kvnr, kvns, kvnt, kvnu, kvnv, kvo, kvol,
kvom, kvon, kvoo, kvop, kvoq, kvor, kvot, kvou, kvov, kvp, kvpl, kvpn, kvpo, kvpp, kvpq, kvpr, kvpt,
kvpv, kvq, kvql, kvqm, kvqn, kvqo, kvqp, kvqq, kvqr, kvqs, kvqt, kvqu, kvqv, kvr, kvrl, kvrn, kvro, kvrp,
kvrq, kvrr, kvrs, kvrt, kvru, kvrv, kvs, kvsp, kvsq, kvsr, kvsv, kvt, kvtl, kvto, kvtp, kvtq, kvtr, kvts, kvtt,
kvtu, kvtv, kvu, kvuq, kvur, kvus, kvuv, kvv, kvvl, kvvm, kvvn, kvvo, kvvp, kvvq, kvvr, kvvs, kvvt,
kvvu, kvvv, lll, llll, lllo, lllp, lllq, lllr, lllt, lllv, llm, llmp, llms, lln, llnp, llnq, llnr, llnt, llnu, llnv, llo, llon,
llop, lloq, llor, llot, llov, llp, llpo, llpp, llpq, llpr, llps, llpt, llpv, llq, llqm, llqn, llqo, llqp, llqq, llqr, llqs,
llqt, llqu, llqv, llr, llrm, llro, llrp, llrq, llrr, llrt, llru, llrv, lls, llso, llsq, llsr, llsv, llt, llto, lltp, lltq, lltr, lltt,
lltv, llu, llup, lluq, lluv, llv, llvm, llvn, llvo, llvp, llvq, llvr, llvs, llvt, llvu, llvv, lmlm, lmlp, lmlq, lmlr,
lmls, lmlt, lmlu, lmlv, lmm, lmn, lmnp, lmnr, lmns, lmnt, lmnv, lmo, lmom, lmop, lmoq, lmor, lmot,
lmou, lmov, lmp, lmpq, lmpr, lmpt, lmpv, lmq, lmqp, lmqq, lmqr, lmqs, lmqt, lmqu, lmr, lmrp, lmrq,
lmrr, lmrs, lmrt, lmru, lmrv, lms, lmsv, lmt, lmtr, lmtt, lmtu, lmu, lmv, lmvp, lmvq, lmvr, lmvt, lmvv,
lnln, lnlq, lnlt, lnlv, lnm, lnmr, lnmv, lnn, lno, lnp, lnpn, lnpo, lnpp, lnpq, lnpr, lnpt, lnpv, lnq, lnqn, lnqo,
lnqp, lnqq, lnqr, lnqt, lnqv, lnr, lnrn, lnro, lnrp, lnrq, lnrr, lnrt, lnrv, lns, lnsn, lnso, lnsq, lnsr, lnst, lnsv,
lnt, lnto, lntp, lntq, lntr, lntt, lntv, lnu, lnuo, lnuq, lnur, lnut, lnuv, lnv, lnvn, lnvo, lnvp, lnvq, lnvr, lnvt,
lnvv, lolo, lolp, lolq, lolr, lolt, lolv, lom, lon, lonp, long, lonr, lont, lonv, loo, lop, lopo, lopq, lopt, lopv,
loq, loqo, loqp, loqq, loqr, loqt, loqv, lor, loro, lorq, lorr, lort, lorv, los, lot, loto, lotp, lotq, lotr, lott, lotv,
lou, louq, lov, lovo, lovq, lovr, lovv, lplp, lplq, lplr, lpls, lplt, lplu, lplv, lpln, lpmp, lpmr, lpmv, lpn, lpnm,
lpnp, lpnq, lpnr, lpns, lpnt, lpnu, lpnv, lpo, lpom, lpop, lpoq, lpor, lpos, lpot, lpou, lpov, lppp, lppq, lppr,
lpps, lppt, lppu, lppv, lpqm, lpqo, lpqp, lpqq, lpqr, lpqs, lpqt, lpqu, lpqv, lpr, lprm, lpro, lprp, lprq, lprr,
lprs, lprt, lpru, lprv, lps, lpsq, lpsr, lpst, lpsv, lpt, lptm, lptp, lptq, lptr, lpts, lptt, lptu, lptv, lpu, lpuq, lpur,
lput, lpuv, lpvm, lpvn, lpvo, lpvp, lpvq, lpvr, lpvs, lpvt, lpvu, lpvv, lqlq, lqlr, lqls, lqlt, lqlu, lqlv, lqm,
lqmo, lqmp, lqmq, lqmr, lqmt, lqmv, lqn, lqnm, lqnp, lqnq, lqnr, lqns, lqnt, lqnu, lqnv, lqo, lqon, lqoo,
lqop, lqoq, lqor, lqos, lqot, lqou, lqov, lqpn, lqpo, lqpp, lqpq, lqpr, lqps, lqpt, lqpv, lqqn, lqqo, lqqp, lqqq,
lqqr, lqqs, lqqt, lqqu, lqqv, lqr, lqrm, lqrn, lqro, lqrp, lqrq, lqrr, lqrs, lqrt, lqru, lqrv, lqs, lqsn, lqso, lqsp,
lqsq, lqsr, lqst, lqsv, lqt, lqtm, lqto, lqtp, lqtq, lqtr, lqts, lqtt, lqtu, lqtv, lqu, lquo, lqur, lqut, lquv, lqvn,
lqvo, lqvp, lqvq, lqvr, lqvs, lqvt, lqvu, lqvv, lrlr, lrls, lrlt, lrlu, lrlv, lrm, lrmo, lrmp, lrmr, lrms, lrn, lrnm,
lrnp, lrnq, lrnr, lrns, lrnt, lrnu, lrnv, lro, lrom, lrop, lroq, lror, lros, lrot, lrou, lrov, lrp, lrpo, lrpp, lrpq,
lrpr, lrps, lrpt, lrpv, lrq, lrqm, lrqn, lrqo, lrqp, lrqq, lrqr, lrqs, lrqt, lrqu, lrqv, lrr, lrrm, lrro, lrrp, lrrq, lrrr,
lrrs, lrrt, lrru, lrrv, lrs, lrso, lrsq, lrsr, lrst, lrsv, lrt, lrtm, lrto, lrtp, lrtq, lrtr, lrts, lrtt, lrtu, lrtv, lru, lruo,
lrup, lruq, lrur, lrut, lruv, lrv, lrvm, lrvn, lrvo, lrvp, lrvq, lrvr, lrvs, lrvt, lrvu, lrvv, lsls, lslt, lslu, lslv, lsm,
lsn, lsnp, lsnq, lsnr, lsns, lsnt, lsnu, lsnv, lso, lsop, lsoq, lsor, lsot, lsov, lsp, lspq, lspr, lspt, lspv, lsq, lsqp,
lsqq, lsqr, lsqs, lsqt, lsqu, lsqv, lsr, lsrm, lsrp, lsrq, lsrr, lsrs, lsrt, lsru, lsrv, lss, lssp, lst, lstp, lstq, lstr,
lsts, lstt, lstu, lstv, lsu, lsuq, lsuv, lsv, lsvm, lsvp, lsvq, lsvr, lsvs, lsvt, lsvv, ltlt, ltlu, ltlv, ltm, ltmo, ltmp,
ltms, ltn, ltnp, ltnq, ltnr, ltns, ltnt, ltnu, ltnv, lto, ltom, ltop, ltoq, ltor, ltos, ltot, ltou, ltov, ltp, ltpn, ltpo,
ltpp, ltpq, ltpr, ltpt, ltpv, ltq, ltqn, ltqo, ltqp, ltqq, ltqr, ltqs, ltqt, ltqu, ltqv, ltr, ltrm, ltro, ltrp, ltrq, ltrr, ltrs,
ltrt, ltru, ltrv, lts, ltsp, ltsq, ltsr, ltss, ltst, ltsv, ltt, lttm, ltto, lttp, lttq, lttr, ltts, lttt, lttu, lttv, ltu, ltuv, ltv,
ltvn, ltvo, ltvp, ltvq, ltvr, ltvs, ltvt, ltvu, ltvv, lulu, lulv, lum, lun, lunq, lunr, lunt, lunu, luo, luop, luoq,
luor, luos, luot, luou, luov, lup, lupq, lupr, lupt, lupv, luq, luqp, luqq, luqr, luqt, luqv, lur, lurp, lurq, lurr,
lurs, lurt, luru, lurv, lus, lusp, lusq, lusv, lut, lutm, lutp, lutq, lutr, luts, lutt, lutu, lutv, luu, luv, luvo, luvp,
luvq, luvr, luvs, luvt, luvu, luvv, lvlv, lvm, lvmo, lvmp, lvmr, lvmt, lvmv, lvn, lvnm, lvnp, lvnq, lvnr,
lvnt, lvnu, lvnv, lvo, lvom, lvon, lvoo, lvop, lvoq, lvor, lvos, lvot, lvou, lvov, lvp, lvpn, lvpo, lvpp, lvpq,
lvpr, lvps, lvpt, lvpu, lvpv, lvq, lvqm, lvqn, lvqo, lvqp, lvqq, lvqr, lvqs, lvqt, lvqu, lvqv, lvr, lvrm, lvrn,
lvro, lvrp, lvrq, lvrr, lvrs, lvrt, lvru, lvrv, lvs, lvso, lvsq, lvsr, lvst, lvsv, lvt, lvtm, lvto, lvtp, lvtq, lvtr,
lvts, lvtt, lvtu, lvtv, lvu, lvuo, lvuq, lvur, lvut, lvuv, lvv, lvvm, lvvn, lvvo, lvvp, lvvq, lvvr, lvvs, lvvt,
lvvu, lvvv, mmmm, mmn, mmo, mmr, mmrp, mms, mmt, mmtp, mmu, mmup, mnmn, mnmo, mnmp,
mnmq, mnmr, mnmt, mnmv, mnn, mno, mnp, mnpq, mnpr, mnq, mnr, mns, mnsn, mnsr, mnst, mnt,
mnu, mnv, mnvp, mnvq, mnvv, momo, momr, momt, mon, moo, mop, mopn, mopq, mopr, mopt, moq,
moqq, moqr, moqt, moqv, mor, morn, morr, mort, mos, mosn, mosr, mot, motn, motp, motq, motr, mott,
mou, mour, mov, movn, movp, movq, movr, movt, movv, mpmp, mpmq, mpmr, mpmt, mpmv, mpn,
mpo, mppn, mppp, mppq, mppr, mpps, mppt, mppv, mpqn, mpqp, mpqq, mpqr, mpqs, mpqt, mpqv, mpr,
mprn, mpro, mprp, mprq, mprr, mprt, mprv, mps, mpsp, mpsq, mpsr, mpst, mpsv, mpt, mptn, mpto,
mptp, mptq, mptr, mptt, mptv, mpu, mpup, mpuq, mpur, mput, mpuv, mpvn, mpvo, mpvp, mpvq, mpvr,
mpvt, mpvu, mpvv, mqmq, mqmr, mqms, mqmt, mqmv, mqn, mqo, mqpo, mqpp, mqpq, mqpr, mqpt,
mqpv, mqqn, mqqo, mqqq, mqqt, mqr, mqm, mqro, mqrp, mqrq, mqrr, mqrt, mqs, mqsn, mqso, mqsp,
mqsq, mqsr, mqst, mqsv, mqt, mqtn, mqto, mqtp, mqtq, mqtr, mqtt, mqtv, mqu, mquo, mqup, mquq,
mqus, mqut, mquv, mqvn, mqvo, mqvp, mqvq, mqvr, mqvt, mqvv, mrmr, mrms, mrmt, mrmv, mrm, mro,
mrp, mrpn, mrpq, mrpr, mrps, mrpt, mrpv, mrq, mrqu, mrqr, mrqt, mrr, mrrn, mrrp, mrrq, mrrr, mrrt,
mrs, mrsn, mrso, mrsq, mrsr, mrst, mrsv, mrt, mrtn, mrto, mrtp, mrtq, mrtr, mrtt, mru, mrun, mruo, mruq,
mrur, mrut, mrv, mrvn, mrvp, mrvq, mrvr, mrvt, mrvv, msms, msn, mso, msp, msq, msqp, msqq, msqr,
msqt, msqu, msr, msrr, msrt, mss, mssr, mst, mstt, msu, msv, msvq, msvr, msvt, msvu, msvv, mtmt,
mtmv, mtn, mto, mtp, mtpn, mtpq, mtpr, mtpv, mtq, mtr, mtrn, mtrp, mtrr, mtrt, mts, mtsn, mtso, mtsq,
mtsr, mtst, mtsv, mtt, mttn, mttp, mttq, mttr, mttt, mtu, mtun, mtuq, mtur, mtut, mtv, mtvq, mtvv, mun,
muo, mup, muq, mur, mus, mut, muu, muv, mvmv, mvn, mvo, mvpo, mvpp, mvpq, mvpr, mvpt, mvpv,
mvqn, mvqo, mvqp, mvqq, mvqr, mvqt, mvqv, mvr, mvrn, mvro, mvrp, mvrq, mvrr, mvrs, mvrt, mvrv,
mvs, mvsn, mvso, mvsp, mvsq, mvsr, mvsv, mvt, mvtn, mvto, mvtp, mvtq, mvtr, mvts, mvtt, mvtv, mvu,
mvuo, mvup, mvuq, mvur, mvus, mvut, mvuv, mvvn, mvvo, mvvp, mvvq, mvvr, mvvs, mvvt, mvvu,
mvvv, nno, nnp, nnq, nnqo, nnr, nns, nnt, nnto, nnu, nnv, noo, nop, noq, nor, nos, not, nou, nov, npnp,
npnq, npnr, npns, npnt, npnu, npnv, npo, npoo, npop, npoq, npor, npot, npou, npov, nppp, nppq, nppr,
npps, nppt, nppu, nppv, npqp, npqq, npqr, npqs, npqt, npqu, npqv, npr, nprp, nprq, nprr, nprs, nprt, npru,
nprv, nps, npt, nptp, nptq, nptr, npts, nptt, nptv, npu, npv, npvp, npvq, npvr, npvs, npvt, npvu, npvv,
nqnq, nqnr, nqns, nqnt, nqnu, nqnv, nqo, nqoo, nqop, nqoq, nqor, nqot, nqov, nqpo, nqpp, nqpq, nqpr,
nqpt, nqpv, nqqo, nqqp, nqqq, nqqr, nqqs, nqqt, nqqu, nqqv, nqr, nqro, nqrp, nqrq, nqrr, nqrs, nqrt, nqru,
nqrv, nqs, nqt, nqto, nqtp, nqtq, nqtr, nqts, nqtt, nqtv, nqu, nquv, nqv, nqvo, nqvp, nqvq, nqvr, nqvs, nqvt,
nqvu, nqvv, nrnr, nrns, nrnt, nrnu, nrnv, nro, nrop, nroq, nror, nrot, nrou, nrov, nrp, nrpo, nrpp, nrpq,
nrpr, nrps, nrpt, nrpv, nrq, nrqo, nrqp, nrqq, nrqr, nrqt, nrqv, nrr, nrro, nrrp, nrrq, nrrr, nrrs, nrrt, nrru,
nrrv, nrs, nrt, nrto, nrtp, nrtq, nrtr, nrtt, nrtu, nrtv, nru, nrv, nrvo, nrvp, nrvq, nrvr, nrvt, nrvu, nrvv, nsns,
nsnt, nsnu, nsnv, nso, nsop, nsoq, nsor, nsot, nsov, nsp, nspq, nspr, nspt, nspv, nsq, nsqp, nsqq, nsqr,
nsqt, nsqu, nsqv, nsr, nsrp, nsrq, nsrr, nsrt, nsrv, nss, nst, nstp, nstq, nstr, nstt, nstu, nstv, nsu, nsv, nsvp,
nsvq, nsvr, nsvt, nsvv, ntnt, ntnu, ntnv, nto, ntop, ntoq, ntor, ntot, ntou, ntov, ntp, ntpo, ntpp, ntpq, ntpr,
ntps, ntpt, ntpv, ntq, ntqo, ntqp, ntqq, ntqr, ntqt, ntqu, ntqv, ntr, ntro, ntrp, ntrq, ntrr, ntrs, ntrt, ntru, ntrv,
nts, ntt, ntto, nttp, nttq, nttr, ntts, nttt, nttu, nttv, ntu, ntv, ntvo, ntvp, ntvq, ntvr, ntvs, ntvt, ntvu, ntvv,
nunu, nunv, nuop, nuoq, nuor, nuot, nuov, nup, nupp, nupq, nupr, nupt, nupv, nuq, nuqp, nuqq, nuqr,
nuqt, nuqv, nur, nurp, nurq, nurr, nurs, nurt, nuru, nurv, nus, nut, nutp, nutq, nutr, nuts, nutt, nutu, nutv,
nuv, nuvq, nuvr, nuvt, nuvv, nvnv, nvo, nvoo, nvop, nvoq, nvor, nvot, nvov, nvp, nvpo, nvpp, nvpq,
nvpr, nvps, nvpt, nvpv, nvq, nvqo, nvqp, nvqq, nvqr, nvqs, nvqt, nvqu, nvqv, nvr, nvro, nvrp, nvrq, nvrr,
nvrs, nvrt, nvru, nvrv, nvs, nvsv, nvt, nvto, nvtp, nvtq, nvtr, nvts, nvtt, nvtv, nvu, nvv, nvvo, nvvp, nvvq,
nvvr, nvvs, nvvt, nvvu, nvvv, ooo, oop, ooq, ooqq, ooqv, oor, oos, oot, ootq, ootv, oou, oov, opop, opoq,
opor, opos, opot, opou, opov, oppq, oppr, oppt, oppu, oppv, opqp, opqq, opqr, opqs, opqt, opqu, opqv,
opr, oprp, oprq, oprr, oprs, oprt, opru, oprv, ops, opsv, opt, optp, optq, optr, opts, optt, optu, optv, opu,
opv, opvp, opvq, opvr, opvs, opvt, opvu, opvv, oqoq, oqor, oqot, oqou, oqov, oqpp, oqpq, oqpr, oqps,
oqpt, oqpu, oqpv, oqqp, oqqq, oqqr, oqqs, oqqt, oqqu, oqqv, oqr, oqrp, oqrq, oqrr, oqrs, oqrt, ogru, oqrv,
oqs, oqsv, oqt, oqtp, oqtq, oqtr, oqts, oqtt, oqtu, oqtv, oqu, oqv, oqvp, oqvq, oqvr, oqvs, oqvt, oqvu, oqvv,
oror, oros, orot, orou, orov, orp, orpp, orpq, orpr, orpt, orpu, orpv, orq, orqp, orqq, orqr, orqs, orqt, orqu,
orqv, orr, orrp, orrq, orrr, orrs, orrt, orru, orrv, ors, ort, ortp, ortq, ortr, orts, ortt, ortu, ortv, oru, orv, orvp,
orvq, orvr, orvs, orvt, orvu, orvv, osos, osot, osp, ospr, ospt, ospu, osq, osqq, osqr, osqs, osqt, osqv, osr,
osrp, osrq, osrr, osrt, osrv, oss, ost, ostt, ostu, osu, osv, osvq, osvr, osvt, osvv, otot, otou, otov, otp, otpp,
otpq, otpr, otps, otpt, otpu, otpv, otq, otqp, otqq, otqr, otqs, otqt, otqu, otqv, otr, otrp, otrq, otrr, otrs, otrt,
otru, otrv, ots, otsq, otsv, ott, ottp, ottq, ottr, otts, ottt, ottu, ottv, otu, otv, otvp, otvq, otvr, otvs, otvt, otvu,
otvv, ouou, ouov, oup, oupv, ouq, ouqq, ouqr, ouqt, ouqv, our, ourp, ourr, ours, ourt, ouru, ourv, ous,
out, outr, outt, outu, outv, ouv, ouvq, ouvr, ouvs, ouvt, ovov, ovp, ovpp, ovpq, ovpr, ovps, ovpt, ovpu,
ovpv, ovq, ovqp, ovqq, ovqr, ovqs, ovqt, ovqu, ovqv, ovr, ovrp, ovrq, ovrr, ovrs, ovrt, ovru, ovrv, ovs,
ovt, ovtp, ovtq, ovtr, ovts, ovtt, ovtu, ovtv, ovu, ovv, ovvp, ovvq, ovvr, ovvs, ovvt, ovvu, ovvv, pppp,
pppq, pppr, pppt, pppu, pppv, ppqq, ppqr, ppqs, ppqt, ppqv, ppr, pprq, pprr, pprt, pprv, pps, ppsq, ppst,
ppsv, ppt, pptq, pptr, ppts, pptt, pptu, pptv, ppu, ppuq, ppur, ppus, pput, ppuv, ppvq, ppvr, ppvs, ppvt,
ppvu, ppvv, pqpq, pqpr, pqps, pqpt, pqpv, pqqq, pqqr, pqqs, pqqt, pqqu, pqqv, pqr, pqrq, pqrr, pqrs, pqrt,
pqru, pqrv, pqs, pqsq, pqsr, pqst, pqsv, pqt, pqtq, pqtr, pqts, pqtt, pqtu, pqtv, pqu, pquq, pqur, pqus, pqut,
pquv, pqvq, pqvr, pqvs, pqvt, pqvu, pqvv, prpr, prpt, prpu, prpv, prq, prqq, prqr, prqs, prqt, prqu, prqv,
prr, prrq, prrr, prrs, prrt, prru, prrv, prs, prsq, prsr, prst, prsu, prsv, prt, prtq, prtr, prts, prtt, prtu, prtv, pru,
pruq, prur, prut, pruv, prv, prvq, prvr, prvt, prvu, prvv, psps, pspv, psq, psqq, psqr, psqs, psqt, psqu, psqv,
psr, psrq, psrr, psrv, pss, pst, pstq, psts, pstt, pstv, psu, psv, psvq, psvr, psvv, ptpt, ptpu, ptpv, ptq, ptqq,
ptqr, ptqs, ptqt, ptqu, ptqv, ptr, ptrq, ptrr, ptrs, ptrt, ptru, ptrv, pts, ptsq, ptsr, ptst, ptsv, ptt, pttq, pttr, ptts,
pttt, pttu, pttv, ptu, ptuq, ptur, ptut, ptuv, ptv, ptvq, ptvr, ptvs, ptvt, ptvu, ptvv, pupu, pupv, puq, puqq,
puqr, puqt, puqu, pur, purq, purr, purt, purv, pus, pusq, pusr, pust, put, putt, puu, puv, puvq, puvr, puvs,
puvt, puvu, puvv, pvpv, pvqq, pvqr, pvqs, pvqt, pvqu, pvqv, pvr, pvrq, pvrr, pvrs, pvrt, pvru, pvrv, pvs,
pvsq, pvsr, pvst, pvsv, pvt, pvtq, pvtr, pvts, pvtt, pvtu, pvtv, pvu, pvuq, pvur, pvus, pvut, pvuv, pvvq,
pvvr, pvvs, pvvt, pvvu, pvvv, qqqr, qqqs, qqqt, qqqu, qqqv, qqr, qqrr, qqrs, qqrt, qqru, qqrv, qqs, qqsr,
qqsv, qqt, qqtr, qqts, qqtt, qqtu, qqtv, qqu, qqur, qqus, qqut, qquv, qqvr, qqvs, qqvt, qqvu, qqvv, qrqr,
qrqs, qrqt, qrqu, qrqv, qrr, qrrr, qrrs, qrrt, qrru, qrrv, qrs, qrsr, qrst, qrsv, qrt, qrtr, qrts, qrtt, qrtu, qrtv,
qru, qrur, qrus, qrut, qruv, qrv, qrvr, qrvs, qrvt, qrvu, qrvv, qsqs, qsqt, qsqu, qsqv, qsr, qsrr, qsrs, qsrt,
qsrv, qss, qst, qstr, qsts, qstt, qstv, qsu, qsuv, qsv, qsvr, qsvs, qsvt, qsvu, qsvv, qtqt, qtqu, qtqv, qtr, qtrr,
qtrs, qtrt, qtru, qtrv, qts, qtsr, qtst, qtsv, qtt, qttr, qtts, qttt, qttu, qttv, qtu, qtur, qtus, qtuv, qtv, qtvr, qtvs,
qtvt, qtvu, qtvv, ququ, quqv, qur, qurr, qurs, qurt, quru, qurv, qus, qusr, qust, qusv, qut, qutr, quts, qutt,
qutu, qutv, quu, quv, quvr, quvs, quvt, quvu, quvv, qvqv, qvr, qvrr, qvrs, qvrt, qvru, qvrv, qvs, qvsr, qvst,
qvsv, qvt, qvtr, qvts, qvtt, qvtu, qvtv, qvu, qvur, qvus, qvut, qvuv, qvvr, qvvs, qvvt, qvvu, qvvv, rrrs,
rrrt, rrru, rrrv, rrs, rrst, rrsv, rrts, rrtt, rrtu, rrtv, rru, rrut, rruv, rrv, rrvs, rrvt, rrvu, rrvv, rsrs, rsrt, rsru, rsrv,
rss, rst, rsts, rstt, rstu, rstv, rsu, rsut, rsuv, rsv, rsvs, rsvt, rsvu, rsvv, rtru, rtrv, rts, rtst, rtsv, rtts, rttu, rttv,
rtu, rtut, rtuv, rtv, rtvs, rtvt, rtvu, rtvv, ruru, rurv, rus, rut, ruts, rutt, rutu, rutv, ruu, ruv, ruvt, ruvu, ruvv,
rvrv, rvs, rvst, rvsv, rvt, rvts, rvtt, rvtu, rvtv, rvu, rvus, rvut, rvuv, rvv, rvvs, rvvt, rvvu, rvvv, sss, sst,
sstv, ssu, ssv, stst, stsu, stt, sttt, sttv, stu, stut, stv, stvt, stvu, stvv, susu, sut, suu, suv, suvt, suvv, svsv,
svt, svtt, svtv, svu, svut, svuv, svv, svvt, svvu, svvv, tttu, tttv, ttu, ttuv, ttv, ttvu, ttvv, tutu, tuu, tuv, tuvu,
tuvv, tvtv, tvu, tvuv, tvv, tvvu, tvvv, uuv, uvuv, uvv, uvvv

As will be understood by those of skill in the art, the monomers may be substituted in any manner appropriate for an intended use, so long as the resulting monomer comprises the required backbone structure shown in FIG. 1A.

In one embodiment, the macrocyclic oligoamide includes at least one residue not falling within monomer designation a. In another embodiment, the macrocyclic oligoamide includes at least one non-amino acid residue (i.e.: at least one residue not falling within monomer designation a, b, g, d, e). In one embodiment, the macrocyclic oligoamides has 3 residues. In other embodiments, the macrocyclic oligoamides has 4 residues.

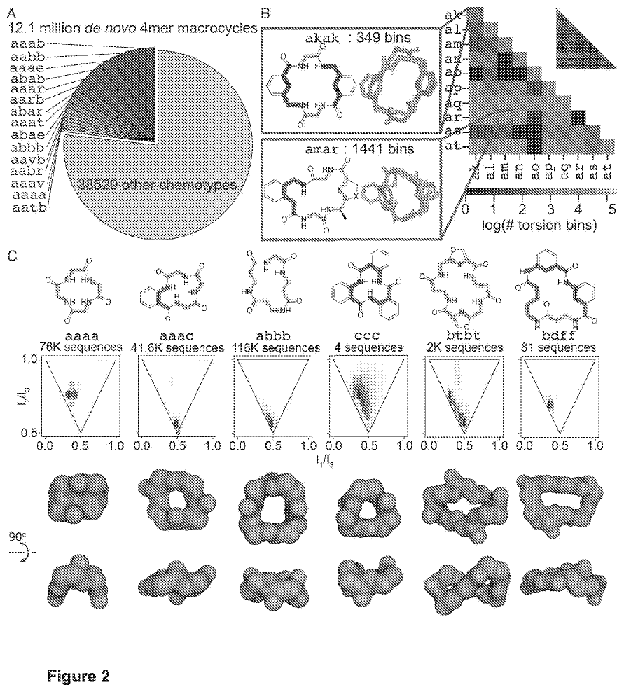

In a further embodiment, the macrocycle oligoamide has a chemotype selected from the group consisting of aaar, aarb, abar, aavb, aabr, and aatb, or circularly permuted versions thereof. These represent some of the most populated chemotypes designed using the methods described in the examples (see FIG. 2).

In one embodiment, the macrocycle oligoamide has a chemotype selected from the group consisting of akak, aaaq, and aabc, or circularly permuted versions thereof. These embodiments represent chemotypes for which X-ray and NMR structures are provided for exemplary members of the chemotype as described in the examples (see FIG. 3). In a non-limiting embodiment, aaam and aaaq macrocycles provide two different exemplary ways to mimic the extended conformations of poly-α-amino acid peptides, like those adopted by many protease and kinase substrates, and are useful for targeting these enzymes.

In another embodiment, the macrocycle oligoamide has an aas chemotype, or a circularly permuted version thereof, wherein the macrocycle oligoamide comprises two hydrogen bonds, one between backbone amides, and one involving the “s” monomer primary amide. In one embodiment, the one or more hydrogen bonds comprise a hydrogen bond between backbone amides involving non α-amino acids, which help stabilize the macrocycles. In other embodiments, the macrocycles are constructed from eight different monomer chemotypes (a, b, g, h, i, l, m, p) arranged into six unique macrocyclic chemotypes (aaam, aaap, aabi, aagb, aalm, ahah), and are stabilized by a transannular hydrogen bond between backbone amides involving non α-amino acids. As described in the examples, the aalm-based macrocycle contains a hydrogen-bonding fragment built from monomers with predominantly sp2 hybridized atoms in the backbone (lm), the aagb-based macrocycle contains a hydrogen-bonding fragment whose backbone contains many more sp3 hybridized atoms than present in α-amino acid backbones (gb), and the aabi-based macrocycle contains a hydrogen-bonding fragment that blends these two features (bi). Six of the macrocycles contain contiguous fragments of α-amino acids that mimic β-turns common in protein-protein interfaces: the aagb-, aabi-, and aaam-based macrocycles contain turns akin to type-I β-turns, and the aalm-based macrocycle contains a turn akin to the type-II β-turn. The N-methylated amino acid residues present in both aaap-based macrocycles adopt cis amides, resulting in type-VI like turns. Despite the presence of these obvious β-turn-like features in the aaam macrocycle, the spacing and orientation of the two phenylalanine side chains mimic the spacing and orientation of side chains positioned at i and i+4 of an α-helix (FIG. S10; this spacing is not present in either of the two aaap-based macrocycles which are isomers of aaam at the level of the backbone atoms). This mimicry of beta turn and helical arrangements permits use of macrocycles of this embodiment to target proteins recognizing these structural elements.

In a further embodiment, the macrocycle oligoamide has an aaam or akak chemotype, or circularly permuted version thereof, wherein any nitrogen in the backbone is in a tertiary amide.

In one embodiment, the macrocycle oligoamide has an aaaq chemotype, or a circularly permuted version thereof, wherein one of the “a” monomers comprises a pentafluorophenylalanine residue or enantiomer thereof, and the “q” residue comprises a 4-aminomethylphenylacetic acid residue or enantiomer thereof.

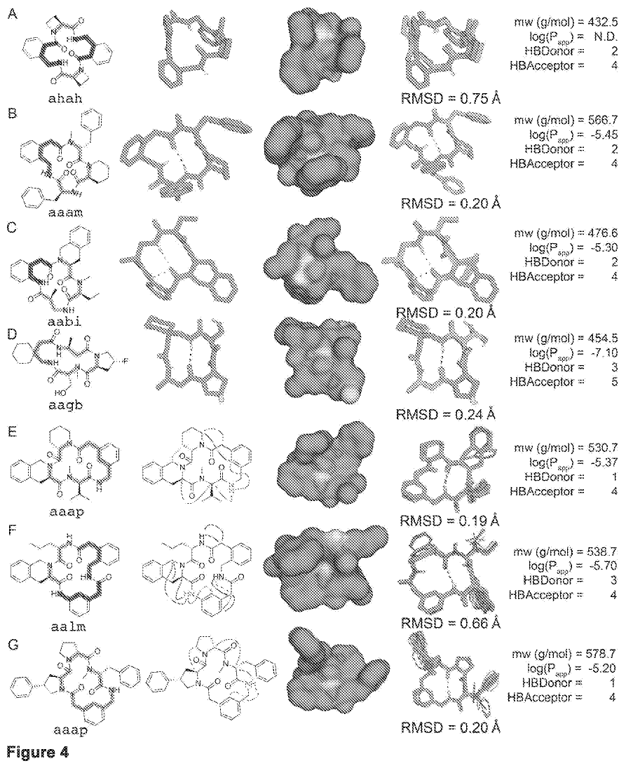

In a further embodiment, the macrocycle oligoamide has a chemotype selected from the group consisting of ahah, aabi, and aalm, or circularly permuted versions thereof. These chemotypes are some for which X-ray and NMR structures are provided for exemplary members of the chemotype as described in the examples (see FIG. 4).

In one embodiment of any embodiment or combination of embodiments herein, the macrocyclic oligoamide is membrane permeable. As described in the examples, a large percentage of the macrocycles tested were quite membrane permeable. In one embodiment, the macrocyclic oligoamide have membrane permeability of log(Pₐₚₚ)'s greater than −6, as determined using the parallel artificial membrane permeability assay (PAMPA) described in the examples. In one embodiment, the macrocyclic oligoamide does not comprise exposed polar groups, such as side chain hydroxyls and/or primary amides. Such embodiments tended to have lower permeabilities.

In various further embodiments, the macrocyclic oligoamide comprises or consists of a structure of any macrocyclic oligoamide shown in any one of Figures enantiomer thereof, circularly permuted versions thereof, or salts thereof. In one embodiment, the macrocyclic oligoamide comprises or consists of a structure of any macrocyclic oligoamide shown in any one of FIGS. 3-4, 12, and 19-47, or salts thereof. As noted herein, each monomer within these macrocyclic oligoamides may independently be substituted in any manner appropriate for an intended use, so long as the resulting monomer comprises the required backbone structure shown in Table 1.

In one embodiment ofany embodiment herein, the macrocyclic oligoamide comprises a substitution of 1, 2, 3, or all 4 monomer subunits. Any substitution may be made as suitable for an intended purpose. As described in the examples that follow, a large percentage of the macrocyclic oligoamides of the disclosure are membrane permeable, and thus may be conjugated to any moiety of interest for which such membrane permeability is useful. In non-limiting embodiments, the moiety may comprise a therapeutic agent, a diagnostic agent, a marker, linkers, dyes, purification tags, peptides, small molecules, nucleic acids, etc.

In a further embodiment, the macrocyclic oligoanmide comprises or consists of the structure of any one of compounds 1-218 in Table 3, or salts thereof. The compounds shown in Table 3 were designed for binding to specific targets, as described in the examples.

TABLE 3

| Compound | | |
|---|---|---|
| No. | Compound name | Compound structure |
| 1 | PJS-BCLM-1 | 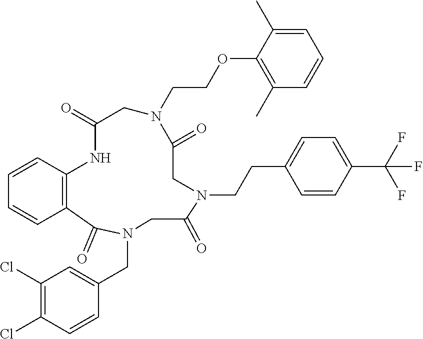 [SMILES: Cc1cccc(C)c1OCCN1CC(=O)Nc2ccccc2C(=O)N(Cc2ccc(Cl)c(Cl)c2)CC(=O)N(CCc2ccc(C(F)(F)F)cc2)CC1=O] |
| 2 | PJS-BCLM-2 | 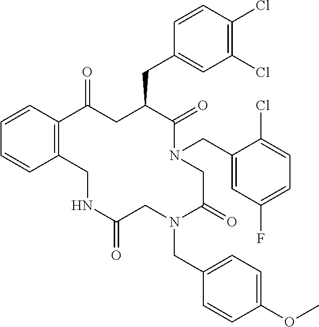 [SMILES: COc1ccc(CN2CC(=O)NCc3ccccc3C(=O)C[C@@H](Cc3ccc(Cl)c(Cl)c3)C(=O)N(Cc3cc(F)ccc3Cl)CC2=O)cc1] |
| 3 | PJS-BCLM-3 | 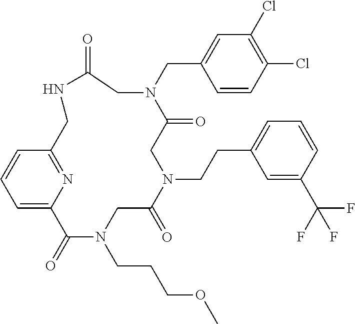 [SMILES: COCCCN1CC(=O)N(CCc2cccc(C(F)(F)F)c2)CC(=O)N(Cc2ccc(Cl)c(Cl)c2)CC(=O)NCc2cccc(n2)C1=O] |
| 4 | PJS-BCLM-4 | 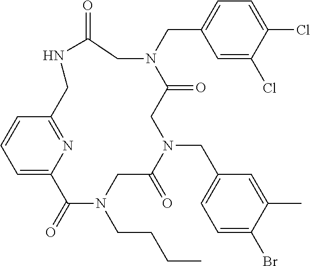 [SMILES: CCCCN1CC(=O)N(Cc2ccc(Br)c(C)c2)CC(=O)N(Cc2ccc(Cl)c(Cl)c2)CC(=O)NCc2cccc(n2)C1=O] |
| 5 | PJS-BCLM-5 | 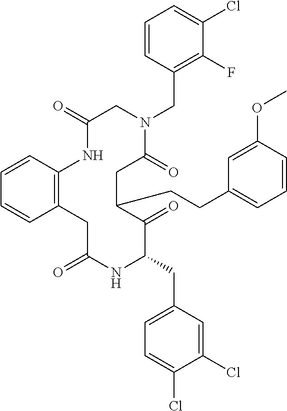 [SMILES: COc1cccc(CCC2CC(=O)N(Cc3cccc(Cl)c3F)CC(=O)Nc3ccccc3CC(=O)N[C@@H](Cc3ccc(Cl)c(Cl)c3)C2=O)c1] |
| 6 | PJS-BCLM-6 | 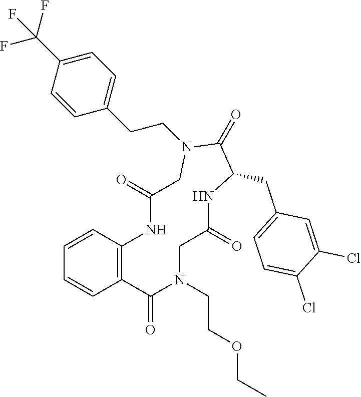 [SMILES: CCOCCN1CC(=O)N[C@@H](Cc2ccc(Cl)c(Cl)c2)C(=O)N(CCc2ccc(C(F)(F)F)cc2)CC(=O)Nc2ccccc2C1=O] |
| 7 | PJS-BCLM-7 | 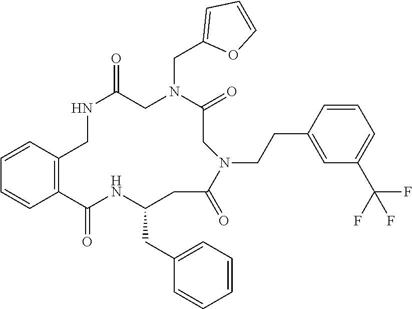 [SMILES: O=C1CN(Cc2ccco2)C(=O)CN(CCc2cccc(C(F)(F)F)c2)C(=O)C[C@H](Cc2ccccc2)NC(=O)c2ccccc2CN1] |
| 8 | PJS-BCLM-8 | 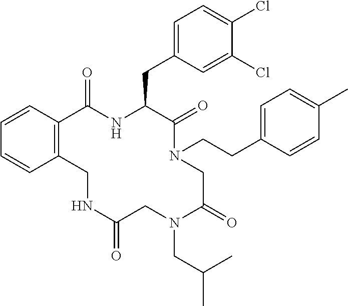 [SMILES: Cc1ccc(CCN2CC(=O)N(CC(C)C)CC(=O)NCc3ccccc3C(=O)N[C@@H](Cc3ccc(Cl)c(Cl)c3)C2=O)cc1] |
| 9 | PJS-BCLM-9 | 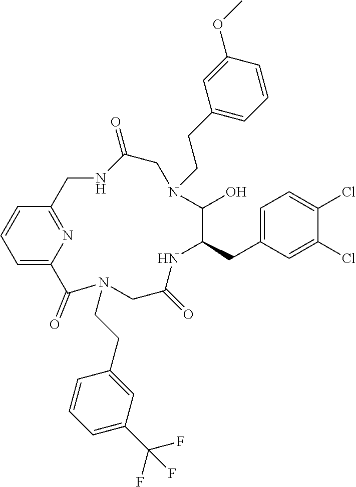 [SMILES: COc1cccc(CCN2CC(=O)NCc3cccc(n3)C(=O)N(CCc3cccc(C(F)(F)F)c3)CC(=O)N[C@H](Cc3ccc(Cl)c(Cl)c3)C2O)c1] |
| 10 | PJS-BCLM-10 | 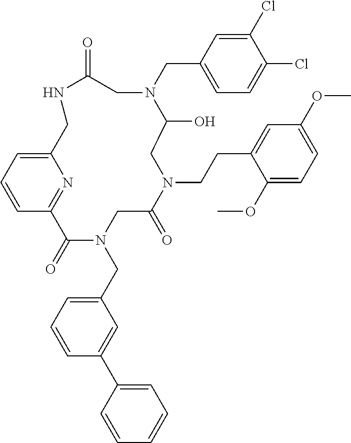 [SMILES: COc1ccc(OC)c(CCN2CC(O)N(Cc3ccc(Cl)c(Cl)c3)CC(=O)NCc3cccc(n3)C(=O)N(Cc3cccc(-c4ccccc4)c3)CC2=O)c1] |
| 11 | PJS-BCLM-11 | 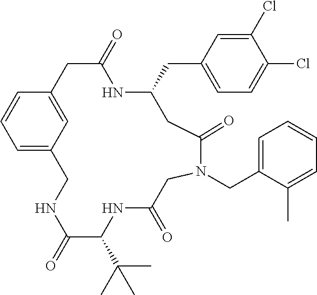 [SMILES: Cc1ccccc1CN1CC(=O)N[C@H](C(C)(C)C)C(=O)NCc2cccc(c2)CC(=O)N[C@H](Cc2ccc(Cl)c(Cl)c2)CC1=O] |
| 12 | PJS-BCLM-12 | 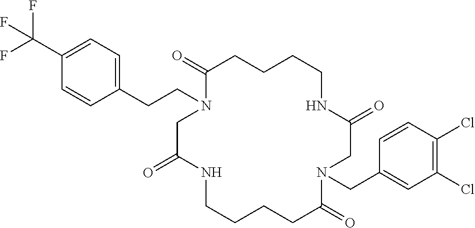 [SMILES: O=C1CN(CCc2ccc(C(F)(F)F)cc2)C(=O)CCCCNC(=O)CN(Cc2ccc(Cl)c(Cl)c2)C(=O)CCCCN1] |
| 13 | PJS-BCLM-13 | 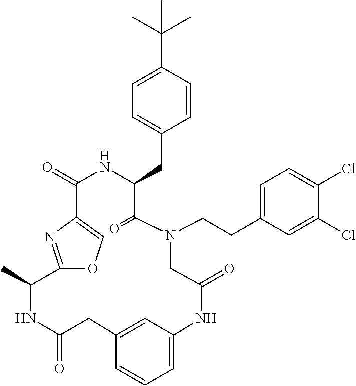 [SMILES: C[C@@H]1NC(=O)Cc2cccc(c2)NC(=O)CN(CCc2ccc(Cl)c(Cl)c2)C(=O)[C@H](Cc2ccc(C(C)(C)C)cc2)NC(=O)c2coc1n2] |
| 14 | PJS-BCLM-14 | 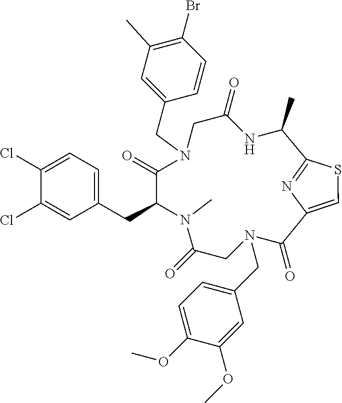 [SMILES: COc1ccc(CN2CC(=O)N(C)[C@@H](Cc3ccc(Cl)c(Cl)c3)C(=O)N(Cc3ccc(Br)c(C)c3)CC(=O)N[C@@H](C)c3nc(cs3)C2=O)cc1OC] |
| 15 | PJS-BCLM-15 | 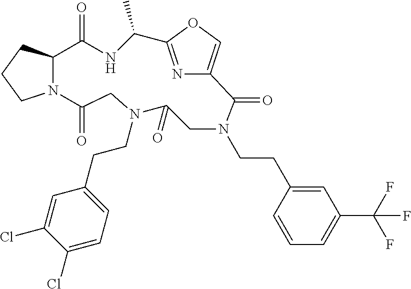 [SMILES: C[C@H]1NC(=O)[C@@H]2CCCN2C(=O)CN(CCc2ccc(Cl)c(Cl)c2)C(=O)CN(CCc2cccc(C(F)(F)F)c2)C(=O)c2coc1n2] |
| 16 | PJS-BCLM-16 | 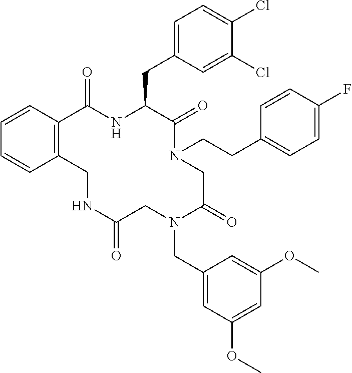 [SMILES: COc1cc(CN2CC(=O)NCc3ccccc3C(=O)N[C@@H](Cc3ccc(Cl)c(Cl)c3)C(=O)N(CCc3ccc(F)cc3)CC2=O)cc(OC)c1] |
| 17 | PJS-BCLM-17 | 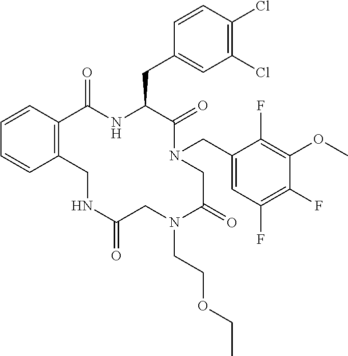 [SMILES: CCOCCN1CC(=O)NCc2ccccc2C(=O)N[C@@H](Cc2ccc(Cl)c(Cl)c2)C(=O)N(Cc2cc(F)c(F)c(OC)c2F)CC1=O] |
| 18 | PJS-BCLM-18 | 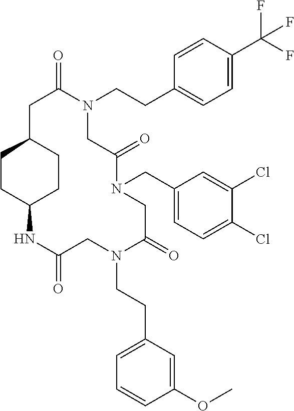 [SMILES: COc1cccc(CCN2CC(=O)N[C@H]3CC[C@H](CC3)CC(=O)N(CCc3ccc(C(F)(F)F)cc3)CC(=O)N(Cc3ccc(Cl)c(Cl)c3)CC2=O)c1] |
| 19 | PJS-BCLM-19 | 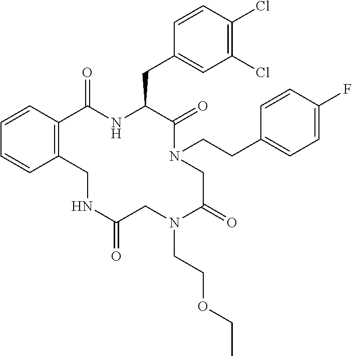 [SMILES: CCOCCN1CC(=O)NCc2ccccc2C(=O)N[C@@H](Cc2ccc(Cl)c(Cl)c2)C(=O)N(CCc2ccc(F)cc2)CC1=O] |
| 20 | PJS-BCLM-20 | 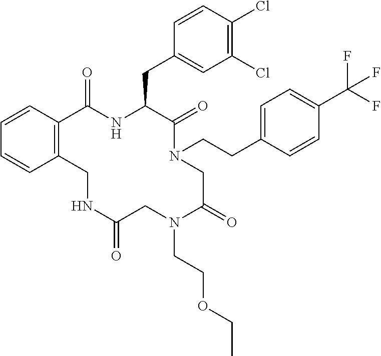 [SMILES: CCOCCN1CC(=O)NCc2ccccc2C(=O)N[C@@H](Cc2ccc(Cl)c(Cl)c2)C(=O)N(CCc2ccc(C(F)(F)F)cc2)CC1=O] |
| 21 | PJS-BCLM-21 | 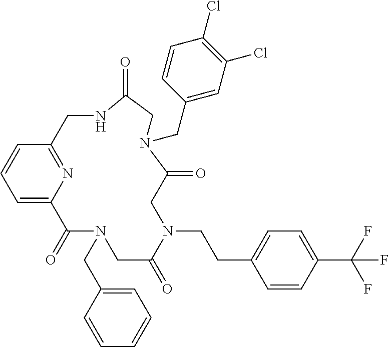 [SMILES: O=C1CN(Cc2ccc(Cl)c(Cl)c2)C(=O)CN(CCc2ccc(C(F)(F)F)cc2)C(=O)CN(Cc2ccccc2)C(=O)c2cccc(n2)CN1] |
| 22 | PJS-BCLM-22 | 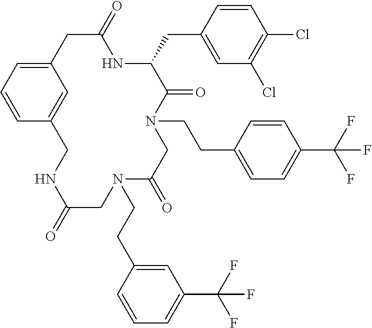 [SMILES: O=C1CN(CCc2cccc(C(F)(F)F)c2)C(=O)CN(CCc2ccc(C(F)(F)F)cc2)C(=O)[C@@H](Cc2ccc(Cl)c(Cl)c2)NC(=O)Cc2cccc(c2)CN1] |
| 23 | PJS-BCLM-23 | 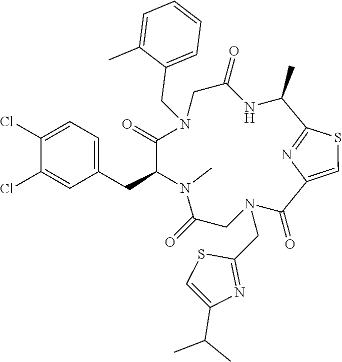 [SMILES: Cc1ccccc1CN1CC(=O)N[C@@H](C)c2nc(cs2)C(=O)N(Cc2nc(C(C)C)cs2)CC(=O)N(C)[C@@H](Cc2ccc(Cl)c(Cl)c2)C1=O] |
| 24 | PJS-BCLM-24 | 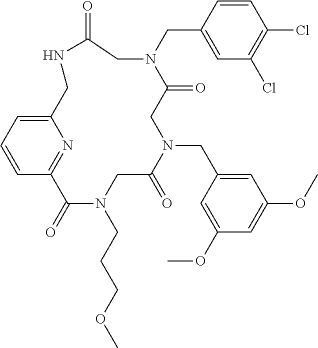 [SMILES: COCCCN1CC(=O)N(Cc2cc(OC)cc(OC)c2)CC(=O)N(Cc2ccc(Cl)c(Cl)c2)CC(=O)NCc2cccc(n2)C1=O] |
| 25 | PJS-BCLM-25 | 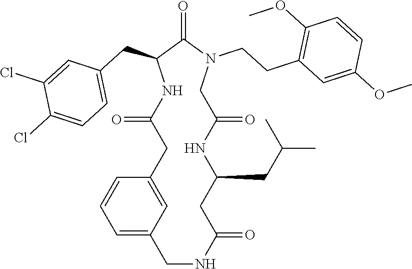 [SMILES: COc1ccc(OC)c(CCN2CC(=O)N[C@@H](CC(C)C)CC(=O)NCc3cccc(c3)CC(=O)N[C@@H](Cc3ccc(Cl)c(Cl)c3)C2=O)c1] |
| 26 | PJS-BCLM-26 | 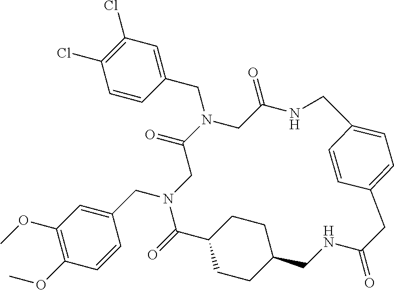 [SMILES: COc1ccc(CN2CC(=O)N(Cc3ccc(Cl)c(Cl)c3)CC(=O)NCc3ccc(cc3)CC(=O)NC[C@H]3CC[C@@H](CC3)C2=O)cc1OC] |
| 27 | PJS-BCLM-27 | 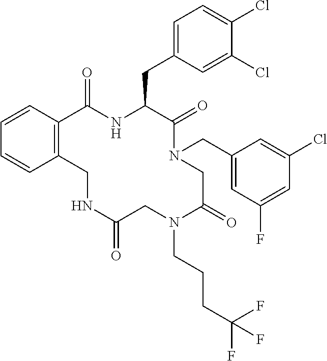 [SMILES: O=C1CN(CCCC(F)(F)F)C(=O)CN(Cc2cc(F)cc(Cl)c2)C(=O)[C@H](Cc2ccc(Cl)c(Cl)c2)NC(=O)c2ccccc2CN1] |
| 28 | PJS-BCLM-28 | 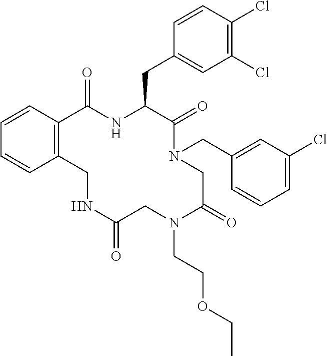 [SMILES: CCOCCN1CC(=O)NCc2ccccc2C(=O)N[C@@H](Cc2ccc(Cl)c(Cl)c2)C(=O)N(Cc2cccc(Cl)c2)CC1=O] |
| 29 | PJS-BCLM-29 | 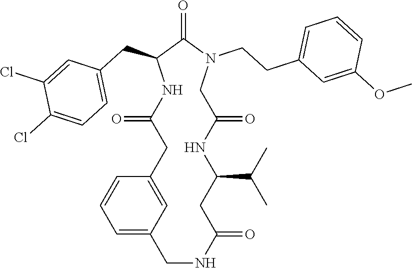 [SMILES: COc1cccc(CCN2CC(=O)N[C@@H](C(C)C)CC(=O)NCc3cccc(c3)CC(=O)N[C@@H](Cc3ccc(Cl)c(Cl)c3)C2=O)c1] |
| 30 | PJS-BCLM-30 | 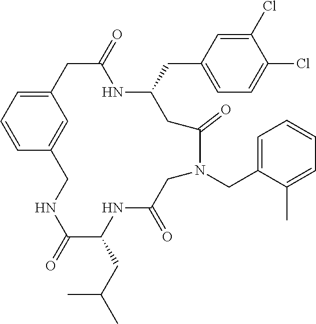 [SMILES: Cc1ccccc1CN1CC(=O)N[C@H](CC(C)C)C(=O)NCc2cccc(c2)CC(=O)N[C@H](Cc2ccc(Cl)c(Cl)c2)CC1=O] |
| 31 | PJS-MDM2-chin-1 | 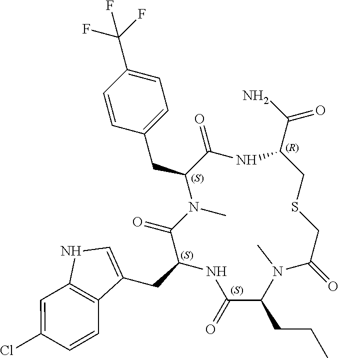 |
| 32 | PJS-MDM2-chin-2 | 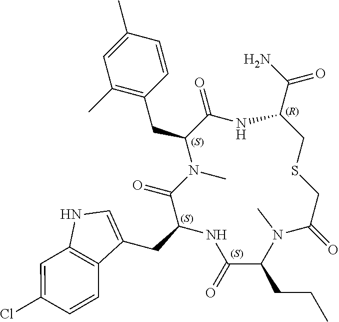 |
| 33 | PJS-MDM2-chin-3 | 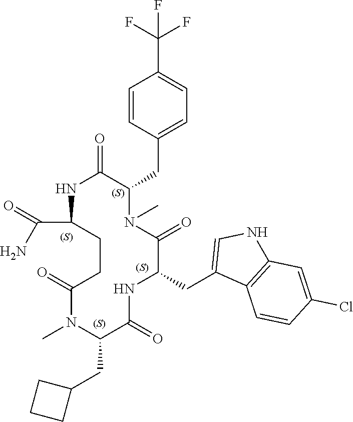 |
| 34 | PJS-MDM2-chin-4 | 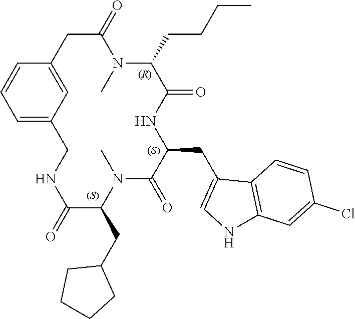 |
| 35 | PJS-MDM2-chin-5 | 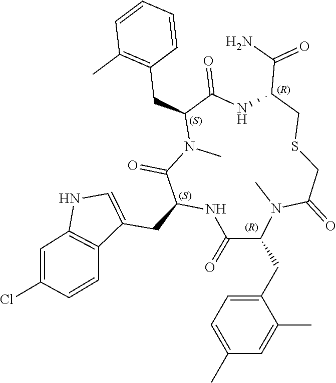 |
| 36 | PJS-MDM2-chin-6 | 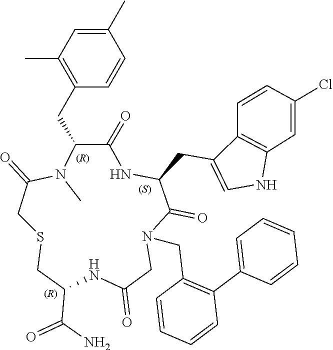 |
| 37 | PJS-MDM2-chin-7 | 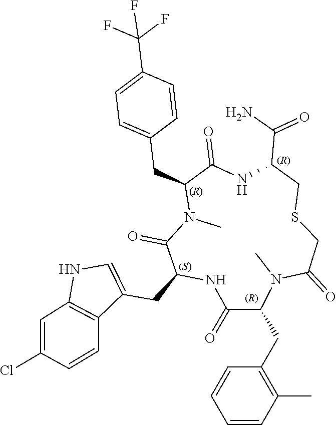 |
| 38 | PJS-MDM2-chin-8 | 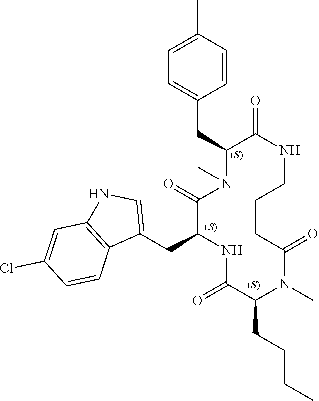 |
| 39 | PJS-MDM2-chin-9 | 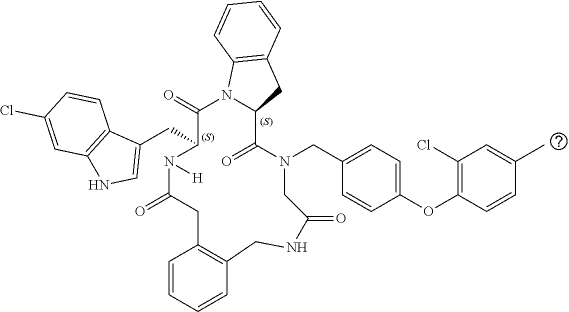 |
| 40 | PJS-MDM2-chin-10 | 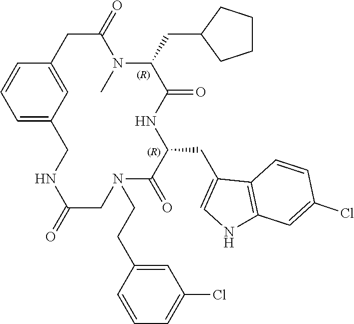 |
| 41 | PJS-MDM2-mCl-1 | 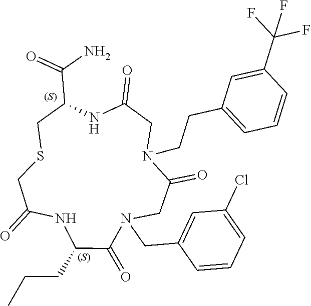 |
| 42 | PJS-MDM2-mCl-2 | 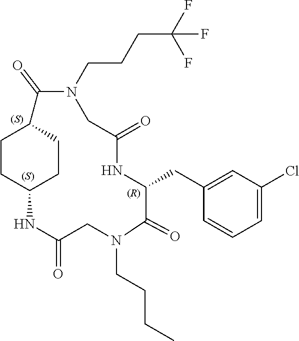 |
| 43 | PJS-MDM2-mCl-3 | 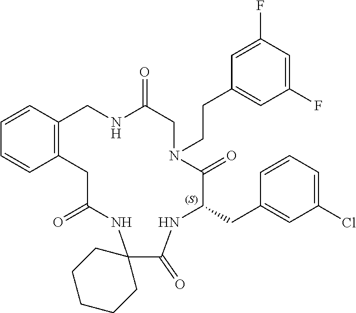 |
| 44 | PJS-MDM2-mCl-4 | 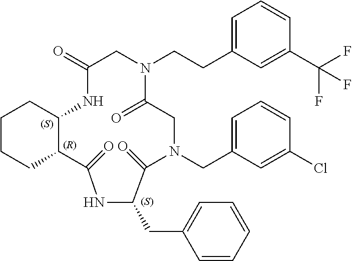 |
| 45 | PJS-MDM2-mCl-5 | 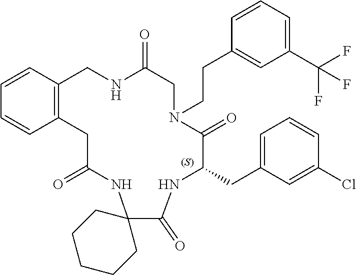 |
| 46 | PJS-MDM2-mCl-6 | 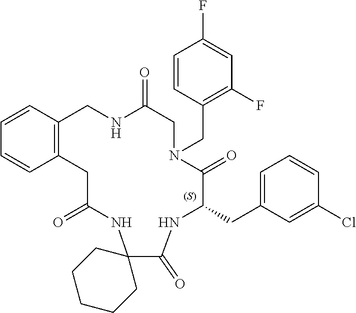 |
| 47 | PJS-MDM2-mCl-7 | 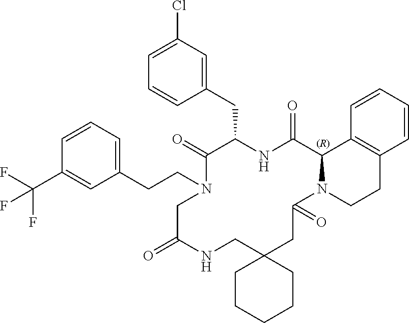 |
| 48 | PJS-MDM2-mCl-8 | 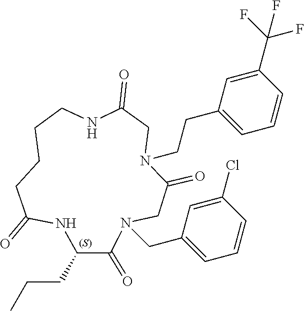 |
| 49 | PJS-MDM2-mCl-9 | 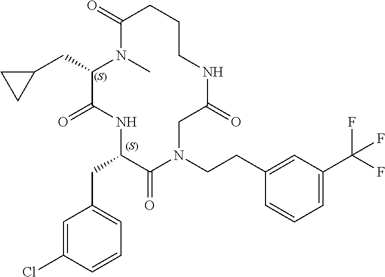 |
| 50 | PJS-MDM2-mCl-10 | 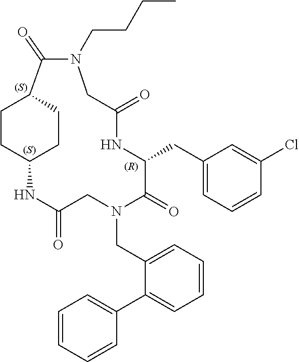 |
| 51 | PJS-MDM2-mCl-11 | 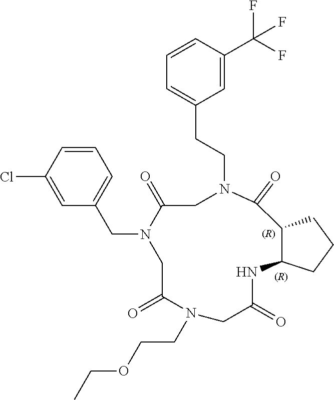 |
| 52 | PJS-MDM2-pCl-1 | 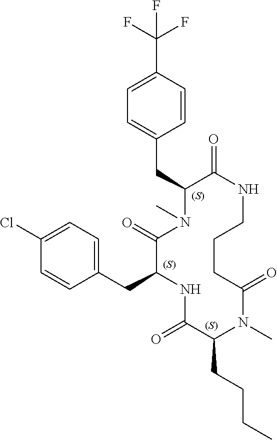 |
| 53 | PJS-MDM2-pCl-2 | 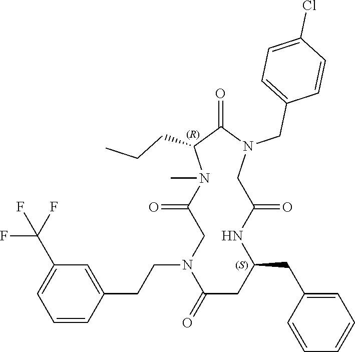 |
| 54 | PJS-MDM2-pCl-3 | 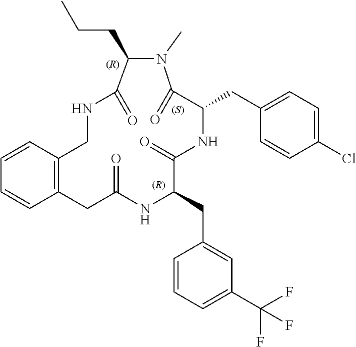 |
| 55 | PJS-MDM2-pCl-4 | 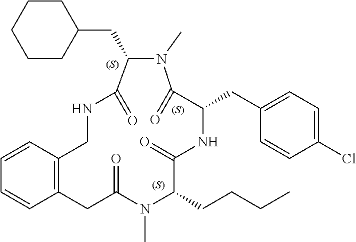 |
| 56 | PJS-MDM2-pCl-5 | 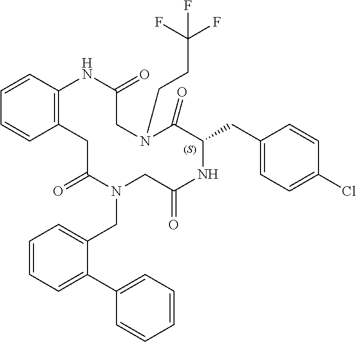 |
| 57 | PJS-MDM2-pCl-6 | 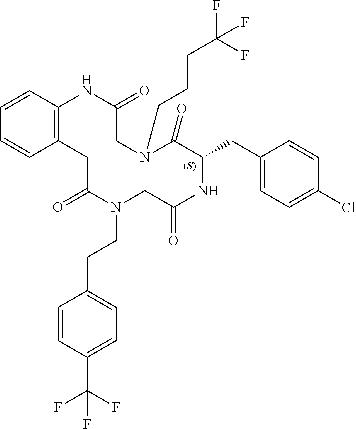 |
| 58 | PJS-MDM2-pCl-7 | 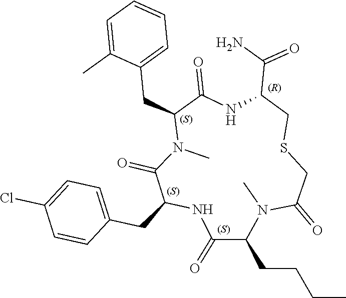 |
| 59 | PJS-MDM2-pCl-8 | 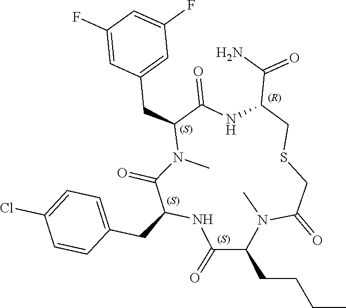 |
| 60 | PJS-FXM-1 | 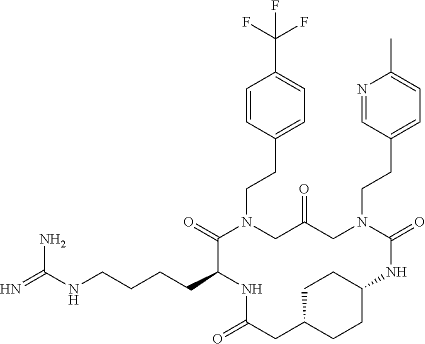 [SMILES: Cc1ccc(CCN2CC(=O)CN(CCc3ccc(C(F)(F)F)cc3)C(=O)[C@H](CCCCNC(=N)N)NC(=O)C[C@H]3CC[C@H](CC3)NC2=O)cn1] |
| 61 | PJS-FXM-2 | 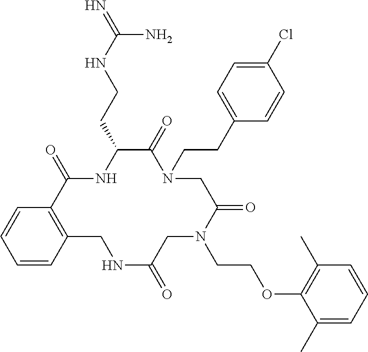 [SMILES: Cc1cccc(C)c1OCCN1CC(=O)NCc2ccccc2C(=O)N[C@H](CCNC(=N)N)C(=O)N(CCc2ccc(Cl)cc2)CC1=O] |
| 62 | PJS-FXM-3 | 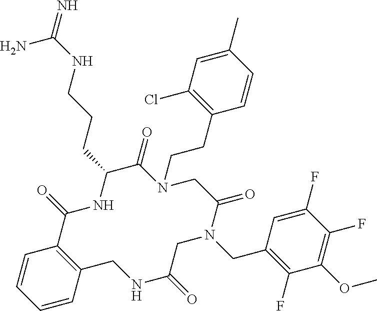 [SMILES: COc1c(F)c(F)cc(CN2CC(=O)NCc3ccccc3C(=O)N[C@H](CCCNC(=N)N)C(=O)N(CCc3ccc(C)cc3Cl)CC2=O)c1F] |
| 63 | PJS-FXM-4 | 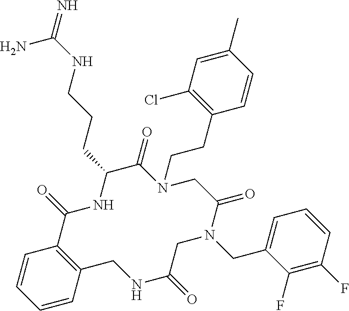 [SMILES: Cc1ccc(CCN2CC(=O)N(Cc3cccc(F)c3F)CC(=O)NCc3ccccc3C(=O)N[C@H](CCCNC(=N)N)C2=O)c(Cl)c1] |
| 64 | PJS-FXM-5 | 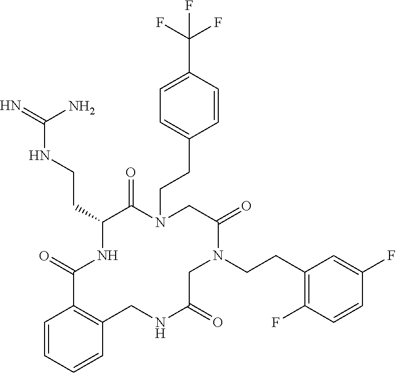 [SMILES: N=C(N)NCC[C@H]1NC(=O)c2ccccc2CNC(=O)CN(CCc2cc(F)ccc2F)C(=O)CN(CCc2ccc(C(F)(F)F)cc2)C1=O] |
| 65 | PJS-FXM-6 | 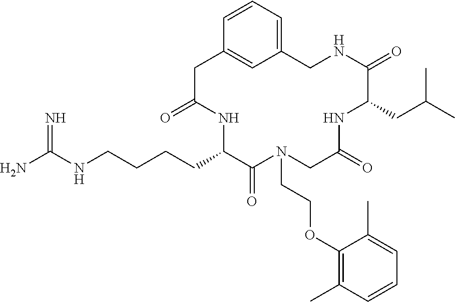 [SMILES: Cc1cccc(C)c1OCCN1CC(=O)N[C@@H](CC(C)C)C(=O)NCc2cccc(c2)CC(=O)N[C@@H](CCCCNC(=N)N)C1=O] |
| 66 | PJS-FXM-7 | 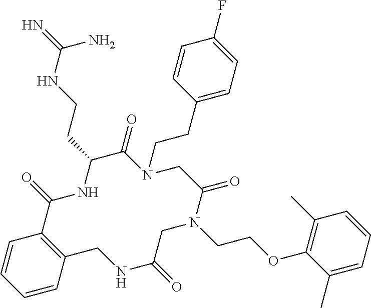 [SMILES: Cc1cccc(C)c1OCCN1CC(=O)NCc2ccccc2C(=O)N[C@H](CCNC(=N)N)C(=O)N(CCc2ccc(F)cc2)CC1=O] |
| 67 | PJS-FXM-8 | 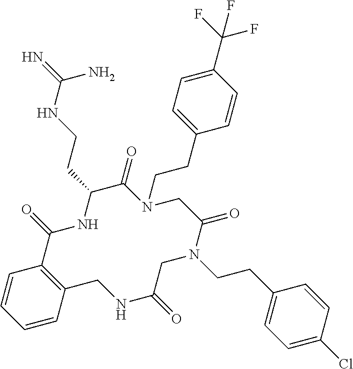 [SMILES: N=C(N)NCC[C@H]1NC(=O)c2ccccc2CNC(=O)CN(CCc2ccc(Cl)cc2)C(=O)CN(CCc2ccc(C(F)(F)F)cc2)C1=O] |
| 68 | PJS-FXM-9 | 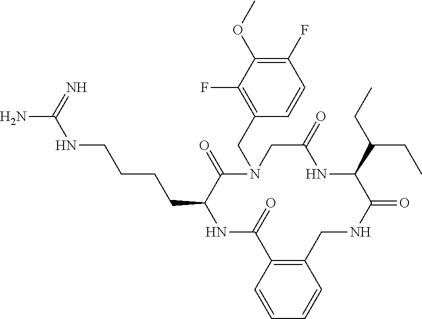 [SMILES: CCC(CC)[C@@H]1NC(=O)CN(Cc2ccc(F)c(OC)c2F)C(=O)[C@H](CCCCNC(=N)N)NC(=O)c2ccccc2CNC1=O] |
| 69 | PJS-FXM-10 | 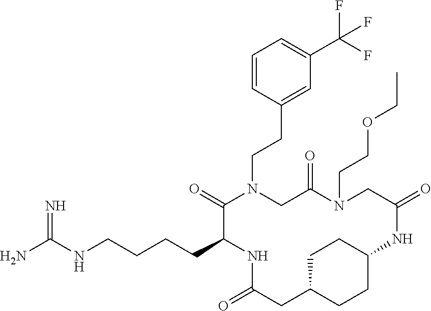 [SMILES: CCOCCN1CC(=O)N[C@H]2CC[C@H](CC2)CC(=O)N[C@@H](CCCCNC(=N)N)C(=O)N(CCc2cccc(C(F)(F)F)c2)CC1=O] |
| 70 | PJS-FXM-11 | 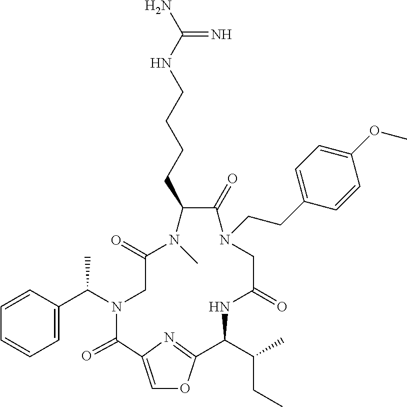 [SMILES: CC[C@@H](C)[C@@H]1NC(=O)CN(CCc2ccc(OC)cc2)C(=O)[C@H](CCCCNC(=N)N)N(C)C(=O)CN([C@@H](C)c2ccccc2)C(=O)c2coc1n2] |
| 71 | PJS-FXM-12 | 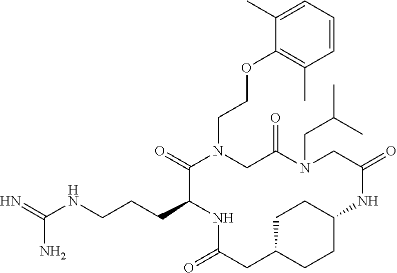 [SMILES: Cc1cccc(C)c1OCCN1CC(=O)N(CC(C)C)CC(=O)N[C@H]2CC[C@H](CC2)CC(=O)N[C@@H](CCCNC(=N)N)C1=O] |
| 72 | PJS-FXM-13 | 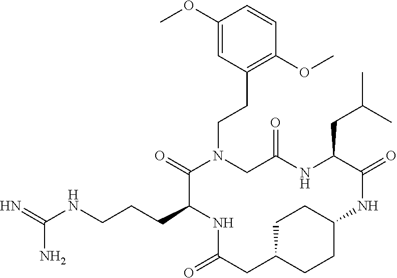 [SMILES: COc1ccc(OC)c(CCN2CC(=O)N[C@@H](CC(C)C)C(=O)N[C@H]3CC[C@H](CC3)CC(=O)N[C@@H](CCCNC(=N)N)C2=O)c1] |
| 73 | PJS-FXM-14 | 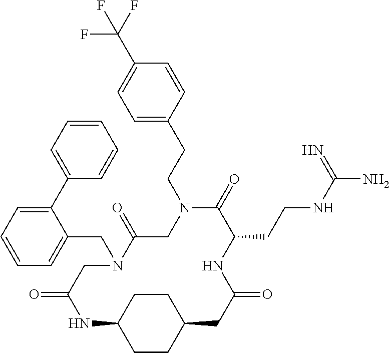 [SMILES: N=C(N)NCC[C@@H]1NC(=O)C[C@H]2CC[C@H](CC2)NC(=O)CN(Cc2ccccc2-c2ccccc2)C(=O)CN(CCc2ccc(C(F)(F)F)cc2)C1=O] |
| 74 | PJS-FXM-15 | 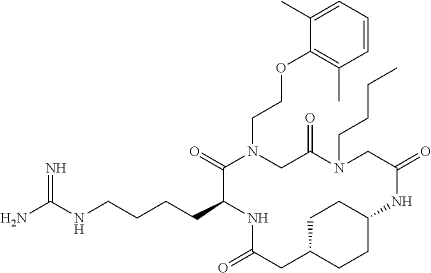 [SMILES: CCCCN1CC(=O)N[C@H]2CC[C@H](CC2)CC(=O)N[C@@H](CCCCNC(=N)N)C(=O)N(CCOc2c(C)cccc2C)CC1=O] |
| 75 | PJS-FXM-16 | 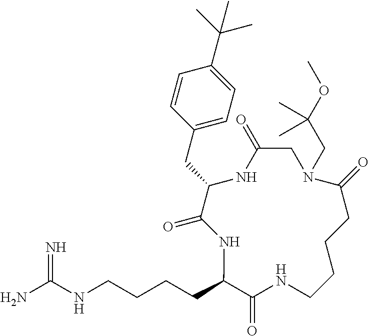 [SMILES: COC(C)(C)CN1CC(=O)N[C@@H](Cc2ccc(C(C)(C)C)cc2)C(=O)N[C@H](CCCCNC(=N)N)C(=O)NCCCCC1=O] |
| 76 | PJS-FXM-17 | 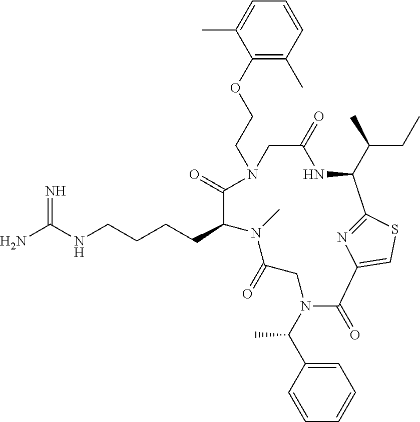 [SMILES: CC[C@H](C)[C@@H]1NC(=O)CN(CCOc2c(C)cccc2C)C(=O)[C@H](CCCCNC(=N)N)N(C)C(=O)CN([C@@H](C)c2ccccc2)C(=O)c2csc1n2] |
| 77 | PJS-FXM-18 | 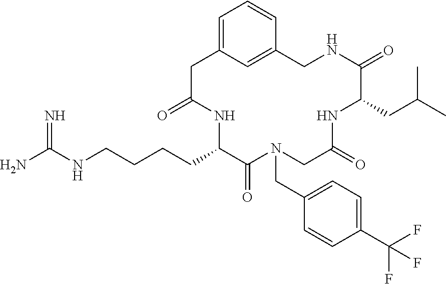 [SMILES: CC(C)C[C@@H]1NC(=O)CN(Cc2ccc(C(F)(F)F)cc2)C(=O)[C@H](CCCCNC(=N)N)NC(=O)Cc2cccc(c2)CNC1=O] |
| 78 | PJS-FXM-19 | 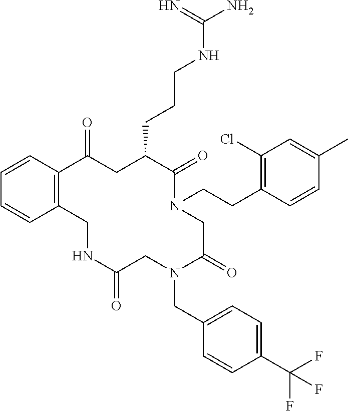 [SMILES: Cc1ccc(CCN2CC(=O)N(Cc3ccc(C(F)(F)F)cc3)CC(=O)NCc3ccccc3C(=O)C[C@H](CCCNC(=N)N)C2=O)c(Cl)c1] |
| 79 | PJS-FXM-20 | 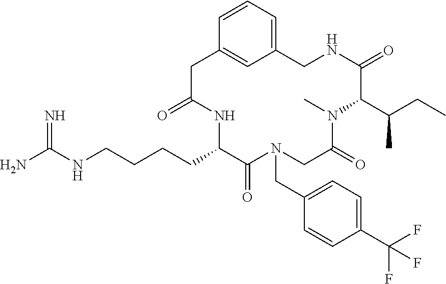 [SMILES: CC[C@@H](C)[C@H]1C(=O)NCc2cccc(c2)CC(=O)N[C@@H](CCCCNC(=N)N)C(=O)N(Cc2ccc(C(F)(F)F)cc2)CC(=O)N1C] |
| 80 | PJS-FXM-21 | 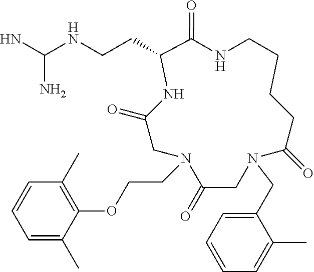 [SMILES: Cc1ccccc1CN1CC(=O)N(CCOc2c(C)cccc2C)CC(=O)N[C@H](CCNC(N)N)C(=O)NCCCCC1=O] |
| 81 | PJS-MDM2M-1 | 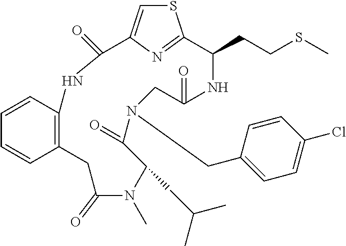 [SMILES: CSCC[C@H]1NC(=O)CN(Cc2ccc(Cl)cc2)C(=O)[C@H](CC(C)C)N(C)C(=O)Cc2ccccc2NC(=O)c2csc1n2] |
| 82 | PJS-MDM2M-2 | 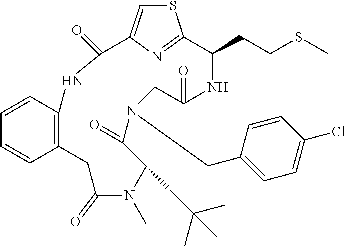 [SMILES: CSCC[C@H]1NC(=O)CN(Cc2ccc(Cl)cc2)C(=O)[C@H](CC(C)(C)C)N(C)C(=O)Cc2ccccc2NC(=O)c2csc1n2] |
| 83 | PJS-MDM2M-3 | 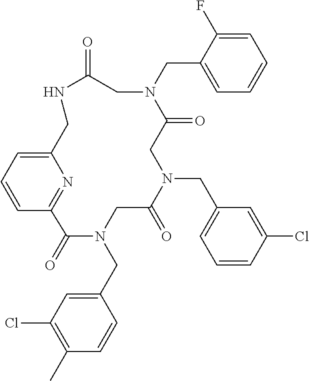 [SMILES: Cc1ccc(CN2CC(=O)N(Cc3cccc(Cl)c3)CC(=O)N(Cc3ccccc3F)CC(=O)NCc3cccc(n3)C2=O)cc1Cl] |
| 84 | PJS-MDM2M-4 | 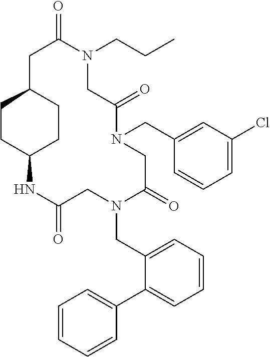 [SMILES: CCCN1CC(=O)N(Cc2cccc(Cl)c2)CC(=O)N(Cc2ccccc2-c2ccccc2)CC(=O)N[C@H]2CC[C@H](CC2)CC1=O] |
| 85 | PJS-MDM2M-5 | 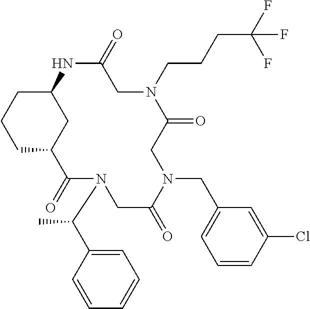 [SMILES: C[C@@H](c1ccccc1)N1CC(=O)N(Cc2cccc(Cl)c2)CC(=O)N(CCCC(F)(F)F)CC(=O)N[C@@H]2CCC[C@H](C2)C1=O] |
| 86 | PJS-MDM2M-6 | 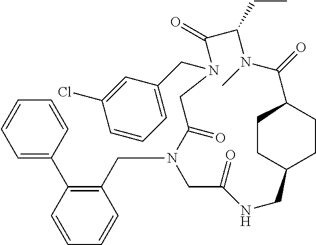 [SMILES: CC[C@H]1C(=O)N(Cc2cccc(Cl)c2)CC(=O)N(Cc2ccccc2-c2ccccc2)CC(=O)NC[C@H]2CC[C@H](CC2)C(=O)N1C] |
| 87 | PJS-MDM2M-7 | 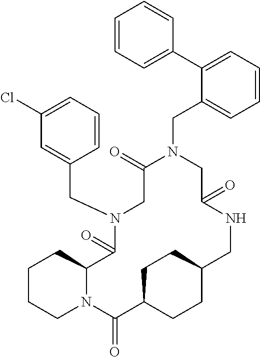 [SMILES: O=C1CN(Cc2ccccc2-c2ccccc2)C(=O)CN(Cc2cccc(Cl)c2)C(=O)[C@@H]2CCCCN2C(=O)[C@H]2CC[C@H](CC2)CN1] |
| 88 | PJS-MDM2M-8 | 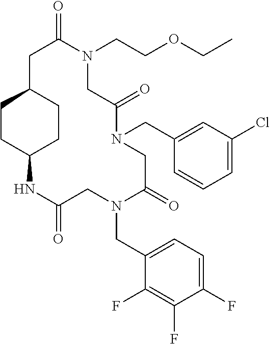 [SMILES: CCOCCN1CC(=O)N(Cc2cccc(Cl)c2)CC(=O)N(Cc2ccc(F)c(F)c2F)CC(=O)N[C@H]2CC[C@H](CC2)CC1=O] |
| 89 | PJS-MDM2M-9 | 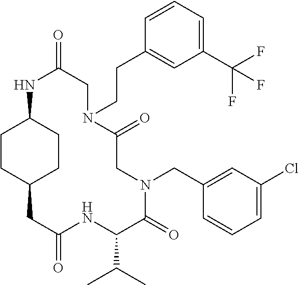 [SMILES: CC(C)[C@@H]1NC(=O)C[C@H]2CC[C@H](CC2)NC(=O)CN(CCc2cccc(C(F)(F)F)c2)C(=O)CN(Cc2cccc(Cl)c2)C1=O] |
| 90 | PJS-MDM2M-10 | 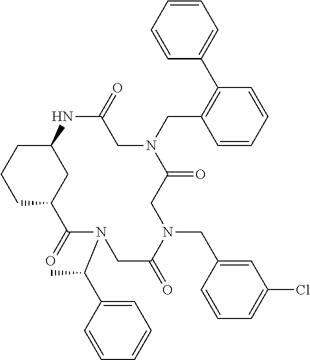 [SMILES: C[C@@H](c1ccccc1)N1CC(=O)N(Cc2cccc(Cl)c2)CC(=O)N(Cc2ccccc2-c2ccccc2)CC(=O)N[C@@H]2CCC[C@H](C2)C1=O] |
| 91 | PJS-MDM2M-11 | 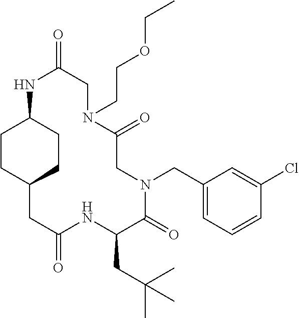 [SMILES: CCOCCN1CC(=O)N[C@H]2CC[C@H](CC2)CC(=O)N[C@H](CC(C)(C)C)C(=O)N(Cc2cccc(Cl)c2)CC1=O] |
| 92 | PJS-MDM2M-12 | 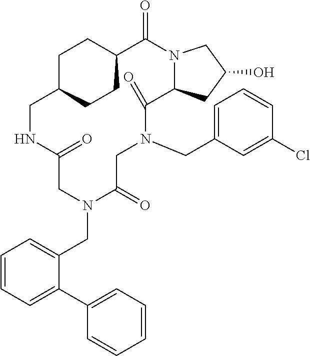 [SMILES: O=C1CN(Cc2ccccc2-c2ccccc2)C(=O)CN(Cc2cccc(Cl)c2)C(=O)[C@@H]2C[C@@H](O)CN2C(=O)[C@H]2CC[C@H](CC2)CN1] |
| 93 | PJS-MDM2M-13 | 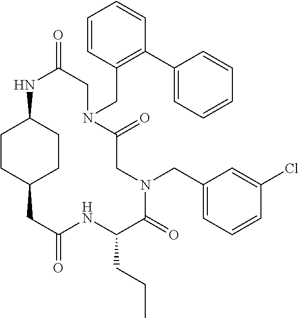 [SMILES: CCC[C@@H]1NC(=O)C[C@H]2CC[C@H](CC2)NC(=O)CN(Cc2ccccc2-c2ccccc2)C(=O)CN(Cc2cccc(Cl)c2)C1=O] |
| 94 | PJS-MDM2M-14 | 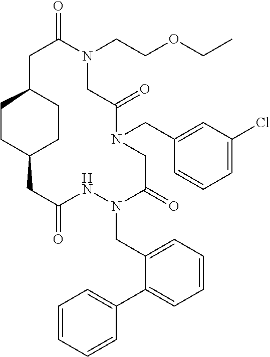 [SMILES: CCOCCN1CC(=O)N(Cc2cccc(Cl)c2)CC(=O)N(Cc2ccccc2-c2ccccc2)NC(=O)C[C@H]2CC[C@H](CC2)CC1=O] |
| 95 | PJS-MDM2M-15 | 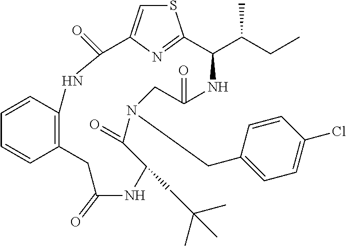 [SMILES: CC[C@@H](C)[C@H]1NC(=O)CN(Cc2ccc(Cl)cc2)C(=O)[C@H](CC(C)(C)C)NC(=O)Cc2ccccc2NC(=O)c2csc1n2] |
| 96 | PJS-MDM2M-16 | 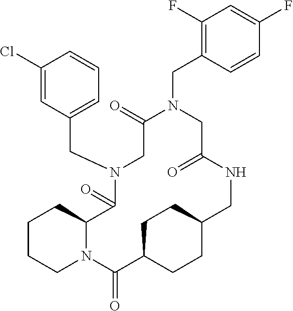 [SMILES: O=C1CN(Cc2ccc(F)cc2F)C(=O)CN(Cc2cccc(Cl)c2)C(=O)[C@@H]2CCCCN2C(=O)[C@H]2CC[C@H](CC2)CN1] |
| 97 | PJS-MDM2M-17 | 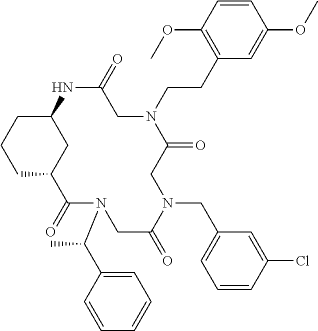 [SMILES: COc1ccc(OC)c(CCN2CC(=O)N[C@@H]3CCC[C@H](C3)C(=O)N([C@@H](C)c3ccccc3)CC(=O)N(Cc3cccc(Cl)c3)CC2=O)c1] |
| 98 | PJS-MDM2M-18 | 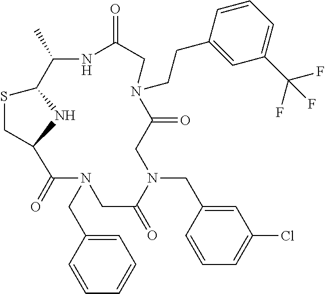 [SMILES: C[C@@H]1NC(=O)CN(CCc2cccc(C(F)(F)F)c2)C(=O)CN(Cc2cccc(Cl)c2)C(=O)CN(Cc2ccccc2)C(=O)[C@H]2CS[C@H]1N2] |
| 99 | PJS-CLPP-aaab~1 | 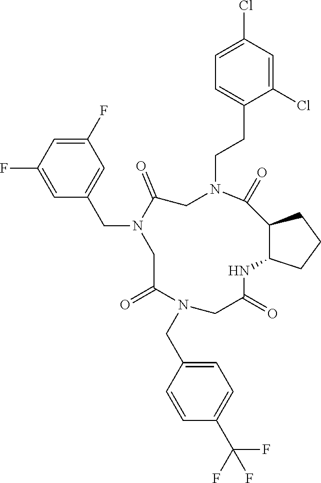 [SMILES: O=C1CN(Cc2ccc(C(F)(F)F)cc2)C(=O)CN(Cc2cc(F)cc(F)c2)C(=O)CN(CCc2ccc(Cl)cc2Cl)C(=O)[C@H]2CCC[C@@H]2N1] |
| 100 | PJS-CLPP-aaai-1 | 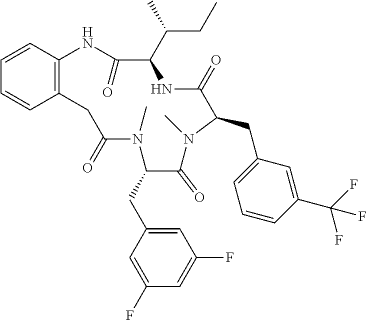 [SMILES: CC[C@@H](C)[C@H]1NC(=O)[C@@H](Cc2cccc(C(F)(F)F)c2)N(C)C(=O)[C@H](Cc2cc(F)cc(F)c2)N(C)C(=O)Cc2ccccc2NC1=O] |
| 101 | PJS-CLPP-aaai-2 | 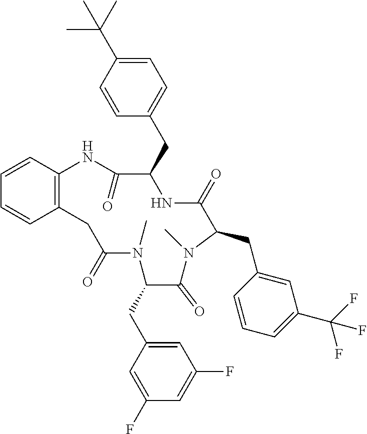 [SMILES: CN1C(=O)[C@H](Cc2cc(F)cc(F)c2)N(C)C(=O)Cc2ccccc2NC(=O)[C@@H](Cc2ccc(C(C)(C)C)cc2)NC(=O)[C@H]1Cc1cccc(C(F)(F)F)c1] |
| 102 | PJS-CLPP-aaab-2 | 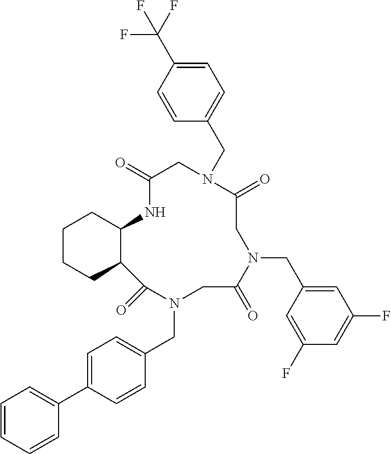 [SMILES: O=C1CN(Cc2ccc(C(F)(F)F)cc2)C(=O)CN(Cc2cc(F)cc(F)c2)C(=O)CN(Cc2ccc(-c3ccccc3)cc2)C(=O)[C@H]2CCCC[C@H]2N1] |
| 103 | PJS-CLPP-aaab-3 | 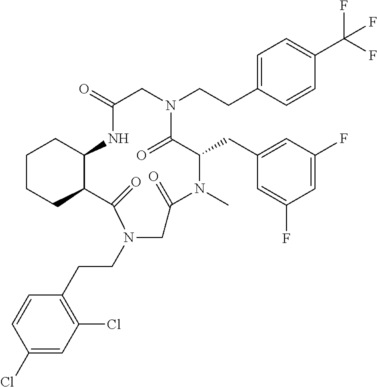 [SMILES: CN1C(=O)CN(CCc2ccc(Cl)cc2Cl)C(=O)[C@H]2CCCC[C@H]2NC(=O)CN(CCc2ccc(C(F)(F)F)cc2)C(=O)[C@@H]1Cc1cc(F)cc(F)c1] |
| 104 | PJS-CLPP-aaad-1 | 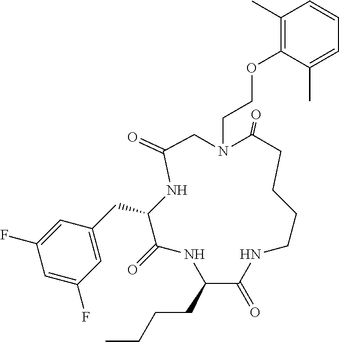 [SMILES: CCCC[C@H]1NC(=O)[C@H](Cc2cc(F)cc(F)c2)NC(=O)CN(CCOc2c(C)cccc2C)C(=O)CCCCNC1=O] |
| 105 | PJS-CLPP-aaam-1 | 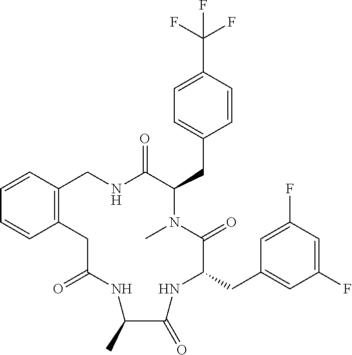 [SMILES: C[C@H]1NC(=O)Cc2ccccc2CNC(=O)[C@@H](Cc2ccc(C(F)(F)F)cc2)N(C)C(=O)[C@H](Cc2cc(F)cc(F)c2)NC1=O] |
| 106 | PJS-CLPP-aaab-4 | 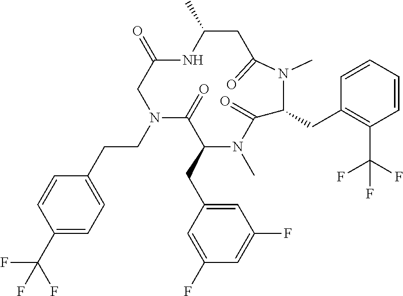 [SMILES: C[C@@H]1CC(=O)N(C)[C@H](Cc2ccccc2C(F)(F)F)C(=O)N(C)[C@@H](Cc2cc(F)cc(F)c2)C(=O)N(CCc2ccc(C(F)(F)F)cc2)CC(=O)N1] |
| 107 | PJS-CLPP-aaad-2 | 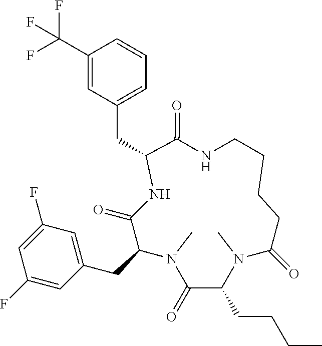 [SMILES: CCCC[C@@H]1C(=O)N(C)[C@@H](Cc2cc(F)cc(F)c2)C(=O)N[C@H](Cc2cccc(C(F)(F)F)c2)C(=O)NCCCCC(=O)N1C] |
| 108 | PJS-CLPP-aaas~1 | 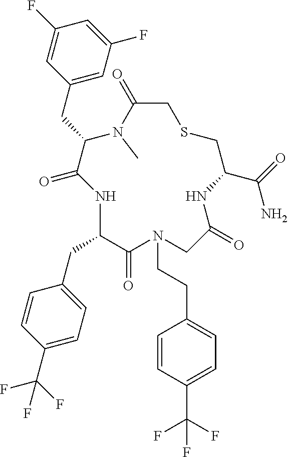 [SMILES: CN1C(=O)CSC[C@H](C(N)=O)NC(=O)CN(CCc2ccc(C(F)(F)F)cc2)C(=O)[C@H](Cc2ccc(C(F)(F)F)cc2)NC(=O)[C@@H]1Cc1cc(F)cc(F)c1] |
| 109 | PJS-CLPP-aaai-3 | 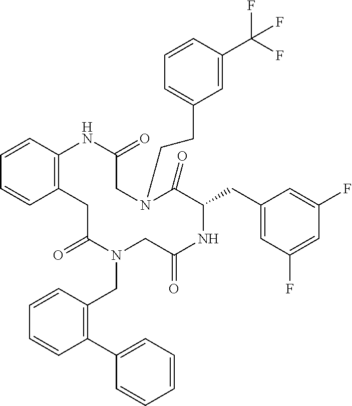 [SMILES: O=C1CN(CCc2cccc(C(F)(F)F)c2)C(=O)[C@H](Cc2cc(F)cc(F)c2)NC(=O)CN(Cc2ccccc2-c2ccccc2)C(=O)Cc2ccccc2N1] |
| 110 | PJS-CLPP-aaag-1 | 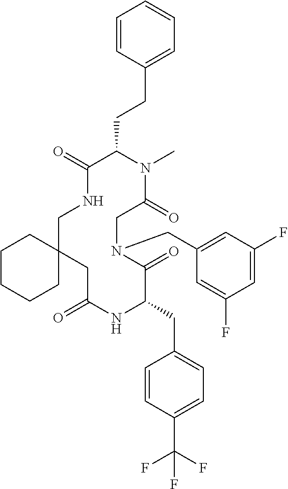 [SMILES: CN1C(=O)CN(Cc2cc(F)cc(F)c2)C(=O)[C@H](Cc2ccc(C(F)(F)F)cc2)NC(=O)CC2(CCCCC2)CNC(=O)[C@@H]1CCc1ccccc1] |
| 111 | PJS-CLPP-aaam-2 | 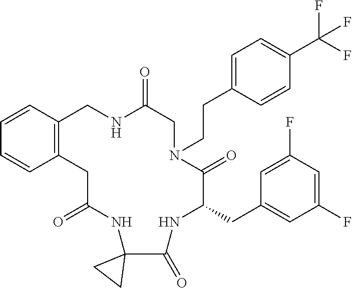 [SMILES: O=C1CN(CCc2ccc(C(F)(F)F)cc2)C(=O)[C@H](Cc2cc(F)cc(F)c2)NC(=O)C2(CC2)NC(=O)Cc2ccccc2CN1] |
| 112 | PJS-CLPP-aaas-2 | 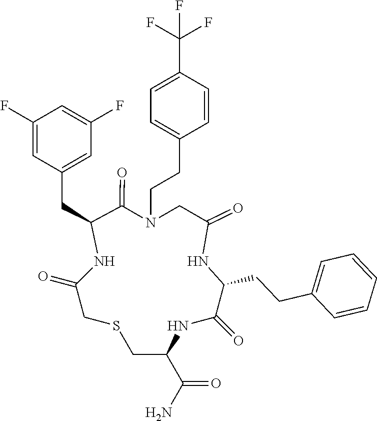 [SMILES: NC(=O)[C@H]1CSCC(=O)N[C@@H](Cc2cc(F)cc(F)c2)C(=O)N(CCc2ccc(C(F)(F)F)cc2)CC(=O)N[C@H](CCc2ccccc2)C(=O)N1] |
| 113 | PJS-BCL-1 | 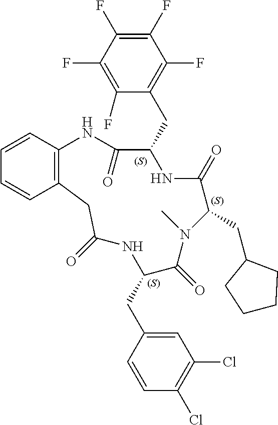 |
| 114 | PJS-BCL-2 | 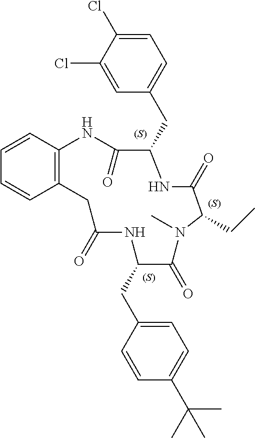 |
| 115 | PJS-BCL-3 | 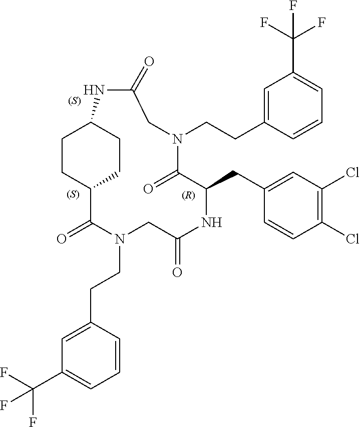 |
| 116 | PJS-BCL-4 | 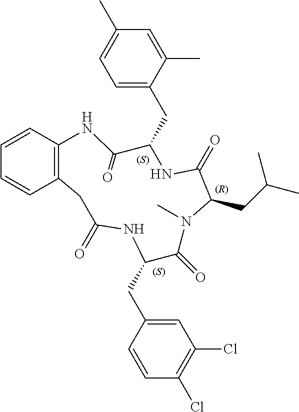 |
| 117 | PJS-BCL-5 | 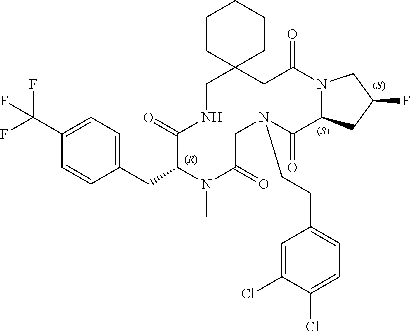 |
| 118 | PJS-BCL-6 | 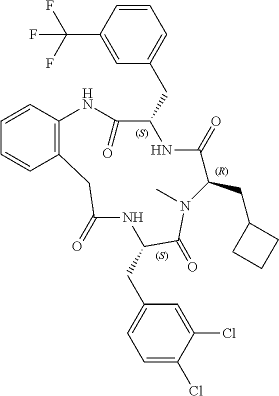 |
| 119 | PJS-BCL-7 | 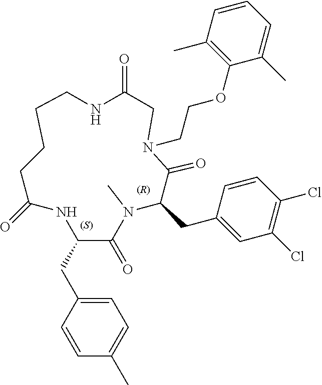 |
| 120 | PJS-BCL-8 | 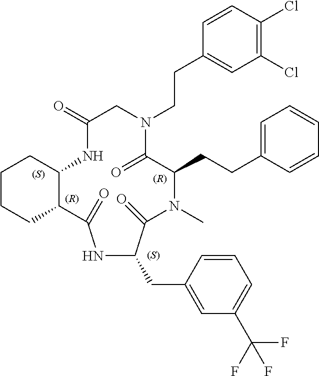 |
| 121 | PJS-BCL-9 | 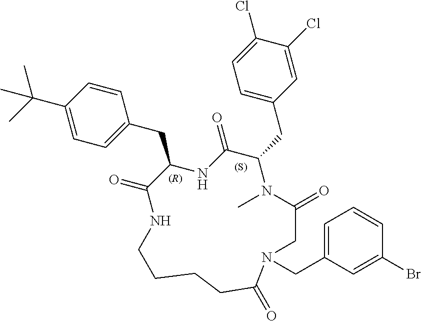 |
| 122 | PJS-BCL-10 | 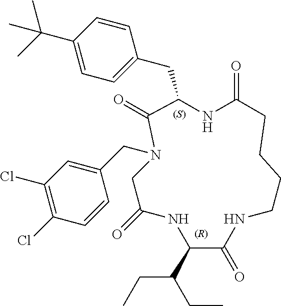 |
| 123 | PJS-BCL-11 | 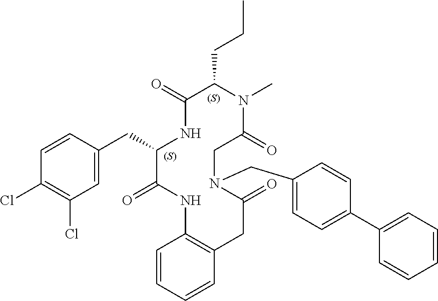 |
| 124 | PJS-BCL-12 | 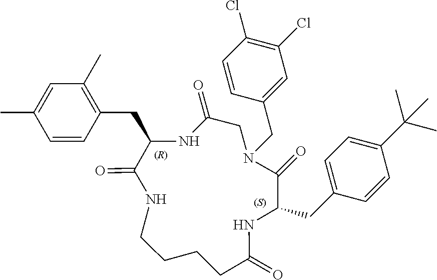 |
| 125 | PJS-BCL-13 | 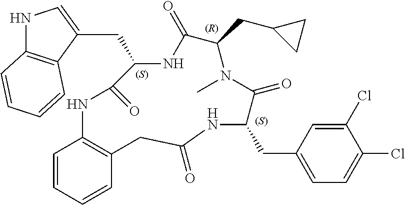 |
| 126 | PJS-BCL-14 | 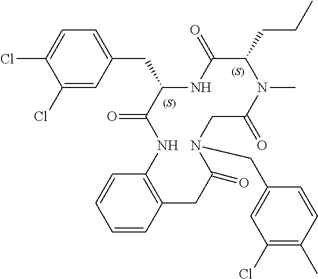 |
| 127 | PJS-BCL-15 | 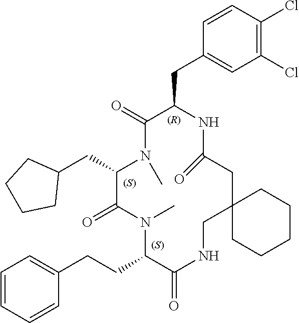 |
| 128 | PJS-BCL-16 | 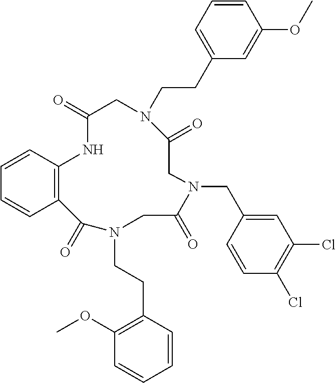 [SMILES: COc1cccc(CCN2CC(=O)Nc3ccccc3C(=O)N(CCc3ccccc3OC)CC(=O)N(Cc3ccc(Cl)c(Cl)c3)CC2=O)c1] |
| 129 | PJS-BCL-17 | 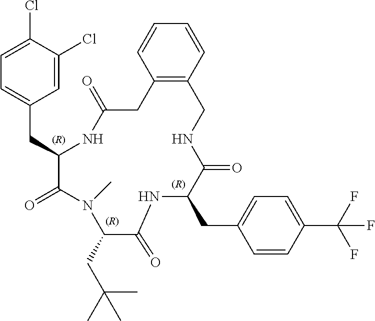 |
| 130 | PJS-BCL-18 | 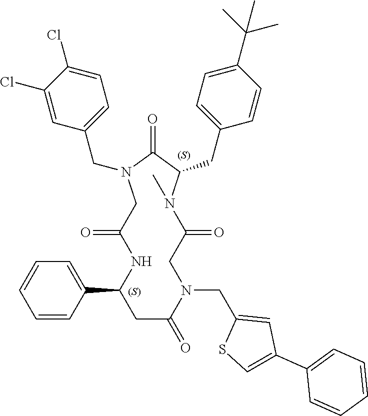 |
| 131 | PJS-MPRO-1-k-a | 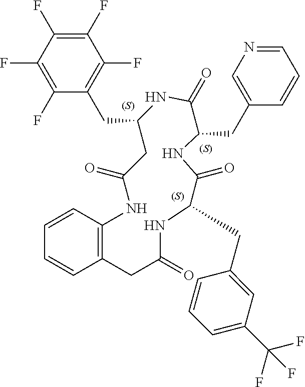 |
| 132 | PJS-MPRO-1-g-a | 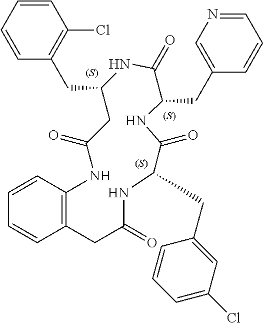 |
| 133 | PJS-MPRO-1-j-a | 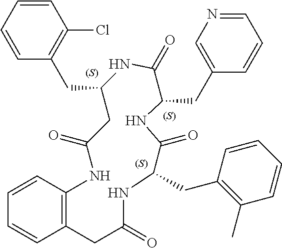 |
| 134 | PJS-MPRO-1-g-b | 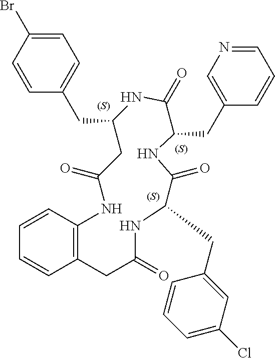 |
| 135 | PJS-MPRO-1-j-b | 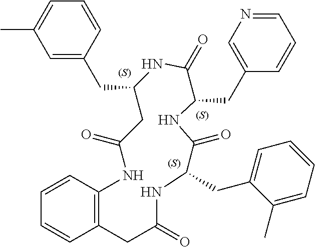 |
| 136 | PJS-MPRO-1-k-b | 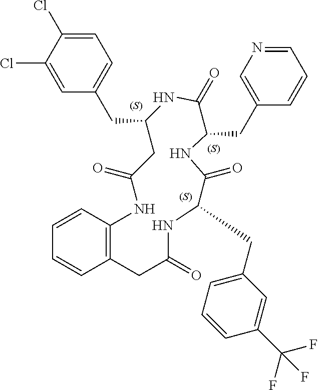 |
| 137 | PJS-MPRO-1-k-c | 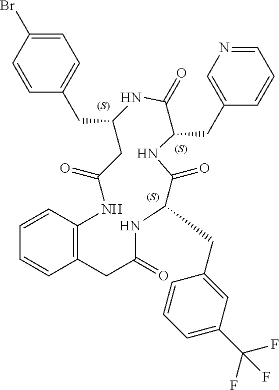 |
| 138 | PJS-MPRO-1-k-d | 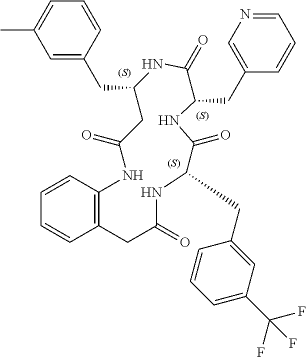 |
| 139 | PJS-MPRO-1-j-c | 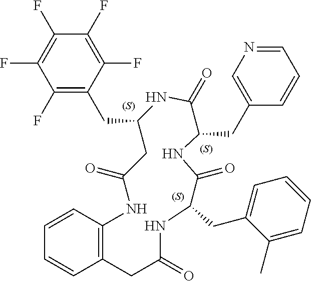 |
| 140 | PJS-MPRO-1-j-d | 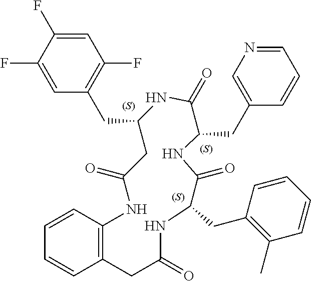 |
| 141 | PJS-MPRO-1-g-c | 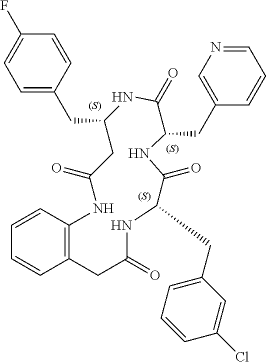 |
| 142 | PJS-MPRO-1-j-e | 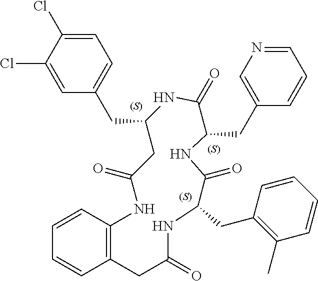 |
| 143 | PJS-MPRO-1-g-d | 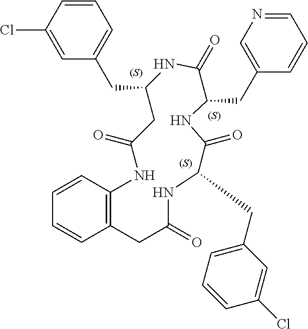 |
| 144 | PJS-MPRO-1-k-e | 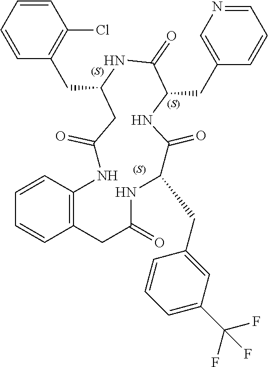 |
| 145 | PJS-FX-1 | 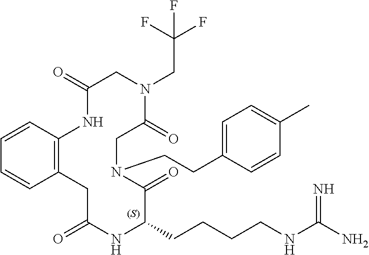 |
| 146 | PJS-FX-2 | 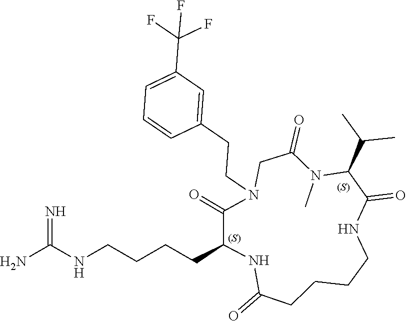 |
| 147 | PJS-FX-3 | 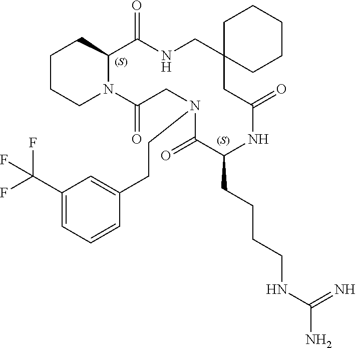 |
| 148 | PJS-FX-4 | 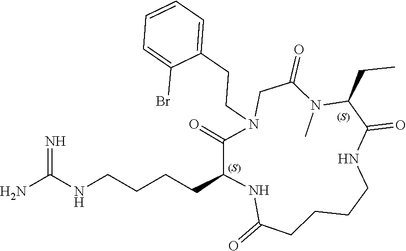 |
| 149 | PJS-FX-5 | 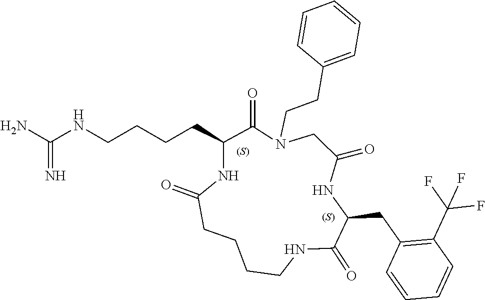 |
| 150 | PJS-FX-6 | 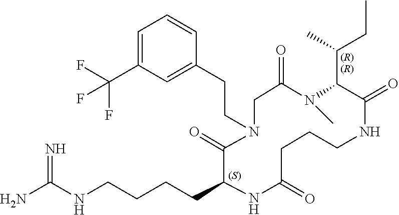 |
| 151 | PJS-FX-7 | 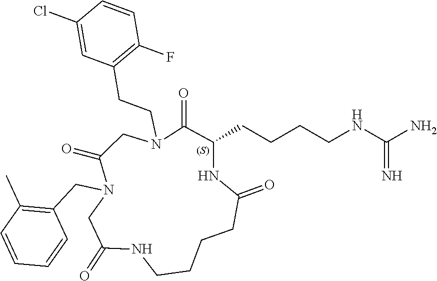 |
| 152 | PJS-FX-8 | 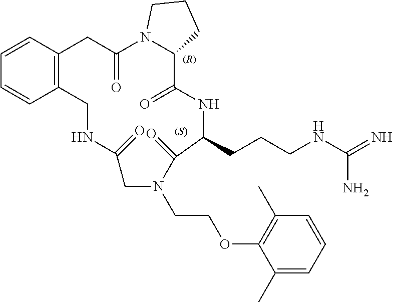 |
| 153 | PJS-FX-9 | 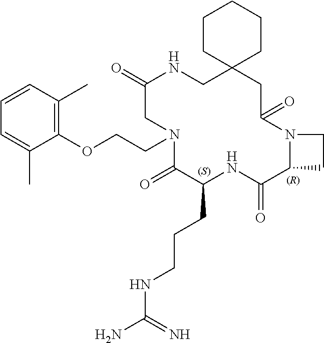 |
| 154 | PJS-FX-10 | 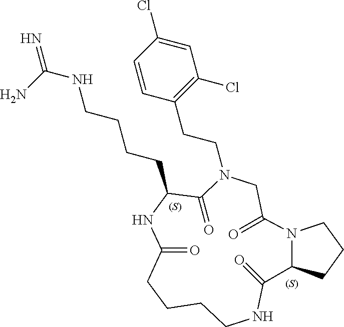 |
| 155 | PJS-FX-11 | 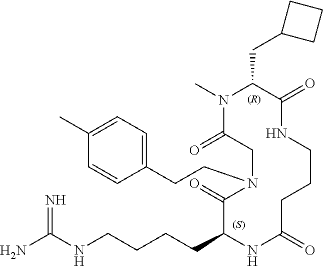 |
| 156 | PJS-FX-12 | 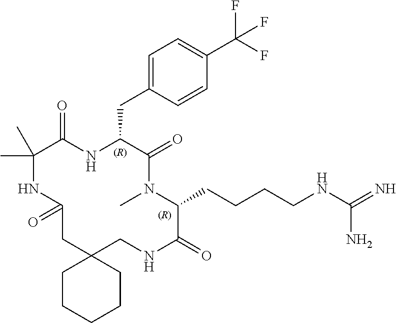 |
| 157 | PJS-FX-13 | 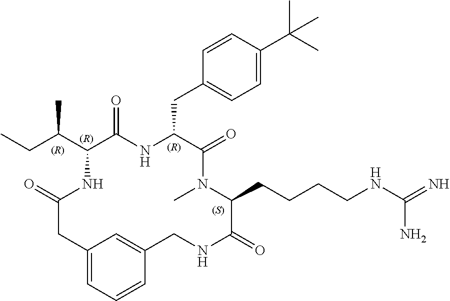 |
| 158 | PJS-FX-14 | 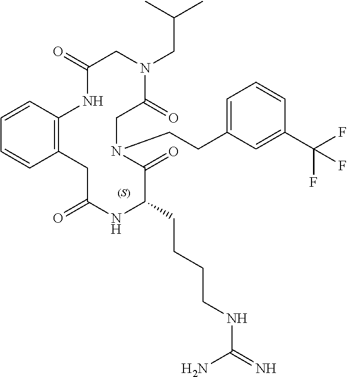 |
| 159 | PJS-FX-15 | 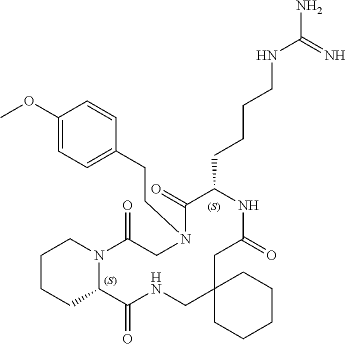 |
| 160 | PJS-FX-16 | 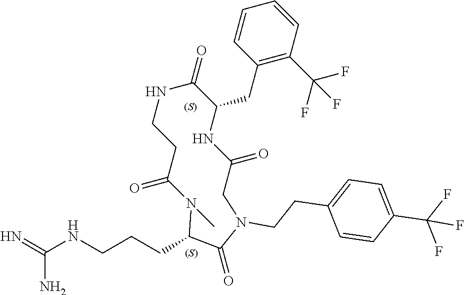 |
| 161 | PJS-IDE-1 | 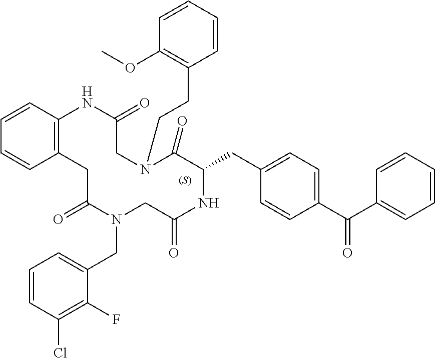 |
| 162 | PJS-IDE-2 | 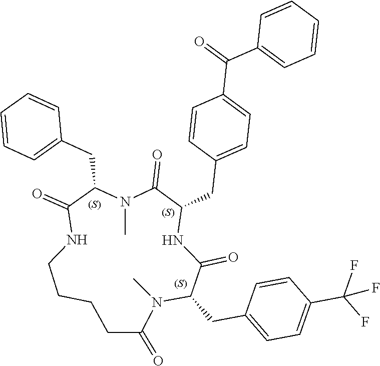 |
| 163 | PJS-IDE-3 | 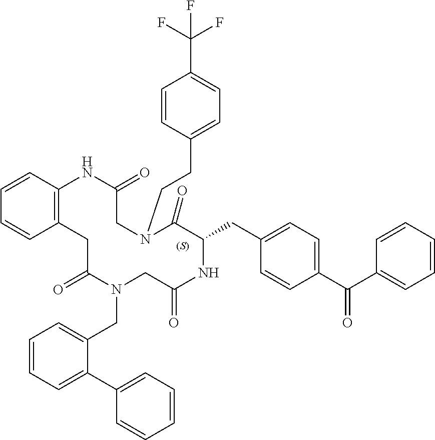 |
| 164 | PJS-IDE-4 | 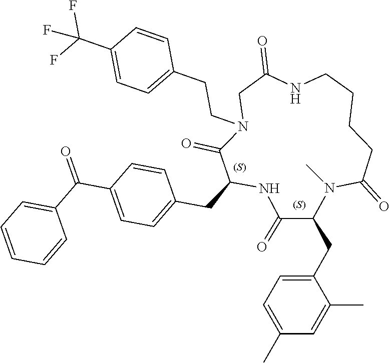 |
| 165 | PJS-IDE-5 | 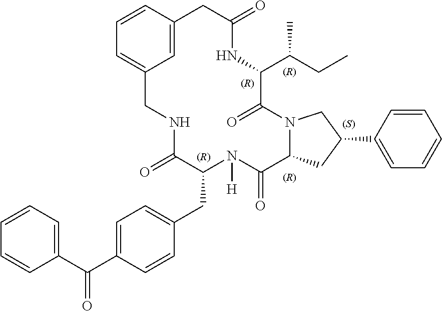 |
| 166 | PJS-IDE-6 | 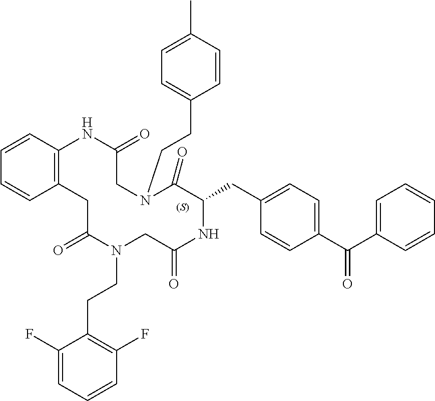 |
| 167 | PJS-IDE-7 | 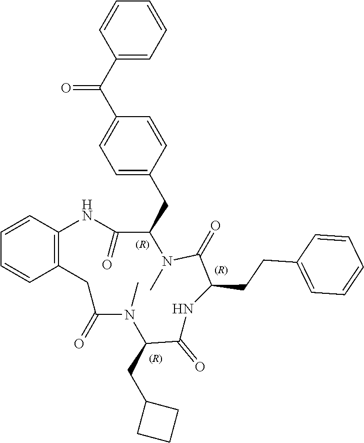 |
| 168 | PJS-IDE-8 | 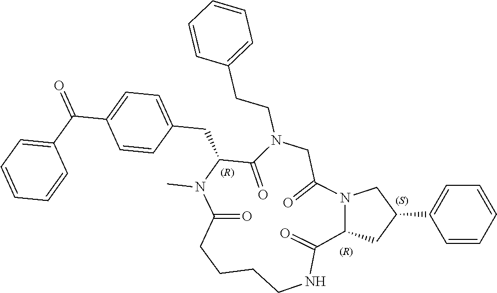 |
| 169 | PJS-IDE-9 | 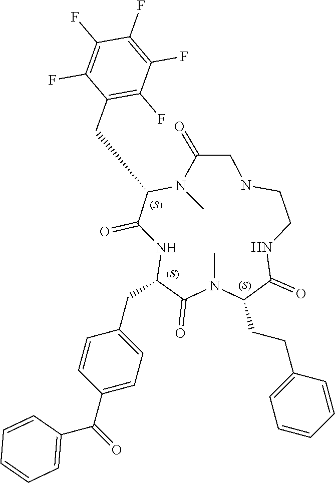 |
| 170 | PJS-IDE-10 | 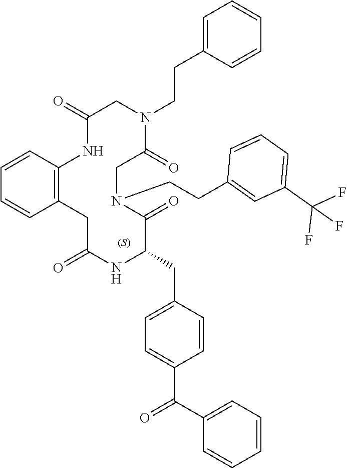 |
| 171 | PJS-TUBU-1 | 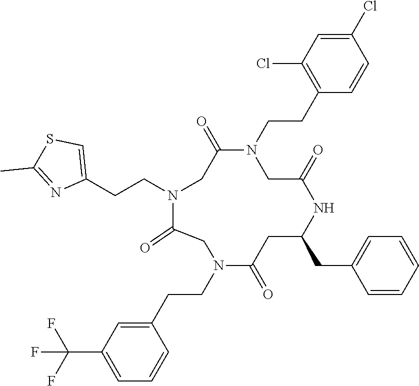 [SMILES: Cc1nc(CCN2CC(=O)N(CCc3ccc(Cl)cc3Cl)CC(=O)N[C@@H](Cc3ccccc3)CC(=O)N(CCc3cccc(C(F)(F)F)c3)CC2=O)cs1] |
| 172 | PJS-TUBU-2 | 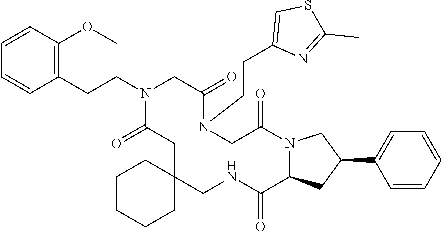 [SMILES: COc1ccccc1CCN1CC(=O)N(CCc2csc(C)n2)CC(=O)N2C[C@@H](c3ccccc3)C[C@H]2C(=O)NCC2(CCCCC2)CC1=O] |
| 173 | PJS-TUBU-3 | 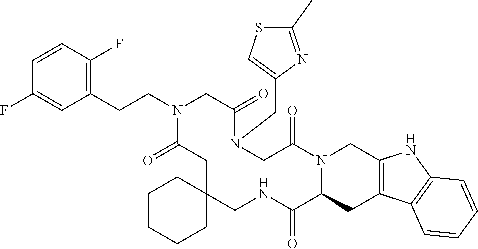 [SMILES: Cc1nc(CN2CC(=O)N3Cc4[nH]c5ccccc5c4C[C@H]3C(=O)NCC3(CCCCC3)CC(=O)N(CCc3cc(F)ccc3F)CC2=O)cs1] |
| 174 | PJS-TUBU-4 | 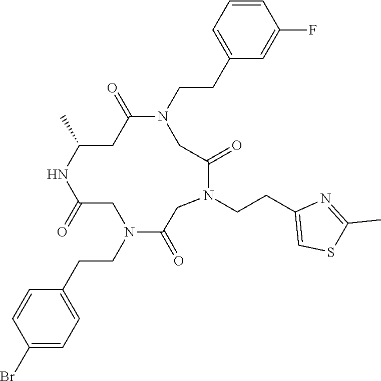 [SMILES: Cc1nc(CCN2CC(=O)N(CCc3ccc(Br)cc3)CC(=O)N[C@H](C)CC(=O)N(CCc3cccc(F)c3)CC2=O)cs1] |
| 175 | PJS-TUBU-5 | 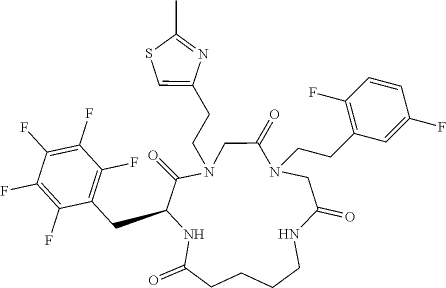 [SMILES: Cc1nc(CCN2CC(=O)N(CCc3cc(F)ccc3F)CC(=O)NCCCCC(=O)N[C@@H](Cc3c(F)c(F)c(F)c(F)c3F)C2=O)cs1] |
| 176 | PJS-TUBU-6 | 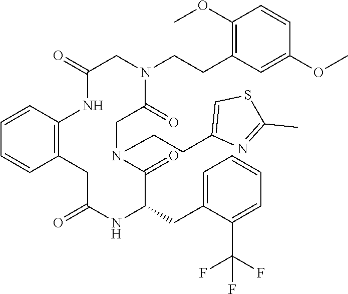 [SMILES: COc1ccc(OC)c(CCN2CC(=O)Nc3ccccc3CC(=O)N[C@@H](Cc3ccccc3C(F)(F)F)C(=O)N(CCc3csc(C)n3)CC2=O)c1] |
| 177 | PJS-TUBU-7 | 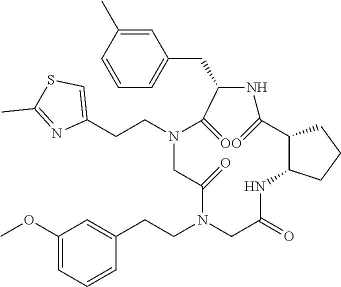 [SMILES: COc1cccc(CCN2CC(=O)N[C@H]3CCC[C@H]3C(=O)N[C@@H](Cc3cccc(C)c3)C(=O)N(CCc3csc(C)n3)CC2=O)c1] |
| 178 | PJS-TUBU-8 | 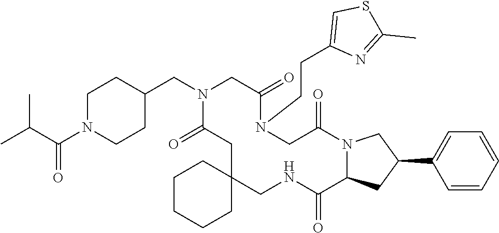 [SMILES: Cc1nc(CCN2CC(=O)N3C[C@@H](c4ccccc4)C[C@H]3C(=O)NCC3(CCCCC3)CC(=O)N(CC3CCN(C(=O)C(C)C)CC3)CC2=O)cs1] |
| 179 | PJS-TUBU-9 | 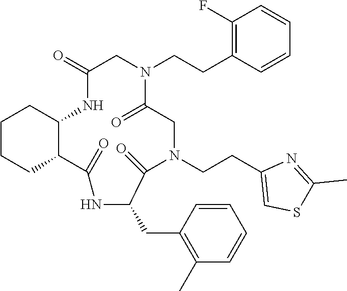 [SMILES: Cc1nc(CCN2CC(=O)N(CCc3ccccc3F)CC(=O)N[C@H]3CCCC[C@H]3C(=O)N[C@@H](Cc3ccccc3C)C2=O)cs1] |
| 180 | PJS-TUBU-10 | 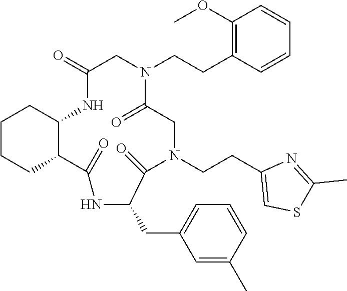 [SMILES: COc1ccccc1CCN1CC(=O)N[C@H]2CCCC[C@H]2C(=O)N[C@@H](Cc2cccc(C)c2)C(=O)N(CCc2csc(C)n2)CC1=O] |
| 181 | PJS-TUBU-11 | 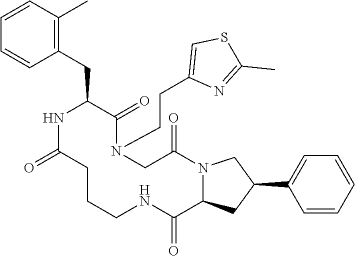 [SMILES: Cc1nc(CCN2CC(=O)N3C[C@@H](c4ccccc4)C[C@H]3C(=O)NCCCC(=O)N[C@@H](Cc3ccccc3C)C2=O)cs1] |
| 182 | PJS-TUBU-12 | 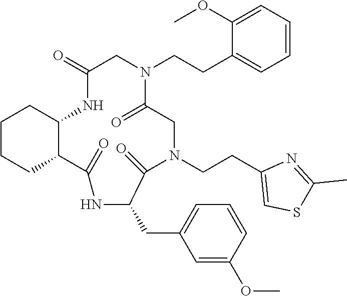 [SMILES: COc1cccc(C[C@@H]2NC(=O)[C@@H]3CCCC[C@@H]3NC(=O)CN(CCc3ccccc3OC)C(=O)CN(CCc3csc(C)n3)C2=O)c1] |
| 183 | PJS-FKBP51-1 | 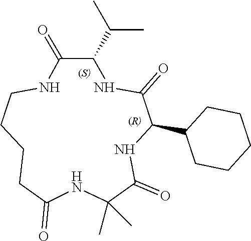 |
| 184 | PJS-FKBP51-2 | 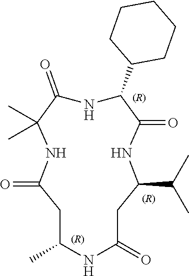 |
| 185 | PJS-FKBP51-3 | 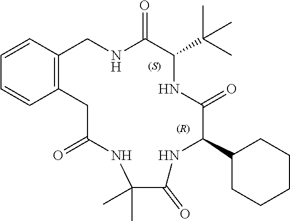 |
| 186 | PJS-FKBP51-4 | 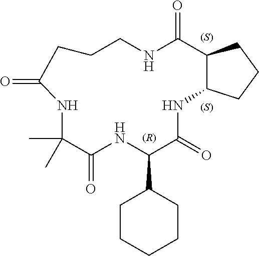 |
| 187 | PJS-FKBP51-5 | 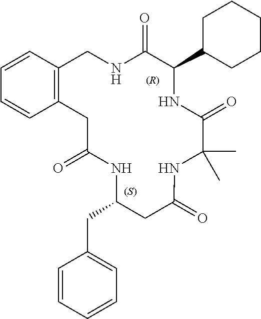 |
| 188 | PJS-FKBP51-6 | 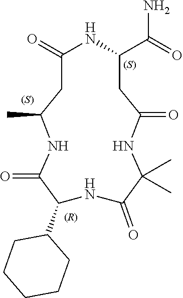 |
| 189 | PJS-FKBP51-7 | 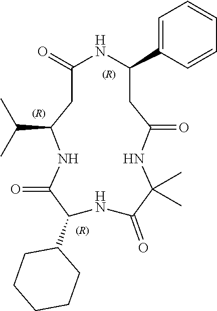 |
| 190 | PJS-FKBP51-8 | 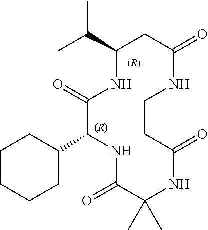 |
| 191 | PJS-FKBP51-9 | 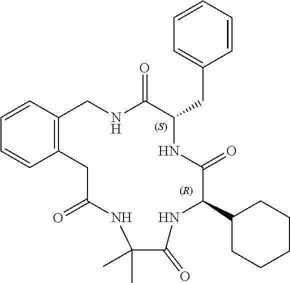 |
| 192 | PJS-FKBP51-10 | 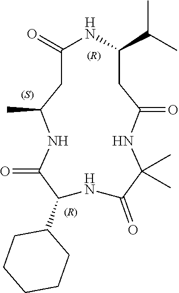 |
| 193 | PJS-FKBP51-11 | 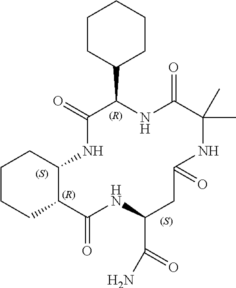 |
| 194 | PJS-FKBP51-12 | 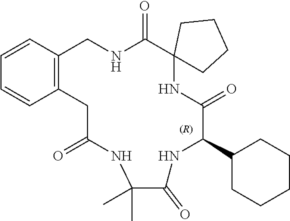 |
| 195 | PJS-FKBP51-13 | 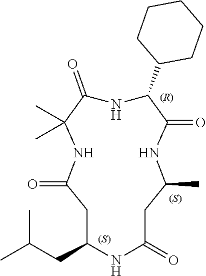 |
| 196 | PJS-FKBP51-14 | 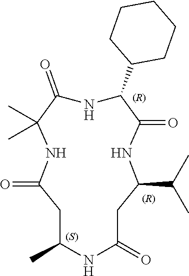 |
| 197 | PJS-FKBP51-15 | 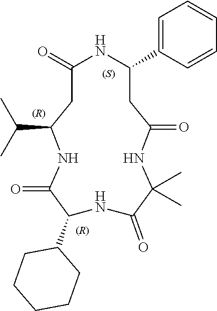 |
| 198 | PJS-FKBP51-16 | 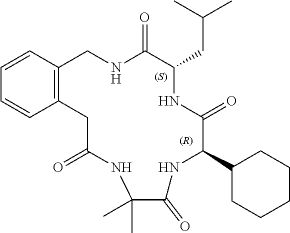 |
| 199 | PJS-FKBP51-17 | 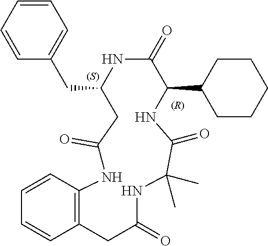 |
| 200 | PJS-FKBP51-18 | 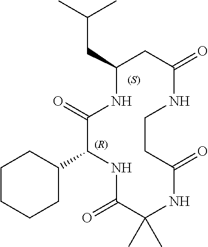 |
| 201 | PJS-FKBP51-19 | 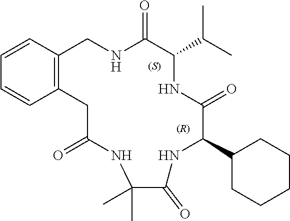 |
| 202 | PJS-FKBP51-20 | 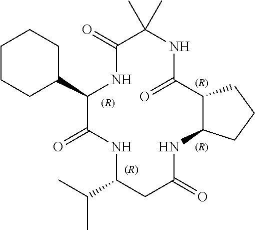 |
| 203 | PJS-DNAN-aaam-1 | 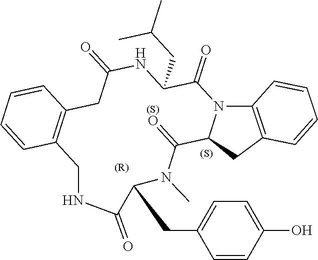 |
| 204 | PJS-DNAN-aaai-1 | 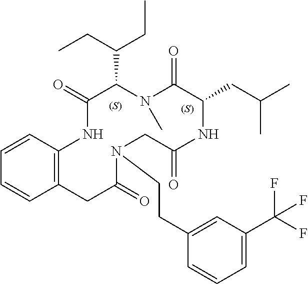 |
| 205 | PJS-DNAN-aaad-1 | 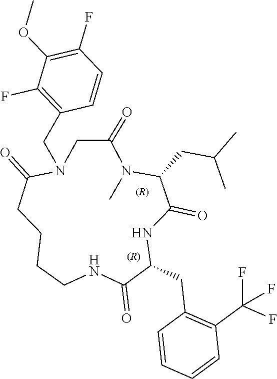 |
| 206 | PJS-DNAN-aaap-1 | 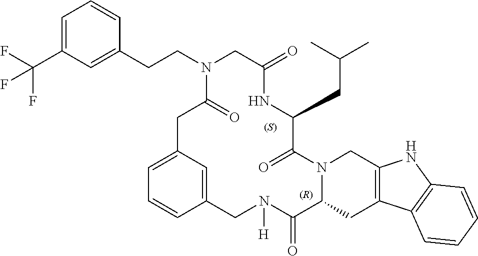 |
| 207 | PJS-DNAN-aaap-2 | 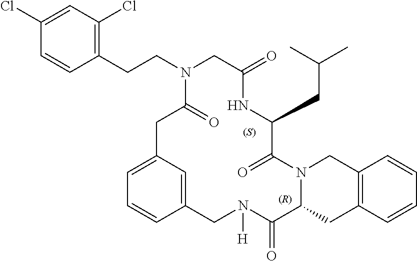 |
| 208 | PJS-DNAN-aaap-3 | 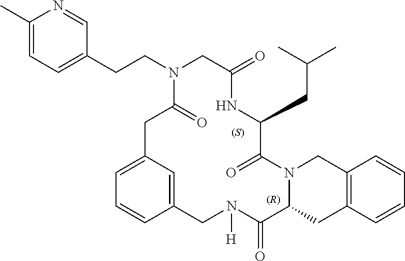 |
| 209 | PJS-DNAN-aaap-4 | 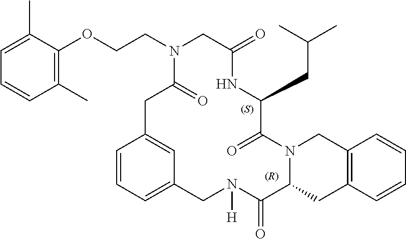 |
| 210 | PJS-DNAN-aaap-5 | 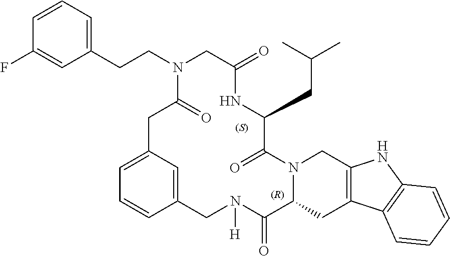 |
| 211 | PJS-DNAN-aaap-6 | 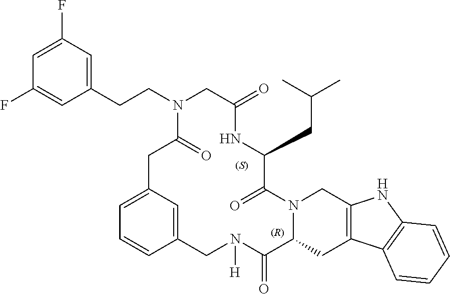 |
| 212 | PJS-DNAN-aaab-1 | 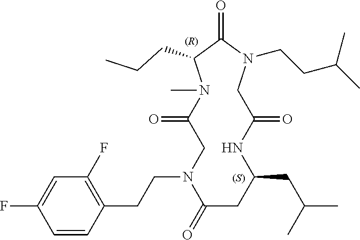 |
| 213 | PJS-DNAN-aaag-1 | 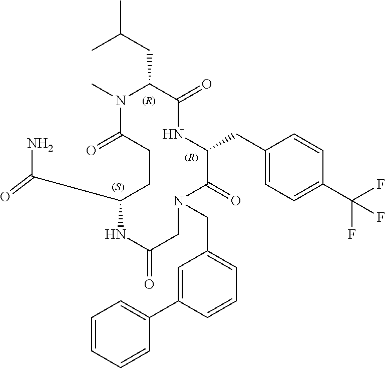 |
| 214 | PJS-DNAN-aaap-7 | 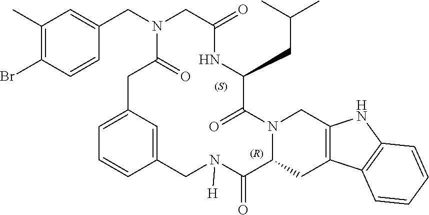 |
| 215 | PJS-DNAN-aaap-8 | 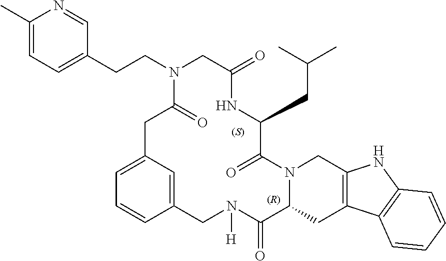 |
| 216 | PJS-DNAN-aaas-1 | 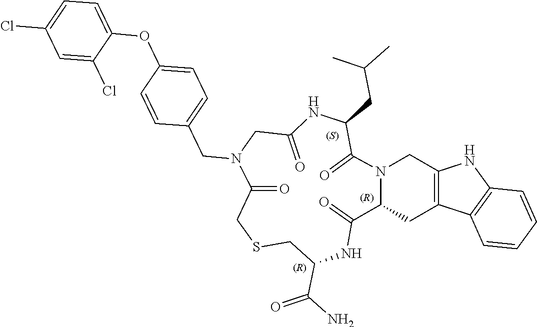 |
| 217 | PJS-DNAN-aaap-9 | 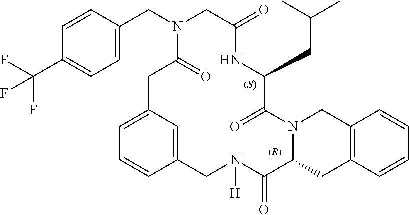 |
| 218 | PJS-MPRO-1-j | 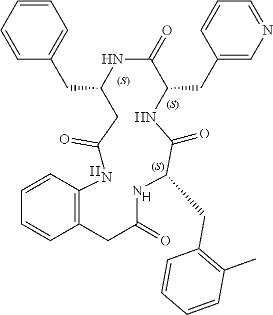 |

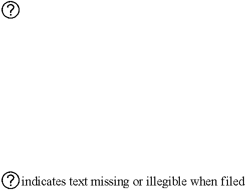

In a further embodiment, the disclosure provides a library of macrocyclic oligoamides, comprising two or more cyclic peptides and/or conjugates according to any embodiment or combination of embodiments disclosed herein. In various embodiments, the library may comprise at least 5, 10, 25, 50, 75, 100, 250, 500, 1000, 5000, 10,000, 25, 000, 35,000, or more macrocyclic oligoamides according to any embodiment or combination of embodiments disclosed herein. The libraries may be used for any suitable use, including but not limited to panning the library to identify macrocyclic oligoamides that bind to a compound of interest.

In another embodiment, the disclosure provides uses and methods for use of the macrocyclic oligoamides and/or library of any embodiment or combination of embodiments disclosed herein. In various non-limiting embodiments, the macrocyclic oligoamides and/or library may be used for panning the library to identify one or more macrocyclic oligoamide that binds to a compound of interest, therapeutic treatments, diagnostic methods, and/or adding reactive moieties for any specific use. In one embodiment, the macrocyclic oligoamides may be used, for example, to carry a substituted moiety across a cell membrane. For example, the methods may comprise administering a macrocyclic oligoamides substituted or otherwise linked to small molecule therapeutic to permit delivery of the therapeutic into cells to effect a desired treatment outcome.

### Macrocycle Design Embodiments

In another aspect, the specification generally describes a system implemented as computer programs on one or more computers in one or more locations that identifies macrocycles.

Throughout this specification, a “molecular fragment” can refer to a sequence of one or more monomers, where each monomer is drawn from a set of monomers. A monomer refers to a molecule that can react together with other monomers to form a sequence (chain) of monomers. The set of monomers can include, e.g., alpha amino acids, beta amino acids, amino benzoic acids, oxazoles, thiazoles, or any other appropriate monomers.

A molecular fragment (or a monomer) can include a first portion that is designated as an “initial end” of the molecular fragment (or the monomer) and a second portion that is designated as a “terminal end” of the molecular fragment (or the monomer). A portion of a molecular fragment (or a monomer) can be designated as an initial end or a terminal end based on any appropriate criteria. In particular, the initial ends and the terminal ends of molecular fragments (or monomers) are generally designated such that the terminal end of one fragment (or monomer) can chemically bond to, or react with, or merge with, the initial end of another fragment (or monomer), and vice versa. For instance, for a molecular fragment that includes a sequence of amino acid monomers, the N-terminus of the first amino acid in the sequence can be designated as the initial end of the molecular fragment, and the C-terminus of the final amino acid in the sequence can be designated as the terminal end of the molecular fragment. As another example, for an amino acid monomer, the N-terminus of the amino acid can be designated as the initial end of the monomer, and the C-terminus of the amino acid can be designated as the terminal end of the monomer.

A molecule that includes a first molecular fragment and a second molecular fragment can be referred to as being a macrocycle, i.e., as having a macrocycle conformation, if the conformation of the first molecular fragment and the second molecular fragment jointly form a closed loop. The conformations of the first molecular fragment and the second molecular fragment can jointly form a closed loop, e.g., if the terminal end of the first fragment is bonded to (or reacted with, or merged with) the initial end of the second fragment, and the initial end of the first fragment is bonded to (or reacted with, or merged with) the terminal end of the second fragment.

A conformation of a molecule (or a molecular fragment) refers to the spatial arrangement (e.g., the three-dimensional (3D) spatial arrangement) of the atoms in the molecule (or the molecular fragment). A 3D spatial location of an atom can be represented in any appropriate coordinate system, e.g., a Cartesian coordinate system.

A first set of values (e.g., transformation parameter values defining a transformation) can be referred to as being “equal” to a second set of values if each first value in the first set of values is equal to a corresponding second value in the second set of values.

Particular embodiments of the subject matter described in this specification can be implemented so as to realize one or more of the following advantages.

Conventional methods for identifying macrocycles systematically enumerate large macrocycles by first generating cyclic conformers of poly-glycine backbones and then carrying out sequence optimization. These methods are difficult to extend to generate macrocycles formed from monomers with diverse backbone chemistry because they require randomly sampling backbone torsion angles to facilitate identifying closed conformations of the macrocycle. This random search through combinations of backbone torsion angles and subsequent sequence design can become intractable, e.g., when many different types of monomer building blocks are included in the backbone. For instance, nearly 60,000 two-dimensional chemical structures of 2-, 3-, and 4-residue macrocycles can be constructed from 22 building block monomers, and each of the 60,000 can be further diversified with potentially millions of combinations of different sidechains. The chemical space is thus far too large to rely on random sampling to identify the small fraction of linear sequences that can be closed to form a cycle.

The system described in this specification implements computationally tractable operations for sampling the large chemical space of possible macrocycles. More specifically, to identify possible macrocycle conformations of a molecule composed of a first molecular fragment and a second molecular fragment, the system can characterize each conformation of the first and second molecular fragments by a respective rigid body transformation. A rigid body transformation for a molecular fragment can define a translation and a rotation of one end (terminus) of the molecular fragment relative to the other end (terminus) of the molecular fragment. The system can process the rigid body transformations to identify complementary conformations of the first and second molecular fragments that jointly form a closed loop (and thus a macrocycle).

The system can efficiently identify complementary conformations of the first and second molecular fragments by generating “dictionaries” representing the respective sets of conformations of the first and second molecular fragments. In particular, to generate a dictionary for a molecular fragment, the system can discretize the rigid body transformations representing the conformations of the molecular fragment, and map each discretized rigid body transformation to a respective “key” (e.g., represented by an integer value). The system can then create the dictionary by adding each key to the dictionary, and associating each key with a set of “values,” where each value represents a conformation with a rigid body transformation corresponding to the key.

The system can generate a first dictionary for rigid body transformations from the initial end to the terminal end of conformations of the first molecular fragment, and a second dictionary for rigid body transformations from the terminal end to the initial end of the second molecular fragment. The system can then identify any keys common to the two dictionaries. For each common key, the system can identify any conformation of the first molecular fragment associated with the key in the first dictionary and any conformation of the second molecular fragment associated with the key in the second dictionary as jointly defining a macrocycle. Discretizing the rigid body transformations and representing them in dictionaries can significantly reduce the computational complexity of identifying complementary conformations of the first and second molecular fragments. In particular, the system achieves lower computational complexity by exploiting the observation that rigid body transformations representing conformations are not uniformly distributed (i.e., in the space of possible rigid body transformations), but rather, are often clustered into a small number of groups. Discretizing the space of possible rigid body transformations can greatly reduce the number of unique rigid body transformations representing conformations of a molecular fragment, and dictionaries (as described above) enable the subsequent identification of complementary conformations by efficient operations, e.g., set intersection operations to identify common keys.

The details of one or more embodiments of the subject matter of this specification are set forth in the accompanying drawings and the description below. Other features, aspects, and advantages of the subject matter will become apparent from the description, the drawings, and the claims.

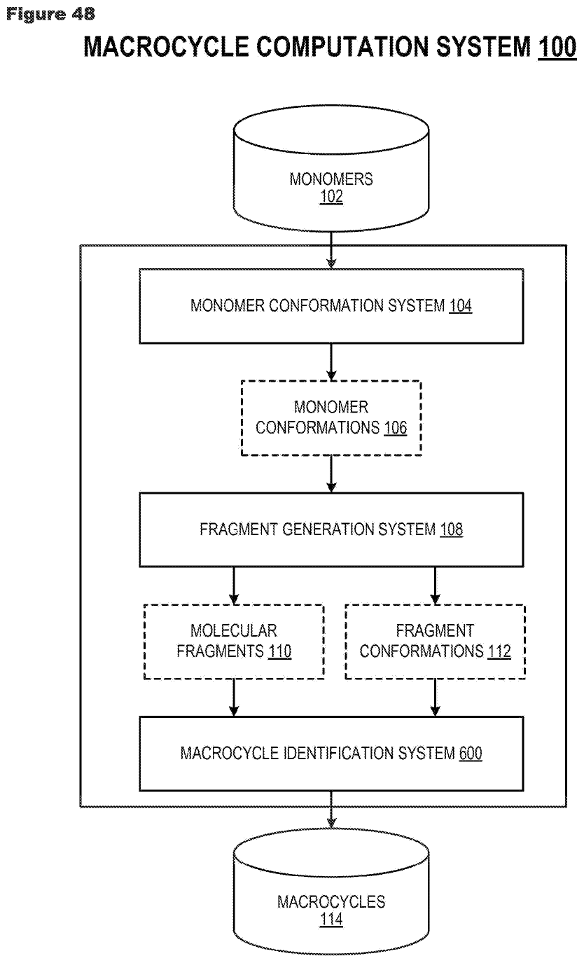

FIG. 48 shows an example macrocycle computation system 100. The macrocycle computation system 100 is an example of a system implemented as computer programs on one or more computers in one or more locations in which the systems, components, and techniques described below are implemented.

The macrocycle computation system 100 is configured to process data defining a set of monomers 102 to identify a set of macrocycle molecules 114 (“macrocycles”).

The set of monomers 102 can include, e.g., alpha amino acids, beta amino acids, amino benzoic acids, oxazoles, thiazoles, or any other appropriate monomers. The set of monomers 102 can be provided to the macrocycle computation system 100, e.g., by way of an application programming interface (API) or user interface made available by the macrocycle computation system 100.

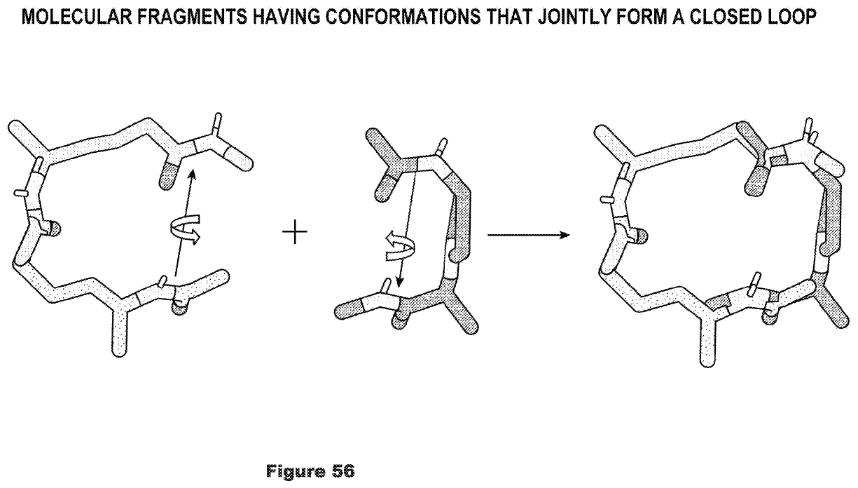

Each macrocycle 114 is a molecule formed from a pair of molecular fragments having respective conformations that jointly form a closed loop. Each of the molecular fragments includes a respective sequence of one or more monomers from the set of monomers 102. FIG. 56, which will be described in more detail below, provides an illustration of a macrocycle formed from a pair of molecular fragments.

The macrocycle computation system 100 includes a monomer conformation system 104, a fragment generation system 108, and a macrocycle identification system 600, which are each described next.

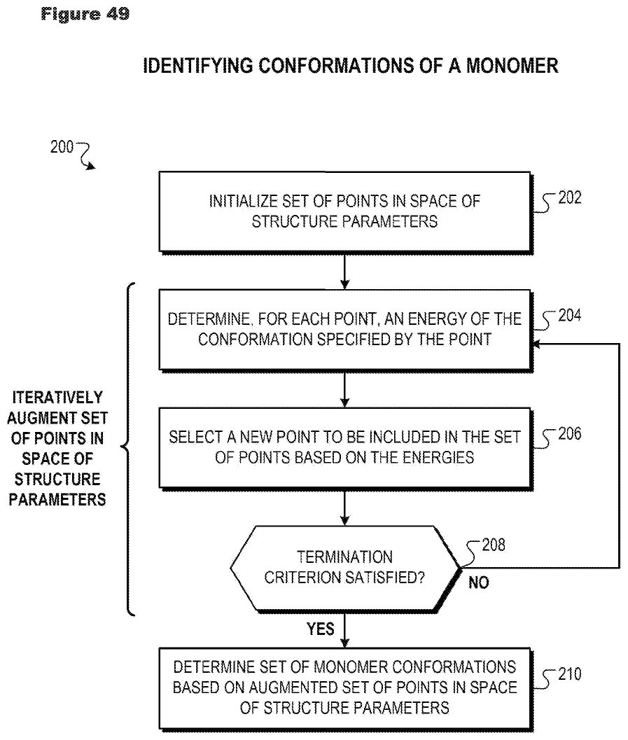

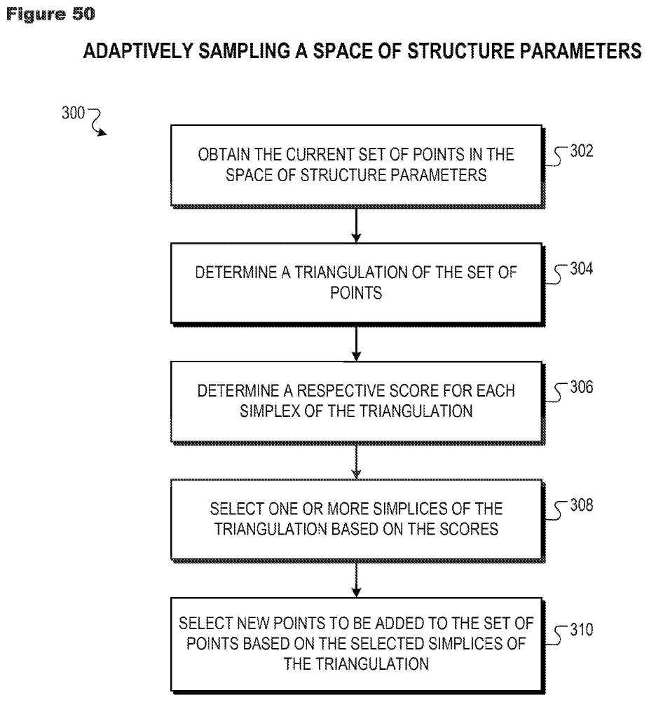

The monomer conformation system 104 is configured to determine a set of one or more conformations of each monomer 102 from the set of monomers 102. The conformation of a monomer refers to the 3D spatial arrangement of the atoms in the monomer. The conformation of a monomer can be represented in any appropriate manner, e.g., by a list of 3D spatial coordinates of the atoms in the monomer. The monomer conformation system 104 can generate any appropriate number of conformations for each monomer, e.g., 5 conformations, 10 conformations, 100 conformations, or 1,000 conformations. The monomer conformation system 104 can generate different numbers of conformations for different monomers. An example process for generating monomer conformations is described with reference to FIG. 49-FIG. 50.

In some implementations, the macrocycle computation system 100 can exclude the monomer conformation system 104, i.e., such that the monomer conformation system 104 is not included in the macrocycle computation system 100. For instance, rather than generating the monomer conformations 106, the macrocycle computation system 100 can receive a predefined library of monomer conformations 106 as an input.

The fragment generation system 108 is configured to generate data defining: (i) a set of molecular fragments 110, and (ii) a respective set of conformations 112 of each molecular fragment 110.

Each molecular fragment 110 includes a sequence of one or more monomers 102 from the set of monomers 102. Certain molecular fragments 110 may include only a single monomer, while other molecular fragments 110 may include multiple monomers, e.g., 2 monomers, 3 monomers, 4 monomers, 10 monomers, 20 monomers, or any other appropriate number of monomers. The fragment generation system 108 can generate any appropriate number of molecular fragments, e.g., 1,000 molecular fragments, 100,000 molecular fragments, 1,000,000 molecular fragments, etc.

To generate a molecular fragment 110, the fragment generation system 108 can select the length of the molecular fragment, i.e., the number of monomers to be included in the monomer sequence of the molecular fragment. The fragment generation system 108 can then select a respective monomer for each position in the monomer sequence of the molecular fragment, e.g., by sampling a monomer 102 from the set of monomers 102.

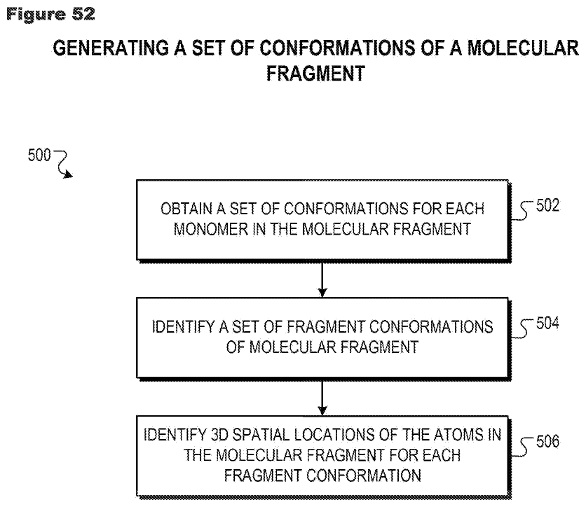

The fragment generation system 108 can generate data defining a set of conformations 112 of a molecular fragment 110 based on, for each position in the monomer sequence of the molecular fragment 110, the set of monomer conformations 106 of the monomer at the position. An example process for generating data defining a set of conformations of a molecular fragment based on the possible conformations of the monomers in the monomer sequence of the molecular fragment is described with reference to FIG. 52.

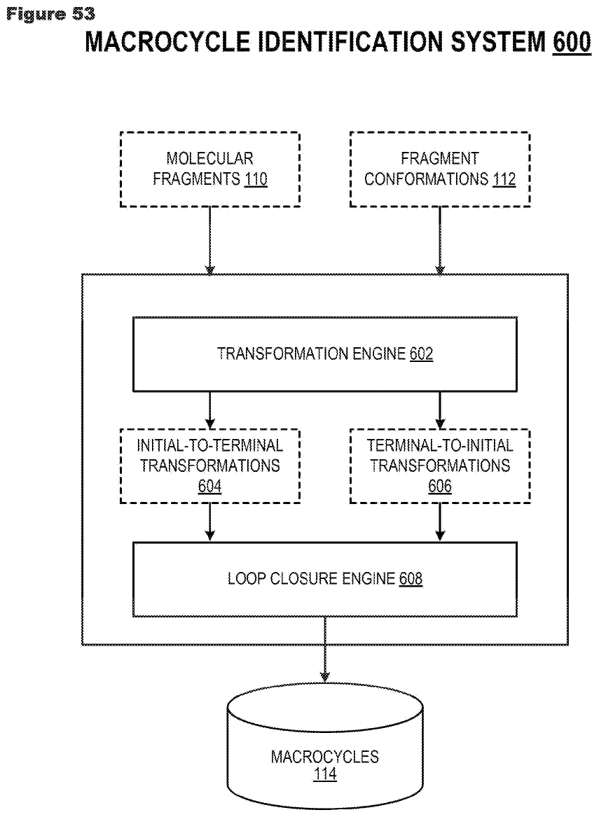

The macrocycle identification system 600 is configured to process the set of molecular fragments 110 and their corresponding conformations 112 to generate data defining a set of macrocycles 114. Each of the macrocycles 114 is a molecule formed from pair of molecular fragments 110 having respective fragment conformations 112 that jointly form a closed loop. An example of a macrocycle identification system 600 is described in more detail with reference to FIG. 53-FIG. 55.

The macrocycles 114 identified by the macrocycle computation system 100 can be used in any of a variety of possible downstream processes. A few examples of downstream processes that can make use of macrocycles 114 identified by the macrocycle computation system 100 are described next.

In some implementations, a macrocycle 114 identified by the macrocycle computation system 100 can be physically synthesized, e.g., in a laboratory. The physically synthesized macrocycle can be used in any of a variety of ways. For example, properties of the physically synthesized macrocycle, e.g., chemical stability, toxicity, solubility, reactivity, etc., can be determined by appropriate experimental techniques. As another example, the macrocycle can be physically synthesized for use in a therapeutic, e.g., a drug. The therapeutic can be administered to a subject, e.g., a mouse, a cat, a dog, a pig, or a human, e.g., to achieve a therapeutic effect in the subject. As another example, the conformation of the physically synthesized macrocycle can be determined through experimental techniques, e.g., x-ray crystallography, and compared to a conformation predicted by the macrocycle computation system 100, e.g., as part of validating the macrocycle computation system 100. As another example, the physically synthesized macrocycle can be brought into proximity of a binding site, e.g., on the surface of a protein, to determine a binding affinity of the macrocycle for the binding site.

In some implementations, a macrocycle 114 identified by the macrocycle computation system 100 can be provided for use in a downstream computational simulation or computational analysis process. For example, a high-fidelity computational simulation, e.g., based on density functional theory (DFT), can be performed to determine the energy of the macrocycle. As another example, a computational analysis system can process a representation of the macrocycle to predict the affinity of the macrocycle for a binding site, e.g., on the surface of a protein.

FIG. 49 is a flow diagram of an example process 200 for generating a set of conformations of a monomer. For convenience, the process 200 will be described as being performed by a system of one or more computers located in one or more locations. For example, a macrocycle computation system, e.g., the macrocycle computation system 100 of FIG. 48, appropriately programmed in accordance with this specification, can perform the process 200.

The system initializes a set of points in a space of structure parameters, where each point in the space of structure parameters defines a respective candidate conformation of the monomer (202). More specifically, each point in the space of structure parameters can specify a respective value for each structure parameter in a set of structure parameters that jointly define a candidate conformation of the monomer. The set of structure parameters can include, e.g., a set of torsion (dihedral) angles that define the angles of bonds between atoms in the monomer. The set of structure parameters can include any appropriate number of structure parameters, e.g., 3 structure parameters, 10 structure parameters, or 30 structure parameters.

The system can initialize the set of points in the space of structure parameters in any appropriate way. For instance, the system can initialize the set of points in the space of structure parameters by randomly sampling a predefined number of points in the space of structure parameters. As another example, the system can initialize the set of points in the space of structure parameters by selecting points that form a uniform grid over the space of structure parameters. The system can initialize the set of points in the space of structure parameters to include any appropriate number of points, e.g., 100 points, 1,000 points, or 100,000 points.

In steps 204-208, the system iteratively augments the set of points in the space of structure parameters to include additional points. Iteratively augmenting the set of points has the effect of adaptively sampling points from the space of structure parameters in order to efficiently identify candidate conformations having low energies. For convenience, steps 204-208 will be described with reference to a “current” set of points in the space of structure parameters at a “current” iteration, i.e., in a sequence of iterations that are performed by the system.

The system determines, for each point in the current set of points, an energy of the candidate conformation specified by the point (204). If the current iteration is the first iteration in the sequence of iterations, then the system determines a respective energy of the candidate conformation corresponding to each point in the initial set of points. For iterations after the first iteration, the system may only be required to determine energies of candidate conformations corresponding to points that were added to the current set of points at the preceding iteration. More specifically, after determining the energy of a candidate conformation corresponding to a point, the system can store the energy value and is not required to regenerate the energy value for the point at each iteration.

The system can determine the energy of a candidate conformation in any appropriate way. For instance, to determine the energy of a candidate conformation, the system can process a model input based on a set of structure parameters defining the candidate conformation using an energy prediction machine learning model to generate a model output that define a predicted energy of the candidate conformation. The energy prediction machine learning model can have any appropriate machine learning model architecture that enables the energy prediction machine learning model to perform its described functions. For instance, the energy prediction machine learning model can be a neural network model, a random forest model, a support vector machine model, etc. In implementations where the energy prediction machine learning model is a neural network model, the neural network model can include any appropriate types of neural network layers (e.g., fully connected layers, convolutional layers, attention layers, etc.) in any appropriate number (e.g., 5 layers, 10 layers, or 50 layers) and connected in any appropriate configuration (e.g., as a linear sequence of layers).

An example of an energy prediction machine learning model that can be used to determine the energies of candidate conformations is described with reference to R Zubatyuk, J. S. Smith, J. Leszczynski, O. Isayev, “Accurate and transferable multitask prediction of chemical properties with an atoms-in-molecules neural network,” Science Advances, 9 Aug. 2019, Volume 5, Issue 8.

The energy prediction machine learning model can be trained on a set of training examples, where each training example corresponds to a training conformation and includes. (i) a training input to the energy prediction machine learning model, and (ii) a target output of the energy prediction machine learning model. The training input of a training example can be based on structure parameters that define the corresponding training conformation. The target output of a training example can define the energy of the corresponding training conformation. The energy of a training conformation can be determined, e.g., using high-fidelity computational simulations, e.g., based on density functional theory (DFT).

The system selects one or more new points to be included in the current set of points based on the energies of the conformations corresponding to the points in the current set of points (206). The system can select new points in a manner that encourages dense sampling of regions of the space of structure parameters that have been determined to correspond to low energy conformations while simultaneously encouraging exploration of under-sampled parts of the space of structure parameters. An example process for selecting new points to be included in the current set of points is described with reference to FIG. 50.

The system determines whether a termination criterion is satisfied (208). The termination criterion can be, e.g., that the system has performed a predefined number of iterations of steps 204-208, or that the current set of points includes at least a threshold number of points.

In response to determining that the termination criterion is not satisfied, the system returns to step 204 and performs another iteration of augmenting the current set of points in the space of structure parameters.

In response to determining that the termination criterion is satisfied, the system determines a set of conformations of the monomer based on the current set of points in the space of structure parameters, i.e., as of the last iteration of steps 204-208 (210). For instance, the system can identify each point in the current set of points that corresponds to candidate conformation having an energy that satisfies (e.g., is below) a threshold as being a respective conformation of the monomer.

FIG. 50 is a flow diagram of an example process 300 for adaptively sampling a space of structure parameters, where each point in the space of structure parameters defines a respective candidate conformation of a monomer. For convenience, the process 300 will be described as being performed by a system of one or more computers located in one or more locations. For example, a macrocycle computation system, e.g., the macrocycle computation system 100 of FIG. 48, appropriately programmed in accordance with this specification, can perform the process 300.

The system obtains a current set of points in the space of structure parameters, i.e., that defines a current sampling of the space of structure parameters (302).

The system determines a triangulation, e.g., a Delaunay triangulation, of the current set of points (304). A triangulation of a set of points refers to a simplicial complex that has vertices defined by the set of points and that covers the convex hull of the set of points. A triangulation can partition a region of the space of structure parameters included in the convex hull of the set of points into a set of sub-regions, referred to for convenience as “simplices” of the triangulation.

The system determines a respective score for each simplex of the triangulation (306). For instance, the system can determine a score for a simplex of the triangulation based on; (i) the volume of the simplex of the triangulation, and (ii) the energies of the points at the vertices of the simplex of the triangulation. In a particular example, the system can determine a score for a simplex of the triangulation based on a product of; (i) the volume of the simplex of the triangulation, and (ii) an exponential of the negative of the product of the energies of the points at the vertices of the simplex of the triangulation.

The system selects one or more simplices of the triangulation based on the scores for the simplices of the triangulation (308). For instance, the system can select a predefined number of the simplices of the triangulation having the highest scores, or the system can select each simplex of the triangulation having a score that exceeds a threshold.

The system can generate one or more new points to be added to the set of points based on the selected simplices of the triangulation (310). For instance, for each selected simplex of the triangulation, the system can determine a point at a center of the simplex of the triangulation, and then add the point at the center of the simplex of the triangulation to the set of points.

Augmenting the set of points in this manner encourages exploration of the space of structure parameters, e.g., by scoring simplices of the triangulation based on their volume, thus increasing the likelihood that new points are selected from simplices of the triangulation with high volume. Further, the system encourages dense sampling of regions of the space of structure parameters that are determined to include points with low energies, e.g., by scoring simplices of the triangulation based on the energies of the points at the vertices of the simplices of the triangulation.

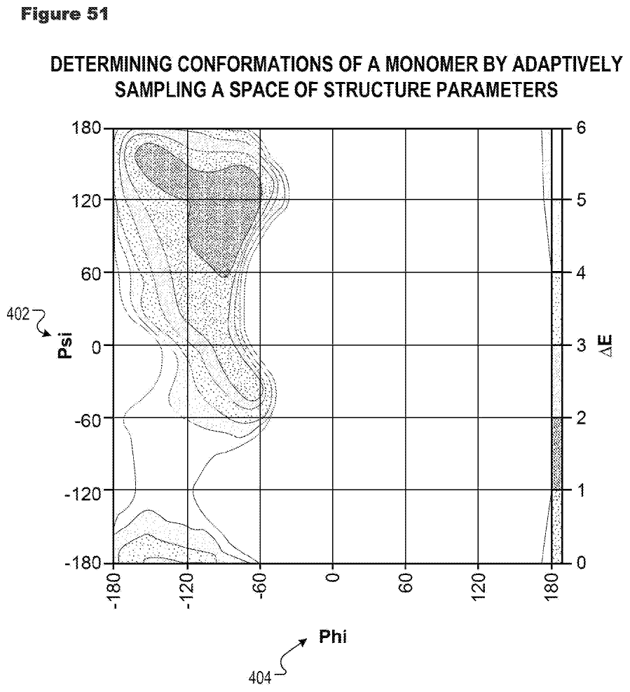

FIG. 51 illustrates an example of adaptively sampled a space of structure parameters for a monomer to determine a set of conformations of the monomer, e.g., as described with reference to FIG. 49-FIG. 50. In this example, space of structure parameters includes one dimension corresponding to a phi torsion angle 404 of the monomer, and another dimension corresponding to a psi torsion angle 402 of the monomer. The sampling is performed in a manner that efficiently identifies low energy conformations of the monomer, as described above.

FIG. 52 is a flow diagram of an example process 500 for generating data defining a set of conformations of a molecular fragment based on possible conformations of the monomers in the monomer sequence of the molecular fragment. For convenience, the process 500 will be described as being performed by a system of one or more computers located in one or more locations. For example, a macrocycle computation system, e.g., the macrocycle computation system 100 of FIG. 48, appropriately programmed in accordance with this specification, can perform the process 500.

The system obtains a respective set of conformations of each monomer in the monomer sequence of the molecular fragment (502).

The system identifies a set of fragment conformations of the molecular fragment (504). Each fragment conformation corresponds to a particular choice of a monomer conformation for each monomer in the monomer sequence of the molecular fragment. The system can identify a number of fragment conformations given by: Π(i=1)\hat{ }N n_i, where N denotes the number of positions in the monomer sequence of the molecular fragment, i indexes the positions in the monomer sequence of the molecular fragment, and n_i denotes the number of monomer conformations of the monomer at position i in the monomer sequence of the molecular fragment.

For each identified fragment conformation of the molecular fragment, the system identifies 3D spatial locations of the atoms in the molecular fragment when the molecular fragment assumes the fragment conformation (506). More specifically, for a given fragment conformation, the system can sequentially determine sequentially the 3D spatial locations of the atoms included in the respective monomer at each position in the monomer sequence of the molecular fragment, starting from the first position. For the first position in the monomer sequence, the spatial location of each atom in the monomer at the position can be directly defined by the conformation of the monomer at the position. For each position after the first position in the monomer sequence, the spatial location of each atom in the monomer can be determined by applying a rotation and translation operation to the spatial locations of the atoms defined by the conformation of the monomer at the position. The rotation and translation operations can be selected to align the initial end of the monomer at the current position with the terminal end of the monomer at the preceding position.

FIG. 53 shows an example macrocycle identification system 600. The macrocycle identification system 600 is an example of a system implemented as computer programs on one or more computers in one or more locations in which the systems, components, and techniques described below are implemented.

The macrocycle identification system 600 is configured to process a set of molecular fragments 110 and their corresponding conformations 112 to generate data identifying a set of macrocycles 114. Each of the macrocycles 114 is a molecule formed from a pair of molecular fragments 110 having respective fragment conformations 112 that jointly form a closed loop.

The macrocycle identification system 600 includes a transformation engine 602 and a loop closure engine 608, which are each described in more detail next.

The transformation engine 602 is configured to generate data defining a “initial-to-terminal” transformation 604 and a “terminal-to-initial” transformation 606 for each fragment conformation 112.

An initial-to-terminal transformation 604 for a fragment conformation 112 of a molecular fragment 110 defines a translation and rotation of the initial end of the molecular fragment 110 relative to the terminal end of the molecular fragment 110. That is, an initial-to-terminal transformation defines a translation operation and a rotation operation that, if applied to the coordinates of the atoms in the initial end of the molecular fragment, generates transformed coordinates that are aligned with the coordinates of the atoms in the terminal end of the molecular fragment.

An initial-to-terminal transformation is parameterized by a set of transformation parameters from a space of transformation parameters. For instance, the set of transformation parameters parameterizing an initial-to-terminal transformation can define a rotation matrix (e.g., a 3×3 rotation matrix) that defines a 3D spatial rotation operation and a translation vector (e.g., a 3×1 translation vector) that defines a 3D spatial translation operation.

An terminal-to-initial transformation 606 for a fragment conformation 112 of a molecular fragment 110 defines a translation and rotation of the terminal end of the molecular fragment 110 relative to the initial end of the molecular fragment 110. That is, a terminal-to-initial transformation defines a translation operation and a rotation operation that, if applied to the coordinates of the atoms in the terminal end of the molecular fragment, generates transformed coordinates that are aligned with the coordinates of the atoms in the initial end of the molecular fragment.

A terminal-to-initial transformation is parameterized by a set of transformation parameters from a space of transformation parameters. For instance, the set of transformation parameters parameterizing a terminal-to-initial transformation can define a rotation matrix (e.g., a 3×3 rotation matrix) that defines a 3D spatial rotation operation and a translation vector (e.g., a 3×1 translation vector) that defines a 3D spatial translation operation.

The loop closure engine 608 is configured to process the initial-to-terminal transformations 604 and the terminal-to-initial transformations 606 to identify the set of macrocycles 114. To this end, the loop closure engine 608 identifies pairs of fragment conformations 112, i.e., including a first fragment conformation and a second fragment conformation, where the initial-to-terminal transformation of the first fragment conformation satisfies a loop closure criterion with the terminal-to-initial transformation of the second fragment conformation.

Generally, if a first fragment conformation and a second fragment conformation satisfy a loop closure criterion, then the first fragment conformation and the second fragment conformation jointly form an (at least approximately) closed loop. A few examples of loop closure criteria are described next.

In some implementations, a loop closure criterion is satisfied for an initial-to-terminal transformation and a terminal-to-initial transformation if the transformation parameter values defining the initial-to-terminal transformation are equal to the transformation parameter values defining the terminal-to-initial transformation. In these implementations, if the initial-to-terminal transformation for a first fragment conformation and the terminal-to-initial transformation for a second fragment conformation satisfy the loop closure criterion, then the pair of fragment conformations jointly form a closed loop.

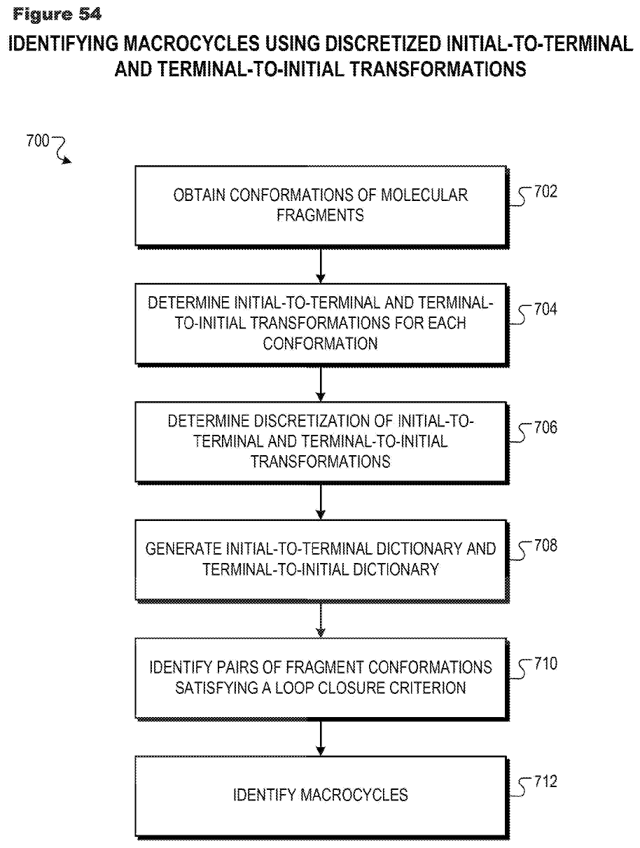

In some implementations, a loop closure criterion is satisfied for an initial-to-terminal transformation and a terminal-to-initial transformation if a discretization of the transformation parameter values defining the initial-to-terminal transformation is equal to a discretization of the transformation parameter values defining the terminal-to-initial transformation. In these implementations, if the initial-to-terminal transformation for a first fragment conformation and the terminal-to-initial transformation for a second fragment conformation satisfy the loop closure criterion, then the pair of fragment conformations jointly form an at least approximately closed loop. (The closeness of the approximation is determined by the fineness of the discretization of the transformation parameter values defining the initial-to-terminal transformation and the terminal-to-initial transformation). Discretizing the transformation parameter values defining the initial-to-terminal and the terminal-to-initial transformations can enable the loop closure criterion to be efficiently evaluated over a large number of fragment conformations, as will be described below with reference to FIG. 54.

Discretizing a value from a first set of possible values can refer to mapping the value to a discrete value drawn from a second set of possible values, where the second set of possible values includes fewer values than the first set of possible values. Parameter values defining initial-to-terminal transformations and terminal-to-initial transformations are drawn from a continuous space of possible values, e.g., a continuous Euclidean space. Discretizing the parameter values defining a transformation refers to mapping the continuous transformation parameter values defining the transformation to corresponding discrete transformation parameter values from a discrete (i.e., non-continuous) set of transformation parameter values. (The “fineness” of the discretization characterizes how densely the discrete set of transformation parameter values covers the continuous space of transformation parameter values).

The conformation of a molecule formed from a pair of molecular fragments (i.e., including a first molecular fragment and a second molecular fragment) is defined by the conformation of the first molecular fragment and the conformation of the second molecular fragment. The first and second molecular fragments can each assume conformations from a set of conformations, and thus the molecule can assume multiple possible conformations. A conformation of a molecule formed from a pair of molecular fragments 110 (including a first molecular fragment and a second molecular fragment) is said to satisfy a loop closure criterion if the conformation of the first molecular fragment and the conformation of the second molecular fragment satisfy the loop closure criterion.

The macrocycle identification system 600 can identify a molecule formed from a first molecular fragment and a second molecular fragment as being a macrocycle 114 based on whether the possible conformations of the molecule satisfy the loop closure criterion. In some cases, the macrocycle identification system 600 can identify a molecule as being a macrocycle 114 if at least one conformation of the molecule satisfies the loop closure criterion. In some cases, the macrocycle identification system 600 can identify a molecule as being a macrocycle if at least a predefined number of conformations of the molecule satisfy the loop closure criterion. In some cases, the macrocycle identification system 600 can identify a molecule as being a macrocycle 114 if at least a predefined fraction of the conformations of the molecule satisfy the loop closure criterion.

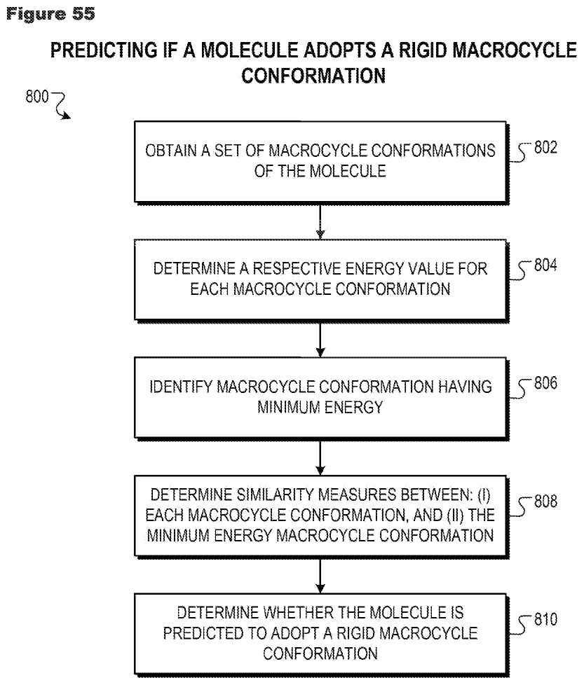

In some implementations, the macrocycle identification system 600 predicts whether a molecule that can assume a set of macrocycle conformations will form a rigid macrocycle based on the distribution of energies and conformations of the molecule that satisfy the loop closure criterion. An example process for predicting whether a molecule will form a rigid macrocycle is described with reference to FIG. 55.

The set of fragment conformations 112 can include a large number of fragment conformations, e.g., billions of fragment conformations. Therefore directly evaluating the loop closure criterion for each pair of fragment conformations may be computationally infeasible. An example of an efficient and computationally tractable process for identifying macrocycles by evaluating the loop closure criterion for discretized transformation parameter values is described with reference to FIG. 54.

FIG. 54 is a flow diagram of an example process 700 for identifying macrocycles using discretized initial-to-terminal transformations and discretized terminal-to-initial transformations. For convenience, the process 700 will be described as being performed by a system of one or more computers located in one or more locations. For example, a macrocycle identification system, e.g., the macrocycle identification system 600 of FIG. 53, appropriately programmed in accordance with this specification, can perform the process 700.

The system obtains a collection of fragment conformations that includes a respective set of fragment conformations for each of multiple molecular fragments (702). The collection of fragment conformations can include any appropriate number of fragment conformations, e.g., 1 million fragment conformations, 1 billion fragment conformations, or 1 trillion fragment conformations. The molecular fragments and fragment conformations can be generated, e.g., by the fragment generation system described with reference to FIG. 48.

The system determines an initial-to-terminal transformation and a terminal-to-initial transformation for each fragment conformation in the collection of fragment conformations (704). An initial-to-terminal transformation for a fragment conformation defines a translation and a rotation of the initial end of the fragment conformation relative to the terminal end of the fragment conformation. A terminal-to-initial transformation for a fragment conformation defines a translation and a rotation of the terminal end of the fragment conformation relative to the initial end of the fragment conformation.

The system discretizes the initial-to-terminal transformations and the terminal-to-initial transformations (706). More specifically, for each transformation (i.e., each initial-to-terminal transformation and each terminal-to-initial transformation), the system discretizes a set of transformation parameter values defining the transformation. The system can discretize a set of transformation parameter values defining a transformation using any appropriate discretization technique. For instance, the space of transformation parameters can be a Euclidean space, and the system can partition the Euclidean space into hyper-cubes, e.g., having predefined side lengths and volumes. The system can discretely represent a set of transformation parameter values (i.e., represented in the continuous Euclidean space) by an index of the hyper-cube that includes set of transformation parameter values.

The system generates a dictionary representing the initial-to-terminal transformations (an “initial-to-terminal” dictionary) and a dictionary representing the terminal-to-initial transformations (a “terminal-to-initial” dictionary) (708).

The initial-to-terminal dictionary includes: (i) a set of keys, and (ii) one or more values associated with each key. Each key represents a respective discretized initial-to-terminal transformation. Each value associated with a key identifies a fragment conformation with a discretized initial-to-terminal transformation represented by the key.

To generate the initial-to-terminal dictionary, the system can instantiate a copy of the set of discretized initial-to-terminal transformations, and then de-duplicate the set of discretized initial-to-terminal transformations, i.e., by removing duplicate copies of discretized initial-to-terminal transformations. The system can then process each discretized initial-to-terminal transformations from the de-duplicated set to generate a corresponding key for the initial-to-terminal dictionary. The system can generate a key from an initial-to-terminal transformation in any of a variety of ways. For instance, the system can process the set of discretized parameter values representing the initial-to-terminal transformation using a hash function to generate a corresponding key. The hash function can be, e.g., a multiplicative hash function, an algebraic coding hash function, a Fibonacci hash function, etc. Each key can represented by appropriate numerical data, e.g., an integer value. In addition to generating the keys for the initial-to-terminal dictionary, the system can associate each key with a set of values identifying each fragment conformation with the discretized initial-to-terminal transformation represented by the key.

To generate the terminal-to-initial dictionary, the system can instantiate a copy of the set of discretized terminal-to-initial transformations, and then de-duplicate the set of discretized terminal-to-initial transformations, i.e., by removing duplicate copies of discretized terminal-to-initial transformations. The system can then process each discretized terminal-to-initial transformations from the de-duplicated set to generate a corresponding key for the terminal-to-initial dictionary. The system can generate a key from a terminal-to-initial transformation in any of a variety of ways. For instance, the system can process the set of discretized parameter values representing the terminal-to-initial transformation using a hash function to generate a corresponding key. The hash function can be, e.g., a multiplicative hash function, an algebraic coding hash function, a Fibonacci hash function, etc. Each key can represented by appropriate numerical data, e.g., an integer value. In addition to generating the keys for the terminal-to-initial dictionary, the system can associate each key with a set of values identifying each fragment conformation with the discretized terminal-to-initial transformation represented by the key.

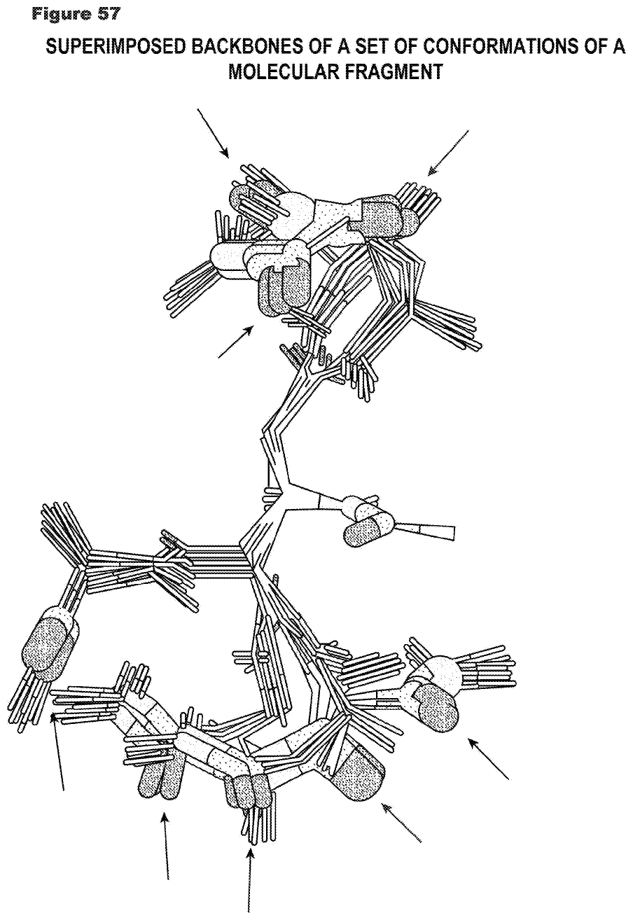

The number of keys in the initial-to-terminal dictionary can be significantly less than the original number of initial-to-terminal transformations, e.g., by one or more orders of magnitude. In particular, the sets of parameter values representing respective initial-to-terminal transformations are generally not uniformly distributed throughout the space of transformation parameter values, but rather are clustered into a number of groups. Thus discretizing the initial-to-terminal transformations can cause the large number of continuous-valued initial-to-terminal transformations to be mapped onto a significantly smaller set of discretized initial-to-terminal transformations. Each key in the initial-to-terminal dictionary may therefore be associated with multiple discretized initial-to-terminal transformations, e.g., 10 transformations, 100 transformations, or 1,000 transformations. FIG. 57, which will be described in more detail below, illustrates the clustering of the initial-to-terminal transformations and the terminal-to-initial transformations in the space of transformations.

Similarly, the number of keys in the terminal-to-initial dictionary can be significantly less than the original number of terminal-to-initial transformations, e.g., by one or more orders of magnitude. Each key in the terminal-to-initial dictionary can be associated with multiple discretized terminal-to-initial transformations, e.g., 10 transformations, 100 transformations, or 1,000 transformations.

The system uses the initial-to-terminal dictionary and the terminal-to-initial dictionary to identify pairs of fragment conformations satisfying the loop closure criterion (710). In particular, the system can identify a set of one or more “joint” keys that are common to both the initial-to-terminal dictionary and to the terminal-to-initial dictionary. For example, the system can identify joint keys by performing a set intersection operation on: (i) the set of keys of the initial-to-terminal dictionary, and (ii) the set of keys of the terminal-to-initial dictionary. For each joint key, the system can identify each pair of fragment conformations that includes: (i) a first fragment conformation associated with the joint key in the initial-to-terminal dictionary, and (ii) a second fragment conformation associated with the joint key in the terminal-to-initial dictionary, as satisfying the loop closure criterion.

Discretizing the transformations and forming dictionaries, as described above, can reduce the computational complexity of identifying pairs of transformations satisfying the loop closure criterion by one or more orders of magnitude, e.g., as compared to directly evaluating the loop closure criterion for every pair of continuous-valued transformations. In particular, discretizing the transformations can collapse the number of unique transformations to a significantly smaller number of unique discrete transformations, and set intersection operations can be used to efficiently compare dictionaries to identify common keys and thus pairs of transformations satisfying the loop closure criterion.

The system identifies one or more macrocycles based on the identified pairs of fragment conformations satisfying the loop closure criterion (712). For instance, the system can identify a molecule formed from a pair of molecular fragments as being a macrocycle if the molecule has a set of conformations that satisfy the loop closure criterion.

FIG. 55 is a flow diagram of an example process 800 for predicting whether a molecule that can adopt a set of macrocycle conformations will adopt a rigid macrocycle conformation. For convenience, the process 800 will be described as being performed by a system of one or more computers located in one or more locations. For example, a macrocycle identification system, e.g., the macrocycle identification system 600 of FIG. 53, appropriately programmed in accordance with this specification, can perform the process 800.

The system obtains a set of macrocycle conformations of the molecule (802). The system can obtain the set of macrocycle conformations of the molecule, e.g., by the process described with reference to FIG. 54.

The system determines, for each macrocycle conformation, a respective energy value associated with the macrocycle conformation (804). The system can determine an energy value for a macrocycle conformation, e.g., using an energy prediction machine learning model, as described with reference to FIG. 49.

The system identifies a macrocycle conformation having a minimum energy value among the macrocycle conformations (806).

The system determines, for each macrocycle conformation, a respective similarity measure between: (i) the macrocycle conformation, and (ii) the macrocycle conformation having the minimum energy value (808). The system can evaluate a similarity between a pair of macrocycle conformations, e.g., based on a root-mean-square deviation (RMSD) of atomic positions between the pair of macrocycle conformations.

The system determines whether the molecule is predicted to adopt a rigid macrocycle conformation based on a fraction of the macrocycle conformations of the molecule having: (i) an energy that satisfies an energy threshold, and (ii) a similarity to the minimum energy macrocycle conformation that satisfies a similarity threshold (810). The system can determine that the energy of a macrocycle conformation satisfies the energy threshold, e.g., if the energy of the macrocycle conformation is less than the energy threshold. The system can determine that the similarity of a macrocycle conformation to the minimum energy macrocycle conformation satisfies a similarity threshold, e.g., if the similarity is greater than the similarity threshold. The system can determine that the molecule is predicted to adopt a rigid macrocycle conformation, e.g., if the fraction of macrocycle conformations that simultaneously satisfy the energy threshold and the similarity threshold has at least as threshold value.

FIG. 56 illustrates a first molecular fragment and a second molecular fragment having conformations that jointly form a closed loop.

FIG. 57 illustrates a superposition of the backbones of a set of conformations of a molecular fragment. The initial ends of the fragment conformations are aligned, and the terminal ends of the fragment conformations cluster into a small number of groups. The system described in this specification can exploit the clustered distribution of fragment conformations in order to efficiently identify macrocycle conformations, as described above with reference to FIG. 54.

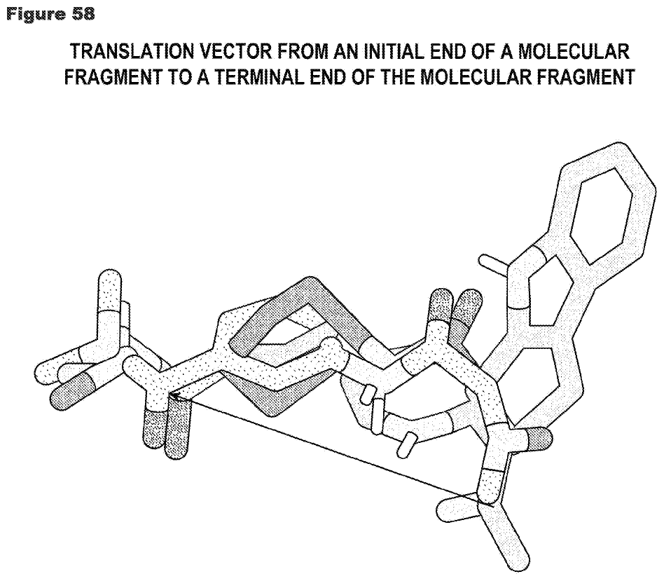

FIG. 58 shows a translation vector defining a translation from an initial end of a molecular fragment to a terminal end of the molecular fragment. The translation vector can form part of an initial-to-terminal transformation that the system described in this specification can use to identify macrocycle conformations, as described above with reference to FIG. 54.

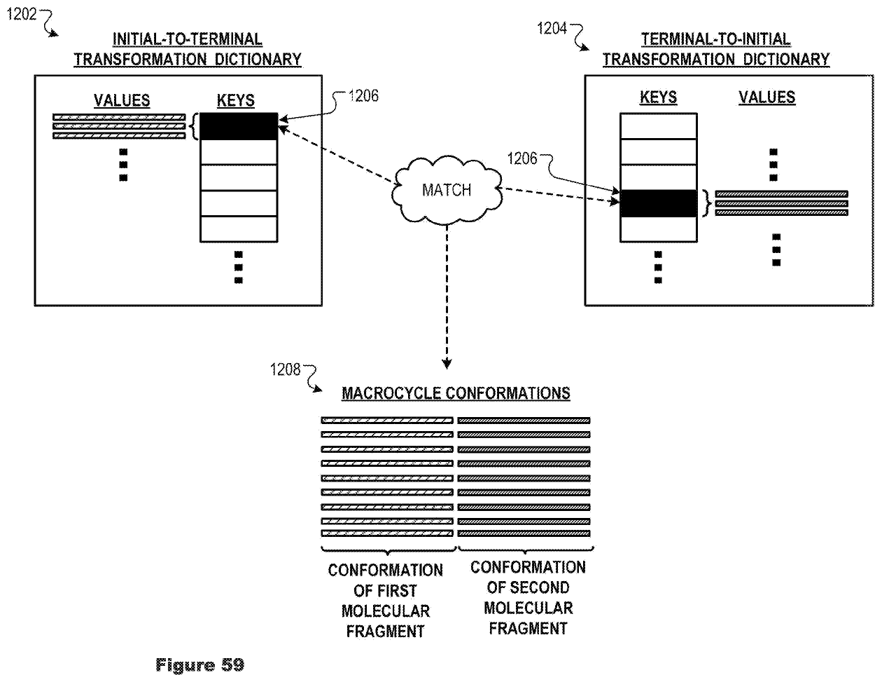

FIG. 59 shows an initial-to-terminal transformation dictionary 1202 and a terminal-to-initial transformation dictionary 1204. Each key in the initial-to-terminal dictionary 1202 represents a discretized initial-to-terminal transformation, and is associated with a set of values that specify fragment conformations having the discretized initial-to-terminal transformation represented by the key. Each key in the terminal-to-initial dictionary 1204 represents a discretized terminal-to-initial transformation, and is associated with a set of values that specify fragment conformations having the discretized terminal-to-initial transformation represented by the key. The system described in this specification can efficiently identify macrocycle conformations 1208 by the values associated with keys common to both dictionaries, e.g., the key 1206, as described above with reference to FIG. 54.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices, magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover. although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

### EXAMPLES

Small macrocycles composed of only 3 or 4 canonical and non-canonical amino acids are among the most pharmacologically potent natural products yet isolated, but there are very few of these in nature, and there is currently no way to systematically explore the structural space that they encompass. Here, we report a general computational method for identifying, by large scale sampling, closed macrocycles formed from combinations of alpha, beta, gamma, delta, epsilon, aminobenzoic, aminophenylacetic, aminomethylbenzoic, and aminomethylpicolinic acids along with oxazoles, thiazoles, oxazolidines, thiazolidines, triazoles and thioethers. To make the search computationally tractable, the rigid body transformations between terminal amides of one and two monomer units are rapidly compared by hashing to identify combinations of conformers that close to form a macrocycle. We use this method to identify 14.9 million macrocycles built from 3 or 4 monomers that contain over 42,000 unique combinations of canonical and non-canonical backbones, and up to 32-membered rings; approximately 7.4 million of these macrocycles satisfy the rule of five criteria for drug-like compounds. We chemically synthesized 30 macrocycles predicted to adopt single low-energy states, and determined X-ray or NMR structures of 18, 15 of which were very close to the corresponding computational models. Our results vastly increase the number of known small macrocycles, and should enhance structure based drug design and other applications that rely on small molecule libraries.

Macrocycles composed of up to four alpha or beta amino acids, amino benzoic acids, oxazoles, or thiazoles have bioactivities ranging from antifungal or antibiotic properties to cancer cytotoxicity to pain relief. Despite the diversity of bioactivities observed in natural products of this class, chemists and biologists have primarily limited their exploration of this space to homologues that are very similar to those already sampled by nature-indeed, the majority of macrocyclic drugs currently approved for use in humans are primarily derived from natural products. Computational methods that enable the rapid and comprehensive exploration of the space of possible macrocycles could greatly facilitate the discovery of natural product-like compounds with novel bioactivities, but such methods do not currently exist. Methods have been developed for systematically enumerating large macrocycles formed from all alpha amino acids by first generating cyclic conformers of poly-glycine backbones and then carrying out sequence optimization, but these methods cannot be readily extend to generate macrocycles formed from monomers with diverse backbone chemistry because they require randomly sampling backbone torsion angles to facilitate identifying closed conformers of the macrocycle. This random search through combinations of backbone torsion angles and subsequent sequence design is tractable when the macrocycle is constructed from a single backbone building block (e.g. only alpha-amino acids), but it becomes intractable when many different backbones are included: there are nearly 60,000 two dimensional chemical structures of 2-, 3-, and 4-residue macrocycles that can be constructed from the 22 building blocks depicted in FIG. 1A, and each of the 60,000 can be further diversified with potentially millions of combinations of different sidechains. This chemical space is far too large to rely on random sampling to identify the small fraction of linear sequences that can be closed to form a cycle.

We set out to develop a computationally tractable method for sampling the very large chemical space of possible small macrocycles (FIG. 1). We explored a three step approach for carrying out this search given an input set of monomers with widely varying chemical structures. In the first step, low energy conformers of each of the monomers are generated. In the second step, the rigid body transformations associated with these monomer conformations and all pairwise combinations of the monomer conformations into dipeptide like structures are computed and stored in a hash table. In the third step, macrocycles are rapidly and systematically generated by identifying pairs of entries in the hash table for which the sum of the rigid body transforms is close to zero (so that together the fragments form a cycle).

To identify low-energy conformers for a given set of monomers, we developed an adaptive grid-based search of backbone and side chain torsion angles to generate potential energy surfaces for each of the monomers. For energy evaluation, we utilized the atoms-in-molecules deep neural network, AIMNet, modified with a London dispersion correction; this requires only atomic coordinates and atomic numbers as inputs and thus does not require complex atom typing like many molecular mechanics-based energy functions, and has accuracy comparable to DFT-based methods that are orders of magnitudes more expensive to evaluate. From these energy surfaces, we extract low-energy conformers to use as building blocks for assembling macrocycles. To enable rapid identification of pairs of one and two monomer conformers that when combined close to generate a macrocycle, we compute the 6-dimensional rigid-body transform between the terminal amides of each low energy monomer and dipeptide conformer in both the N-to-C and C-to-N directions, and store these in a hash table. Simply obtaining the intersection of hash values that exists in the N-to-C hash table of one building block and the C-to-N hash table of another building block nearly instantly identifies the pairs of these building blocks that share the complementary terminal orientations required to form a closed macrocycle (for example, the pair of green hash entries on the left and right in FIG. 1b). This hashing approach requires far less compute time than would be required to explicitly build backbone coordinates and evaluate closure for the very large number of possible conformers for each of the different monomer combination possibilities, and is agnostic to the number, identity, and connectivity of atoms between the terminal amides, as only their relative orientations in space are considered for determining closure. The generality of this approach allows macrocycles to be built from every possible unique combination of the monomers depicted in FIGS. 1A and 5. While FIG. 1B outlines this process for constructing a 4-residue macrocycle from building blocks of dipeptide conformers, the approach is generalizable to any size; e.g. 3-residue macrocycles are found by searching for dimers that are closed by monomers and vice versa, while two-residue macrocycles are found by searching for monomers that are closed by other monomers.

### Enumeration of Closed Macrocycle Backbones

We used our approach to explore the very large space of 3- and 4-residue macrocycles constructed from 130 unique monomers, and their enantiomers where appropriate, along with C-terminal dimethylamide variants (FIG. 5). These monomers fall into 22 classes, hereafter referred to as chemotypes, which are distinguished by the atomic number and hybridization of the atoms in the backbone of each monomer (FIG. 1A); we refer to each chemotype by a letter (e.g. a for alpha-amino acids, b for beta-amino acids, d for delta-amino acids, etc.; see FIG. 1A legend). From the potential energy surfaces for each monomer, we constructed hash tables for both monomers and dimers, and then systematically searched through all combinations of monomer-dimer, dimer-monomer, and dimer-dimer hash tables, generating ensembles for each chemical for which matching hash values could be found. These hash tables contain nearly 9 billion unique conformations of the monomer and dimer subunits; searching for closures by explicit conformer generation over all combinations would have required building ca. 10¹⁹ macrocycles, which is far beyond what is possible; our hashing approach reduces the complexity required to evaluate all of these combinations from O(n2) to O(n).

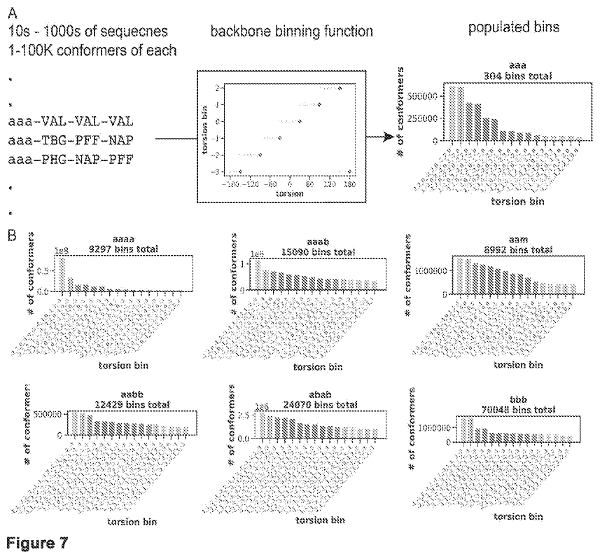

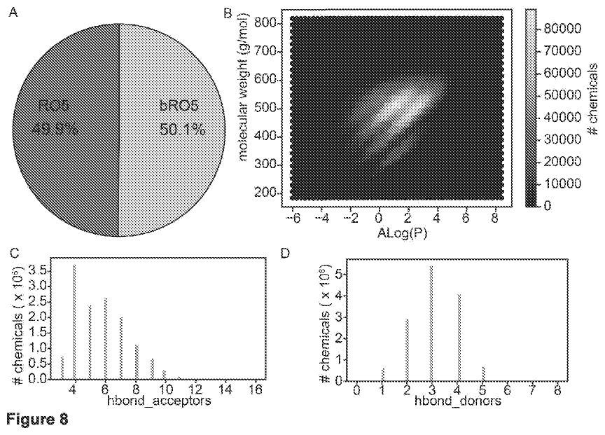

Systematically searching through the hash tables yielded 14.9 million closed macrocycles containing 9- to 32-membered rings belonging to 3,494 3-residue and 38,544 4-residue chemotypes (accounting for all unique circular permutations of the cyclic chemotypes). The chemical space spanned by the 3- and 4-residue chemotypes (FIG. 2A) greatly expands upon what has been previously explored (FIG. 7): 206 unique chemicals belonging to 23 chemotypes have high resolution structures deposited in the Cambridge Structural Database (CSD) and 13,932 chemicals belonging to 397 chemotypes are described in the pubchem database. Previous computationally designed macrocycles have been built from just one chemotype (alpha amino acids). Our 14.9 million closed macrocycles span a range of molecular weights and calculated octanol-water partition coefficients; roughly half satisfy the rule-of-five (Ro5) criteria for drug-like compounds (FIG. 8)(13).

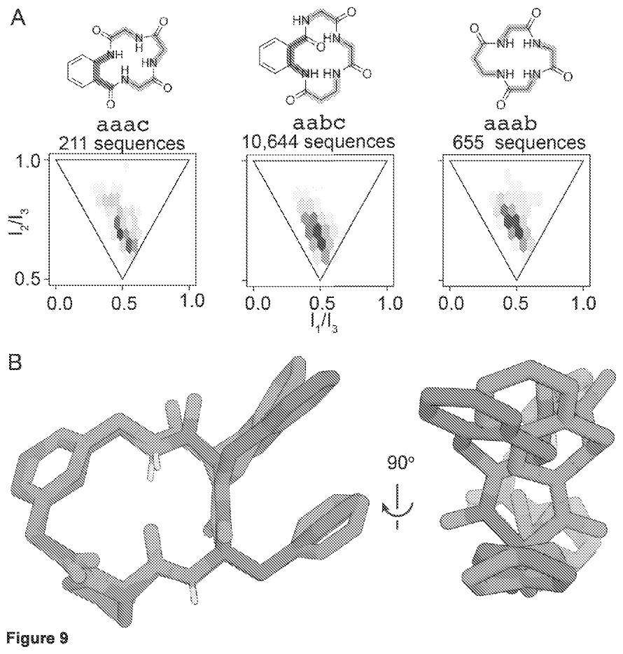

Each chemotype samples distinct three dimensional shapes (FIG. 2B and C). To characterize the structural diversity for each chemotype, we binned backbone torsion angles into 60 degree bins, and represented each macrocycle conformer by a string of these bins. The many thousands of conformers generated for each chemotype for the most part have only a handful of bin strings (FIG. 9), which likely reflect the torsional preferences of the constituent amino acids. Principal moment of inertia (PMI) analysis of conformers spanning all the bin strings sampled for each sequence revealed that different macrocycle chemotypes sample distinct regions of shape-space (14). In some cases—e.g. aaaa and aaac—the specific 3D shapes sampled are quite restricted by the closure constraint, regardless of sequence or torsional diversity (FIG. 9).

The hash-based closure generates for each monomer sequence an ensemble of macrocycle structure models built from low energy monomer conformers. Given sufficient compute power, for each of these ensembles, we could carry out minimization in AIMNet (to incorporate monomer-monomer interactions, closure strain, etc.), and evaluate the degree to which the sequence of monomers uniquely encodes a single low-energy conformer by considering the energy landscape mapped by the set of all low energy conformers for the sequence (using for example the Pₙₑₐᵣ approximation to the Boltzmann weight; (15)).

To evaluate this hash-based conformational sampling followed by AIMNet minimization approach, we used it to map the energy landscapes of eight macrocycles with known structure built from 3 or 4 alpha-amino, beta-amino, and amino benzoic acid monomers. The backbone RMSD of the lowest energy conformer in the hash-generated ensembles following AIMNet minimization to the corresponding X-ray crystallographic structure was 0.3 angstroms or better in all eight cases (FIG. 10). Thus, the hash sampling procedure coupled with AIMNet calculations can predict the structures of small macrocycles from their sequence of monomers. Carrying out the full energy calculations on all 14 million generated ensembles is, however, not computationally tractable: approximately 10⁸ cpu-hours would be needed to minimize the ca. 23 billion conformers sampled across the 14 million ensembles. Instead, we focused on two subclasses of closed macrocycles we hypothesized would be particularly likely to have single unique ground states: first, those with local interactions strongly favoring one or a small number of closed states, and second, those containing non-local hydrogen bonds between backbone amides.

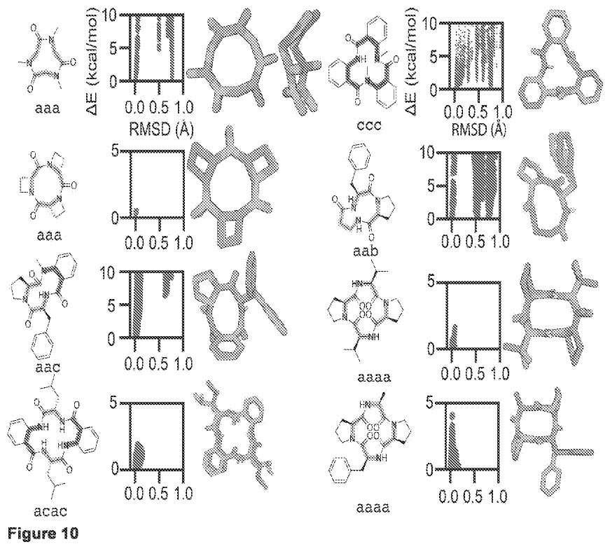

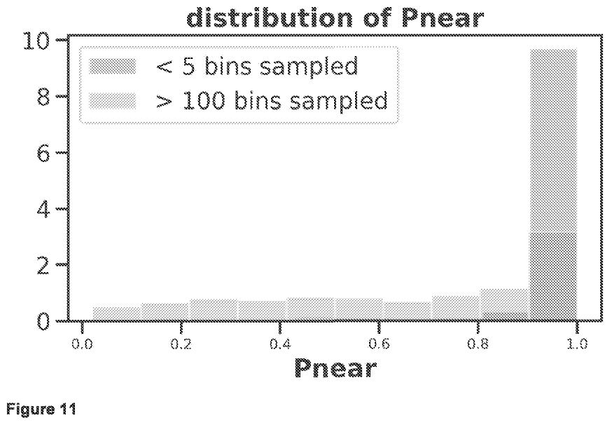

To identify the first locally-encoded subclass, we generated conformational ensembles from hash tables containing only monomers within 1 kcal/mol of their lowest energy state; such macrocycles are torsionally optimized from the perspective of the composite building blocks. As the resulting macrocycle space is still very large, for computational tractability we explored the use of the number of different torsion bin strings sampled in these ensembles as an indicator of the extent to which the torsional biases together with the closure constraint are sufficient to specify a single low energy minimum. We found following AIMNet minimizations of a subset of the ensembles that in cases where five or fewer torsion bin strings were sampled there were generally deep energy minima in the AIMNet landscapes around a single state with Pnear>0.9 (FIG. 11). We identified ca. 380,000 3- and 4-residue sequences for which the hash generated ensembles contained more than 50 closed conformers that spanned five or fewer torsion bins, and carried out AIMNet minimization on ca. 4800; roughly 85% of these had single low energy structures with Pnear>0.9.

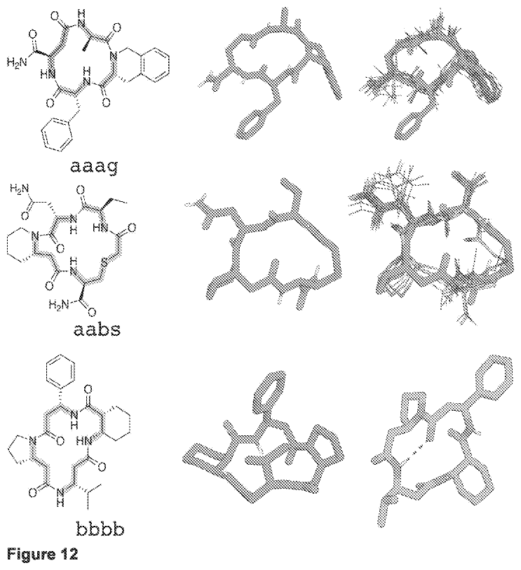

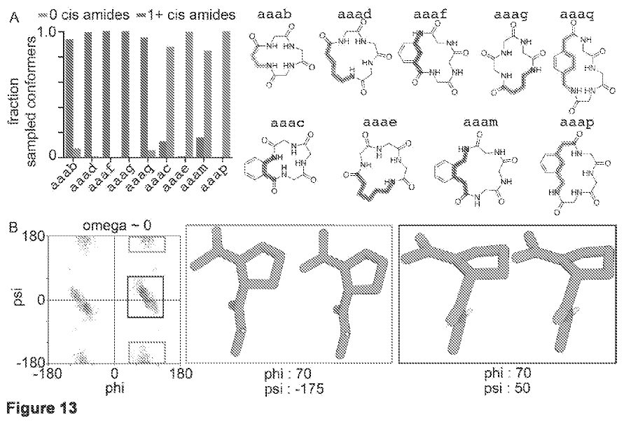

We synthesized 13 such macrocycles and were able to determine structures of ten using X-ray crystallography and NMR spectroscopy (FIG. 3, 12; 19-47). Six macrocycles grew crystals in vapor diffusion experiments of sufficient quality for structure determination using direct methods; seven macrocycles had NMR spectra in DMSO-d6 with sufficient dispersion to enable the unambiguous assignment of key ROEs in ROESY spectra—for these we generated 3D models by relaxing conformers generated from integrated ROE based distance constraints in unconstrained molecular dynamics (MD) simulations, and selecting the lowest-energy conformers (see Methods). Eight of the 1 experimentally determined structures superimpose on their respective design models with RMSDs of 0.8 Å or less, with small differences in ring puckering of 5- and 6-membered rings, in single side chain torsion angles amongst the side chains, and in pairs of backbone torsion angles (larger differences were observed for the other three macrocycles (FIG. 12)). We were able to determine both X-ray crystallographic structures and NMR structures for two of the 11 macrocycles. In these two cases (aabc and aaam) both the NMR structure and the X-ray crystallographic structures are very similar to their respective design models and to each other (FIG. 13).

The macrocycles with solved structures (FIG. 3) are built from a total of eight different monomer chemotypes (a, b, c, h, k, m, q, s) arranged into six unique macrocycle chemotypes (aas, aaam, aaaq, aabc, ahah, akak). The aas design contains two hydrogen bond interactions; one between backbone amides; the second involving the primary amide of the s monomer. Both aaam macrocycles and the akak macrocycle contain proline-like monomers sampling cis amides. In the NMR structures of both aaam macrocycles these motifs lead to strong ROEs between the Cα proton of the proline-like monomer and the Cα proton of the monomer immediately N-terminal to it; these macrocycles contain α-amino acids in very extended β-sheet-like conformations and the cis amides rapidly turn the backbone to enable closure (FIG. 14). The aaaq macrocycle contains an extended pentafluorophenylalanine residue that buries its amide NH against the 4-aminomethylphenylacetic acid thereby shielding it from solvent and causing the amide NH to shift downfield with a temperature shift coefficient (Tcoeff) of 0.9 ppb/K (the backbone amides in the remaining monomers of this design shift upfield with Tcoeffs all less than −5.0 ppb/K, see supplementary data). The aaam and aaaq macrocycles provide two different ways to mimic the extended conformations of poly-α-amino acid peptides, like those adopted by many protease and kinase substrates (16, 17), and is useful for targeting these enzymes.

To identify the second subclass of macrocycles—those containing transannular hydrogen bonds—we constructed dimer hash-tables with conformers for which the terminal amides make a hydrogen bond, increasing the monomer energy cut-off for hash table inclusion to within 4.0 kcal/mol of the global minima to increase sampling of these rare interactions. To reduce local strain, following identification of macrocycle chemotypes that contained long range hydrogen bonding interactions, we chose monomer identities and sidechain rotamers with low energy given the backbone torsion angles. Following hash table-based ensemble enumeration and AIMNet minimization, we selected for experimental characterization designs with much lower energy than predicted by a simple monomer composition based model (Methods).

We prepared 17 macrocycles designed to contain long-range hydrogen bonding interactions and were able to determine the high resolution structures of seven (FIGS. 4 and 19-47). Four macrocycles grew crystals in vapor diffusion experiments of sufficient quality for structure determination using direct methods; three macrocycles displayed NMR spectra with sufficient dispersion to facilitate the unambiguous assignment of key ROEs in ROESY spectra. Eight of the seventeen macrocycles displayed 1D-NMR in DMSO-d6 indicative of a mixture of states; six of these collapse into a single major species in CDCl₃. While only three macrocycles showed sufficient dispersion for unambiguous assignment, ten of the 17 had sufficient dispersion to enable measuring amide Tcoeffs which are consistent with the presence of the designed transannular hydrogen bonds in all ten cases. All seven of the experimentally determined structures superimpose on their respective design models with RMSDs less than 0.8 angstroms. Like the macrocycles described in FIG. 3, small differences between the designed model and the experimental structures of these hydrogen bond-containing macrocycles involve differences in ring-puckering and 180 degree rotation of backbone amides.

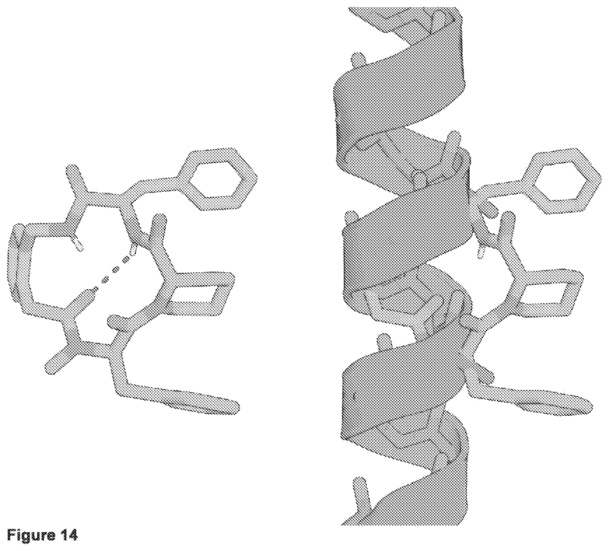

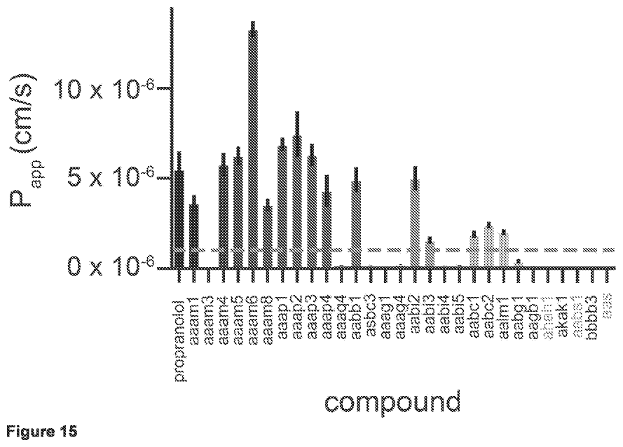

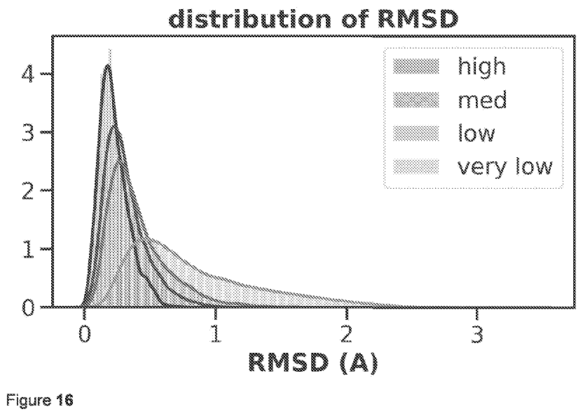

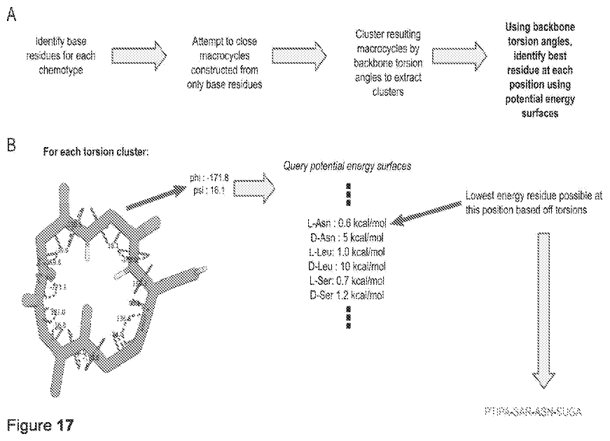

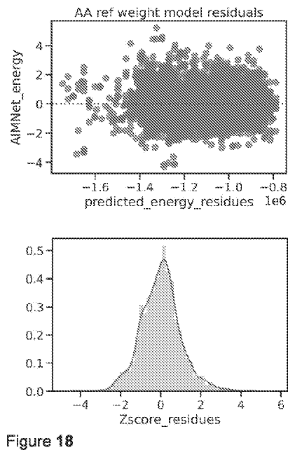

The macrocycles depicted in FIG. 4 are constructed from eight different monomer chemotypes (a, b, g, h, i, l, m, p) arranged into six unique macrocyclic chemotypes (aaam, aaap, aabi, aagb, aalm, ahah), and are stabilized by a transannular hydrogen bond between backbone amides involving non α-amino acids. The aalm-based macrocycle contains a hydrogen-bonding fragment built from monomers with predominantly sp2 hybridized atoms in the backbone (lm), the aagb-based macrocycle contains a hydrogen-bonding fragment whose backbone contains many more sp3 hybridized atoms than present in α-amino acid backbones (gb), and the aabi-based macrocycle contains a hydrogen-bonding fragment that blends these two features (bi). Six of the macrocycles contain contiguous fragments of α-amino acids that mimic β-turns common in protein-protein interfaces: the aagb-, aabi-, and aaam-based macrocycles contain turns akin to type-I β-turns, and the aalm-based macrocycle contains a turn akin to the type-II β-turn. The N-methylated amino acid residues present in both aaap-based macrocycles adopt cis amides, resulting in type-VI like turns. Despite the presence of these obvious β-turn-like features in the aaam macrocycle, the spacing and orientation of the two phenylalanine side chains mimic the spacing and orientation of side chains positioned at i and i+4 of an α-helix (FIG. 15; this spacing is not present in either of the two aaap-based macrocycles which are isomers of aaam at the level of the backbone atoms). This mimicry of beta turn and helical arrangements is useful in targeting proteins recognizing these structural elements.

We measured the passive membrane diffusion of all 29 macrocycles designed with the two approaches using the parallel artificial membrane permeability assay (PAMPA) (FIGS. 3, 4, and 16). Almost all of the macrocycles were quite membrane permeable: 16 of the 29 had log(Pₐₚₚ)'s greater than −6, and only three were not detected in the acceptor well. Under the same conditions, the small molecule drug propranolol achieved log(Pₐₚₚ) of −5.36 (FIG. 16). As expected, designs with exposed polar groups, particularly side chain hydroxyls and primary amides, had lower permeabilities. Overall, the hydrogen-bond containing designs were more permeable than the torsionally-optimized based designs, likely due to fewer exposed backbone polar groups.

### Compound Synthesis

All macrocycles in Table 3 were synthesized. Below is a detailed protocol for the synthesis of PJS-MPRO-1-j (Compound 218 in Table 3). A similar protocol was used to prepare other macrocycles. Dichloromethane was added to a vessel containing CTC resin (0.1 mmol, 0.65 mmol/g, 0.15 g) and 2-(2-((((9H-Fluoren-9-yl)methoxy)carbonyl)amino)phenyl)acetic acid (0.38 mg, 0.10 mmol, 1.00 eq) and bubbled with nitrogen. Diisopropylethylamine (4 eq.) (DIEA) was added dropwise and mixed for 2 hours at which point methanol (0.15 mL) was added. This mixture was allowed to bubble for an additional 30 minutes. The reaction vessel was drained of solvent, and washed with dimethylformamide (DMF) five times. After washing, a solution of 20% piperidine in DMF was added to the reaction vessel and bubbled for 30 minutes, at which point the vessel was drained of solvent, and washed 5× with DMF. To the reaction vessel, a solution of Fmoc-protected amino acids was added, followed by a solution of activating reagents. This reaction proceeded for 1 hour with mixing by nitrogen bubbling, at which point the reaction vessel is drained of solvent, and washed with 5× with DMF. This cycle of deprotect, wash, couple, wash is repeated until the full-length linear peptide is completed, and the N-terminal Fmoc-protecting group is removed. After completion of the linear peptide, the resin was washed with methanol thrice and dried under vacuum. The reagents used in the coupling steps for synthesis of PJS-MPRO-1-j are depicted in Table 4.

TABLE 4

| # | Materials | Coupling reagent |
|---|---|---|
| 1 | 2-(2-((((9H-Fluoren-9- | DIEA (4.00 eq) |
| | yl)methoxy)carbonyl)amino)phenyl)acetic | |
| | acid (1.00 eq) | |
| 2 | Fmoc-L-β-Homophenylalanine (3.00 eq) | HATU (2.85 eq) and |
| | | 2,4,6-Trimethyl- |
| | | pyridine (6.00 eq) |
| 3 | Fmoc-3-Pal-OH (3.00 eq) | HATU (2.85 eq) and |
| | | DIEA (6.00 eq) |
| 4 | Fmoc-Phe(2-Me)-OH (3.00 eq) | HATU (2.85 eq) and |
| | | DIEA (6.00 eq) |

The linear side-chain protected peptide was cleaved from the resin by addition of a cleavage solution (1% trifluoroacetic acid in dichloromethane) to the resin at room temperature. The cleavage was carried out twice for 3 minutes each with continuous nitrogen bubbling. The Filtrate of the cleavage reaction was collected, pooled, and diluted with 100 mL of dichloromethane. To the dilute linear protected peptide, TBTU (64.2 mg, 2 eq), and HOBt (27 mg, 2 eq.) was added. The pH of this solution was adjusted to ca. pH 8.0 with DTEA. The mixture was stirred for 30 minutes at 25 degrees celsius. The reaction mixture was then washed with 1 M HCl, and the organic layer was concentrated to dryness under reduced pressure. The resulting residue was treated with global-deprotection solution (92/5% trifluoroacetic acid, 2.5% 3-mercaptopropionic acid, 2.5% TIS, 2.5% water) and stirred for 30 minutes. The mixture was precipitated with cold isopropyl ether and centrifuged for 3 minutes at 3000 rpm. The resulting insoluble material was subsequently precipitated with additional isopropyl ether two additional times, each time discarding the solvent. The resulting solid material containing the crude cyclic peptide was dried under vacuum for 2 hours to remove any residual isopropyl ether.

The peptide was then purified from this material by reverse-phase preparative HPLC (A: 0.075% TFA in water, B: ACN) to give PJS-MPRO-1-j (3.1 mg, 94.6% purity, 5.14% yield) as white solid.

### Activity Testing

Compounds were tested for the ability to alter the function of one of the following protein targets: M. tuberculosis ClpP1P2 protease, MCL-1, SARS-CoV-2 main protease, FKBP51, M. tuberculosis DnaN, tubulin, insulin degrading enzyme, thrombin, MDM2.

### Assays

SARS-CoV-2 main protease: The compounds were tested in 10-dose IC50 singlet with a 3-fold serial dilution starting at 200 uM. The protease activities were monitored as a time-course measurement of the increase in fluorescence signal.

CLPP1P2: The compounds were tested in 10-dose IC50 singlet with a 3-fold serial dilution starting at 200 uM. The protease activities were monitored as a time-course measurement of the increase in fluorescence signal.

MCL-1: The compounds were tested in 10-dose IC50 singlet with a 3-fold serial dilution starting at 200 uM. Compounds in DMSO were added to enzyme solution by using Acoustic Technology. Compounds were incubated with enzyme for 10 min. at room temperature. Respective compounds were then added, and the mixture incubated for another 10 min. Finally, Anti-GST-Tb was added. After 60 min incubation, the HTRF signal was measured.

Tubulin: The assay was performed using a commercially available tubulin polymerization assay kit from Cytoskeleton (SKU: BKO11) following the manufacturer's instructions.

Thrombin: The assay was performed using a commercially available thrombin activity assay kit from Abcam (SKU: ab197007) following the manufacturer's instructions.

MDM2: The assay was performed using a commercially available alphalisa assay kit from PerkinElmer (part number AL3168C) following the manufacturer's instructions.

DanN: The assay was conducted as a competition assay with results observed using biolayer inferotometry.

FKBP51: The compounds were tested in 10-dose IC50 singlet with a 3-fold serial dilution starting at 200 uM.

### Activity

Compounds 11 (4.58 uM), 16 (13.5 uM), 18 (28.2 uM), 19 (40.6 uM), 29 (22.8 uM), 119 (18.9 uM), 121 (50.1 uM), and 122 (12.3 uM) were most active in the MCL-1 assay.

Compounds 32 (100 uM) and 35 (20 uM) were most active in the MDM2 assay.

Compounds 131 (4.07 uM), 132 (1.25 uM), 133 (0.844 uM), 134 (1.22 uM), 134 (1.22 uM), 135 (1.47 uM), 136 (1.47 uM), 137 (1.27 uM), 138 (2.14 uM), 139 (2.37 uM), 140 (1.53 uM), 141 (1.84 uM), 142 (1.89 uM), 143 (2.65 uM), 144 (1.26 uM), and 218 (17.8 uM) were most active in the SARS-CoV-2 main protease assay.

### CONCLUSION

Small macrocycles found in nature generally populate a mixture of conformers in solution. Our approach enables the rapid exploration of the large and chemically diverse macrocycle space to identify those that populate primarily a single conformer. The two strategies employed here—using conformationally restricted building blocks to favor specific geometries, or to incorporate longer range hydrogen bonding interactions—both resulted in macrocycles with well-defined structures. Our observation that the chemotype of the macrocycles, regardless of its side-chain substitution, defines its globular shape suggests that diversity oriented synthesis methodology to span a wide range of shapes should focus on expanding the number of chemotypes that can be synthesized combinatorially as opposed to the diversity of side chains arrayed on a single or small number of chemotypes. Encouragingly for future therapeutic applications, nearly half of the macrocycles we characterized are membrane permeable, with log(Papp)>−6.0 in PAMPA, and this fraction would likely be considerably higher if we had explicitly designed for permeability, for example by disfavoring compounds with exposed NH groups. The chemical space can readily be expanded by including more diverse sidechains or additional non-canonical backbones in the hashing step, and almost all compounds in this class can be readily synthesized using standard manual Fmoc-based solid-phase peptide synthesis followed by solution-phase cyclization (see methods).

The very large and diverse set of compounds described here provides exciting new avenues in drug discovery. The rigidity of the molecules should translate into higher on-target affinity due to the lower entropy loss upon target binding, and lower off-target binding as there are fewer alternative states; rule-of-5 compliance during generation should lead to membrane permeability and other desirable pharmacological properties. Libraries of rule-of-5 compliant macrocycles populating single states can be screened in silico and/or experimentally to identify new lead compounds binding targets of interest. For targets for which small molecule fragments are already known to bind, custom rigid macrocycle libraries incorporating this specific functionality can be readily generated by searching the hash tables for all closed macrocycles that incorporate the fragments.

### REFERENCES

1. V. Sarojini, A. J. Cameron, K. G. Varnava, W. A. Denny, G. Sanjayan, Cyclic Tetrapeptides from Nature and Design: A Review of Synthetic Methodologies, Structure, and Function. Chem. Rev. 119, 10318-10359 (2019).
2. J. T. Mhlongo, E. Brasil, B. G. de la Torre, F. Albericio, Naturally Occurring Oxazole-Containing Peptides. Mar. Drugs. 18 (2020), doi:10.3390/md18040203.
3. T. Degenkolb, W. Gams, H. Brückner, Natural cyclopeptaibiotics and related cyclic tetrapeptides: structural diversity and future prospects. Chem. Biodivers. 5, 693-706 (2008).
4. M. J. Ferracane, A. C. Brice-Tutt, J. S. Coleman, G. G. Simpson, L. L. Wilson, S. O. Eans, H. M. Stacy, T. F. Murray, J. P. McLaughlin, J. V. Aldrich, Design, Synthesis, and Characterization of the Macrocyclic Tetrapeptide cyclo[Pro-Sar-Phe-d-Phe]: A Mixed Opioid Receptor Agonist-Antagonist Following Oral Administration. ACS Chem. Neurosci. 11, 1324-1336 (2020).
5. E. M. Driggers, S. P. Hale, J. Lee, N. K. Terrett, The exploration of macrocycles for drug discovery—an underexploited structural class. Nat. Rev. Drug Discov. 7, 608-624 (2008).
6. M. Muttenthaler, G. F. King, D. J. Adams, P. F. Alewood, Trends in peptide drug discovery. Nat. Rev. Drug Discov. 20, 309-325 (2021).
7. K. T. Mortensen, T. J. Osberger, T. A. King, H. F. Sore, D. R. Spring, Strategies for the Diversity-Oriented Synthesis of Macrocycles. Chem. Rev. 119, 10288-10317 (2019).
8. G. Sangouard, A. Zorzi, Y. Wu, E. Ehret, M. Schüttel, S. Kale, C. Diaz-Perlas, J. Vesin, J. Bortoli Chapalay, G. Turcatti, C. Heinis, Picomole-Scale Synthesis and Screening of Macrocyclic Compound Libraries by Acoustic Liquid Transfer. Angew Chem Int Ed Engl. 60, 21702-21707 (2021).
9. P. Hosseinzadeh, G. Bhardwaj, V. K. Mulligan, M. D. Shortridge, T. W. Craven, F. Pardo-Avila, S. A. Rettie, D. E. Kim, D.-A. Silva, Y. M. Ibrahim, I. K. Webb, J. R. Cort, J. N. Adkins, G. Varani, D. Baker, Comprehensive computational design of ordered peptide macrocycles. Science. 358, 1461-1466 (2017).
10. E. A. Coutsias, K. W. Lexa, M. J. Wester, S. N. Pollock, M. P. Jacobson, Exhaustive conformational sampling of complex fused ring macrocycles using inverse kinematics. J. Chem. Theory Comput. 12, 4674-4687 (2016).
11. R. Zubatyuk, J. S. Smith, J. Leszczynski, O. Isayev, Accurate and transferable multitask prediction of chemical properties with an atoms-in-molecules neural network. Sci. Adv. 5, eaav6490 (2019).
12. E. Caldeweyher, S. Ehlert, A. Hansen, H. Neugebauer, S. Spicher, C. Bannwarth, S. Grimme, A generally applicable atomic-charge dependent London dispersion correction. J. Chem. Phys. 150, 154122 (2019).
13. C. A. Lipinski, F. Lombardo, B. W. Dominy, P. J. Feeney, Experimental and computational approaches to estimate solubility and permeability in drug discovery and development settings. Adv. Drug Deliv. Rev. 46, 3-26 (2001).
14. W. H. B. Sauer, M. K. Schwarz, Molecular shape diversity of combinatorial libraries: a prerequisite for broad bioactivity. J. Chem. Inf Comput. Sci. 43, 987-1003 (2003).
15. G. Bhardwaj, V. K. Mulligan, C. D. Bahl, J. M. Gilmore, P. J. Harvey, O. Cheneval, G. W. Buchko, S. V. S. R. K. Pulavarti, Q. Kaas, A. Eletsky, P.-S. Huang, W. A. Johnsen, P. J. Greisen, G. J. Rocklin, Y. Song, T. W. Linsky, A. Watkins, S. A. Rettie, X. Xu, L. P. Carter, D. Baker, Accurate de novo design of hyperstable constrained peptides. Nature. 538, 329-335 (2016).
16. P. K. Madala, J. D. A. Tyndall, T. Nall, D. P. Fairlie, Update 1 of: Proteases universally recognize beta strands in their active sites. Chem. Rev. 110, PR1-31 (2010).
17. C. J. Miller, B. E. Turk, Homing in: mechanisms of substrate targeting by protein kinases. Trends Biochem. Sci. 43, 380-394 (2018).
18. J. Gavenonis, B. A. Sheneman, T. R Siegert, M. R. Eshelman, J. A. Kritzer, Comprehensive analysis of loops at protein-protein interfaces for macrocycle design. Nat. Chem. Biol. 10, 716-722 (2014).
19. M. Guharoy, P. Chakrabarti, Secondary structure based analysis and classification of biological interfaces: identification of binding motifs in protein-protein interactions. Bioinformatics. 23, 1909-1918 (2007).
20. B. L. Sibanda, T. L Blundell, J. M. Thornton, Conformation of beta-hairpins in protein structures. A systematic classification with applications to modelling by homology, electron density fitting and protein engineering. J. Mol. Biol. 206, 759-777 (1989).
21. P. N. Lewis, F. A. Momany, H. A. Scheraga, Chain reversals in proteins. Biochim. Biophys. Acta. 303, 211-229 (1973).
22. D. B. Diaz, S. D. Appavoo, A. F. Bogdanchikova, Y. Lebedev, T. J. McTiernan, G. Dos Passos Gomes, A. K. Yudin, Illuminating the dark conformational space of macrocycles using dominant rotors. Nat. Chem. 13, 218-225 (2021).

### Computational Methods

The entire computational pipeline is implemented using several python packages. The following sections detail their execution and high-level details of their operations.

Residue parameters: To facilitate constructing and manipulating coordinates of both monomers and macrocycles, we generated a python dictionary that contains myriad parameters for each residue. These parameters store several boolean values concerning the chemical identity of the residue, python lists of various atom indices, and SMILES strings of the residue. These residue parameters are used in myriad places throughout the scripts and workflows below. The following is a simplified version of the residue_params dictionary that contains only glycine.

```
residue_params = {
  ‘AGLY’ : {
      ‘smiles’ : ‘CC(=0)NCC(=0)NC’,
      ‘dofs’ : [[1, 3, 4, 5], [3, 4, 5, 7]],
      ‘proline_like’ : False,
      ‘preproline’ : False,
      ‘chiral’ : False,
      ‘lower_smiles’ : ‘CC(=0)’,
      ‘residue_smiles’ : ‘NCC(=0)’,
      ‘upper_smiles’ : ‘NC’,
      ‘lower_indices’ : [0, 1, 2, 9, 10, 11],
      ‘residue_indices’ : [3, 4, 5, 6, 12, 13, 14],
      ‘upper_indices’ : [7, 8, 15, 16, 17, 18],
      ‘lower_connect_indices’ : [1, 2, 3],
      ‘upper_connect_indices’ : [5, 6, 7],
      ‘backbone_indices’ : [0, 1, 3, 4, 5, 7, 8],
      ‘atoms’ : [6, 6, 8, 7, 6, 6, 8, 7, 6, 1, 1, 1,
1, 1, 1, 1, 1, 1, 1],
    ‘atom_types’ : [‘SP3’, ‘SP2’, ‘SP2’, ‘SP2’, ‘SP3’,
    ‘SP2’, ‘SP2’, ‘SP2’, ‘SP3’, ‘S’, ‘S’, ‘S’, ‘S’,
    ‘S’, ‘S’, ‘S’, ‘S’, ‘S’, ‘S’],
    ‘bonds’ : [[0, 1], [1, 2], [1, 3], [3, 4], [4, 5],
    [5, 6], [5, 7], [7, 8], [0, 9], [0, 10], [0, 11],
    [3, 12], [4, 13], [4, 14], [7, 15], [8, 16], [8,
    17], [8, 18]],
    ‘bond_types’ : [‘SINGLE’, ‘DOUBLE’, ‘SINGLE’,
    ‘SINGLE’, ‘SINGLE’, ‘DOUBLE’, ‘SINGLE’, ‘SINGLE’,
    ‘SINGLE’, ‘SINGLE’, ‘SINGLE’, ‘SINGLE’, ‘SINGLE’,
    ‘SINGLE’, ‘SINGLE’, ‘SINGLE’, ‘SINGLE’, ‘SINGLE’],
      ‘rotamers’ : 2,
    ‘charges’ : [−0.18, 0.25, −0.38, −0.5, 0.03, 0.27,
    −0.39, −0.47, −0.08, 0.12, 0.1, 0.11, 0.23, 0.15,
    0.11, 0.25, 0.14, 0.13, 0.11],
  },
}
```

Boltzmann adaptive sampling: We developed a new sampling protocol to generate the entire potential energy surface of the monomeric residues which we have called Boltzmann Adaptive Sampling (BAS).

The iterative process of building the Delaunay triangulation, selecting points, and evaluating the energy of the molecule is repeated until the potential energy surface of the molecule has converged. The BAS scheme utilizes Delaunay triangulation to maintain the relationship between all of the points that have been sampled. For this protocol, all points of the Delaunay triangulation are the dihedral values of conformations that have been sampled. We store the conformation and the energy of the molecule that is associated with the point. Bond torsion with many possible values, such as phi and psi of alpha amino acids, were sampled with the BAS; bond torsions with very few possible angles like amide bonds were considered as “rotamers” that were not adaptively sampled but instead they were fixed in a cis or trans configuration and evaluated in two separate runs. Likewise dihedrals that must change in concert with one another (i.e. ring flips) were treated as individual “rotamers” and evaluated in two separate runs.

The sampling protocol is initialized by sampling a sparse regular grid with 7 points per dimension (i.e. rotatable bond). This was found to sample the most common dihedral angles (−180°, −120°, −60°, 0°, 60°, 120°, 180°). Note that the first and last dihedral angles are identical, but Delaunay triangulation implementations that work in higher dimensions cannot account for periodicity. After the initial grid conformations are evaluated, we create the Delaunay triangulation that will guide further sampling. Every simplex of the Delaunay triangulation is evaluated with a loss function which is inspired by the Boltzmann probability (see below). The simplex with the highest loss is then chosen. Once a simplex is chosen, we select the center of the simplex with small random perturbations to avoid deterministic sampling. That point corresponds to the next set of torsions that will be evaluated. After evaluating that point by minimizing the conformation with the new dihedral angles, that point is added back into the Delaunay triangulation. This results in the selected simplex to divide into multiple new simplices. This process of identifying simplices, evaluating the centers of the simplices, and updating the Delaunay triangulation can be done individually or in batches. While performing this process one simplex at a time is optimal for sampling the simplex with the highest loss, we perform the process in small batches (<32) because the time to update the Delaunay triangulation is significant. We developed a dynamic controller that adjusted the batch size to ensure that 99% of the runtime was spent on evaluating the molecular energy. This controller was especially necessary in high dimensions because the Delaunay triangulation calculation scales poorly with number of dimensions and number of samples. If a controller is not used to adjust the batch size, the protocol will spend most of the time selecting which points to sample instead of evaluating the energy of these points. The loss function that determines which simplices to sample next is the most critical part of this protocol. We devised what we term the volume adjusted Boltzmann probability, which guides the adaptive grid to sporadically sample both small simplicities whose points are very low in relative energy, or large simplicities that have not been explored. This ensures that the adaptive grid does not continually subdivide the lowest energy simplicity which would result in narrow sampling.

Once the new points have been selected for evaluation, the monomers are constructed and subjected to minimization in AIMNet. We found that without minimization, the resulting potential energy surfaces of the molecules become sharp and discontinuous. We found that minimizing the conformation with bond torsion constraints produced smooth potential energy surfaces that qualitatively recapitulated known potential energy surfaces like alanine. It was essential to perform a full minimization on the molecule because minute changes in bond lengths and bond angles (which are not constrained in the minimization) can significantly affect the energy of a conformation. We also found that using the conformation of the nearest neighbor as the starting point before setting the major degrees of freedom and applying constraints improved the run time and convergence of the minimization trajectories. Presumably this is because the nearest neighbor point has similar perturbations to the minor degrees of freedom.

The key metric for performance of the new sampling protocol is the rate of convergence of the potential energy surface. The potential energy surface can be generated by linearly interpolating the Delaunay triangulation. As more points are sampled in the Delaunay triangulation, a linear interpolation of the points will become more and more accurate at predicting new interpolated points. Once enough points have been sampled, the potential energy surface for a given molecule will converge and adding more points will not affect the potential energy surface. Once the potential energy surface is converged, the energy of any conformation can be accurately predicted.

We found that the BAS identifies low-energy conformers of monomers compared to naive grid-based searches; particularly for torsionally constrained monomers like aminobutyric acid, or for monomers with many rotatable bonds. While the BAS performed well to generate potential energy surfaces for the monomers used in this study, we found that it struggles to sample the potential energy surface of molecules with more than 6 degrees of freedom. This barrier is due to the time to calculate the Delaunay triangulation and is exacerbated by the fact that higher dimensions require more sampled points in general. For example, in 6 dimensions with 1 million points, updating the Delaunay triangulation with 100 additional points is time equivalent to minimizing the energy of these 100 new points. This discrepancy between updating the triangulation and evaluating the energy of the new points on the surface becomes more dramatic in higher dimensions (i.e. when more bond torsions are sampled). For these reasons, the BAS is ideal for sampling the potential energy surfaces of monomers, but not macrocycles.

We used BAS to generate potential energy surfaces for each monomer depicted in FIG. 5. In these calculations, each monomer surface was generated for each monomer, containing both C-terminal methylamide and C-terminal dimethylamide modifications. The latter are pre-proline and are denoted with _pp appended to the residue's name. The potential energy surfaces are stored on disk as HDF5 files that contain datasets of the AIMNet minimized conformers of the monomer and their associated AIMNet energies (in kcal/mol) for subsequent use in hashing and macrocycle construction (see below). In the below methods, these potential energy surfaces are referred to as “conformer databases”. The following command illustrates how to generate the potential energy surface of glycine:

OMP_NUM_THREADS=1 python run_residue.py --residue AGLY -
-rotamer 0 --number of threads 4

Sequence compatibility check: In the below procedures, the compatibility of two monomers to construct a dimer is frequently checked before proceeding with generating hash tables or ensembles of macrocycles. Two monomers being compatible means that the monomers respect the following rules: In a dipeptide, if monomer 2 is proline_like (e.g. proline, N-methylated, proline-like, etc.) then conformers of monomer 1 must come from the preproline conformer database of monomer 1; If monomer 2 is not proline_like, then conformers of monomer 1 must not come from the preproline conformer database of monomer 1. To facilitate this check, each monomer is ascribed two boolean properties in the residue_params dictionary; one that stores if the monomer is proline_like; the second stores if the monomer is preproline.

Hash table construction: Monomer hash tables are built through a three step process. First, conformers of the specified monomer are loaded into memory from the conformer databases created as described above. Conformers of the monomer can be filtered for inclusion in the hash tables by specifying an energy threshold in kcal/mol. Conformers whose energy relative to the global minimum energy for the monomer that are above this threshold are not included in the hashing. Second, coordinate frames are constructed for the terminal amides using the amide nitrogen, oxygen, and carbon atoms and the relative transformation between these coordinated frames is calculated and binned using the hash function available in the python package xbin. This is done for the N-terminal coordinate frame relative to the C-terminal coordinate frame, as well as for the C-terminal coordinate frame relative to the N-terminal coordinate frame. Third, the binned transformations, and the conformers that belong within the bins, are sorted and ultimately used to construct a getpy dictionary object, and an associated numpy array we term the key-value array. The dictionary contains as keys, the unique set of binned transformations calculated across every conformer brought into the hashing under step 1. The values that are accessed using these keys are tuples of start and stop indices that are used to select sub slices of the key-value array. Indexing the key-value array with this tuple will produce a numpy array of conformer indices that are used to access the conformers of the monomer within its conformer database that belong to the specific binned transformation (detailed below). These dictionaries and their respective key-value arrays are stored on disk as binary files for later use.

In this process, the size of the bins used for hashing is determined by two parameters, the Cartesian resolution, and the origin resolution. Increasing either will discretize the 6D space into few larger bins whereas decreasing either will discretize the 6D space into many smaller bins. This can dramatically impact the quality and numbers of closures found in subsequent steps. Large bins in the 6D space will result in hashtables with fewer binned transforms that each contain many conformers. Small bins in the 6D space will result in hashtables with many binned transforms that each contain few conformers. To tune the size of these bins, we created hashtables for each monomer and varying resolutions then assessed the quality of the resulting 2-residue macrocycles found from searching these hashtables for closures. For each conformer that this search produced, we built the macrocycle by superimposing the pair of N-terminal amides of one monomer onto the C-terminal amides of the second monomer. We then measured the RMSD between these two pairs of amides in the resulting conformer (FIG. 10). High quality closures are macrocycles where this RMSD is low. This occurs when the two conformers used to construct the macrocycle share very similar relative orientations. Low quality closures are macrocycles where this RMSD is high. This occurs when the two conformers have different relative orientations that were placed into the same bin in hashing due to large discretization of the 6D space. We found that as the size of the bins increases the resulting ensembles of macrocycles contain many conformers, however the quality of the closures is often poor. As the size of the bins decreases, the resulting ensembles of macrocycles will contain fewer conformers, but the quality of the closures increases. While poor closures can be identified in AIMNet minimizations, we focused on identifying high quality closures. As such in this study we used either 1.0 angstrom cartesian resolution with 15.0 degree origin resolution or 0.5 angstrom cartesian resolution with 10.0 degree origin resolution.

The below command demonstrates constructing hash tables of each monomer within HDF5_DIR to include conformers no more than 6 kcal/mol above the global minimum using 1.0 angstrom cartesian and 15.0 degree origin binning resolutions, which will be save in HASH_DIR:

python make_monomer_NC-CN_hash.py -kT 6.0 -car_resl 1.0
-ori_resl 15.0 -hash_dir <full path to HASH_DIR> -
hdf5_dir <full path to HDF5_DIR>

Hash tables for dimers are constructed in a nearly identical process. First, conformers for two monomers, monomer 1 and monomer 2, are loaded into memory after assessing their sequence compatibility. Second, each pair of monomer 1 and monomer 2 conformers is aligned to construct a dimer. In this alignment, the C-terminal amide of monomer 1 is aligned to the N-terminal amide of monomer 2. The relative transformation between coordinate frames of the terminal amides (N-termini of monomer 1 and C-termini of monomer 2) are calculated and binned. The resulting getpy dictionary and associated key-value array is stored on disk for future use. Here, the key-value array stores two indices, instead of one index. The first is used to identify the conformer of monomer 1 in the dimer, the second is used to identify the conformer of monomer 2 in the dimer that belongs to the binned transform in question. The following command demonstrates constructing hash tables of a proline-alanine dipeptide:

python make_dimer_NC-CN_hash.py -m1 PRO -m2 ALA -kT 1.0
-cart_resl 0.5 -ori_real 10.0 -hash_dir <full path to
HASH_DIR> -hdf5_dir <full path to HDFS_dir>

Hashtables that contain dimers with hydrogen bonding interaction are constructed by first identifying conformations of the dimer that contain hydrogen bonds, and only using those conformers in hashing. Hydrogen bonds are identified with a rapid geometric score term. This score term ranges between 0 and 1 with higher numbers producing more linear hydrogen bonds between amides. In this study we generated hydrogen bond-containing hashtables that scored above 0.75 with this term, and contained conformers no higher than 4 kcal/mol above the global minimum. The below command demonstrates this for the same proline alanine dipeptide, ensuring that no conformers that contain cis amides are present (unless the monomer is proline_like) and that the quality of the hbond interaction is at least greater than 0.75:

python make_dimer_NC-CN_hash.py -m1 PRO -m2 ALA -kT 4.0 -
cart_resl 0.5 -ori_resl 10.0 --filter_out_cis --
hbond_threshold 0.5 -hash_dir <full path to HASH_DIR> -
hdf5_dir <full path to HDF5_dir>

Chemotype and sequence alphabetization: Each monomer is assigned a single character that defines the number and hybridization of atoms along the backbone, starting from the N-terminal amide nitrogen, and ending at the C-terminal amide carbon. These single characters, or chemotypes, represent the first layer of alphabetization. Additionally, each monomer is assigned a priority based on its arbitrary location in a list of all monomers. This priority represents the second layer of alphabetization. When combined, the first and second layers of alphabetization generate an offset that is used to permute the input sequence into the lowest priority alphabetized chemotype. This ensures that a single sequence of monomers, regardless of their circular permutation, generates a single output macrocycle during ensemble generation. The methods to accomplish this alphabetization are in coarse_cluster.py The first layer of alphabetization is used to contract circularly permuted backbones into the same chemotype. For example, the chemotypes aaab, aaba, abaa, and baaa are all the same chemotype, just permuted circularly. Given an input sequence of monomers we assign the sequence a chemotype, then permute that sequence into the circular permutation that is lowest priority when alphabetized. In the aforementioned example, aaab, aaba, abaa, and baaa will all be permuted into aaab.

The second layer of alphabetization is used to ensure that an input sequence of monomers, regardless of circular permutation, only generates a single macrocyclic sequence. For chemotypes that contain some element of circular symmetry (e.g. aaaa, abab, etc.) there exists degenerate circular permutations. For example, acac can be permuted into the four following chemotypes: acac, caca, acac, and caca. By chemotype alphabetization, the first and third permutation in this are the same. In such cases, the monomer priorities are used to select between these degenerate chemotypes.

Ensemble generation: Ensembles are generated in a four-step procedure. First the requested hash tables (as getpy dictionaries) are loaded into memory. Second, any shared keys between both hashtables are identified and the conformer indices belonging to these shared bins are extracted from their respective key-value arrays. Third, all combinations of conformer indices of the N-to-C shared bin are combined with all combinations of conformer indices of the C-to-N shared bin. This procedure produces two dimensional numpy arrays that we term IJKL. The IJKL arrays are (M_conformers×i_residues) where M_conformers is the number of macrocycle conformers within the ensemble, and i_residues are the number of monomers in that macrocycle. For example, the 100 membered ensemble of the 3-residue cycle aaa-AGLY-AGLY-AGLY will have an IJKL array that is (100, 3), and IJKL[0,:] are the indices of the monomer conformers used to construct the 0th conformer of the macrocycle. IJKL[0,0] is the first conformer index of the first glycine monomer of the macrocycle, IJKL[0,1] is the first conformer index of the second glycine monomer of the macrocycle, etc. Third, the second dimension of the IJKL array is permuted into the lowest priority sequence according to the alphabetization process detailed above. The permuted IJKL array is finally saved to disk within an HDF5 file that is named according to the lowest priority sequence for the macrocycle.

The following commands demonstrate ways to generate 3 and 4 residue macrocycles. In all of these commands, note that the cartesian resolution and origin resolution must be specified and match those used to construct the hash tables. It is also important to segregate the dimer and monomer hashtables into separate HASH_DIR. All of these commands will attempt to generate ensembles and output them to CSV_DIR/SUB_DIR Saving the ensembles into a handful of subdirectories helps ensure the file quota limits are not reached in large scale sampling. Possible write collisions that can arise due to circular permutations in sequence are further avoided by appending a unique string to the output ensemble name.

The following command demonstrates searching for ensembles of every 3-residue macrocycle that contains a proline-alanine dipeptide at 1.0 kcaVmol that can be constructed from monomer hashtables in <HASH_DIR>:

python build_3mers_ensemble.py -m1 PRO -m2 ALA -cart_resl
0.5 -ori_resl 10.0 -kT 1.0 -hash_dir <full path to monomer
HASH_DIR> -csv_dir <full path to where output ensembles
will be saved> -sub_dir <subdirectory within CSV_DIR> -
subfile <unique string> -hdf5_dir <full path to HDF5_dir
containing conformer databases>

The following command demonstrates generating ensembles for every possible 4-residue macrocycle that can be constructed from dimer hashtables in <HASH_DIR> that close the proline alanine dipeptide:

python build_4mers_ensemble.py -m1 PRO -m2 ALA -cart_resl
0.5 -ori_resl 10.0 -hash_dir <full path to HASH_DIR> -
csv_dir <full path to where output ensembles will be
saved> -sub_dir <subdirectory within CSV_DIR> -subfile
<unique string>

The following command demonstrates attempting to generate an ensemble for only the macrocycle aaaa-ALA-ALA-ALA_pp-PRO:

python build_4mers_ensemble.py -m1 PRO -m2 ALA -m3 ALA -
m4 ALA_pp -cart_resl 0.5 -ori_resl 10.0 -hash_dir <full
path to HASH_DIR> -csv_dir <full path to where output
ensembles will be saved> -sub_dir <subdirectory within
CSV_DIR> -subfile <unique string>

After generating these ensembles within subdirectories with unique names appended to the ensembles, the following command will combine the unique conformers of every sequence within a chemotype split across these separate ensembles into a single HDF5 file for each chemotype:

python combine_seperated_ensembles.py <CHEMOTYPE> <full
path to WORK_DIR>

Note that WORK_DIR here should contain CSV_DIR, and a directory named HDF5. The combined ensembles will be saved in WORK_DIR/HDF5/CHEMOTYPE/.

Generation of 3D coordinates of macrocycles from ensembles stored in HDF5s; Prior to AIMNet minimizations or any other analysis, the IJKL array that stores the ensemble is converted into a (M_conformers×N_atoms×3) array of atomic coordinates in addition to a (N_atoms,) array of atomic numbers through a process we term building. We perform building from the ensemble IJKL arrays in memory immediately prior to any analysis/minimization to reduce the amount of space required to store the ensembles on disk. The below paragraphs details the procedure for building a 4-residue macrocycle.

First, all of the conformers from monomers in the given sequence are loaded from their respective conformer databases as (X_conformers, Y_atoms, 3) numpy arrays of atomic coordinates. The IJKL array is then used to index these atomic coordinates on the X_conformers axis, for each monomer. For a four residue macrocycle, this will produce four masked arrays, one for each monomer in the sequence. These arrays are (M_conformers, Y_atoms, 3) in shape, where M matches the number of conformers in the macrocycle ensemble and Y is the number of atoms in that specific monomer. Second, these masked arrays go through an initial build cycle where the N-terminal amide of monomer i+1 is aligned to the C-terminal amide of monomer i. This loop runs from i=1 to i=3, for a four residue macrocycle. Once this 3 residue fragment is constructed, the N-terminal amide of the final monomer is aligned to the C-terminal amide of the third monomer while simultaneously aligning the (C-terminal amide of the final monomer to the N-terminal amide of the first monomer.

Third, each monomer is subsequently realigned into the macrocycle such that the C-terminal amide of monomer i is aligned the N-terminal amide of monomer i+1 while simultaneously aligning the N-terminal amide of monomer i with the C-terminal amide of monomer i−1. This process cycles through a while loop that tracks the average RMSD between the amides that are being spliced together. This cycle completes once the average RMSD is no longer decreasing or a max number of cycles has been reached. Typically, we allowed no more than 200 cycles in this build loop.

At the end of this cycle, the four aligned (M_conformers, Y_atoms, 3) XYZ arrays are spliced together to construct the final (M_conformers, N_atoms, 3) XYZ array of the macrocycle, which we term the macrocycle_xyz array, in addition to the (N_atoms,) array of atomic coordinate, which we term the macrocycle_atoms array. These arrays can be used in subsequent calculations. The methods that accomplish these steps are implemented in utils_build.py.

Torsion-bin clustering: Torsion bin clustering is accomplished through the following steps. First, the 3D coordinates of every conformer in an ensemble are built in memory as described above. The resulting macrocycle_xyz array is masked to only contain the XYZ coordinates of the backbone atoms. These XYZ coordinates are converted into internal coordinates which produce an (M_confomers, N_backbone_atoms, 3) array of bond length, bond angle, and bond torsions that we term the dofs array. The dihedral angles are extracted from the dofs array and converted from an angle into an integer using the following equation:

$$bins = ( ( np . around ( dofs [ : , : , - 1 ] / torsion\_resolution ) + 3 ) \%6 - 3 ) . astype ( np . int 8 )$$

Where the torsion_resolution is set to 60 degrees. These bins are then saved into a new HDF5 for subsequent analysis. The following command is used to accomplish this torsion binning:

python analyze_macrocycle_torsions.py -i <full path to
ensemble> --write_cluster_hdf5 -out_dir <path to save
output HDF5> -pdb_dir <path to save PDB files of cluster
representatives> -hdf5_dir <path to conformer databases>

Principal moment of inertia analysis: The principal moments of inertia ratios for torsion clusters of several chemotypes were calculated as described. We implemented this calculation within the analyze_macrocycle_torsions.py script. The following command adds the two PMI ratios into the output HDF5 from torsion clustering as datasets labeled I1_I3 and I2_I3:
python analyze_macrocycle_torsions.py--moi-i<full path to ensemble>--write_cluster_hdf5-out_dir<path to save output HDF5>-pdb_dir<path to save PDB files of cluster representatives>-hdf5_dir<path to conformer databases>

In our analysis, we only use the backbone heavy atoms to determine the two PMI ratios.

AIMNet Mininizations and Pnear calculations: Ensembles of macrocycles were minimized in the AIMNet score function modified with the dftd4 correction using the ase python package using the BFGS optimizer. Each macrocycle conformer is constrained using the FixInternals constraints to ensure no chemical reactions occurred during minimization. Geometry optimization was run for no more than 100 steps, or until the max force did not exceed 0.005 eV/A. In these optimizations, the maxstep was set to 0.5 angstroms. This minimization scheme typically takes about 1-3 minutes on a CPU per macrocycle conformer to complete, depending on the number of atoms in the macrocycle.

We implemented this minimization in the minimze_macrocyle_ensemble.py script. This script first builds the 3D coordinates of the macrocycle from the input ensemble as described above, then subjects the conformers to minimization. This script enables parallelizing this minimization across many CPU cores using the flag -j. In our testing, each parallel job requires approximately 3 GB of RAM After all conformers have been minimized, the lowest energy conformer from the ensemble is identified. The backbone atoms of all other conformers are aligned to the backbone atoms of the low energy conformer. The RMSD of these alignments are calculated. The RMSD and AIMNet energies are used to construct an energy landscape like those depicted in FIG. 1B and FIG. 10. The Pnear value for the landscape is calculated using the below formula, with kT=0.62 and lamda=0.3 angstroms:

The below command demonstrates minimizing a random 1000 conformers from an ensemble. This command parallelizes this minimization across 20 CPU cores:

python minimize_macrocycle_ensemble.py -i <full path to
ensemble> --endpoint_only --limit 1000 -j 20 --save_hdf5
-hdf5_dir <path for conformer databases> -pdb_dir <path
to PDB_DIR> -png_dir <path to PNG_DIR>

This will produce a SVG file of the energy landscape in PNG_DIR, a PDB file that contains up to the 100 lowest energy conformers of the macrocycle in PDB_DIR, and a HDF5 file that contains the XYZs of the minimized ensemble, their RMSDs to the lowest energy conformer within the ensemble, and their AIMNet minimized energies saved into PDB_DIR. We manually inspected the lowest energy conformers macrocycles if the Pnear of their respective energy landscape was greater than 0.75.

Sequence design of hydrogen bonding macrocycles: We identified specific sequences capable of forming long rang hydrogen bonds using an augmented pipeline from that described above. We first identified a set of base residues within each chemotype. We then generated ensembles for all possible 4-residue macrocycles that contain combinations of only these base residues by searching for closures amongst the hydrogen bonds containing hash tables. We then clustered the resulting ensembles using the analyze_macrocycle_torsions.py script to produce pdb files containing a single representative of each torsion bin sampled in the ensemble. We then used the analyze_compatible_sequences.py script to identify possible low-energy sequences for each of the torsion-bin representatives. We then generate ensembles for each of the candidate low-energy sequences, and subject those ensembles to minimization as described above.

Candidate low-energy sequences are identified in the analyze_compatible_sequences.py script through 1-body sidechain packing algorithm. First, we identify possible side chain substitutions that are possible at each position or the macrocycle from the sequence of the base residue macrocycle (e.g. mutating glycine to alanine, phenylalanine, etc). We then rank-order these modifications based on their compatibility with the backbone torsion angles of the torsion-bin representative being processed. We query the potential energy surface of each possible modification at each position in the macrocycle to identify the relative energy of the lowest energy rotamer possible for each modification given the backbone torsion angles of the base residue in the macrocycle. This process allows identifying the monomers most likely to accommodate the torsion angles present in the torsion-bin representative. This is done for each position in the macrocycle being processed. This process results in a list of sequences, each of which is composed of the ideal modification of the base residue in each of the torsion-bin representatives. We found that this analysis produced sequences that, when minimized, reduced the occurrence of non-ideal torsion angles such as L-amino acids being placed at positions with positive phi torsion angles.

The below command is used to produce lists of candidate sequences for ensemble generation:

python analyze_compatible_sequences.py -i <full path to
pdb from torsion binning> -hdf5_dir <path for conformer
databases> -out_dir <path to save output lists>

We also used this process to identify at each position the 3 lowest energy monomers, and created candidate sequences of each combination at each position by adding the -max_monomers 3 flag.

We minimized approximately 8280 hydrogen bond-containing macrocycles resulting from the above sequence design process. To prioritize macrocycles from this subset for manual inspection and synthesis, we constructed a simple linear regression model that predicts the AIMNet energy of a macrocycle given its sequence composition. We constructed a matrix wherein each column is associated to a specific monomer and each row is associated to a macrocyclic sequence. For a given macrocycle, this matrix counts the number of specific monomers in each sequence. We then constructed a vector wherein each row contains the AIMNet energy of the low energy conformer of the macrocycle. We then used the sklearn python package to train weights associated with each monomer in the matrix using the LinearRegression model. We found that this model predicted the AIMNet energy of the low energy conformer of a given macrocycle within 5 energy units. We then prioritized macrocycles for inspection if the ZScore (calculated from the minimized AIMNet energy+the predicted AIMNet energy) was less than −2. This process identified macrocycles whose AIMNet energy is much more negative (i.e. favorable) than anticipated, given the sequence of the macrocycle.

### Materials and Methods

### General Information

Peptide synthesis: the macrocycles described here were prepared by WuXi apptec. Typically, WuXi assembled peptides on CTC resin using Fmoc-based solid-phase synthesis using iterative coupling with either HBTU/DIEA, HATU/DIEA, or DIC/HOBt. Linear peptides were cleaved from resin and then cyclized in solution. Typically, peptides were cyclized in either DCM or DMF using either TBTU/DTEA, HATU/DIEA or EDCl/HOBt. The crude cyclization reactions were then purified using reverse-phase HPLC. In WuXi's hands, these procedures typically afforded 0.5-20 mg of purified macrocycle as a white or off white powders, after lyophilization. Below is a more detailed protocol for the synthesis of aaap: CTC resin (0.1 g, 0.1 mmol, 0.99 mmol/g) was swelled in DCM under agitation with nitrogen. A solution of Fmoc-3-aminomethyl-phenylaetic acid (38.74 mg, 0.1 mmol, 1.0 eq) in DCM was added to the resin and agitated. DIEA (4.0 eq) was added to the resin dropwise. The resulting mixture was allowed to mix under agitation for 2 hours. The solution was then drained, and the resin was capped with a methanol (0.1 mL) for 30 minutes. The loaded and capped resin was then washed in DMF. The linear peptide was assembled using iterative Fmoc-deprotection reactions using 20% piperidine in DMF followed by coupling reactions using 6 eq of the incoming Fmoc-protected amino acids, 5.7 eq of HATU, and 12 eq of DIEA. The resin was washed 5 times between each deprotection and each coupling reaction using DMF. The progress of these reactions were monitored using the ninhydrin test. After the final Fmoc-deprotection reaction, the linear peptide was cleaved from resin using a 1% (v/v) solution of trifluoroacetic acid in DCM. The resin was subjected to two bouts of cleavage, each lasting 3 minutes. The combined filtrate containing the cleaved linear peptide was then diluted into 100 mL of DCM to which HATU (76 mg, 2 eq) was added. The pH of this solution was adjusted to pH˜8.0 using DIEA. The resulting mixture was stirred at room temperature for 0.5 h after which 1 M HCl was added. The organic phase was collected and concentrated under vacuum to give the crude macrocycle aaap. The crude reaction mixture was dissolved in water:ACN and purified by reverse-phase preparative HPLC (25-55% ACN over 55 minutes. Pure fractions were combined and lyophilized yielding 14.1 mg of aaap at ca. 99.4% purity.

NMR Spectroscopy: 1D ¹H-NMR and 2D ¹H-NMR spectra were recorded on a Bruker NOE 600 MHz spectrometer equipped with a QCI-F cryoprobe. TOCSY spectra were recorded with a 80-ms spin-lock mixing time. ROESY spectra were recorded with a 200-ms spin-lock mixing time. Data were processed in TopSpin.

VT-NMR: Variable temperature NMR experiments were performed to determine amide hydrogen temperature shift coefficients. ¹H-NMR spectra were recorded from 298 to 318 K in either DMSO-d6 or CDCl₃. Temperature shift coefficients less than 4 ppb/K were inferred to represent hydrogens that are shield from solvent, whereas coefficients greater than 4 ppb/K were inferred to represent hydrogens exposed to solvent.

NMR-based structure determination: The 3D structures of peptide macrocycles were determined using NMR-based distance constraints in combination with molecular dynamics simulations. ROE-based distance constraints were determined from the averaged integrated columns of ROE cross peaks across the diagonal. These integrals were converted to atomic distances using an inverse 6th power relationship. A reference distance of 1.78 angstroms for diastereotopic methylene protons or 2.49 angstroms for 1,2-substituted phenyl protons was used to calibrate the conversion of integrated ROEs to atomic distances. The calculated distances were then adjusted by 10% to provide a range of allowed distances for each distance constraint. Ensembles of macrocycle conformers that obeyed each distance constraint were obtained using the geometric hashing scheme detailed above. First, we constructed hash tables that contained monomers up to 6 kcal/mol in energy above the global minima. We then searched for closures in these tables. This search produced 10⁶-10⁸ conformers of the sequences in question. We then filtered those conformers based on their satisfaction of all of the experimentally derived distance constraints, resulting in an ensemble of approximately 400 conformers of the peptide. Each of these 400 conformers was subjected to a 50 picosecond unconstrained molecular dynamics simulation using xTB at 298K in either implicit DMSO or implicit CHCl₃ (Bannwarth et al. 2019). In total this resulted in approximately 20 nanoseconds of simulation time for each peptide. The 20 lowest energy structures across the entire ca. 20 ns simulation were taken as the ensemble structure of the peptide.

Single crystal X-ray diffraction of peptides: Crystals diffraction data were collected from a single crystal at synchrotron (on APS 24ID-C) and at 100 K. Unit cell refinement, and data reduction were performed using XDS and CCP4 suites (Kabsch 2010; Winn et al. 2011). The structure was identified by direct methods and refined by full-matrix least-squares on F² with anisotropic displacement parameters for the non-H atoms using SHELXL-2018/3 (Sheldrick 2015b; Sheldrick 2015a). Structure analysis was aided by using Coot/Shelxle (Emsley and Cowtan 2004; Hübschle et al. 2011). The hydrogen atoms on heavy atoms were calculated in ideal positions with isotropic displacement parameters set to 1.2×U_{eq} of the attached atoms.

PAMPA: Stock solutions of each peptide were prepared gravimetrically by dissolving ca. 1-3 mg of peptide in either 100% MeOH or 100% DMSO to produce ca. 1-10 mM peptide solutions. These stock solutions were stored at 4° C. when not in use. Samples for PAMPA experiments and calibration curves were prepared by diluting these stock solutions to produce 1.2 mL of 20 uM peptide in PBS supplemented with either 5% DMSO, or 10% MeOH. Calibration curves of each peptide were prepared by serial diluting 20 uM peptide 2-fold to obtain 6 samples with the following concentrations: 20, 10, 5, 2.5, 1.25, and 0.625 uM. These standard curves were used to determine the concentration of each peptide in both the donor and acceptor wells of the PAMPA plate. For all PAMPA experiments, we used the commercially available Corning Gentest Precoated PAMPA plate system (product number 353015), following the manufacturer's instructions. Briefly, donor wells were loaded with 300 μL of the 1.2 mL solutions. acceptor wells were loaded with 200 μL of either 5% DMSO in PBS or 10% MeOH in PBS. Each peptide was prepared in triplicate. The concentration of each peptide in the donor and acceptor wells was quantified by LCMS after approximately 19 hours. These concentrations were used to calculate Papp per the manufacturer's instructions.

TABLE 5

| Residue name | SMILES |
|---|---|
| ABOC | CC(═O)NC(CC1)(CCC1C2)[C@@H]2C(═O)NC |
| AMBEN4_pp | CC(═O)NCc1ccc(cc1)C(═O)N(C)C |
| AMC14C_pp | CC(═O)NC[C@@H]1CC[C@@H](CC1)C(═O)N(C)C |
| NMBEN3_pp | CC(═O)N(C)c1cc(ccc1)C(═O)N(C)C |
| ACBC12T | CC(═O)N[C@@H]1[C@H](CC1)C(═O)NC |
| AMC14T | CC(═O)NC[C@@H]1CC[C@H](CC1)C(═O)NC |
| BEN3_pp | CC(═O)Nc1cc(ccc1)C(═O)N(C)C |
| PPR | CC(═O)N1[C@@H](C[C@H](c2ccccc2)C1)C(═O)NC |
| ABU | CC(═O)N[C@@H](CC)C(═O)NC |
| CLICKS_pp | CC(═O)NCc1cn(nn1)[C@@H](C)C(═O)N(C)C |
| NMBEN2 | CC(═O)N(C)c1c(cccc1)C(═O)NC |
| NMPHG | CC(═O)N(C)[C@@H](c1ccccc1)C(═O)NC |
| NVL | CC(═O)N[C@@H](CCC)C(═O)NC |
| DHP_pp | CC(═O)N1[C@@H](C═CC1)C(═O)N(C)C |
| FPS | CC(═O)N1[C@@H](C[C@H](F)C1)C(═O)NC |
| HPR | CC(═O)N1[C@@H](C[C@@H](O)C1)C(═O)NC |
| HUCP | CC(═O)NCc1nc(ccc1)C(═O)NC |
| AGLS | CC(═O)N[C@@H](CC1)C(═O)N1[C@@H](C)C(═O)NC |
| ACBEN4 | CC(═O)Nc1ccc(cc1)CC(═O)NC |
| PTABU_pp | CC(═O)N(CC)CC(═O)N(C)C |
| NMSER | CC(═O)N(C)[C@@H](C[OH])C(═O)NC |
| ABU_pp | CC(═O)N[C@@H](CC)C(═O)NC |
| ASN | CC(═O)N[C@@H](CC(═O)N)C(═O)NC |
| AGLR_pp | CC(═O)N[C@@H](CC(═O)N1[C@H](C)C(═O)N(C)C |
| OZR | CC(═O)N[C@H](C)C1═N[C@@H](CO1)C(═O)NC |
| AMP | CC(═O)N1[C@@](C)(CCC1)C(═O)NC |
| NMVAL_pp | CC(═O)N(C)[C@@H](C(C)C)C(═O)N(C)C |
| ACHC12T | CC(═O)N[C@@H]1[C@H](CCCC1)C(═O)NC |
| OZR_pp | CC(═O)N[C@H](C)C1═N[C@@H](CO1)C(═O)N(C)C |
| PRR | CC(═O)N1C[C@@H](CC1)C(═O)NC |
| DHA | CC(═O)NC(═C)C(═O)NC |
| B3ALA_pp | CC(═O)N[C@@H](C)CC(═O)N(C)C |
| FPA_pp | CC(═O)N[C@@H](Cc1c(F)c(F)c(F)c(F)c1(F))C(═O)N(C)C |
| BGLY | CC(═O)NCCC(═O)NC |
| LARE | CC(═O)N[C@H](C(═O)N)CCC(═O)NC |
| NAP2_pp | CC(═O)N[C@@H](Cc1ccc(cccc2)c2c1)C(═O)N(C)C |
| GPN | CC(═O)NCC1(CCCCC1)CC(═O)NC |
| ANT9 | CC(═O)N[C@@H](Cc1c(cccc2)c2cc(cccc2)c12)C(═O)NC |
| GAPC | CC(═O)N[C@H]1C[C@H](N(C(═O)C)C1)C(═O)NC |
| AGLS_pp | CC(═O)N[C@@H](CC1)C(═O)N1[C@@H](C)C(═O)N(C)C |
| ACPenC13T | CC(═O)N[C@@H]1C[C@H](CC1)C(═O)NC |
| LARD | CC(═O)N[C@H](C(═O)N)CC(═O)NC |
| PTAMBA | CC(═O)N[C@H](Cc1ccccc1)C)CC(═O)NC |
| OZS_pp | CC(═O)N[C@@H](C)C1═N[C@@H](CO1)C(═O)N(C)C |
| BPRO | CC(═O)N1[C@@H](CCC1)CC(═O)NC |
| PHG_pp | CC(═O)N([C@@H](c1ccccc1)C(═O)N(C)C |
| BAZE_pp | CC(═O)N1CC(C1)C(═O)N(C)C |
| ACBC | CC(═O)NC(CC1)(C1)C(═O)NC |
| SUGA | CC(═O)N[C@H](C(═O)N)CSCC(═O)NC |
| ACPC12C_pp | CC(═O)N[C@@H]1[C@@H](C1)C(═O)N(C)C |
| G4ALA | CC(═O)N[C@@H](C)CCC(═O)NC |
| D4ALA_pp | CC(═O)N[C@@H](C)CCCC(═O)N(C)C |
| OZS | CC(═O)N[C@@H](C)C1═N[C@@H](CO1)C(═O)NC |
| PYR3 | CC(═O)N[C@@H](Cc1ccc(ccc3(ccc4))c(c3c4cc2)c12)C(═O)NC |
| PTTBA | CC(═O)N(C(C)(C)C)CC(═O)NC |
| NMBEN2_pp | CC(═O)N(C)c1c(cccc1)C(═O)N(C)C |
| B3VAL_pp | CC(═O)N[C@@H](C(C)C)CC(═O)N(C)C |
| ACHC14T | CC(═O)N[C@@H]1CC[C@H](CC1)C(═O)NC |
| AMP_pp | CC(═O)N1[C@@](C)CCC1)C(═O)N(C)C |
| TBA_pp | CC(═O)N[C@@H](CC(C)(C)C)C(═O)N(C)C |
| AGLR | CC(═O)N[C@@H](CC1)C(═O)N1[C@H](C)C(═O)NC |
| HUCQ | CC(═O)Nc1c2nc(ccc2ccc1)C(═O)NC |
| BEN2_pp | CC(═O)Nc1c(cccc1)C(═O)N(C)C |
| PTTBA_pp | CC(═O)N(C(C)(C)C)CC(═O)N(C)C |
| HPS | CC(═O)N1[C@@H](C[C@H](O)(C1)C(═O)NC |
| INIP_pp | CC(═O)N1CCC(CC1)C(═O)N(C)C |
| FPR | CC(═O)N1[C@@H](C[C@@H](F)C1)C(═O)NC |
| NMBEN3 | CC(═O)N(C)c1cc(ccc1)C(═O)NC |
| PPS | CC(═O)N1[C@@H](C[C@@H](c2ccccc2)C1)C(═O)NC |
| B3PHE | CC(═O)N[C@@H](Cclccccc1)CC(═O)NC |
| NMALA_pp | CC(═O)N(C)[C@@H](C)C(═O)N(C)C |
| B3VAL | CC(═O)N[C@@H](C(C)C)CC(═O)NC |
| TIC_pp | CC(═O)N1[C@@H](Cc(cccc2)c2C1)C(═O)N(C)C |
| INIP | CC(═O)N1CCC(CC1)C(═O)NC |
| PHG | CC(═O)N[C@@H](c1ccccc1)C(═O)NC |
| NMABU | CC(═O)N(C)[C@@H](CC)C(═O)NC |
| ACHC13T | CC(═O)N[C@@H]1C[C@H](CCC1)C(═O)NC |
| PPS_pp | CC(═O)N1[C@@H](C[C@@H](c2ccccc2)C1)C(═O)N(C)C |
| SAR_pp | CC(═O)N(C)CC(═O)N(C)C |
| LARD_pp | CC(═O)N[C@H](C(═O)N)CC(═O)N(C)C |
| NMASN | CC(═O)N(C)[C@@H](CC(═O)N)C(═O)NC |
| PIP_pp | CC(═O)N1[C@@H](CCCC1)C(═O)N(C)C |
| NAP1_pp | CC(═O)N[C@@H](Cc1cccc(cccc2)c12)C(═O)N(C)C |
| AMACBEN4_pp | CC(═O)NCc1ccc(cc1)CC(═O)N(C)C |
| SER | CC(═O)N[C@@H](C[OH])C(═O)NC |
| AIB | CC(═O)NC(C)(C)C(═O)NC |
| CLICK | CC(═O)NCc1cn(nn1)CC(═O)NC |
| AGLY | CC(═O)NCC(═O)NC |
| ACPC12T_pp | CC(═O)N[C@@H]1[C@H](C1)C(═O)N(C)C |
| PHE_pp | CC(═O)N[C@@H](Cc1cccc)C(═O)N(C)C |
| ACBEN2_pp | CC(═O)Nc1c(cccc1)CC(═O)N(C)C |
| ACPC12T | CC(═O)N[C@@H]1[C@H](C1)C(═O)NC |
| NVL_pp | CC(═O)N[C@@H](CCC)C(═O)N(C)C |
| G4ALA_pp | CC(═O)N[C@@H](C)CCC(═O)N(C)C |
| B2ALA | CC(═O)NC[C@@H](C)C(═O)NC |
| HUCP_pp | CC(═O)NCc1nc(ccc1)C(═O)N(C)C |
| NMSER_pp | CC(═O)N(C)[C@@H](C[OH])C(═O)N(C)C |
| BAZE | CC(═O)N1CC(C1)C(═O)NC |
| DGLY_pp | CC(═O)NCCCCC(═O)N(C)C |
| ACBC13T | CC(═O)N[C@@H]1C[C@H](C1)C(═O)NC |
| NMBEN4 | CC(═O)N(C)c1ccc(cc1)C(═O)NC |
| NHM | CC(═O)N1[C@@H](Cc2c3ccccc3[nH]c2C1)C(═O)NC |
| ACHA14T | CC(═O)N[C@@H]1CC[C@H](CC1)CC(═O)NC |
| PTIPA | CC(═O)N(C(C)C)CC(═O)NC |
| ACBEN2 | CC(═O)Nc1c(cccc1)CC(═O)NC |
| NHM_pp | CC(═O)N1[C@@H](Cc2c3ccccc3[nH]c2C1)C(═O)N(C)C |
| B3LEU | CC(═O)N[C@@H](CC(C)C)CC(═O)NC |
| HPS_pp | CC(═O)N1[C@@H](C[C@H](O)C1)C(═O)N(C)C |
| OZL | CC(═O)N[C@@H](C)c1nc(co1)C(═O)NC |
| HUCQ_pp | CC(═O)Nc1c2nc(ccc2ccc1)C(═O)N(C)C |
| AGLY_pp | CC(═O)NCC(═O)N(C)C |
| OZL_pp | CC(═O)N[C@@H](C)c1nc(co1)C(═O)N(C)C |
| AMC14T_pp | CC(═O)NC[C@@H]1CC[C@H](CC1)C(═O)N(C)C |
| B3ALA | CC(═O)N[C@@H](C)CC(═O)NC |
| FPA | CC(═O)N[C@@H](Cc1c(F)c(F)c(F)c(F)c1(F))C(═O)NC |
| BPRO_pp | CC(═O)N1[C@@H](CCC1)CC(═O)N(C)C |
| ACBEN3 | CC(═O)Nc1cc(ccc1)CC(═O)NC |
| BPIP | CC(═O)N1[C@@H](CCCC1)CC(═O)NC |
| HPR_pp | CC(═O)N1[C@@H](C[C@@H](O)C1)C(═O)N(C)C |
| PTANI_pp | CC(═O)N(c1ccccc1)CC(═O)N(C)C |
| SAR | CC(═O)N(C)CC(═O)NC |
| CLICK_pp | CC(═O)NCc1cn(nn1)CC(═O)N(C)C |
| CLA_pp | CC(═O)N[C@@H](C[C1])C(═O)N(C)C |
| NAP2 | CC(═O)N[C@@H](Cc1ccc(cccc2)c2c1)C(═O)NC |
| LARE_pp | CC(═O)N[C@H](C(═O)N(CCC(═O)N(C)C |
| ACPenC12T | CC(═O)N[C@@H]1[C@H](CCC1)C(═O)NC |
| SUGA_pp | CC(═O)N[C@H](C(═O)N)CSCC(═O)N(C)C |
| PPR_pp | CC(═O)N1[C@@H](C[C@H](c2ccccc2)C1)C(═O)N(C)C |
| NIP_pp | CC(═O)N1C[C@@H](CCC1)C(═O)N(C)C |
| PTAMBA_pp | CC(═O)N[C@H](c1ccccc1)C)CC(═O)N(C)C |
| AIB_pp | CC(═O)NC(C)(C)C(═O)N(C)C |
| ANT9_pp | CC(═O)N[C@@H](Cc1c(cccc2)c2cc(cccc2)c12)C(═O)N(C)C |
| LEU_pp | CC(═O)N[C@@H](CC(C)C)C)C(═O)N(C)C |
| ASN_pp | CC(═O)N[C@@H](CC(═O)N)C(═O)N(C)C |
| ACBEN3_pp | CC(═O)Nc1cc(ccc1)CC(═O)N(C)C |
| EGLY | CC(═O)NCCCCCC(═O)NC |
| ACHC13C | CC(═O)N[C@@H]1C[c@@H](CCC1)C(═O)NC |
| FPS_pp | CC(═O)N1[C@@H](C[C@H](F)C1)C(═O)N(C)C |
| NIP | CC(═O)N1C[C@@H](CCC1)C(═O)NC |
| ACPenC | CC(═O)NC(CCC1)(C1)C(═O)NC |
| NAP1 | CC(═O)N[C@@H](Cc1cccc(cccc2)c12)C(═O)NC |
| TIQ | CC(═O)N1[C@@H](c(cccc2)c2CC1)C(═O)NC |
| PTIPA_pp | CC(═O)N(C(C)C)CC(═O)N(C)C |
| OXO_pp | CC(═O)N1[C@@H](CCC1(═O)N(C)C |
| ABOC_pp | CC(═O)NC(CC1)(CCC1C2)[C@H]2C(═O)N(C)C |
| DHP | CC(═O)N1[C@@H](C═CC1)C(═O)NC |
| PYR3_pp | CC(═O)N[C@@H](Cc1ccc(ccc3(ccc4)c(c3e4cc2)c12)C(═O)N(C)C |
| NMPHE_pp | CC(═O)N(C)[C@@H](Cc1ccccc1)C(═O)N(C)C |
| ACPC12C | CC(═O)N[C@@H]1[C@@H](C1)C(═O)NC |
| TZS | CC(═O)N[C@@H](C)C1═N[C@@H](CS1)C(═O)NC |
| BGLY_pp | CC(═O)NCCC(═O)N(C)C |
| OXO | CC(═O)N1[C@@H](CCC1(═O))C(═O)NC |
| PHE | CC(═O)N[C@@H](Cc1ccccc1)C(═O)NC |
| NMALA | CC(═O)N(C)[C@@H](C)C(═O)NC |
| PTABU | CC(═O)N(CC)CC(═O)NC |
| SER_pp | CC(═O)N[C@@H](C[OH])C(═O)N(C)C |
| ACHA14C | CC(═O)N[C@@H]1CC[C@@H](CC1)CC(═O)NC |
| AMACBEN4 | CC(═O)NCc1ccc(cc1)CC(═O)NC |
| ACBC13C | CC(═O)N[C@@H]1C[C@@H](C1)C(═O)NC |
| BEN4_pp | CC(═O)Nc1ccc(cc1)C(═O)N(C)C |
| BEN2 | CC(═O)Nc1c(cccc1)C(═O)NC |
| PRO | CC(═O)N1[C@@H](CCC1)C(═O)NC |
| B3LEU_pp | CC(═O)N[C@@H](CC(C)C)CC(═O)N(C)C |
| TBG_pp | CC(═O)N[C@@H](C(C)(C)C)C(═O)N(C)C |
| D4ALA | CC(═O)N[C@@H](C)CCCC(═O)NC |
| AMBEN3_pp | CC(═O)NCc1cc(ccc1)C(═O)N(C)C |
| ACHA14T_pp | CC(═O)N[C@@H]1CC[C@H](CC1)CC(═O)N(C)C |
| AMBEN2 | CC(═O)NCc1c(cccc1)C(═O)NC |
| NMBEN4_pp | CC(═O)N(C)c1ccc(cc1)C(═O)N(C)C |
| ACPeneC13C | CC(═O)N[C@@H]1C[C@@H](C═C1)C(═O)NC |
| AMBEN3 | CC(═O)NCc1cc(ccc1)C(═O)NC |
| ACPeneC13C_pp | CC(═O)N[C@@H]1C[C@@H](C═C1)C(═O)N(C)C |
| GAPC_pp | CC(═O)N[C@H]1C[C@H](N(C(═O)C)C1)C(═O)N(C)C |
| BEN3 | CC(═O)Nc1cc(ccc1)C(═O)NC |
| TBA | CC(═O)N[C@@H](CC(C)(C)C)C(═O)NC |
| AZE_pp | CC(═O)N1[C@@H](CC1)C(═O)N(C)C |
| B3PHE_pp | CC(═O)N[C@@H](Cc1ccccc1)CC(═O)N(C)C |
| ACHC_pp | CC(═O)NC(CCCC1)(C1)C(═O)N(C)C |
| AMBEN2_pp | CC(═O)NCc1c(cccc1)C(═O)N(C)C |
| NMLEU | CC(═O)N(C)[C@@H](CC(C)C)C(═O)NC |
| FPR_pp | CC(═O)N1[C@@H](C[C@@H](F)C1)C(═O)N(C)C |
| NMVAL | CC(═O)N(C)[C@@H](C(C)C)C(═O)NC |
| DGLY | CC(═O)NCCCCC(═O)NC |
| TZR | CC(═O)N[C@H](C)C1═N[C@@H](CS1)C(═O)NC |
| ACPenC12C_pp | CC(═O)N[C@@H]1[C@@H](CCC1)C(═O)N(C)C |
| ACPenC13C_pp | CC(═O)N[C@@H]1C[C@@H](CC1)C(═O)N(C)C |
| ACPhenC_pp | CC(═O)NC(Cc(cccc2)c12)(C1)C(═O)N(C)C |
| IDC | CC(═O)N1[C@@H](Cc(cccc2)c12)C(═O)NC |
| ACHC12T_pp | CC(═O)N[C@@H]1[C@H](CCCC1)C(═O)N(C)C |
| ACHC13T_pp | CC(═O)N[C@@H]1C[C@H](CCC1)C(═O)N(C)C |
| ACPenC12C | CC(═O)N[C@@H]1[C@@H](CCC1)C(═O)NC |
| ACHC14C_pp | CC(═O)N[C@@H]1CC[C@@H](CC1)C(═O)N(C)C |
| TZL_pp | CC(═O)N[C@@H](C)c1nc(cs1)C(═O)N(C)C |
| GGLY_pp | CC(═O)NCCCC(═O)N(C)C |
| ACPC | CC(═O)NC(C1)(C1)C(═O)NC |
| NMASN_pp | CC(═O)N(C)[C@@H](CC(═O)N)C(═O)N(C)C |
| ACBC_pp | CC(═O)NC(CC1)(C1)C(═O)N(C)C |
| ACBC13C_pp | CC(═O)N[C@@H]1C[C@@H](C1)C(═O)N(C)C |
| ACBC12C_pp | CC(═O)N[C@@H]1[C@@H](CC1)C(═O)N(C)C |
| NMLEU_pp | CC(═O)N(C)[C@@H](CC(C)C)C(═O)N(C)C |
| CLICKS | CC(═O)NCc1cn(nn1)[C@@H](C)C(═O)NC |
| B2ALA_pp | CC(═O)NC[C@@H](C)C(═O)N(C)C |
| ACBC12C | CC(═O)N[C@@H]1[C@@H](CC1)C(═O)NC |
| TZL | CC(═O)N[C@@H](C)c1nc(cs1)C(═O)NC |
| TZR_pp | CC(═O)N[C@H](C)C1═N[C@@H](CS1)C(═O)N(C)C |
| GAPT_pp | CC(═O)N[C@@H]1C[C@H](N(C(═O)C)C1)C(═O)N(C)C |
| AMACBEN2 | CC(═O)NCc1c(cccc1)CC(═O)NC |
| PRR_pp | CC(═O)N1C[C@@H](CC1)C(═O)N(C)C |
| AMC14C | CC(═O)NC[C@@H]1CC[C@@H](CC1)C(═O)NC |
| VIC_pp | CC(═O)N[C@@H](CCNC(═O)CC1)C(═O)N[C@H]1C(═O)N(C)C |
| IDC_pp | CC(═O)N1[C@@H](Cc(cccc2)c12)C(═O)N(C)C |
| NMABU_pp | CC(═O)N(C)[C@@H](CC)C(═O)N(C)C |
| DHA_pp | CC(═O)NC(═C)C(═O)N(C)C |
| BPIP_pp | CC(═O)N1[C@@H](CCCC1)CC(═O)N(C)C |
| NMPHE | CC(═O)N(C)[C@@H](Cc1ccccc1)C(═O)NC |
| BEN4 | CC(═O)Nc1ccc(cc1)C(═O)NC |
| AMBEN4 | CC(═O)NCc1ccc(cc1)C(═O)NC |
| B3PHG_pp | CC(═O)N[C@@H](c1ccccc1)CC(═O)N(C)C |
| ACPenC_pp | CC(═O)NC(CCC1)(C1)C(═O)N(C)C |
| PTANI | CC(═O)N(c1ccccc1)CC(═O)NC |
| ACPenC12T_pp | CC(═O)N[C@@H]1[C@H](CCC1)C(═O)N(C)C |
| ACPenC13T_pp | CC(═O)N[C@@H]1C[C@H](CC1)C(═O)N(C)C |
| VIC | CC(═O)N[C@@H](CCNC(═O)CC1)C(═O)N[C@H]1C(═O)NC |
| VAL_pp | CC(═O)N[C@@H](C(C)C)C(═O)N(C)C |
| PIP | CC(═O)N1[C@@H](CCCC1)C(═O)NC |
| ACHC12C_pp | CC(═O)N[C@@H]1[C@@H](CCCC1)C(═O)N(C)C |
| ACHC13C_pp | CC(═O)N[C@@H]1C[C@@H](CCC1)C(═O)N(C)C |
| AMACBEN3_pp | CC(═O)NCc1cc(ccc1)CC(═O)N(C)C |
| ACHC14T_pp | CC(═O)N[C@@H]1CC[C@H](CC1)C(═O)N(C)C |
| ACHC12C | CC(═O)N[C@@H]1[C@@H](CCCC1)C(═O)NC |
| ALA | CC(═O)N[C@@H](C)C(═O)NC |
| TIQ_pp | CC(═O)N1[C@@H](c(cccc2)c2CC1)C(═O)N(C)C |
| PTPYR3 | CC(═O)N(Cc1ccc(ccc3(ccc4)c(c3c4cc2)c12)CC(═O)NC |
| ACBC13T_pp | CC(═O)N[C@@H]1C[C@H](C1)C(═O)N(C)C |
| ACBC12T_pp | CC(═O)N[C@@H]1[C@H](CC1)C(═O)N(C)C |
| ACPenC13C | CC(═O)N[C@@H]1C[C@@H](CC1)C(═O)NC |
| AZE | CC(═O)N1[C@@H](CC1)C(═O)NC |
| CLA | CC(═O)N[C@@H](C[C1])C(═O)NC |
| GAPT | CC(═O)N[C@@H]1C[C@H](N(C(═O)C)C1)C(═O)NC |
| ACBEN4_pp | CC(═O)Nc1ccc(cc1)CC(═O)N(C)C |
| GGLY | CC(═O)NCCCC(═O)NC |
| TIC | CC(═O)N1[C@@H](Cc(cccc2)c2C1)C(═O)NC |
| PRO_pp | CC(═O)N1[C@@H](CCC1)C(═O)N(C)C |
| GPN_pp | CC(═O)NCC1(CCCCC1)CC(═O)N(C)C |
| AMACBEN2_pp | CC(═O)NCc1c(cccc1)CC(═O)N(C)C |
| LEU | CC(═O)N[C@@H](CC(C)C)C(═O)NC |
| THP_pp | CC(═O)N1[C@@H](CSC1)C(═O)N(C)C |
| THP | CC(═O)N1[C@@H](CSC1)C(═O)NC |
| NMPHG_pp | CC(═O)N(C)[C@@H](c1ccccc1)C(═O)N(C)C |
| ACHC | CC(═O)NC(CCCC1)(C1)C(═O)NC |
| ACPhenC | CC(═O)NC(Cc(cccc2)c12)(C1)C(═O)NC |
| EGLY_pp | CC(═O)NCCCCCC(═O)N(C)C |
| ACHC14C | CC(═O)N[C@@H]1CC[C@@H](CC1)C(═O)NC |
| TZS_pp | CC(═O)N[C@@H](C)C1═N[C@@H](CS1)C(═O)N(C)C |
| PTPYR3_pp | CC(═O)N(Cc1ccc(ccc3(ccc4)c(c3c4cc2)c12)CC(═O)N(C)C |
| ACPC_pp | CC(═O)NC(C1)(C1)C(═O)N(C)C |
| B3PHG | CC(═O)N[C@@H](c1ccccc1)CC(═O)NC |
| VAL | CC(═O)N[C@@H](C(C)C)C(═O)NC |
| TBG | CC(═O)N[C@@H](C(C)(C)C)C(═O)NC |
| AMACBEN3 | CC(═O)NCc1cc(ccc1)CC(═O)NC |
| ALA_pp | CC(═O)N[C@@H](C)C(═O)N(C)C |
| ACHA14C_pp | CC(═O)N[C@@H]1CC[C@@H](CC1)CC(═O)N(C)C |

We claim:

1. A 3 to 4 residue non-naturally occurring macrocycle oligoamide comprising a chemotype of 3 or 4 monomer residues selected from the group consisting of a, b, c, d, e, f, g, h, I, j, k, l, m, n, o, p, q, r, s, t, u, and v monomers as defined in Table 1, or salt thereof.

2. The macrocycle oligoamide of claim 1 wherein
(a) monomer designations a, b, g, d, and e are amino acids, wherein
(i) monomer designation a comprises or consists of all L- and D-proteinogenic amino acids as well as all L- and D-non-canonical alpha amino acids; peptoids (N-alkylated glycines), and enantiomers thereof;
(ii) monomer designation b comprises or consists of all B3-amino acids that contain L- or D-proteinogenic side chains, and enantiomers thereof;
(iii) monomer designation d comprises or consists of delta-amino acids and enantiomers thereof;
(iv) monomer designation g comprises or consists of gamma amino acids and enantiomers thereof; and
(v) monomer designation e comprises or consists of epsilon amino acids and enantiomers thereof;
(b) monomer designations c, f, j comprise or consist of aminobenzoic acids and enantiomers thereof;
(c) monomer designations h, k, n comprise or consist of aminomethylbenzoic acids and enantiomers thereof;
(d) monomer designations i, l, o comprise or consist of aminophenylacetic acids and enantiomers thereof;
(e) monomer designations m, p, q comprise or consist of aminomethylphenylacetic acids and enantiomers thereof;
(f) monomer designation r comprises or consists of oxazolidines and thiazolidines that include all L- and D-proteinogenic side chains substituted on the second atom in the backbone, and enantiomers thereof, both for the oxazolidinone and thiazolidinone;
(g) monomer designation t comprises or consists of oxazole and thiazoles that include all L- and D-proteinogenic side chains substituted on the second atom in the backbone, and enantiomers thereof, both for the oxazolidine and thiazolidinine;
(h) monomer designation s comprises or consists of thioethers and enantiomers thereof;
(i) monomer designation comprises or consists of aminomethylpicolinic acids and enantiomers thereof; and
(j) monomer designation v comprises or consists of triazoles and enantiomers thereof.

3. The macrocycle oligoamides of claim 1 or 2, wherein the monomers are selected from the group consisting of the following, as defined by chemical structure in FIG. 5 and Table 5:
Monomer designation “a” selected from the group consisting of AGLY, ALA (alanine), ABU, SER (serine), ASN (asparagine), CLA, VAL (valine), TBG, NVL(norvaline), LEU (leucine), TBA, PHG (phenylglycine), PHE (phenylalanine), FPA, NAP1, NAP2, ANT9, PYR3, DHA, AIB (aminoisobutyric acid), ACPC, ACBC, ACPenC, ACPhenC, ACHC, SAR (sarcosine), PTABU, PTIPA, PTTBA, PTANI, PTAMBA, NMALA (n-methylalaine), NMABU, NMVAL (n-methylvaline), NMPHG, NMLEU (n-methylleucine), NMPHE (n-methylphenylalanine), NMSER (n-methylserine), NMASN (n-methylasparigine), AZE (azetidine), PRO (proline), PIP, TIQ, TIC, NHM, THP, DHP, FPS, FPR, HPS, HPR, OXO, PPS, PPR, AMP, and IDC, and enantiomers thereof;
Monomer designation “b” selected from the group consisting of BGLY (beta-alanine), B3ALA (beta-3-homoalanine), B3VAL (beta-3-leucine), B3PHG (beta-3-phenylalanine), B3PHE (beta-3-homophenylalanine), B2ALA, BAZE, BPRO, BPIP, PRR, NIP, LARD, ACPC12C, ACPC12T, ACBC12C, ACBC12T, ACPenC12C, ACPenC12T, ACHC12C, ACHC12T, and ABOC, and enantiomers thereof:
Monomer designation “c” selected from the group consisting of BEN2 and NMBEN2:
Monomer designation “d” selected from the group consisting of D4ALA, DGLY, ACHC14C, and ACHC14T;
Monomer designation “e” selected from the group consisting of EGLY, ACHA14C, ACHA14T, AMC14C, and AMC14T;
Monomer designation “f” selected from the group consisting of BEN3 and NMBEN3;
Monomer designation “g” selected from the group consisting of GGLY, G4ALA, GPN, INIP, LARE, ACBC 13C, ACBC13T, ACPenC13C, ACPeneC13C, ACPenC13t, GAPC, GAPT, ACHC13C, and ACHC13T;
Monomer designation “h” is AMBEN2;
Monomer designation “i” is ACBEN2;
Monomer designation “j” selected from the group consisting of BEN4 and NMBEN4;
Monomer designation “k” is AMBEN3;
Monomer designation “l” is ACBEN3;
Monomer designation “in” is AMACBEN2;
Monomer designation “n” is AMBEN4;
Monomer designation “o” is ACBEN4;
Monomer designation “p” is AMACBEN3;
Monomer designation “q” is AMACBEN4;
Monomer designation “r” selected from the group consisting of AGLS, AGLR, OZS, OZR, TZS, TZR, and VIC, and enantiomers thereof;
Monomer designation “s” is SUGA;
Monomer designation “t” selected from the group consisting of OZL, TZL, and HUCP, and enantiomers thereof;
Monomer designation “u” is HUCQ; and/or
Monomer designation “v” selected from the group consisting of CLICK and CLICKS.

4. The macrocycle oligoamide of any one of claims 1-3, comprising a chemotype selected from the group of chemotypes listed in Table 2, or circularly permuted versions thereof.

5. The macrocycle oligoamide of any one of claims 1-4, wherein the macrocyclic oligoamide includes at least one monomer not falling within monomer designation a.

6. The macrocycle oligoamide of any one of claims 1-5, wherein the macrocyclic oligoamide includes at least one residue not falling within monomer designation a, b, g, d, e.

7. The macrocycle of any one of claims 1-6, wherein the macrocyclic oligoamide is a 4 residue macrocycle.

8. The macrocycle oligoamide of any one of claims 1-7, having a chemotype selected from the group consisting of aaar, aarb, abar, aavb, aabr, and aatb, or circularly permuted versions thereof.

9. The macrocycle oligoamide of any one of claims 1-7, having a chemotype selected from the group consisting of akak, aaaq, and aabc, or circularly permuted versions thereof.

10. The macrocycle oligoamide of any one of claims 1-6, having an aas chemotype, or a circularly permuted versions thereof, wherein the macrocycle oligoamide comprises two hydrogen bonds, one between backbone amides, and one involving the “s” monomer primary amide.

11. The macrocycle oligoamide of any one of claims 1-7, having an aaam or akak chemotype, or circularly permuted versions thereof, wherein any nitrogen in the backbone is in a tertiary amide.

12. The macrocycle oligoamide of any one of claims 1-7, having an aaaq chemotype, or a circularly permuted version thereof, wherein one of the “a” monomers comprises a pentafluorophenylalanine residue or enantiomer thereof, and the “q” residue comprises a 4-aminomethylphenylacetic acid residue or enantiomer thereof.

13. The macrocycle oligoamide of any one of claims 1-7, having a chemotype selected from the group consisting of ahah, aaam, aabi, aagb, aaap, and aalm, or circularly permuted versions thereof.

14. The macrocycle oligoamide of any one of claims 1-13, wherein the macrocyclic oligoamide comprises one or more hydrogen bonds.

15. The macrocyclic oligoamide of claim 14, wherein the one or more hydrogen bonds comprise a hydrogen bond between backbone amides involving non α-amino acids.

16. The macrocyclic oligoamide of any one of claims 1-15, wherein the macrocyclic oligoamide is membrane permeable.

17. The macrocyclic oligoamide of any one of claims 1-17, wherein the macrocyclic oligoamide does not comprise exposed polar groups, such as side chain hydroxyls and/or primary amides.

18. The macrocyclic oligoamide of any one of claims 1-17, comprising a structure of any macrocyclic oligoamide disclosed in any figure herein, circularly permuted versions thereof, or salt thereof; or wherein the macrocyclic oligoamide comprises or consists of a structure of any macrocyclic oligoamide shown in any one of FIGS. 3-4, 12, and 19-47, or salt thereof; or comprising or consisting of the structure of any one of compounds 1-218 in Table 3, or salts thereof.

19. The macrocyclic oligoamide of any one of claims 1-18, comprising a substitution of 1, 2, 3, or all 4 monomer subunits.

20. The macrocyclic oligoamide of claim 19, wherein the substitution comprises a functional group.

21. The macrocyclic oligoamide of claim 20, wherein the functional group(s) comprise a therapeutic moiety, a diagnostic moiety, and/or a reactive moiety.

22. A library, comprising 10, 50, 100, 500, 1000, 5000, 10,000, 25, 000, 35,000, or more macrocyclic oligoamides of any one of claims 1-21.

23. A method for using the macrocyclic oligoamides and/or library of any preceding claim for any suitable purpose, including but not limited to panning the library to identify one or more macrocyclic oligoamide that binds to a compound of interest, therapeutic treatments, diagnostic methods, and/or adding reactive moieties for any use.

24. A method performed by one or more computers for identifying macrocycle conformations of a molecule comprising a first molecular fragment and a second molecular fragment, the method comprising:
obtaining a set of conformations of the first molecular fragment;
determining, for each conformation of the first molecular fragment, values of a set of parameters of an initial-to-terminal transformation defining a translation and a rotation of an initial end of the first molecular fragment relative to a terminal end of the first molecular fragment in the conformation;
obtaining a set of conformations of the second molecular fragment;
determining, for each conformation of the second molecular fragment, values of a set of parameters of a terminal-to-initial transformation defining a translation and a rotation of a terminal end of the second molecular fragment relative to an initial end of the second molecular fragment in the conformation; and
processing the respective parameter values of the initial-to-terminal transformations and the terminal-to-initial transformations to identify a set of one or more macrocycle conformations of the molecule,
wherein each macrocycle conformation of the molecule comprises respective conformations of the first molecular fragment and the second molecular fragment that jointly form a closed loop.

25. The method of claim 24, wherein processing the respective parameter values of the initial-to-terminal transformations and the terminal-to-initial transformations to identify the set of one or more macrocycle conformations of the molecule comprises, for each macrocycle conformation:
determining that the parameter values of the initial-to-terminal transformation for the conformation of the first molecular fragment and the parameter values of the terminal-to-initial transformation for the conformation of the second molecular fragment satisfy a loop closure criterion.

26. The method of claim 25, wherein determining that the parameter values of the initial-to-terminal transformation for the conformation of the first molecular fragment and the parameter values of the terminal-to-initial transformation for the conformation of the second molecular fragment satisfy a loop closure criterion comprises:
determining that a discretization of the parameter values of the initial-to-terminal transformation for the conformation of the first molecular fragment are equal to a discretization of the parameter values of the terminal-to-initial transformation for the conformation of the second molecular fragment.

27. The method of any one of claims 24-26, wherein for each conformation of the first molecular fragment, the set of parameters of the corresponding initial-to-terminal transformation define: (i) a rotation matrix that defines the rotation of the initial end of the first molecular fragment relative to the terminal end of the first molecular fragment in the conformation, and (ii) a translation vector that defines the translation of the initial end of the first molecular fragment relative to the terminal end of the first molecular fragment in the conformation.

28. The method of any one of claims 24-27, wherein processing the respective parameter values of the initial-to-terminal transformations and the terminal-to-initial transformations to identify the set of macrocycle conformations of the molecule comprises:
generating a first dictionary that comprises: (i) a plurality of keys, and (ii) one or more values associated with each key, wherein:
each key in the first dictionary represents a respective discretized initial-to-terminal transformation; and
each value associated with a key in the first dictionary specifies a conformation of the first molecular fragment with a discretized initial-to-terminal transformation represented by the key;
generating a second dictionary that comprises: (i) a plurality of keys, and (ii) one or more values associated with each key, wherein:
each key in the second dictionary represents a respective discretized terminal-to-initial transformation; and
each value associated with a key in the second dictionary specifies a conformation of the second molecular fragment with a discretized terminal-to-initial transformation represented by the key; and
identifying the set of macrocycle conformations of the molecule using the first dictionary and the second dictionary.

29. The method of claim 28, wherein generating the first dictionary comprises, for each conformation of the first molecular fragment:
discretizing the parameter values of the initial-to-terminal transformation for the conformation; and
processing the discretized parameter values of the initial-to-terminal transformation for the conformation to generate a corresponding key; and
including the key in the first dictionary and associating the conformation with the key in the first dictionary.

30. The method of any one of claims 28-29, wherein generating the second dictionary comprises, for each conformation of the second molecular fragment:
discretizing the parameter values of the terminal-to-initial transformation for the conformation; and
processing the discretized parameter values of the terminal-to-initial transformation for the conformation to generate a corresponding key; and
including the key in the second dictionary and associating the conformation with the key in the second dictionary.

31. The method of any one of claims 28-30, wherein each key in the first dictionary and each key in the second dictionary are represented by respective integer numerical values.

32. The method of any one of claims 28-31, wherein identifying the set of macrocycle conformations of the molecule using the first dictionary and the second dictionary comprises:
identifying a set of one or more keys that are common to both the first dictionary and the second dictionary; and
identifying the set of macrocycle conformations of the molecule based on the set of keys common to both the first dictionary and the second dictionary.

33. The method of claim 32, wherein identifying the set of macrocycle conformations of the molecule based on the set of keys common to both the first dictionary and the second dictionary comprises, for each key common to both the first dictionary and the second dictionary:
identifying, as defining a macrocycle conformation, each pair of conformations that comprises: (i) a conformation of the first molecular fragment associated with the key in the first dictionary, and (ii) a conformation of the second molecular fragment associated with the key in the second dictionary.

34. The method of any one of claims 24-33, further comprising:
determining, for each macrocycle conformation, a respective energy value associated with the macrocycle conformation;
identifying a macrocycle conformation having a minimum energy value among the macrocycle conformations;
determining, for each macrocycle conformation, a respective similarity measure between: (i) the macrocycle conformation, and (ii) the macrocycle conformation having the minimum energy value; and
generating a prediction for whether the molecule comprising the first molecular fragment and the second molecular fragment adopts a rigid macrocycle conformation based on the energy values and the similarity measures for the macrocycle conformations.

35. The method of claim 34, wherein generating the prediction for whether the molecule comprising the first molecular fragment and the second molecular fragment adopts a rigid macrocycle conformation comprises:
determining a fraction of the macrocycle conformations of the molecule having: (i) an energy that satisfies an energy threshold, and (ii) a similarity to the minimum energy macrocycle conformation that satisfies a similarity threshold; and
predicting that the molecule adopts a rigid macrocycle conformation only if the fraction satisfies a threshold.

36. The method of any one of claims 34-35, wherein determining the energy value associated with a macrocycle conformation comprises:
processing data characterizing the macrocycle conformation using an energy prediction neural network to generate the energy value associated with the macrocycle conformation.

37. The method of any one of claims 24-36, further comprising physically synthesizing the molecule.

38. The method of any one of claims 24-37, further comprising:
determining one or more conformations of the physically synthesized molecule; and
comparing the conformations of the physically synthesized molecule to the identified macrocycle conformations of the molecule.

39. The method of any one of claims 24-38, wherein the first molecular fragment includes a sequence of monomers.

40. The method of claim 39, wherein each monomer in the sequence of monomers of the first molecular fragment is an amino acid.

41. The method of claim 40, wherein the sequence of monomers comprises alpha amino acids, beta amino acids, or both.

42. The method of any one of claims 30-31, wherein the initial end of the first molecular fragment is an N-terminus of a first amino acid in the sequence of monomers of the first molecular fragment, and the terminal end of the first molecular fragment is a C-terminus of a last amino acid in the sequence of monomers of the first molecular fragment.

43. The method of claim 39, wherein obtaining the set of conformations of the first molecular fragment comprises:
obtaining a respective set of conformations for each monomer in the first molecular fragment; and
generating the set of conformations of the first molecular fragments from the sets of conformations of the monomers in the first molecular fragment.

44. The method of claim 43, wherein for each monomer in the first molecular fragment, obtaining the set of conformations of the monomer comprises:
initializing a set of points in a space of structure parameters, wherein each point in the space of structure parameters defines a respective conformation of the monomer;
iteratively augmenting the set of points in the space of structure parameters, comprising, at each iteration in a sequence of iterations:
determining, for each point in the set of points, an energy of the conformation specified by the point; and
selecting a new point to be included in the set of points based on the energies of the points in the set of points; and
determining the set of conformations of the monomer based on the augmented set of points in the space of structure parameters.

45. The method of claim 44, wherein selecting a new point to be included in the set of points based on the energies of the points in the set of points comprises:
determining a triangulation of the set of points;
determining a score for each simplex of the triangulation based on: (i) a volume of the simplex of the triangulation, and (ii) the energies of the points at vertices of the simplex of the triangulation;
selecting a simplex of the triangulation based on the scores for the simplices of the triangulations; and
selecting the new point to be included in the set of points based on the selected simplex of the triangulation.

46. The method of claim 45, wherein selecting the new point to be included in the set of points based on the selected simplex of the triangulation comprises:
selecting the new point as a center of the selected simplex of the triangulation.

47. The method of any one of claims 44-46, wherein the space of structure parameters includes a respective dimension corresponding to each of one or more torsion angles of the monomer.

48. A system comprising:
one or more computers; and
one or more storage devices communicatively coupled to the one or more computers, wherein the one or more storage devices store instructions that, when executed by the one or more computers, cause the one or more computers to perform operations of the respective method of any one of claims 24-47.

49. One or more non-transitory computer storage media storing instructions that when executed by one or more computers cause the one or more computers to perform operations of the respective method of any one of claims 24-47.
